(19) United States
(12) Patent Application Publication
(10) Pub. No.: US 2023/0037660 A1
(43) Pub. Date: Feb. 9, 2023

(54) METHODS OF TREATING VASCULAR LESIONS AND MALFORMATIONS

(71) Applicant: Blaze Bioscience, Inc., Seattle, WA (US)

(72) Inventors: Alan Cohen, Baltimore, MD (US); Andrew Kobets, Baltimore, MD (US); Julia Novak, Sequim, WA (US); Dennis Miller, Seattle, WA (US)

(21) Appl. No.: 17/764,132

(22) PCT Filed: Oct. 1, 2020
(86) PCT No.: PCT/US2020/053720

§ 371 Date: Mar. 25, 2022

Related U.S. Application Data
(60) Provisional application No. 62/950,876, filed on Dec. 19, 2019.

(51) Int. Cl. A61K 38/17; C07K 14/435; A61P 9/00; A61K 49/00; G01N 33/50; G01N 33/58

(52) U.S. Cl. CPC A61K 38/1767; C07K 14/43522; A61P 9/00; A61K 49/0034; G01N 33/5008; G01N 33/582

(57) ABSTRACT

Compositions and formulations comprising peptide conjugate compounds are provided, including native and modified variants of chlorotoxin peptide conjugated to detectable agents or active agents. Methods of detecting and treating vascular lesions, vascular malformations, and vascular abnormalities including cerebral cavernous malformation (CCM) with peptide conjugate compounds are also provided, including methods of imaging and resecting vascular lesions tissues and cells.

### CROSS-REFERENCE

The present application claims the benefit of U.S. Provisional Application No. 62/950,876, entitled “METHODS OF TREATING VASCULAR LESIONS AND MALFORMATIONS”, filed on Dec. 19, 2019, which application is herein incorporated by reference in its entirety for all purposes.

### BACKGROUND

Vascular lesions and malformations include anomalies of the arteries, veins, and/or lymph vessels. They can occur anywhere in the body, including the central nervous system. Cavernomas, also referred to as cavernous angiomas, cavernous hemangiomas, or cerebral cavernous malformation (CCM), are a type of vascular malformation usually found in the central nervous system, commonly in the brain and spinal cord. They can vary in size from microscopic to several centimeters or inches in diameter. Although sometimes asymptomatic, they are common, with approximately one in 200 people have a cavernoma at some point in their life. Cavernomas are benign (i.e., non-neoplastic or non-cancerous) vascular malformations or lesions that may cause seizures and/or hemorrhage when they develop in the brain. Some cavernous angiomas bleed slowly enough that the body can re-absorb the blood. Others bleed more profusely and can put dangerous pressure on the surrounding brain tissue and/or cause an obvious hemorrhage. Symptoms include bleeding (hemorrhage), fits (seizures), headaches, neurological problems, such as dizziness, loss of impaired vision, blurred vision, slurred speech (dysarthria), double vision, loss or impaired sense of smell (anosmia), other focal neurological deficits, or balance problems and tremor, weakness, numbness, tiredness, memory problems and difficulty concentrating. Moreover, they can produce a hemorrhagic stroke and other complications that are life-threatening or create chronic problems. Environmental factors, such as radiation treatment, can affect the incidence of cavernomas by increasing damage to tissue and incidence of bleeding. Treatment for symptomatic cavernoma usually includes surgery, and the precision of surgical resection directly influences patient prognosis. As with any surgery in the central nervous system, the goal is complete removal of the malformation or lesion with minimal disruption to the surrounding normal tissue. Unfortunately, intra-operative identification of lesion margins or small foci remains imprecise, and these lesions if located in organs and organ substructures, such as the brain and other organs and organ structures such as brain, heart, lung, kidney, liver, CNS (e.g., spine) or pancreas can be debilitating or life-threatening. In addition to improving treatment and surgical outcome of cavernomas, there are similar needs for improving treatment and surgical outcome in other vascular lesions such as aneurysm, arteriovenous malformation, venous malformation, lymphatic malformation, capillary telangiectasia, mixed vascular malformation, spinal dural arteriovenous fistula, and the like.

### SUMMARY

In various aspects, the present disclosure provides a method of treating a subject with a vascular lesion, the method comprising, administering to the subject a polypeptide comprising: a) at least 3 disulfide bonds, b) a length of no less than 20 amino acid residues, c) an isoelectric point of no less than 7.5, or d) combinations thereof, thereby treating the vascular lesion in the subject.

In some aspects, the polypeptide comprises: a) a sequence of any one of SEQ ID NO: 482-SEQ ID NO: 485, or a fragment thereof, b) at least 80%, at least 85%, at least 90%, or at least 95% sequence identity with any one of SEQ ID NO: 1-SEQ ID NO: 481, or a fragment thereof, or c) at least 80%, at least 85%, at least 90%, or at least 95% sequence identity to SEQ ID NO: 9, or a fragment thereof.

In various aspects, the present disclosure provides a method of treating a subject with a one or more of a cavernoma, an arteriovenous malformation, a venous malformation, a lymphatic malformation, a capillary telangiectasia, a mixed vascular malformation, an aneurysm, or a spinal dural arteriovenous fistula, the method comprising, administering to the subject a polypeptide comprising: a) at least 3 disulfide bonds, b) a length of no less than 20 amino acid residues, c) an isoelectric point of no less than 7.5, or d) combinations thereof, thereby treating the arteriovenous malformation, the venous malformation, the lymphatic malformation, the capillary telangiectasia, the mixed vascular malformation, the aneurysm, or the spinal dural arteriovenous fistula in the subject.

In some aspects, the polypeptide comprises: a) a sequence of any one of any one of SEQ ID NO: 482-SEQ ID NO: 485 or a fragment thereof, b) at least 80%, at least 85%, at least 90%, or at least 95% sequence identity with any one of SEQ ID NO: 1-SEQ ID NO: 481 or a fragment thereof, or c) at least 80%, at least 85%, at least 90%, or at least 95% sequence identity to SEQ ID NO: 9, or a fragment thereof.

In various aspects, the present disclosure provides a method of administering a polypeptide comprising: a) at least 3 disulfide bonds, b) a length of no less than 20 amino acid residues, c) an isoelectric point of no less than 7.5, or d) combinations thereof, to a subject with one or more of a vascular lesion, a cavernoma, an arteriovenous malformation, a venous malformation, a lymphatic malformation, a capillary telangiectasia, a mixed vascular malformation, an aneurysm, or a spinal dural arteriovenous fistula.

In some aspects, the polypeptide comprises: a) a sequence of any one of any one of SEQ ID NO: 482-SEQ ID NO: 485 or a fragment thereof, b) at least 80%, at least 85%, at least 90%, or at least 95% sequence identity with any one of SEQ ID NO: 1-SEQ ID NO: 481 or a fragment thereof, or c) at least 80%, at least 85%, at least 90%, or at least 95% sequence identity to SEQ ID NO: 9, or a fragment thereof.

In various aspects, the present disclosure provides a method of treating a subject with a vascular lesion, the method comprising, administering a polypeptide having a sequence of any one of SEQ ID NO: 482-SEQ ID NO: 485, or a fragment thereof, to the subject; and treating the vascular lesion.

In various aspects, the present disclosure provides a method of treating a subject with a vascular lesion, the method comprising, administering a polypeptide having at least 80%, at least 85%, at least 90%, or at least 95% sequence identity with any one of SEQ ID NO: 1-SEQ ID NO: 481, or a fragment thereof, to the subject; and treating the vascular lesion.

In various aspects, the present disclosure provides a method of treating a subject with a vascular lesion, the method comprising, administering a polypeptide having at least 80%, at least 85%, at least 90%, or at least 95% sequence identity to MCMPCFTTDHQMARRCDDCCGGRGRGKCYGPQCLCR (SEQ ID NO: 9), or a fragment thereof, to the subject; and treating the vascular lesion.

In various aspects, the present disclosure provides a method of treating a subject with a one or more of a cavernoma, an arteriovenous malformation, a venous malformation, a lymphatic malformation, a capillary telangiectasia, a mixed vascular malformation, an aneurysm, or a spinal dural arteriovenous fistula, the method comprising, administering a polypeptide to the subject, wherein the polypeptide is any one of SEQ ID NO: 482-SEQ ID NO: 485 or a fragment thereof, and treating the arteriovenous malformation, the venous malformation, the lymphatic malformation, the capillary telangiectasia, the mixed vascular malformation, the aneurysm, or the spinal dural arteriovenous fistula.

In various aspects, the present disclosure provides a method of treating a subject with one or more of a cavernoma, an arteriovenous malformation, a venous malformation, a lymphatic malformation, a capillary telangiectasia, a mixed vascular malformation, an aneurysm, or a spinal dural arteriovenous fistula, the method comprising, administering a polypeptide to the subject, wherein the polypeptide has at least 80%, at least 85%, at least 90%, or at least 95% sequence identity with any one of SEQ ID NO: 1-SEQ ID NO: 481 or a fragment thereof, and treating the arteriovenous malformation, the venous malformation, the lymphatic malformation, the capillary telangiectasia, the mixed vascular malformation, the aneurysm, or the spinal dural arteriovenous fistula.

In various aspects, the present disclosure provides a method of treating a subject with one or more of a cavernoma, an arteriovenous malformation, a venous malformation, a lymphatic malformation, a capillary telangiectasia, a mixed vascular malformation, an aneurysm, or a spinal dural arteriovenous fistula, the method comprising, administering a polypeptide to the subject, wherein the polypeptide has at least 80%, at least 85%, at least 90%, or at least 95% sequence identity with MCMPCFTTDHQMARRCDDCCGGRGRGKCYGPQCLCR (SEQ ID NO: 9) or a fragment thereof, and treating the arteriovenous malformation, the venous malformation, the lymphatic malformation, the capillary telangiectasia, the mixed vascular malformation, the aneurysm, or the spinal dural arteriovenous fistula.

In various aspects, the present disclosure provides a method of administering a polypeptide having a sequence of any one of SEQ ID NO: 482-SEQ ID NO: 485, or a fragment thereof, to a subject with one or more of a vascular lesion, a cavernoma, an arteriovenous malformation, a venous malformation, a lymphatic malformation, a capillary telangiectasia, a mixed vascular malformation, an aneurysm, or a spinal dural arteriovenous fistula.

In various aspects, the present disclosure provides a method of administering a polypeptide having at least 80%, at least 85%, at least 90%, or at least 95% sequence identity with any one of SEQ ID NO: 1-SEQ ID NO: 481, or a fragment thereof, to a subject having a vascular lesion, a cavernoma, an arteriovenous malformation, a venous malformation, a lymphatic malformation, a capillary telangiectasia, a mixed vascular malformation, an aneurysm, or a spinal dural arteriovenous fistula.

In various aspects, the present disclosure provides a method of administering a polypeptide to the subject, wherein the polypeptide has at least 80%, at least 85%, at least 90%, or at least 95% sequence identity with MCMPCFTTDHQMARRCDDCCGGRGRGKCYGPQCLCR (SEQ ID NO. 9) or a fragment thereof to a subject having a vascular lesion, a cavernoma, an arteriovenous malformation, a venous malformation, a lymphatic malformation, a capillary telangiectasia, a mixed vascular malformation, an aneurysm, or a spinal dural arteriovenous fistula.

In some aspects, the method further comprises treating the vascular lesion, the cavernoma, the arteriovenous malformation, the venous malformation, the lymphatic malformation, the capillary telangiectasia, the mixed vascular malformation, the aneurysm, or the spinal dural arteriovenous fistula. In some aspects, the method further comprises detecting the vascular lesion, the cavernoma, the arteriovenous malformation, the venous malformation, the lymphatic malformation, the capillary telangiectasia, the mixed vascular malformation, the aneurysm, or the spinal dural arteriovenous fistula.

In some aspects, the subject has one or more of a cavernoma, an arteriovenous malformation, a venous malformation, a lymphatic malformation, a capillary telangiectasia, a mixed vascular malformation, an aneurysm, or a spinal dural arteriovenous fistula.

In further aspects, the cavernoma comprises a cavernous angioma, a cavernous hemangioma, or a cerebral cavernous malformation (CCM). In further aspects, the arteriovenous malformation comprises an arteriovenous angioma, an arteriovenous hemangioma, or a cerebral arteriovenous malformation (CAM). In further aspects, the aneurysm comprises abdominal aortic, thoracic aortic, or cerebral aneurysm.

In some aspects, the treating comprises reducing a symptom of the vascular lesion, the cavernoma, the arteriovenous malformation, the venous malformation, the lymphatic malformation, the capillary telangiectasia, the mixed vascular malformation, the aneurysm, or the spinal dural arteriovenous fistula, reducing the size or presence of the vascular lesion, the cavernoma, the arteriovenous malformation, the venous malformation, the lymphatic malformation, the capillary telangiectasia, the mixed vascular malformation, the aneurysm, or the spinal dural arteriovenous fistula, or eliminating the vascular lesion, the cavernoma, the arteriovenous malformation, the venous malformation, the lymphatic malformation, the capillary telangiectasia, the mixed vascular malformation, the aneurysm, or the spinal dural arteriovenous fistula in the subject.

In some aspects, the symptom comprises: bleeding (hemorrhage), fits (seizures), headaches, neurological problems, such as dizziness, slurred speech (dysarthria), loss or impaired vision, blurred vision, double vision, loss or impaired sense of smell (anosmia), other focal neurological deficits, or balance problems and tremor, weakness, numbness, tiredness, memory problems, difficulty concentrating, or any combination thereof. In some aspects, the reducing the size or presence or the eliminating is determined by magnetic resonance imaging of the subject.

In some aspects, the fragment of the polypeptide has a length of at least 25 residues. In some aspects, each amino acid of the polypeptide is independently selected as an L- or D-enantiomer. In some aspects, the polypeptide contains no lysine residues.

In other aspects, the polypeptide contains a single lysine residue. In further aspects, the single lysine residue is located at a position corresponding to K-27 of native chlorotoxin, K-23 of native chlorotoxin, or K-15 of native chlorotoxin. In some aspects, one, two, or three methionine residues of the polypeptide are replaced with other amino acids. In some aspects, the N-terminus of the polypeptide is blocked by acetylation or cyclization. In some aspects, the polypeptide comprises at least 1, at least 2, at least 3, at least 4, at least 5, or at least 6 disulfide bonds. In some aspects, the polypeptide comprises an isoelectric point of at least 6.0, at least 6.5, at least 7.0, at least 7.5, at least 8.0, at least 8.5, or at least 9.0. In some aspects, the polypeptide binds to or accumulates in a vascular lesion tissue or cell.

In further aspects, the method further comprises detecting the presence or absence of the polypeptide in a tissue or cell, wherein the presence of the polypeptide in the tissue or cell indicates the presence of a vascular lesion tissue or cell. In some aspects, the vascular lesion is associated with one or more of the cavernoma, the venous malformation, the lymphatic malformation, the capillary telangiectasia, the mixed vascular malformation, the arteriovenous malformation, the aneurysm, or the spinal dural arteriovenous fistula. In some aspects, the detecting is performed using fluorescence imaging. In some aspects, the method further comprises surgically removing the vascular lesion tissue or vascular lesion cells from the human subject.

In some aspects, the polypeptide is intravenously administered about 1 hr, about 2 hrs, about 3 hrs, about 4 hrs, about 5 hrs, about 6 hrs, about 7 hrs, about 8 hrs, about 9 hrs, about 10 hrs, about 11 hrs, about 12 hrs, about 13 hrs, about 14 hrs, about 15 hrs, about 16 hrs, about 17 hrs, about 18 hrs, about 19 hrs, about 20 hrs, about 21 hrs, about 22 hrs, about 23 hrs, about 24 hrs, about 36 hrs, about 48 hrs, about 60 hrs, or about 72 hrs prior surgically removing the vascular lesion tissue or vascular lesion cells from the human subject. In some aspects, the polypeptide is administered at a dosage sufficient to treat the vascular lesion in the human subject. In some aspects, the polypeptide is administered at a dosage sufficient to treat one or more of the cavernoma, the arteriovenous malformation, the venous malformation, the lymphatic malformation, the capillary telangiectasia, the mixed vascular malformation, the aneurysm, or the spinal dural arteriovenous fistula in the human subject.

In some aspects, the polypeptide is conjugated to an agent. In some aspects, the polypeptide is conjugated to the agent via a cleavable linker or a stable linker. In some aspects, the polypeptide comprises a single lysine residue and the agent is conjugated to the polypeptide at the single lysine residue. In some aspects, the polypeptide comprises no lysine residues and the agent is conjugated to the polypeptide at the N-terminus of the polypeptide. In some aspects, the polypeptide and agent comprises the structure of Formula (IV), or a pharmaceutically acceptable salt thereof:

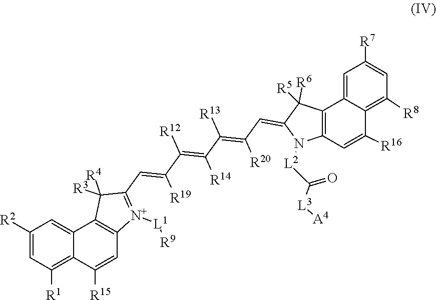

wherein: R1, R2, R3, R4, R5, R6, R7, R8, R15, and R16 are each independently selected from hydrogen, C1-C6 alkyl, C1-C6 alkylene-COOH, sulfonate, C1-C6 alkylene-sulfonate, —COOH, —SO2-NH2, or C1-C6 alkoxy; R9 is hydrogen, sulfonate, amine, or —COOH; L1 is C3-C6 alkylene; L2 is C1-C10 alkylene; L3 is a bond, —O—, —NR10-, —NR10-C1-C6 alkylene-, —O—NR10-, —NR10-C1-C6 alkylene-(O—C1-C6 alkylene)n-, —NR10-L4-, —NR10-C1-C6 alkylene-NR11—(C(═O)—C1-C6 alkylene-O-)m-, or —NR10-C1-C6 alkylene-NR10-C1-C6 alkylene-NR10-C1-C6 alkylene-; L4 is a bond, -heterocyclyl-, or -heterocyclyl-C1-C6 alkylene-; R10 is hydrogen or C1-C6 alkyl; R11 is hydrogen or C1-C6 alkyl; R12 and R13 are independently selected from hydrogen, C1-C6 alkyl, or R12 and R13 are joined together along with the other atoms to which they are attached to form a 5-membered or 6-membered carbocyclic or heterocyclic ring; R14 is hydrogen or C1-C6 alkylene, -(L5)-aryl, -(L5)-aryl-R21, -(L5)-heteroaryl, -(L5)-heteroaryl-R21, —NR17 R18, R14 and R19 are joined together along with the other atoms to which they are attached to form a 5-membered or 6-membered carbocyclic or heterocyclic ring, or R14 and R20 are joined together along with the other atoms to which they are attached to form a 5-membered or 6-membered carbocyclic or heterocyclic ring; L5 is a bond, C1-C10 alkylene, —O—, —NR10-; R17 and R18 are each independently hydrogen or aryl; R19 and R20 are independently selected from hydrogen, C1-C6 alkyl, R14 and R19 are joined together along with the other atoms to which they are attached to form a 5-membered or 6-membered carbocyclic or heterocyclic ring, or R14 and R20 are joined together along with the other atoms to which they are attached to form a 5-membered or 6-membered carbocyclic or heterocyclic ring; R21 is hydrogen, sulfonate, or —COOH; n is 0, 1, 2, or 3; m is 0, 1, 2, or 3; p is 0, 1, 2, or 3; q is 0, 1, 2, or 3; and A4 is the polypeptide.

In some aspects, R3, R4, R5, R6 are each independently methyl; R1, R2, R7, R8, R15, and R16 are each independently hydrogen; R12, R13, R14, R19, and R20 are each independently hydrogen; R9 is sulfonate; R10 is hydrogen; L1 is butylene; L2 is pentylene; or L3 is selected from a bond, —O—, —NR10-, —NR10-C1-C6 alkylene-, —O—NR10-, or —NR10-L4-.

In some aspects, the polypeptide and agent comprises the structure of any one of Formulas (IX), (X), (XI), (XII), (XIII), (XIV), (XV), or (XVI), wherein A4 is the polypeptide:

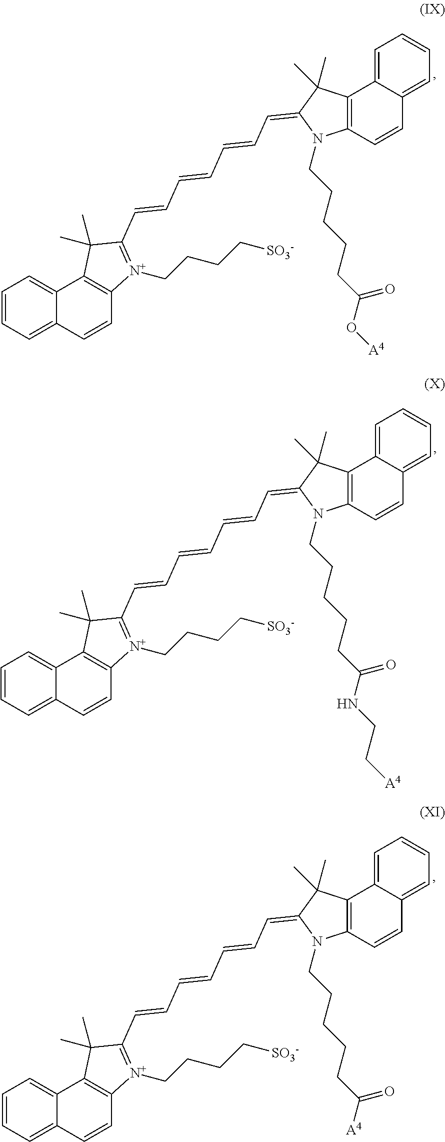

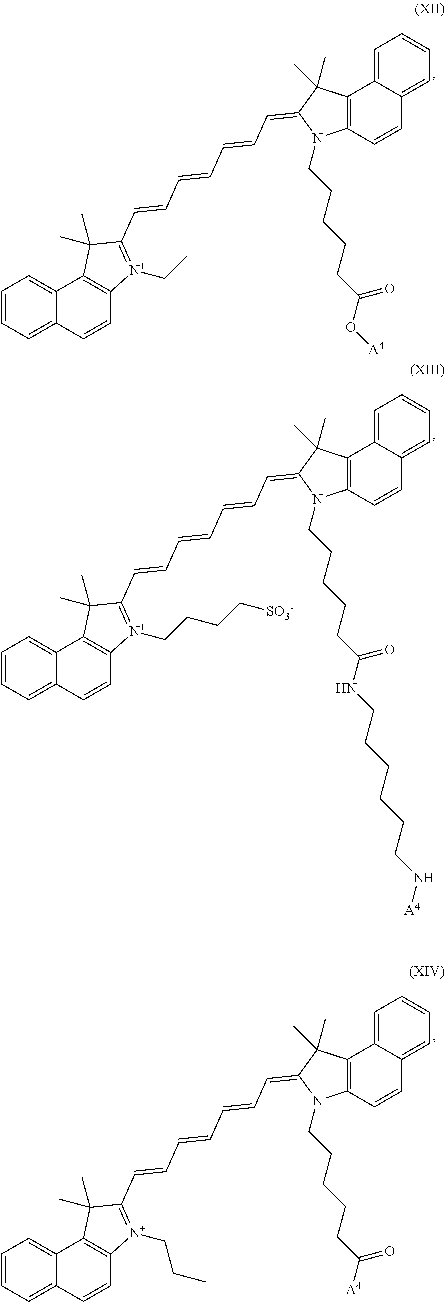

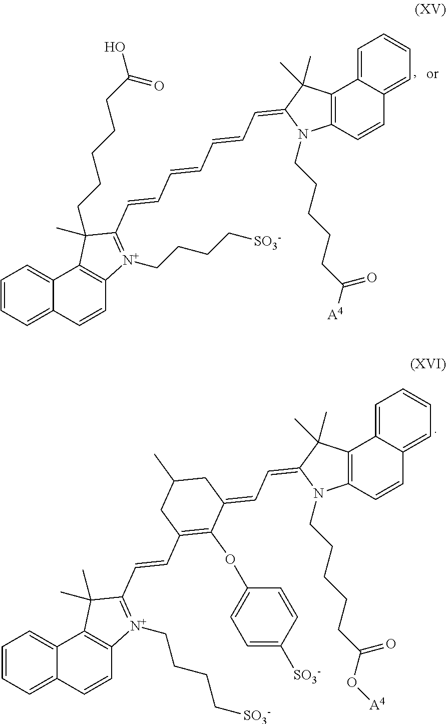

In some aspects, the polypeptide is conjugated to a detectable agent. In some aspects, the polypeptide is conjugated to the detectable agent via a cleavable linker or a stable linker. In some aspects, the detectable agent comprises a dye, a fluorophore, a fluorescent biotin compound, a luminescent compound, a chemiluminescent compound, a radioisotope, nanoparticle, a paramagnetic metal ion, or a combination thereof.

In some aspects, the dye comprises DyLight-680, DyLight-750, VivoTag-750, DyLight-800, JIRDye-800, VivoTag-680, Cy5.5, an indocyanine green (ICG), near infrared dyes, acradine orange or yellow, 7-actinomycin D, 8-anilinonaphthalene-1-sulfonic acid, ATTO dye and any derivative thereof, auramine-rhodamine stain and any derivative thereof, bensantrhone, bimane, 9-10-bis(phenylethynyl)anthracene, 5,12-bis(phenylethynyl)naththacene, bisbenzimide, brainbow, calcein, carbodyfluorescein and any derivative thereof, 1-chloro-9,10-bis(phenylethynyl)anthracene and any derivative thereof, DAPI, DiOC6, DyLight Fluors and any derivative thereof, epicocconone, ethidium bromide, FIAsH-EDT2, Fluo dye and any derivative thereof, FluoProbe and any derivative thereof, Fluorescein and any derivative thereof, Fura and any derivative thereof, GelGreen and any derivative thereof, GelRed and any derivative thereof, fluorescent proteins and any derivative thereof, m isoform proteins and any derivative thereof, hetamethine dye and any derivative thereof, hoeschst stain, iminocoumarin, indian yellow, indo-1 and any derivative thereof, laurdan, lucifer yellow and any derivative thereof, luciferin and any derivative thereof, luciferase and any derivative thereof, mercocyanine and any derivative thereof, methylene blue and any derivative thereof, nile dyes and any derivative thereof, OS680, OS750, perylene, phloxine, phyco dye and any derivative thereof, propium iodide, pyranine, rhodamine and any derivative thereof, ribogreen, RoGFP, rubrene, stilbene and any derivative thereof, sulforhodamine and any derivative thereof, SYBR and any derivative thereof, synapto-pHluorin, tetraphenyl butadiene, tetrasodium tris, Titan Yellow, topotecan, TSQ, umbelliferone, violanthrone, yellow fluroescent protein, YOYO-1 and ZW800, fluorescein and fluorescein dyes, carbocyanine, merocyanine, styryl dyes, oxonol dyes, phycoerythrin, erythrosin, eosin, rhodamine dyes, coumarin and coumarin dyes, Oregon Green Dyes, Texas Red, Texas Red-X, SPECTRUM RED, SPECTRUM GREEN, cyanine dyes, ALEXA FLUOR dyes and any derivative thereof, BODIPY dyes, IRDyes, or any combination thereof.

In further aspects, the near infrared dye comprises a cyanine dyes. In further aspects, the m isoform proteins and any derivative thereof comprises mCherry. In further aspects, the fluorescein and fluorescein dyes comprise fluorescein isothiocyanine or FITC, naphthofluorescein, 4′,5′-dichloro-2′,7′-dimethoxyfluorescein, or 6-carboxyfluorescein or FAM. In further aspects, the rhodamine dyes comprise carboxytetramethyl-rhodamine or TAMRA, carboxyrhodamine 6G, carboxy-X-rhodamine (ROX), lissamine rhodamine B, rhodamine 6G, rhodamine Green, rhodamine Red, or tetramethylrhodamine (TMR). In further aspects, the coumarin and coumarin dyes comprise methoxycoumarin, dialkylaminocoumarin, hydroxycoumarin, or aminomethylcoumarin (AMCA). In further aspects, the Oregon Green Dyes comprise Oregon Green 488, Oregon Green 500, or Oregon Green 514. In further aspects, the SPECTRUM GREEN comprises a cyanine dye comprising CY-3, Cy-5, CY-3.5, or CY-5.5. In further aspects, the ALEXA FLUOR dyes comprise ALEXA FLUOR 350, ALEXA FLUOR 488, ALEXA FLUOR 532, ALEXA FLUOR 546, ALEXA FLUOR 568, ALEXA FLUOR 594, ALEXA FLUOR 633, ALEXA FLUOR 660, or ALEXA FLUOR 680. In further aspects, the BODIPY dyes comprise BODIPY FL, BODIPY R6G, BODIPY TMR, BODIPY TR, BODIPY 530/550, BODIPY 558/568, BODIPY 564/570, BODIPY 576/589, BODIPY 581/591, BODIPY 630/650, or BODIPY 650/665. In further aspects, the IR Dyes comprise IRD40, IRD 700, or IRD 800.

In some aspects, the radioisotope comprises iodine-131, iodine-125, bismuth-212, bismuth-213, lutetium-177, rhenium-186, rhenium-188, yttrium-90, astatine-211, phosphorus-32 and/or samarium-153, or an isotope having one or more atoms having an atomic mass or mass number different from the atomic mass or mass number usually found in nature.

In further aspects, the isotope having one or more atoms having an atomic mass or mass number different from the atomic mass or mass number usually found in nature comprises hydrogen, carbon, fluorine, phosphorous, copper, gallium, yttrium, technetium, indium, iodine, rhenium, thallium, bismuth, astatine, samarium, and lutetium, or any combination thereof. In still further aspects, the lutetium comprises 3H, 3H, 13C, 14C, 18F, 32P, 35S, 64Cu, 67Ga, 90Y, 99MTc, 111In, 125I, 123I, 131I, 135I, 186Re, 187Re, 201Tl, 212Bi, 211At, 153Sm, or 177Lu.

In some aspects, the polypeptide is conjugated to a therapeutic agent. In some aspects, the polypeptide is conjugated to the therapeutic agent via a cleavable linker or a stable linker. In some aspects, the therapeutic agent comprises a radioisotope, nanoparticle, toxin, enzyme, sensitizing drug, radiosensitizer, photosensitizer, nucleic acid, interfering RNA, antibody, antibody fragment, aptamer, anti-angiogenic agent, anti-metabolite, mitotic inhibitor, growth factor inhibitor, or a combination thereof. In some aspects, the radioisotope comprises iodine-131, iodine-125, bismuth-212, bismuth-213, lutetium-177, rhenium-186, rhenium-188, yttrium-90, astatine-211, phosphorus-32 and/or samarium-153, or an isotope having one or more atoms having an atomic mass or mass number different from the atomic mass or mass number usually found in nature.

In further aspects, the isotope having one or more atoms having an atomic mass or mass number different from the atomic mass or mass number usually found in nature comprises hydrogen, carbon, fluorine, phosphorous, copper, gallium, yttrium, technetium, indium, iodine, rhenium, thallium, bismuth, astatine, samarium, and lutetium, or any combination thereof. In still further aspects, the lutetium comprises 3H, 3H, 13C, 14C, 18F, 32P, 35S, 64Cu, 67Ga, 90Y, 99MTc, 111In, 125I, 123I, 131I, 135I, 186Re, 187Re, 201Tl, 212Bi, 211At, 153Sm, or 177Lu.

In some aspects, administering the polypeptide comprises intravenously administering a composition comprising the polypeptide and a pharmaceutically acceptable carrier. In some aspects, the composition comprises a pH within a range from about 6 to about 7.5. In some aspects, the composition comprises an ionic strength less than or equal to about 50 mM. In some aspects, the composition further comprises a buffer comprising histidine, tris, HEPES, ethylene diamine, or a combination thereof. In some aspects, the composition further comprises a sugar alcohol. In some aspects, the composition comprises from about 0 mM to about 50 mM histidine, from about 0 mM to about 20 mM tris, about 20 mM methionine, from about 3% to about 10% sugar alcohol, and a pH within a range from about 6 to about 7.5.

In various aspects, the present disclosure provides a method of imaging an organ, organ substructure, or body region of a subject, the method comprising: administering to the subject a compound comprising a polypeptide conjugated to a detectable marker, wherein the polypeptide comprises: a) any one of SEQ ID NO: 482-SEQ ID NO: 485 or a fragment thereof; b) at least 80%, at least 85%, at least 90%, or at least 95% sequence identity with any one of SEQ ID NO: 1-SEQ ID NO: 481 or a fragment thereof; or c) at least 80%, at least 85%, at least 90%, or at least 95% sequence identity with MCMPCFTTDHQMARRCDDCCGGRGRGKCYGPQCLCR (SEQ ID NO: 9) or a fragment thereof, and imaging an organ or organ substructure comprising a vascular lesion, a cavernoma, an arteriovenous malformation, a venous malformation, a lymphatic malformation, a capillary telangiectasia, a mixed vascular malformation, an aneurysm, or a spinal dural arteriovenous fistula.

In some aspects, the cavernoma comprises a cavernous angioma, a cavernous hemangioma, or a cerebral cavernous malformation (CCM). In some aspects, the arteriovenous malformation comprises an arteriovenous angioma, an arteriovenous hemangioma, or a cerebral arteriovenous malformation (CAM). In some aspects, the aneurysm comprises abdominal aortic, thoracic aortic, or cerebral aneurysm.

In some aspects, the method further comprises detecting the presence or absence of the compound in a tissue or cell, wherein the presence of the compound in the tissue or cell indicates the presence of a vascular lesion in a diseased region, tissue, structure, or cell of the subject. In some aspects, the method further comprises detecting the presence or absence of the compound in a tissue or cell, wherein the presence of the compound in the tissue or cell indicates the presence of one or more of a cavernoma, an arteriovenous malformation, a venous malformation, a lymphatic malformation, a capillary telangiectasia, a mixed vascular malformation, an aneurysm, or a spinal dural arteriovenous fistula in a diseased region, tissue, structure, or cell of the subject.

In some aspects, the method further comprises performing surgery on the subject. In some aspects, the method further comprises treating the vascular lesion. In some aspects, the method further comprises treating the one or more of a cavernoma, an arteriovenous malformation, a venous malformation, a lymphatic malformation, a capillary telangiectasia, a mixed vascular malformation, an aneurysm, or a spinal dural arteriovenous fistula. In some aspects, the method further comprises treating the diseased region, tissue, structure or cell of the subject. In some aspects, the surgery comprises removing the vascular lesion. In some aspects, the surgery comprises removing one or more of a cavernoma, an arteriovenous malformation, a venous malformation, a lymphatic malformation, a capillary telangiectasia, a mixed vascular malformation, an aneurysm, or a spinal dural arteriovenous fistula.

In some aspects, the surgery comprises removing the diseased region, tissue, structure or cell of the subject. In some aspects, the method further comprises imaging the vascular lesion after surgical removal. In some aspects, the method further comprises imaging the one or more of a cavernoma, an arteriovenous malformation, a venous malformation, a lymphatic malformation, a capillary telangiectasia, a mixed vascular malformation, an aneurysm, or a spinal dural arteriovenous fistula after surgical removal.

In some aspects, the method further comprises imaging the diseased region, tissue, structure, or cell of the subject after surgical removal. In some aspects, the method further comprises imaging the vascular lesion surgical bed. In some aspects, the method further comprises detecting residual vascular lesion. In some aspects, the method further comprises surgical removal of the residual vascular lesion.

In some aspects, the polypeptide is conjugated to the detectable agent via a cleavable linker or a stable linker. In some aspects, the detectable agent comprises a dye, a fluorophore, a fluorescent biotin compound, a luminescent compound, a chemiluminescent compound, a radioisotope, nanoparticle, a paramagnetic metal ion, or a combination thereof. In some aspects, the polypeptide is further conjugated to a therapeutic agent. In some aspects, the therapeutic agent comprises a radioisotope, nanoparticle, toxin, enzyme, sensitizing drug, radiosensitizer, photosensitizer, nucleic acid, interfering RNA, antibody, antibody fragment, aptamer, anti-angiogenic agent, anti-metabolite, mitotic inhibitor, growth factor inhibitor, or a combination thereof.

In some aspects, the dye comprises DyLight-680, DyLight-750, VivoTag-750, DyLight-800, IRDye-800, VivoTag-680, Cy5.5, an indocyanine green (ICG), near infrared dyes, acradine orange or yellow, 7-actinomycin D, 8-anilinonaphthalene-1-sulfonic acid, ATTO dye and any derivative thereof, auramine-rhodamine stain and any derivative thereof, bensantrhone, bimane, 9-10-bis(phenylethynyl)anthracene, 5,12-bis(phenylethynyl)naththacene, bisbenzimide, brainbow, calcein, carbodyfluorescein and any derivative thereof, 1-chloro-9,10-bis(phenylethynyl)anthracene and any derivative thereof, DAPI, DiOC6, DyLight Fluors and any derivative thereof, epicocconone, ethidium bromide, FIAsH-EDT2, Fluo dye and any derivative thereof, FluoProbe and any derivative thereof, Fluorescein and any derivative thereof, Fura and any derivative thereof, GelGreen and any derivative thereof, GelRed and any derivative thereof, fluorescent proteins and any derivative thereof, m isoform proteins and any derivative thereof, hetamethine dye and any derivative thereof, hoeschst stain, iminocoumarin, indian yellow, indo-1 and any derivative thereof, laurdan, lucifer yellow and any derivative thereof, luciferin and any derivative thereof, luciferase and any derivative thereof, mercocyanine and any derivative thereof, methylene blue and any derivative thereof, nile dyes and any derivative thereof, OS680, OS750, perylene, phloxine, phyco dye and any derivative thereof, propium iodide, pyranine, rhodamine and any derivative thereof, ribogreen, RoGFP, rubrene, stilbene and any derivative thereof, sulforhodamine and any derivative thereof, SYBR and any derivative thereof, synapto-pHluorin, tetraphenyl butadiene, tetrasodium tris, Titan Yellow, topotecan, TSQ, umbelliferone, violanthrone, yellow fluroescent protein, YOYO-1 and ZW800, fluorescein and fluorescein dyes, carbocyanine, merocyanine, styryl dyes, oxonol dyes, phycoerythrin, erythrosin, eosin, rhodamine dyes, coumarin and coumarin dyes, Oregon Green Dyes, Texas Red, Texas Red-X, SPECTRUM RED, SPECTRUM GREEN, cyanine dyes, ALEXA FLUOR dyes and any derivative thereof, BODIPY dyes, IRDyes, or any combination thereof.

In further aspects, the near infrared dye comprises a cyanine dyes. In further aspects, the m isoform proteins and any derivative thereof comprises mCherry. In further aspects, fluorescein and fluorescein dyes comprise fluorescein isothiocyanine or FITC, naphthofluorescein, 4′,5′-dichloro-2′,7′-dimethoxyfluorescein, or 6-carboxyfluorescein or FAM. In further aspects, rhodamine dyes comprise carboxytetramethyl-rhodamine or TAMRA, carboxyrhodamine 6G, carboxy-X-rhodamine (ROX), lissamine rhodamine B, rhodamine 6G, rhodamine Green, rhodamine Red, or tetramethylrhodamine (TMR).

In further aspects, coumarin and coumarin dyes comprise methoxycoumarin, dialkylaminocoumarin, hydroxycoumarin, or aminomethylcoumarin (AMCA). In further aspects, Oregon Green Dyes comprise Oregon Green 488, Oregon Green 500, or Oregon Green 514. In further aspects, SPECTRUM GREEN comprises a cyanine dye comprising CY-3, Cy-5, CY-3.5, or CY-5.5. In further aspects, ALEXA FLUOR dyes comprise ALEXA FLUOR 350, ALEXA FLUOR 488, ALEXA FLUOR 532, ALEXA FLUOR 546, ALEXA FLUOR 568, ALEXA FLUOR 594, ALEXA FLUOR 633, ALEXA FLUOR 660, or ALEXA FLUOR 680. In further aspects, BODIPY dyes comprise BODIPY FL, BODIPY R6G, BODIPY TMR, BODIPY TR, BODIPY 530/550, BODIPY 558/568, BODIPY 564/570, BODIPY 576/589, BODIPY 581/591, BODIPY 630/650, or BODIPY 650/665. In further aspects, IR Dyes comprise IRD40, IRD 700, or IRD 800. In some aspects, the radioisotope comprises iodine-131, iodine-125, bismuth-212, bismuth-213, lutetium-177, rhenium-186, rhenium-188, yttrium-90, astatine-211, phosphorus-32 and/or samarium-153, or an isotope having one or more atoms having an atomic mass or mass number different from the atomic mass or mass number usually found in nature.

In further aspects, the isotope having one or more atoms having an atomic mass or mass number different from the atomic mass or mass number usually found in nature comprises hydrogen, carbon, fluorine, phosphorous, copper, gallium, yttrium, technetium, indium, iodine, rhenium, thallium, bismuth, astatine, samarium, and lutetium, or any combination thereof. In further aspects, lutetium comprises 3H, 3H, 13C, 14C, 18F, 32P, 35S, 64Cu, 67Ga, 90Y, 99MTc, 111In, 125I, 123I, 131I, 135I, 186Re, 187Re, 201Tl, 212Bi, 211At, 153Sm, or 177Lu.

In some aspects, the radioisotope comprises iodine-131, iodine-125, bismuth-212, bismuth-213, lutetium-177, rhenium-186, rhenium-188, yttrium-90, astatine-211, phosphorus-32 and/or samarium-153, or an isotope having one or more atoms having an atomic mass or mass number different from the atomic mass or mass number usually found in nature.

In further aspects, the isotope having one or more atoms having an atomic mass or mass number different from the atomic mass or mass number usually found in nature comprises hydrogen, carbon, fluorine, phosphorous, copper, gallium, yttrium, technetium, indium, iodine, rhenium, thallium, bismuth, astatine, samarium, and lutetium, or any combination thereof. In still further aspects, the lutetium comprises 3H, 3H, 13C, 14C, 18F, 32P, 35S, 64Cu, 67Ga, 90Y, 99MTc, 111In, 125I, 123I, 131I, 135I, 186Re, 187Re, 201Tl, 212Bi, 211 At, 153Sm, or 177Lu.

In some aspects, the fragment of the polypeptide has a length of at least 25 residues. In some aspects, each amino acid of the polypeptide is independently selected as an L- or D-enantiomer. In some aspects, the polypeptide contains no lysine residues. In some aspects, the polypeptide contains a single lysine residue. In some aspects, the single lysine residue is located at a position corresponding to K-27 of native chlorotoxin, K-23 of native chlorotoxin, or K-15 of native chlorotoxin. In some aspects, one, two, or three methionine residues of the polypeptide are replaced with other amino acids. In some aspects, the N-terminus of the polypeptide is blocked by acetylation or cyclization. In some aspects, the polypeptide comprises at least 1, at least 2, at least 3, at least 4, at least 5, or at least 6 disulfide bonds.

In some aspects, the polypeptide comprises an isoelectric point of at least 6.0, at least 6.5, at least 7.0, at least 7.5, at least 8.0, at least 8.5, or at least 9.0. In some aspects, the polypeptide binds to or accumulates in a vascular lesion tissue or vascular lesion cell. In some aspects, the polypeptide and detectable agent comprises the structure of Formula (IV), or a pharmaceutically acceptable salt thereof:

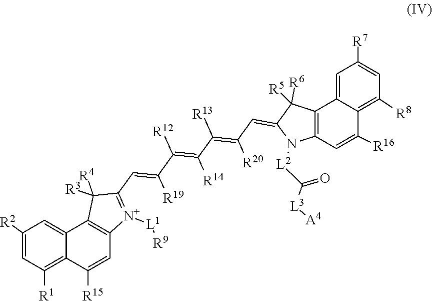

wherein: R1, R2, R3, R4, R5, R6, R7, R8, R15, and R16 are each independently selected from hydrogen, C1-C6 alkyl, C1-C6 alkylene-COOH, sulfonate, C1-C6 alkylene-sulfonate, —COOH, —SO2-NH2, or C1-C6 alkoxy; R9 is hydrogen, sulfonate, amine, or —COOH; L1 is C3-C6 alkylene; L2 is C1-C10 alkylene; L3 is a bond, —O—, —NR10-, —NR10-C1-C6 alkylene-, —O—NR10-, —NR10-C1-C6 alkylene-(O—C1-C6 alkylene)n-, —NR10-L4-, —NR10-C1-C6 alkylene-NR11—(C(═O)—C1-C6 alkylene-O-)m-, or —NR10-C1-C6 alkylene-NR10-C1-C6 alkylene-NR10-C1-C6 alkylene-; L4 is a bond, -heterocyclyl-, or -heterocyclyl-C1-C6 alkylene-; R10 is hydrogen or C1-C6 alkyl; R11 is hydrogen or C1-C6 alkyl; R12 and R13 are independently selected from hydrogen, C1-C6 alkyl, or R12 and R13 are joined together along with the other atoms to which they are attached to form a 5-membered or 6-membered carbocyclic or heterocyclic ring; R14 is hydrogen or C1-C6 alkylene, -(L5)-aryl, -(L5)-aryl-R21, -(L5)-heteroaryl, -(L5)-heteroaryl-R21, —NR17 R18, R14 and R19 are joined together along with the other atoms to which they are attached to form a 5-membered or 6-membered carbocyclic or heterocyclic ring, or R14 and R20 are joined together along with the other atoms to which they are attached to form a 5-membered or 6-membered carbocyclic or heterocyclic ring; L5 is a bond, C1-C10 alkylene, —O—, —NR10-; R17 and R18 are each independently hydrogen or aryl; R19 and R20 are independently selected from hydrogen, C1-C6 alkyl, R14 and R19 are joined together along with the other atoms to which they are attached to form a 5-membered or 6-membered carbocyclic or heterocyclic ring, or R14 and R20 are joined together along with the other atoms to which they are attached to form a 5-membered or 6-membered carbocyclic or heterocyclic ring; R21 is hydrogen, sulfonate, or —COOH; n is 0, 1, 2, or 3; m is 0, 1, 2, or 3; p is 0, 1, 2, or 3; q is 0, 1, 2, or 3; and A4 is the polypeptide.

In some aspects, R3, R4, R5, R6 are each independently methyl; R1, R2, R7, R8, R15, and R16 are each independently hydrogen; R12, R13, R14, R19, and R20 are each independently hydrogen; R9 is sulfonate; R10 is hydrogen; L1 is butylene; L2 is pentylene; or L3 is selected from a bond, —O—, —NR10-, —NR10-C1-C6 alkylene-, —O—NR10-, or —NR10-L4-.

In some aspects, the polypeptide and detectable agent comprises the structure of any one of Formulas (IX), (X), (XI), (XII), (XIII), (XIV), (XV), or (XVI), wherein A4 is the polypeptide:

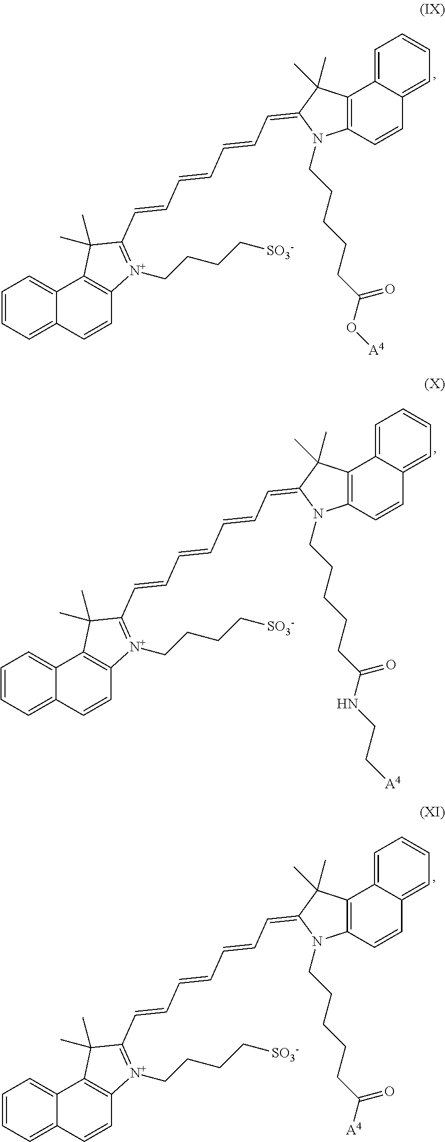

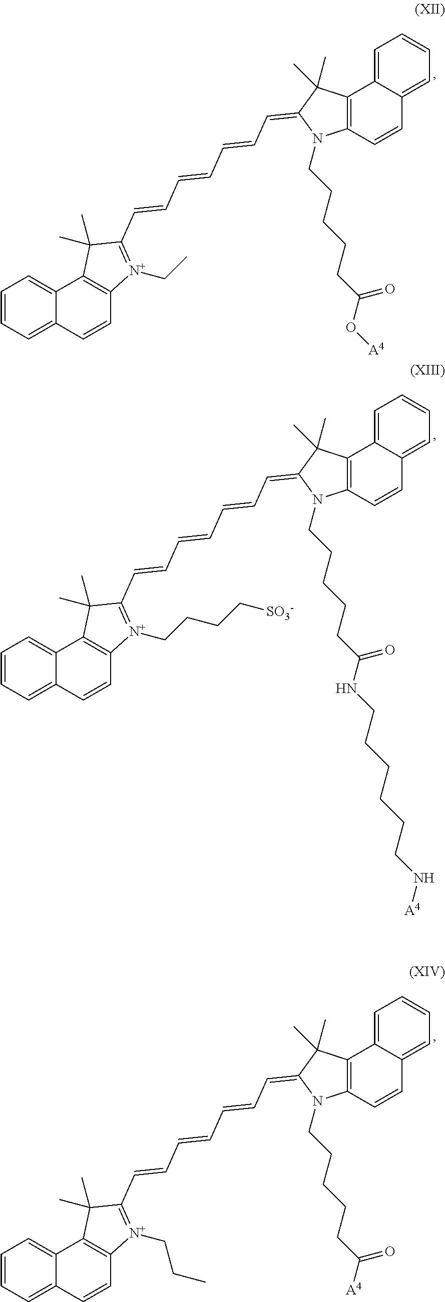

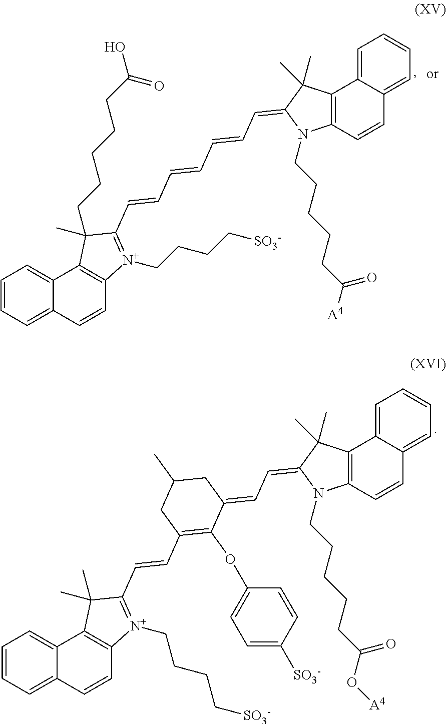

In some aspects, the polypeptide comprises a single lysine residue and the detectable agent is conjugated to the polypeptide at the single lysine residue. In some aspects, the polypeptide comprises no lysine residues and the detectable agent is conjugated to the polypeptide at the N-terminus of the polypeptide. In some aspects, the compound is administered as a composition comprising the compound and a pharmaceutically acceptable carrier. In some aspects, the composition comprises a pH within a range from about 6 to about 7.5. In some aspects, the composition comprises an ionic strength less than or equal to about 50 mM. In some aspects, the composition further comprises a buffer comprising histidine, tris, HEPES, ethylene diamine, or a combination thereof.

In some aspects, the composition further comprises a sugar alcohol. In some aspects, the composition comprises from about 0 mM to about 50 mM histidine, from about 0 mM to about 20 mM tris, about 20 mM methionine, from about 3% to about 10% sugar alcohol, and a pH within a range from about 6 to about 7.5. In some aspects, the polypeptide and the detectable agent are conjugated via a cleavable linker or stable linker. In some aspects, the polypeptide and the therapeutic agent are conjugated via a cleavable linker or stable linker.

In various aspects, the present disclosure provides a method of determining the effect of treating a subject, the method comprising: treating the subject with any of the methods described above; administering any polypeptide as described above; and determining the treatment is efficacious when a signal from the polypeptide is lower compared to a baseline measurement.

In some aspects, the baseline measurement is obtained by administering any polypeptide described above before the treating the subject; and detecting a baseline signal from the polypeptide.

### INCORPORATION BY REFERENCE

All publications, patents, and patent applications mentioned in this specification are herein incorporated by reference to the same extent as if each individual publication, patent, or patent application was specifically and individually indicated to be incorporated by reference.

### BRIEF DESCRIPTION OF THE DRAWINGS

The patent or application file contains at least one drawing executed in color. Copies of this patent or patent application publication with color drawing(s) will be provided by the Office upon request and payment of the necessary fee. The novel features of the invention are set forth with particularity in the appended claims. A better understanding of the features and advantages of the present invention will be obtained by reference to the following detailed description that sets forth illustrative embodiments, in which the principles of the invention are utilized, and the accompanying drawings of which:

FIG. 1 and FIG. 2 show exemplary visible images of a tissue sample of a vascular lesion, vascular malformation, or vascular abnormality acquired using the imaging systems and methods herein, in accordance in some embodiments. Representative images of in situ or intra-operative tissue during surgery on a vascular lesion in a patient, wherein 22 mg (15 mg/m²) of Compound 76 was administered to the human subject.

FIG. 1A shows a near-infrared (NIR) image of the in situ specimen. Fluorescence signal, corresponding to lighter and brighter areas in the NIR images, is indicative of the presence of Compound 76 in the vascular lesion. Labeled arrows indicate non-fluorescent regions of normal blood vessels (“BV”) and normal brain tissue (“NB”). In contrast, fluorescence signal corresponding to lighter and brighter areas in the NIR image was indicative of the presence of Compound 76 on the abnormal vascular lesion (“VL”), and not in normal tissue.

FIG. 1B shows the white light image corresponding to FIG. 1A that represents what the surgeon would normally see without fluorescence guidance. The arrows mark the same locations as shown in the NIR image in FIG. 1A. The vascular lesion (“VL”) had a similar appearance to the normal blood vessels (“BV”) in this image.

FIG. 1C shows the NIR fluorescence and white light composite image of FIG. 1A and FIG. 1B, with arrows marking the same locations as shown in FIG. 1A and FIG. 1B. Fluorescence in the vascular lesion (“VL”) clearly differentiated it from the surrounding normal tissues, including normal blood vessels (“BV”).

FIG. 2A shows a near-infrared (NIR) image of the vascular lesion during the surgery. Arrows indicate the vascular lesion (labeled “VL”) and adjacent normal brain (labeled “NB”), which is non-fluorescent.

FIG. 2B shows the white light image corresponding to FIG. 2A. While the normal brain has a light tan to pink color (light gray in a gray scale image), it is perfused with normal blood vessels that can be differentiated from the vascular lesion by the absence of fluorescence.

FIG. 2C shows the composite white light and NIR image shown in FIG. 2A and FIG. 2B.

### DETAILED DESCRIPTION

The present disclosure provides compositions and methods for the detection and/or treatment of vascular lesions including cavernomas (also referred to as cavernous angiomas, cavernous hemangiomas, or cerebral cavernous malformation (CCM)) and other vascular lesions such as aneurysm, arteriovenous malformation, spinal dural arteriovenous fistula, venous malformation, lymphatic malformation, capillary telangiectasia, mixed vascular malformation, and the like. The compositions and methods described herein comprise peptide complexes comprising a detectable label, which are suitable for the detection and treatment of vascular lesions. In some aspects, the type of vascular lesion is a cavernoma (also referred to as cavernous angiomas, cavernous hemangiomas, or cerebral cavernous malformation (CCM)). In other aspects, the type of vascular lesion is an arteriovenous malformation (also referred to as arteriovenous angiomas, arteriovenous hemangiomas, or cerebral arteriovenous malformation (CAM)). In still other aspects, the type of vascular lesion is an aneurysm (e.g., including abdominal aortic, thoracic aortic, and cerebral aneurysms). Cerebral aneurysms include saccular aneurysm (e.g., a rounded sac containing blood that is attached to an artery), fusiform aneurysm (e.g., one that balloons or bulges out of an artery) and mycotic aneurysm (e.g., one that presents as a dilation of an artery due to damage of the vessel wall by, for example, an infection).

In still other aspects, the type of vascular lesion is a spinal dural arteriovenous fistula (e.g., an abnormal connection between an artery and a vein that are located near the covering of the spinal cord). In certain aspects, the compositions are provided in combination with a pharmaceutically acceptable carrier, which can be administered to a subject by any route of administration. Following administration of the compositions described herein, the peptides or peptide complexes bind selectively to vascular lesions. The vascular lesions can then be detected, for example, by imaging or other visualization or detection method suitable for detecting the detectable label of the peptide conjugate. In further aspects, the presently described compositions can be used to treat the type of vascular lesion or malformation by way of a therapeutic agent, which is attached to the conjugate and which acts on the vascular lesions following binding by the peptide portion of the conjugate. These and other aspects are described in detail herein.

Cavernomas are benign (i.e., non-neoplastic or non-cancerous) vascular malformations or lesions that may cause seizures and/or hemorrhage when they develop in the brain. Some cavernous angiomas bleed slowly enough that the body can re-absorb the blood. Others bleed more profusely and can put dangerous pressure on the surrounding brain tissue and/or cause an obvious hemorrhage. Symptoms include bleeding (hemorrhage), fits (seizures), headaches, neurological problems, such as dizziness, slurred speech (dysarthria), loss or impaired vision, blurred vision, double vision, loss or impaired sense of smell (anosmia), other focal neurological deficits, or balance problems and tremor, weakness, numbness, tiredness, memory problems and difficulty concentrating. Moreover, they can produce a hemorrhagic stroke and other complications that are life-threatening or create chronic problems. Environmental factors, such as radiation treatment can affect the incidence of cavernomas by increasing damage to tissue and incidence of bleeding. Treatment usually includes surgery and the precision of surgical resection directly influences patient prognosis. Unfortunately, intra-operative identification of lesion margins or small foci remains imprecise, and these lesions if located in organs and organ substructures, such as the brain and other organs and organ structures such as brain, heart, lung, kidney, liver, CNS (e.g., spine) or pancreas can be debilitating or life-threatening. In addition to improving treatment and surgical outcome of cavernomas, there are similar needs for improving treatment and surgical outcome in other vascular lesions such as aneurysm, arteriovenous malformation, spinal dural arteriovenous fistula venous malformation, lymphatic malformation, capillary telangiectasia, mixed vascular malformation, and the like.

The invention will best be understood by reference to the following detailed description of the aspects and embodiments of the invention, taken in conjunction with the accompanying drawings and figures. The discussion below is descriptive, illustrative and exemplary and is not to be taken as limiting the scope defined by any appended claims.

As used in the specification and appended claims, unless specified to the contrary, the following terms have the meaning indicated.

As used herein and in the appended claims, the singular forms “a,” “and,” and “the” include plural referents unless the context clearly dictates otherwise. Thus, for example, reference to “a compound” includes a plurality of such compounds, reference to “an agent” includes a plurality of such agents, and reference to “the cell” includes reference to one or more cells (or to a plurality of cells) and equivalents thereof known to those skilled in the art, and so forth. When ranges are used herein for physical properties, such as molecular weight, or chemical properties, such as chemical formulae, all combinations and subcombinations of ranges and specific embodiments therein are intended to be included. The term “about” when referring to a number or a numerical range means that the number or numerical range referred to is an approximation within experimental variability (or within statistical experimental error), and thus the number or numerical range may vary between 1% and 15% of the stated number or numerical range. The term “comprising” (and related terms such as “comprise” or “comprises” or “having” or “including”) is not intended to exclude that in other certain embodiments, for example, an embodiment of any composition of matter, composition, method, or process, or the like, described herein, may “consist of” or “consist essentially of” the described features.

“Cyano” refers to the —CN radical.

“Nitro” refers to the —NO₂ radical.

“Oxa” refers to the —O— radical.

“Oxo” refers to the ═O radical.

“Thioxo” refers to the ═S radical.

“Imino” refers to the ═N—H radical.

“Hydrazino” refers to the ═N—NH₂ radical.

“Alkyl” refers to a straight or branched hydrocarbon chain radical consisting solely of carbon and hydrogen atoms, containing no unsaturation, having from one to fifteen carbon atoms (e.g., C₁-C₁₅ alkyl). In certain embodiments, an alkyl comprises one to thirteen carbon atoms (e.g., C₁-C₁₃ alkyl). In certain embodiments, an alkyl comprises one to eight carbon atoms (e.g., C₁-C₈ alkyl). In other embodiments, an alkyl comprises five to fifteen carbon atoms (e.g., C₅-C₁₅ alkyl). In other embodiments, an alkyl comprises five to eight carbon atoms (e.g., C₅-C₈alkyl). The alkyl is attached to the rest of the molecule by a single bond, for example, methyl (Me), ethyl (Et), n-propyl, 1-methylethyl (iso-propyl), n-butyl, n-pentyl, 1,1-dimethylethyl (t-butyl), 3-methylhexyl, 2-methylhexyl, and the like. Unless stated otherwise specifically in the specification, an alkyl group is optionally substituted by one or more of the following substituents: halo, cyano, nitro, oxo, thioxo, trimethylsilanyl, —OR^{a}, —SR^{a}, —OC(O)—R^{a}, —N(R^{a})₂, —C(O)R^{a}, —C(O)OR^{a}, —C(O)N(R^{a})₂, —N(R^{a})C(O)OR^{a}, —N(R^{a})C(O)R^{a}, —N(R^{a})S(O)ₜR^{a} (where t is 1 or 2), —S(O)ₜOR^{a} (where t is 1 or 2) and —S(O)ₜN(R^{a})₂ (where t is 1 or 2) where each R^{a} is independently hydrogen, alkyl, fluoroalkyl, carbocyclyl, carbocyclylalkyl, aryl, aralkyl, heterocyclyl, heterocyclylalkyl, heteroaryl or heteroarylalkyl.

“Alkenyl” refers to a straight or branched hydrocarbon chain radical group consisting solely of carbon and hydrogen atoms, containing at least one double bond, and having from two to twelve carbon atoms. In certain embodiments, an alkenyl comprises two to eight carbon atoms. In other embodiments, an alkenyl comprises two to four carbon atoms. The alkenyl is attached to the rest of the molecule by a single bond, for example, ethenyl (i.e., vinyl), prop-1-enyl (i.e., allyl), but-1-enyl, pent-1-enyl, penta-1,4-dienyl, and the like. Unless stated otherwise specifically in the specification, an alkenyl group is optionally substituted by one or more of the following substituents: halo, cyano, nitro, oxo, thioxo, trimethylsilanyl, —OR^{a}, —SR^{a}, —OC(O)—R^{a}, —N(R^{a})₂, —C(O)R^{a}, —C(O)OR^{a}, —C(O)N(R^{a})₂, —N(R^{a})C(O)OR^{a}, —N(R^{a})C(O)R^{a}, —N(R^{a})S(O)ₜR^{a} (where t is 1 or 2), —S(O)ₜOR^{a} (where t is 1 or 2) and —S(O)ₜN(R^{a})₂ (where t is 1 or 2) where each R^{a} is independently hydrogen, alkyl, fluoroalkyl, carbocyclyl, carbocyclylalkyl, aryl, aralkyl, heterocyclyl, heterocyclylalkyl, heteroaryl or heteroarylalkyl.

“Alkynyl” refers to a straight or branched hydrocarbon chain radical group consisting solely of carbon and hydrogen atoms, containing at least one triple bond, having from two to twelve carbon atoms. In certain embodiments, an alkynyl comprises two to eight carbon atoms. In other embodiments, an alkynyl has two to four carbon atoms. The alkynyl is attached to the rest of the molecule by a single bond, for example, ethynyl, propynyl, butynyl, pentynyl, hexynyl, and the like. Unless stated otherwise specifically in the specification, an alkynyl group is optionally substituted by one or more of the following substituents: halo, cyano, nitro, oxo, thioxo, trimethylsilanyl, —OR^{a}, —SR^{a}, —OC(O)—R^{a}, —N(R^{a})₂, —C(O)R^{a}, —C(O)OR^{a}, —C(O)N(R^{a})₂, —N(R^{a})C(O)OR^{a}, —N(R^{a})C(O)R^{a}, —N(R^{a})S(O)ₜR^{a} (where t is 1 or 2), —S(O)ₜOR^{a} (where t is 1 or 2) and —S(O)ₜN(R^{a})₂ (where t is 1 or 2) where each R^{a} is independently hydrogen, alkyl, fluoroalkyl, carbocyclyl, carbocyclylalkyl, aryl, aralkyl, heterocyclyl, heterocyclylalkyl, heteroaryl or heteroarylalkyl.

“Alkylene” or “alkylene chain” refers to a straight or branched divalent hydrocarbon chain linking the rest of the molecule to a radical group, consisting solely of carbon and hydrogen, containing no unsaturation and having from one to twelve carbon atoms, for example, methylene, ethylene, propylene, n-butylene, and the like. The alkylene chain is attached to the rest of the molecule through a single bond and to the radical group through a single bond. The points of attachment of the alkylene chain to the rest of the molecule and to the radical group can be through one carbon in the alkylene chain or through any two carbons within the chain. Unless stated otherwise specifically in the specification, an alkylene chain is optionally substituted by one or more of the following substituents: halo, cyano, nitro, aryl, cycloalkyl, heterocyclyl, heteroaryl, oxo, thioxo, trimethylsilanyl, —OR^{a}, —SR^{a}, —OC(O)—R^{a}, —N(R^{a})₂, —C(O)R^{a}, —C(O)OR^{a}, —C(O)N(R^{a})₂, —N(R^{a})C(O)OR^{a}, —N(R^{a})C(O)R^{a}, —N(R^{a})S(O)ₜR^{a} (where t is 1 or 2), —S(O)ₜOR^{a} (where t is 1 or 2) and —S(O)ₜN(R^{a})₂ (where t is 1 or 2) where each R^{a} is independently hydrogen, alkyl, fluoroalkyl, carbocyclyl, carbocyclylalkyl, aryl, aralkyl, heterocyclyl, heterocyclylalkyl, heteroaryl or heteroarylalkyl.

“Alkenylene” or “alkenylene chain” refers to a straight or branched divalent hydrocarbon chain linking the rest of the molecule to a radical group, consisting solely of carbon and hydrogen, containing at least one double bond and having from two to twelve carbon atoms, for example, ethenylene, propenylene, n-butenylene, and the like. The alkenylene chain is attached to the rest of the molecule through a double bond or a single bond and to the radical group through a double bond or a single bond. The points of attachment of the alkenylene chain to the rest of the molecule and to the radical group can be through one carbon or any two carbons within the chain. Unless stated otherwise specifically in the specification, an alkenylene chain is optionally substituted by one or more of the following substituents: halo, cyano, nitro, aryl, cycloalkyl, heterocyclyl, heteroaryl, oxo, thioxo, trimethylsilanyl, —OR^{a}, —SR^{a}, —OC(O)—R^{a}, —N(R^{a})₂, —C(O)R^{a}, —C(O)OR^{a}, —C(O)N(R^{a})₂, —N(R^{a})C(O)OR^{a}, —N(R^{a})C(O)R^{a}, —N(R^{a})S(O)ₜR^{a} (where t is 1 or 2), —S(O)ₜOR^{a} (where t is 1 or 2) and —S(O)ₜN(R^{a})₂ (where t is 1 or 2) where each R^{a} is independently hydrogen, alkyl, fluoroalkyl, cycloalkyl, cycloalkylalkyl, aryl (optionally substituted with one or more halo groups), aralkyl, heterocyclyl, heterocyclylalkyl, heteroaryl or heteroarylalkyl, and where each of the above substituents is unsubstituted unless otherwise indicated.

“Aryl” refers to a radical derived from an aromatic monocyclic or multicyclic hydrocarbon ring system by removing a hydrogen atom from a ring carbon atom. The aromatic monocyclic or multicyclic hydrocarbon ring system contains only hydrogen and carbon from six to eighteen carbon atoms, where at least one of the rings in the ring system is fully unsaturated, i.e., it contains a cyclic, delocalized (4n+2) π-electron system in accordance with the Hückel theory. Aryl groups include, but are not limited to, groups such as phenyl, fluorenyl, and naphthyl. Unless stated otherwise specifically in the specification, the term “aryl” or the prefix “ar-” (such as in “aralkyl”) is meant to include aryl radicals optionally substituted by one or more substituents independently selected from alkyl, alkenyl, alkynyl, halo, fluoroalkyl, cyano, nitro, optionally substituted aryl, optionally substituted aralkyl, optionally substituted aralkenyl, optionally substituted aralkynyl, optionally substituted carbocyclyl, optionally substituted carbocyclylalkyl, optionally substituted heterocyclyl, optionally substituted heterocyclylalkyl, optionally substituted heteroaryl, optionally substituted heteroarylalkyl, —R^{b}—OR^{a}, —R^{b}—OC(O)—R^{a}, —R^{b}—N(R^{a})₂, —R^{b}—C(O)R^{a}, —R^{b}—C(O)OR^{a}, —R^{b}—C(O)N(R^{a})₂, —R^{b}—O—R^{c}—C(O)N(R^{a})₂, —R^{b}—N(R^{a})C(O)OR^{a}, —R^{b}—N(R^{a})C(O)R^{a}, —R^{b}—N(R^{a})S(O)ₜR^{a} (where t is 1 or 2), —R^{b}—S(O)OR^{a} (where t is 1 or 2) and —R^{b}—S(O)ₜN(R^{a})₂ (where t is 1 or 2), where each R^{a} is independently hydrogen, alkyl, fluoroalkyl, cycloalkyl, cycloalkylalkyl, aryl (optionally substituted with one or more halo groups), aralkyl, heterocyclyl, heterocyclylalkyl, heteroaryl or heteroarylalkyl, each R^{a} is independently a direct bond or a straight or branched alkylene or alkenylene chain, and R^{C} is a straight or branched alkylene or alkenylene chain, and where each of the above substituents is unsubstituted unless otherwise indicated.

“Aralkyl” refers to a radical of the formula —R^{C}-aryl where R^{C} is an alkylene chain as defined above, for example, benzyl, diphenylmethyl and the like. The alkylene chain part of the aralkyl radical is optionally substituted as described above for an alkylene chain. The aryl part of the aralkyl radical is optionally substituted as described above for an aryl group.

“Aralkenyl” refers to a radical of the formula —R^{d}-aryl where R^{d} is an alkenylene chain as defined above. The aryl part of the aralkenyl radical is optionally substituted as described above for an aryl group. The alkenylene chain part of the aralkenyl radical is optionally substituted as defined above for an alkenylene group.

“Aralkynyl” refers to a radical of the formula —R-aryl, where R is an alkynylene chain as defined above. The aryl part of the aralkynyl radical is optionally substituted as described above for an aryl group. The alkynylene chain part of the aralkynyl radical is optionally substituted as defined above for an alkynylene chain.

“Carbocyclyl” refers to a stable non-aromatic monocyclic or polycyclic hydrocarbon radical consisting solely of carbon and hydrogen atoms, which may include fused or bridged ring systems, having from three to fifteen carbon atoms. In certain embodiments, a carbocyclyl comprises three to ten carbon atoms. In other embodiments, a carbocyclyl comprises five to seven carbon atoms. The carbocyclyl is attached to the rest of the molecule by a single bond. Carbocyclyl may be saturated, (i.e., containing single C—C bonds only) or unsaturated (i.e., containing one or more double bonds or triple bonds.) A fully saturated carbocyclyl radical is also referred to as “cycloalkyl.” Examples of monocyclic cycloalkyls include, e.g., cyclopropyl, cyclobutyl, cyclopentyl, cyclohexyl, cycloheptyl, and cyclooctyl. An unsaturated carbocyclyl is also referred to as “cycloalkenyl.” Examples of monocyclic cycloalkenyls include, e.g., cyclopentenyl, cyclohexenyl, cycloheptenyl, and cyclooctenyl. Polycyclic carbocyclyl radicals include, for example, adamantyl, norbornyl (i.e., bicyclo[2.2.1]heptanyl), norbornenyl, decalinyl, 7,7-dimethyl-bicyclo[2.2.1]heptanyl, and the like. Unless otherwise stated specifically in the specification, the term “carbocyclyl” is meant to include carbocyclyl radicals that are optionally substituted by one or more substituents independently selected from alkyl, alkenyl, alkynyl, halo, fluoroalkyl, oxo, thioxo, cyano, nitro, optionally substituted aryl, optionally substituted aralkyl, optionally substituted aralkenyl, optionally substituted aralkynyl, optionally substituted carbocyclyl, optionally substituted carbocyclylalkyl, optionally substituted heterocyclyl, optionally substituted heterocyclylalkyl, optionally substituted heteroaryl, optionally substituted heteroarylalkyl, —R^{b}—OR^{a}, —R^{b}—SR^{a}, —R^{b}—OC(O)—R^{a}, —R^{b}—N(R^{a})₂, —R^{b}—C(O)R^{a}, —R^{b}—C(O)OR^{a}, —R—C(O)N(R^{a})₂, —R^{b}—O—R^{c}—C(O)N(R^{a})₂, —R^{b}—N(R^{a})C(O)OR^{a}, —R^{b}—N(R^{a})C(O)R^{a}, —R^{b}—N(R^{a})S(O)R^{a} (where t is 1 or 2), —R^{b}—S(O)ₜOR^{a} (where t is 1 or 2) and —R^{b}S(O)ₜN(R^{a})₂ (where t is 1 or 2), where each R^{a} is independently hydrogen, alkyl, fluoroalkyl, cycloalkyl, cycloalkylalkyl, aryl, aralkyl, heterocyclyl, heterocyclylalkyl, heteroaryl or heteroarylalkyl, each R^{a} is independently a direct bond or a straight or branched alkylene or alkenylene chain, and R^{C} is a straight or branched alkylene or alkenylene chain, and where each of the above substituents is unsubstituted unless otherwise indicated.

“Carbocyclylalkyl” refers to a radical of the formula —R^{c}-carbocyclyl where R^{C} is an alkylene chain as defined above. The alkylene chain and the carbocyclyl radical is optionally substituted as defined above.

“Halo” or “halogen” refers to bromo, chloro, fluoro or iodo substituents.

“Fluoroalkyl” refers to an alkyl radical, as defined above, that is substituted by one or more fluoro radicals, as defined above, for example, trifluoromethyl, difluoromethyl, 2,2,2-trifluoroethyl, 1-fluoromethyl-2-fluoroethyl, and the like. The alkyl part of the fluoroalkyl radical is optionally substituted as defined above for an alkyl group.

“Heterocyclyl” refers to a 3- to 18-membered non-aromatic ring radical that comprises two to twelve carbon atoms and from one to six heteroatoms selected from nitrogen, oxygen and sulfur. Unless stated otherwise specifically in the specification, the heterocyclyl radical is a monocyclic, bicyclic, tricyclic or tetracyclic ring system, which may include fused or bridged ring systems. The heteroatoms in the heterocyclyl radical may be optionally oxidized. One or more nitrogen atoms, if present, are optionally quaternized. The heterocyclyl radical is partially or fully saturated. The heterocyclyl may be attached to the rest of the molecule through any atom of the ring(s). Examples of such heterocyclyl radicals include, but are not limited to, dioxolanyl, thienyl[1,3]dithianyl, decahydroisoquinolyl, imidazolinyl, imidazolidinyl, isothiazolidinyl, isoxazolidinyl, morpholinyl, octahydroindolyl, octahydroisoindolyl, 2-oxopiperazinyl, 2-oxopiperidinyl, 2-oxopyrrolidinyl, oxazolidinyl, piperidinyl, piperazinyl, 4-piperidonyl, pyrrolidinyl, pyrazolidinyl, quinuclidinyl, thiazolidinyl, tetrahydrofuryl, trithianyl, tetrahydropyranyl, thiomorpholinyl, thiamorpholinyl, 1-oxo-thiomorpholinyl, and 1,1-dioxo-thiomorpholinyl. Unless stated otherwise specifically in the specification, the term “heterocyclyl” is meant to include heterocyclyl radicals as defined above that are optionally substituted by one or more substituents selected from alkyl, alkenyl, alkynyl, halo, fluoroalkyl, oxo, thioxo, cyano, nitro, optionally substituted aryl, optionally substituted aralkyl, optionally substituted aralkenyl, optionally substituted aralkynyl, optionally substituted carbocyclyl, optionally substituted carbocyclylalkyl, optionally substituted heterocyclyl, optionally substituted heterocyclylalkyl, optionally substituted heteroaryl, optionally substituted heteroarylalkyl, —R^{b}—OR^{a}, —R^{b}—SR^{a}, —R^{b}—OC(O)—R^{a}, —R^{b}—N(R^{a})₂, —R^{b}—C(O)R^{a}, —R^{b}—C(O)OR^{a}, —R^{b}—C(O)N(R^{a})₂, —R^{b}—O—R^{c}—C(O)N(R^{a})₂, —R^{b}—N(R^{a})C(O)OR^{a}, —R^{b}—N(R^{a})C(O)R^{a}, —R^{b}—N(R^{a})S(O)ₜR^{a} (where t is 1 or 2), —R^{b}—S(O)ₜOR^{a} (where t is 1 or 2) and —R^{b}—S(O)ₜN(R^{a})₂ (where t is 1 or 2), where each R^{a} is independently hydrogen, alkyl, fluoroalkyl, cycloalkyl, cycloalkylalkyl, aryl, aralkyl, heterocyclyl, heterocyclylalkyl, heteroaryl or heteroarylalkyl, each R^{a} is independently a direct bond or a straight or branched alkylene or alkenylene chain, and R^{C} is a straight or branched alkylene or alkenylene chain, and where each of the above substituents is unsubstituted unless otherwise indicated.

“N-heterocyclyl” or “N-attached heterocyclyl” refers to a heterocyclyl radical as defined above containing at least one nitrogen and where the point of attachment of the heterocyclyl radical to the rest of the molecule is through a nitrogen atom in the heterocyclyl radical. An N-heterocyclyl radical is optionally substituted as described above for heterocyclyl radicals. Examples of such N-heterocyclyl radicals include, but are not limited to, 1-morpholinyl, 1-piperidinyl, 1-piperazinyl, 1-pyrrolidinyl, pyrazolidinyl, imidazolinyl, and imidazolidinyl.

“C-heterocyclyl” or “C-attached heterocyclyl” refers to a heterocyclyl radical as defined above containing at least one heteroatom and where the point of attachment of the heterocyclyl radical to the rest of the molecule is through a carbon atom in the heterocyclyl radical. A C-heterocyclyl radical is optionally substituted as described above for heterocyclyl radicals. Examples of such C-heterocyclyl radicals include, but are not limited to, 2-morpholinyl, 2- or 3- or 4-piperidinyl, 2-piperazinyl, 2- or 3-pyrrolidinyl, and the like.

“Heterocyclylalkyl” refers to a radical of the formula —R^{c}-heterocyclyl where R^{C} is an alkylene chain as defined above. If the heterocyclyl is a nitrogen-containing heterocyclyl, the heterocyclyl is optionally attached to the alkyl radical at the nitrogen atom. The alkylene chain of the heterocyclylalkyl radical is optionally substituted as defined above for an alkylene chain. The heterocyclyl part of the heterocyclylalkyl radical is optionally substituted as defined above for a heterocyclyl group.

“Heteroaryl” refers to a radical derived from a 3- to 18-membered aromatic ring radical that comprises two to seventeen carbon atoms and from one to six heteroatoms selected from nitrogen, oxygen and sulfur. As used herein, the heteroaryl radical may be a monocyclic, bicyclic, tricyclic or tetracyclic ring system, wherein at least one of the rings in the ring system is fully unsaturated, i.e., it contains a cyclic, delocalized (4n+2) π-electron system in accordance with the Hückel theory. Heteroaryl includes fused or bridged ring systems. The heteroatom(s) in the heteroaryl radical is optionally oxidized. One or more nitrogen atoms, if present, are optionally quaternized. The heteroaryl is attached to the rest of the molecule through any atom of the ring(s). Examples of heteroaryls include, but are not limited to, azepinyl, acridinyl, benzimidazolyl, benzindolyl, 1,3-benzodioxolyl, benzofuranyl, benzooxazolyl, benzo[d]thiazolyl, benzothiadiazolyl, benzo[b][1,4]dioxepinyl, benzo[b][1,4]oxazinyl, 1,4-benzodioxanyl, benzonaphthofuranyl, benzoxazolyl, benzodioxolyl, benzodioxinyl, benzopyranyl, benzopyranonyl, benzofuranyl, benzofuranonyl, benzothienyl (benzothiophenyl), benzothieno[3,2-d]pyrimidinyl, benzotriazolyl, benzo[4,6]imidazo[1,2-a]pyridinyl, carbazolyl, cinnolinyl, cyclopenta[d]pyrimidinyl, 6,7-dihydro-5H-cyclopenta[4,5]thieno[2,3-d]pyrimidinyl, 5,6-dihydrobenzo[h]quinazolinyl, 5,6-dihydrobenzo[h]cinnolinyl, 6,7-dihydro-5H-benzo[6,7]cyclohepta[1,2-c]pyridazinyl, dibenzofuranyl, dibenzothiophenyl, furanyl, furanonyl, furo[3,2-c]pyridinyl, 5,6,7,8,9,10-hexahydrocycloocta[d]pyrimidinyl, 5,6,7,8,9,10-hexahydrocycloocta[d]pyridazinyl, 5,6,7,8,9,10-hexahydrocycloocta[d]pyridinyl,isothiazolyl, imidazolyl, indazolyl, indolyl, indazolyl, isoindolyl, indolinyl, isoindolinyl, isoquinolyl, indolizinyl, isoxazolyl, 5,8-methano-5,6,7,8-tetrahydroquinazolinyl, naphthyridinyl, 1,6-naphthyridinonyl, oxadiazolyl, 2-oxoazepinyl, oxazolyl, oxiranyl, 5,6,6a,7,8,9,10,10a-octahydrobenzo[h]quinazolinyl, 1-phenyl-1H-pyrrolyl, phenazinyl, phenothiazinyl, phenoxazinyl, phthalazinyl, pteridinyl, purinyl, pyrrolyl, pyrazolyl, pyrazolo[3,4-d]pyrimidinyl, pyridinyl, pyrido[3,2-d]pyrimidinyl, pyrido[3,4-d]pyrimidinyl, pyrazinyl, pyrimidinyl, pyridazinyl, pyrrolyl, quinazolinyl, quinoxalinyl, quinolinyl, isoquinolinyl, tetrahydroquinolinyl, 5,6,7,8-tetrahydroquinazolinyl, 5,6,7,8-tetrahydrobenzo[4,5]thieno[2,3-d]pyrimidinyl, 6,7,8,9-tetrahydro-5H-cyclohepta[4,5]thieno[2,3-d]pyrimidinyl, 5,6,7,8-tetrahydropyrido[4,5-c]pyridazinyl, thiazolyl, thiadiazolyl, triazolyl, tetrazolyl, triazinyl, thieno[2,3-d]pyrimidinyl, thieno[3,2-d]pyrimidinyl, thieno[2,3-c]pridinyl, and thiophenyl (i.e., thienyl). Unless stated otherwise specifically in the specification, the term “heteroaryl” is meant to include heteroaryl radicals as defined above which are optionally substituted by one or more substituents selected from alkyl, alkenyl, alkynyl, halo, fluoroalkyl, haloalkenyl, haloalkynyl, oxo, thioxo, cyano, nitro, optionally substituted aryl, optionally substituted aralkyl, optionally substituted aralkenyl, optionally substituted aralkynyl, optionally substituted carbocyclyl, optionally substituted carbocyclylalkyl, optionally substituted heterocyclyl, optionally substituted heterocyclylalkyl, optionally substituted heteroaryl, optionally substituted heteroarylalkyl, —R^{b}—OR^{a}, —R^{b}—SR^{a}, —R^{b}—OC(O)—R^{a}, —R^{b}—N(R^{a})₂, —R^{b}—C(O)R^{a}, —R^{b}—C(O)OR^{a}, —R^{b}—C(O)N(R^{a})₂, —R^{b}—O—R^{c}—C(O)N(R^{a})₂, —R^{b}—N(R^{a})C(O)OR^{a}, —R^{b}—N(R^{a})C(O)R^{a}, —R^{b}—N(R^{a})S(O)Rₐ (where t is 1 or 2), —R^{b}—S(O)ₜOR^{a} (where t is 1 or 2) and —R^{b}—S(O)ₜN(R^{a})₂ (where t is 1 or 2), where each R^{a} is independently hydrogen, alkyl, fluoroalkyl, cycloalkyl, cycloalkylalkyl, aryl, aralkyl, heterocyclyl, heterocyclylalkyl, heteroaryl or heteroarylalkyl, each R^{b} is independently a direct bond or a straight or branched alkylene or alkenylene chain, and R^{C} is a straight or branched alkylene or alkenylene chain, and where each of the above substituents is unsubstituted unless otherwise indicated.

“N-heteroaryl” refers to a heteroaryl radical as defined above containing at least one nitrogen and where the point of attachment of the heteroaryl radical to the rest of the molecule is through a nitrogen atom in the heteroaryl radical. An N-heteroaryl radical is optionally substituted as described above for heteroaryl radicals.

“C-heteroaryl” refers to a heteroaryl radical as defined above and where the point of attachment of the heteroaryl radical to the rest of the molecule is through a carbon atom in the heteroaryl radical. A C-heteroaryl radical is optionally substituted as described above for heteroaryl radicals.

“Heteroarylalkyl” refers to a radical of the formula —R^{c}-heteroaryl, where R^{c} is an alkylene chain as defined above. If the heteroaryl is a nitrogen-containing heteroaryl, the heteroaryl is optionally attached to the alkyl radical at the nitrogen atom. The alkylene chain of the heteroarylalkyl radical is optionally substituted as defined above for an alkylene chain. The heteroaryl part of the heteroarylalkyl radical is optionally substituted as defined above for a heteroaryl group.

The compounds, or their pharmaceutically acceptable salts may contain one or more asymmetric centers and may thus give rise to enantiomers, diastereomers, and other stereoisomeric forms that may be defined, in terms of absolute stereochemistry, as (R)- or (S)- or, as (D)- or (L)- for amino acids. When the compounds described herein contain olefinic double bonds or other centers of geometric asymmetry, and unless specified otherwise, it is intended that the compounds include both E (or trans) and Z (cis) geometric isomers. Likewise, all possible isomers, as well as their racemic and optically pure forms, and all tautomeric forms are also intended to be included.

A “stereoisomer” refers to a compound made up of the same atoms bonded by the same bonds but having different three-dimensional structures, which are not interchangeable. It is therefore contemplated that various stereoisomers and mixtures thereof and includes “enantiomers,” which refers to two stereoisomers whose molecules are nonsuperimposeable mirror images of one another.

A “tautomer” refers to a proton shift from one atom of a molecule to another atom of the same molecule. The compounds presented herein may exist as tautomers. Tautomers are compounds that are interconvertible by migration of a hydrogen atom, accompanied by a switch of a single bond and adjacent double bond. In solutions where tautomerization is possible, a chemical equilibrium of the tautomers will exist. The exact ratio of the tautomers depends on several factors, including temperature, solvent, and pH. Some examples of tautomeric pairs include:

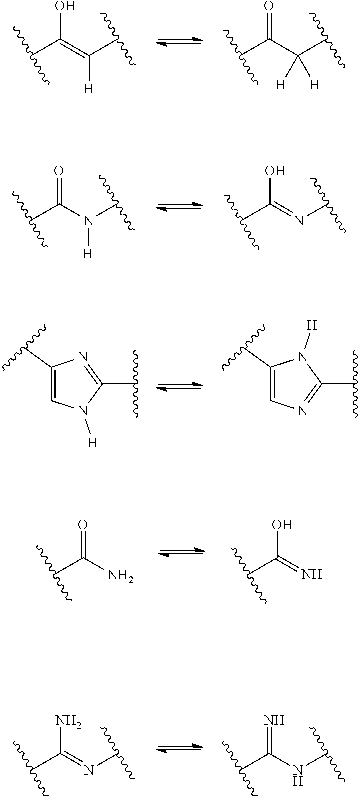

“Optional” or “optionally” means that a subsequently described event or circumstance may or may not occur and that the description includes instances when the event or circumstance occurs and instances in which it does not.

“Pharmaceutically acceptable salt” includes both acid and base addition salts. A pharmaceutically acceptable salt of any one of the alkoxyphenyl-linked amine derivative compounds described herein is intended to encompass any and all pharmaceutically suitable salt forms. Preferred pharmaceutically acceptable salts of the compounds described herein are pharmaceutically acceptable acid addition salts and pharmaceutically acceptable base addition salts.

“Pharmaceutically acceptable acid addition salt” refers to those salts which retain the biological effectiveness and properties of the free bases, which are not biologically or otherwise undesirable, and which are formed with inorganic acids such as hydrochloric acid, hydrobromic acid, sulfuric acid, nitric acid, phosphoric acid, hydroiodic acid, hydrofluoric acid, phosphorous acid, and the like. Also included are salts that are formed with organic acids such as aliphatic mono- and dicarboxylic acids, phenyl-substituted alkanoic acids, hydroxy alkanoic acids, alkanedioic acids, aromatic acids, aliphatic and aromatic sulfonic acids, etc. and include, for example, acetic acid, trifluoroacetic acid, propionic acid, glycolic acid, pyruvic acid, oxalic acid, maleic acid, malonic acid, succinic acid, fumaric acid, tartaric acid, citric acid, benzoic acid, cinnamic acid, mandelic acid, methanesulfonic acid, ethanesulfonic acid, p-toluenesulfonic acid, salicylic acid, and the like. Exemplary salts thus include sulfates, pyrosulfates, bisulfates, sulfites, bisulfites, nitrates, phosphates, monohydrogenphosphates, dihydrogenphosphates, metaphosphates, pyrophosphates, chlorides, bromides, iodides, acetates, trifluoroacetates, propionates, caprylates, isobutyrates, oxalates, malonates, succinate suberates, sebacates, fumarates, maleates, mandelates, benzoates, chlorobenzoates, methylbenzoates, dinitrobenzoates, phthalates, benzenesulfonates, toluenesulfonates, phenylacetates, citrates, lactates, malates, tartrates, methanesulfonates, and the like. Also contemplated are salts of amino acids, such as arginates, gluconates, and galacturonates (see, for example, Berge S. M. et al., “Pharmaceutical Salts,” Journal of Pharmaceutical Science, 66:1-19 (1997), which is hereby incorporated by reference in its entirety). Acid addition salts of basic compounds may be prepared by contacting the free base forms with a sufficient amount of the desired acid to produce the salt according to methods and techniques with which a skilled artisan is familiar.

“Pharmaceutically acceptable base addition salt” refers to those salts that retain the biological effectiveness and properties of the free acids, which are not biologically or otherwise undesirable. These salts are prepared from addition of an inorganic base or an organic base to the free acid. Pharmaceutically acceptable base addition salts may be formed with metals or amines, such as alkali and alkaline earth metals or organic amines. Salts derived from inorganic bases include, but are not limited to, sodium, potassium, lithium, ammonium, calcium, magnesium, iron, zinc, copper, manganese, aluminum salts and the like. Salts derived from organic bases include, but are not limited to, salts of primary, secondary, and tertiary amines, substituted amines including naturally occurring substituted amines, cyclic amines and basic ion exchange resins, for example, isopropylamine, trimethylamine, diethylamine, triethylamine, tripropylamine, ethanolamine, diethanolamine, 2-dimethylaminoethanol, 2-diethylaminoethanol, dicyclohexylamine, lysine, arginine, histidine, caffeine, procaine, N,N-dibenzylethylenediamine, chloroprocaine, hydrabamine, choline, betaine, ethylenediamine, ethylenedianiline, N-methylglucamine, glucosamine, methylglucamine, theobromine, purines, piperazine, piperidine, N-ethylpiperidine, polyamine resins and the like. See Berge et al., supra.

As used herein, “treatment” or “treating,” or “palliating” or “ameliorating” are used interchangeably herein. These terms refer to an approach for obtaining beneficial or desired results including but not limited to therapeutic benefit and/or a prophylactic benefit. By “therapeutic benefit” is meant eradication, reduction, or amelioration of the underlying disorder being treated. Also, a therapeutic benefit is achieved with the eradication, reduction, or amelioration of one or more of the physiological symptoms associated with the underlying disorder such that an improvement is observed in the patient, notwithstanding that the patient may still be afflicted with the underlying disorder. For prophylactic benefit, the compositions may be administered to a patient at risk of developing a particular disease, or to a patient reporting one or more of the physiological symptoms of a disease, even though a diagnosis of this disease may not have been made.

“Prodrug” is meant to indicate a compound that may be converted under physiological conditions or by solvolysis to a biologically active compound described herein. Thus, the term “prodrug” refers to a precursor of a biologically active compound that is pharmaceutically acceptable. A prodrug may be inactive when administered to a subject, but is converted in vivo to an active compound, for example, by hydrolysis. The prodrug compound often offers advantages of solubility, tissue compatibility or delayed release in a mammalian organism (see, e.g., Bundgard, H., Design of Prodrugs (1985), pp. 7-9, 21-24 (Elsevier, Amsterdam).

A discussion of prodrugs is provided in Higuchi, T., et al., “Pro-drugs as Novel Delivery Systems,” A.C.S. Symposium Series, Vol. 14, and in Bioreversible Carriers in Drug Design, ed. Edward B. Roche, American Pharmaceutical Association and Pergamon Press, 1987, both of which are incorporated in full by reference herein.

The term “prodrug” is also meant to include any covalently bonded carriers, which release the active compound in vivo when such prodrug is administered to a mammalian subject. Prodrugs of an active compound, as described herein, may be prepared by modifying functional groups present in the active compound in such a way that the modifications are cleaved, either in routine manipulation or in vivo, to the parent active compound. Prodrugs include compounds wherein a hydroxy, amino or mercapto group is bonded to any group that, when the prodrug of the active compound is administered to a mammalian subject, cleaves to form a free hydroxy, free amino or free mercapto group, respectively. Examples of prodrugs include, but are not limited to, acetate, formate and benzoate derivatives of alcohol or amine functional groups in the active compounds and the like.

### Peptides Complexes

The present disclosure provides methods for administering compounds that selectively bind to certain types of vascular lesion cells and tissues. As yet another example, the present disclosure provides a method for administering compounds that selectively bind to a cavernoma (also referred to as cavernous angiomas, cavernous hemangiomas, or cerebral cavernous malformation (CCM)) cells and tissues, arteriovenous malformation (also referred to as arteriovenous angiomas, arteriovenous hemangiomas, or cerebral arteriovenous malformation (CAM)) cells and tissues, an aneurysm (e.g., including abdominal aortic, thoracic aortic, and cerebral aneurysm) cells and tissues, or a spinal dural arteriovenous fistula cells and tissues. In various aspects, these compounds can comprise a peptide portion and a detectable agent conjugated together.

In various aspects of the compounds used in the present disclosure, the peptide portions of the compounds described herein have certain features in common with the native chlorotoxin (CTX) peptide. The native chlorotoxin peptide was originally isolated from the scorpion Leiurus quinquestriatus. Chlorotoxin is a 36 amino acid peptide that selectively binds to or accumulates in cancerous cells. Although the peptide portions of the present compounds have retained at least some of the cancer-cell binding activity of chlorotoxin, they also unexpectedly bind and accumulate in vascular lesions. The vascular lesion accumulation or binding activity of the compounds used in the present disclosure provides certain advantages for the detection and treatment of vascular lesions because it facilitates the selective localization of detectable agents and therapeutic agents to the vascular lesion cells for the detection and treatment of vascular lesion. In certain aspects, peptides used in the present disclosure are conjugated to moieties, such as detectable labels (e.g., dyes or radiolabels) that are detected (e.g., visualized) in a subject. In some aspects, the peptides including chlorotoxin and/or chlorotoxin variants are conjugated to detectable labels to enable tracking of the bio-distribution of a conjugated peptide. The fluorescent moiety can be covalently coupled to the peptide and/or peptide variants to allow for the visualization of the conjugate by fluorescence imaging, either directly or through a linker as described herein and known to one of ordinary skill in the art. Linker moieties can include cleavable (e.g., pH sensitive or enzyme-labile linkers) or stable linkers.

In some aspects, the fluorescent label used has emission characteristics that are desired for a particular application. Fluorophores can be conjugated or fused to another moiety as described herein and be used to home, target, migrate to, be retained by, accumulate in, and/or bind to, or be directed to specific organs, substructures within organs, tissues, targets or cells and used in conjunction with the compounds and methods herein. Exemplary organs and organ substructures include the brain and other organs and organ structures such as brain, heart, lung, kidney, liver, CNS (e.g., spine) or pancreas or in the extremities (e.g., legs, neck, and arms). The fluorophore emission can comprise an infrared, near infrared, blue or ultraviolet emission.

In some embodiments, compounds and methods herein are used as imaging agents to detect their fluorophores have an absorption wavelength of about 10 nm to about 200 nm. In some embodiments, compounds and methods herein are used as imaging agents to detect their fluorophores have an absorption wavelength of about 10 nm to about 20 nm, about 10 nm to about 30 nm, about 10 nm to about 40 nm, about 10 nm to about 50 nm, about 10 nm to about 75 nm, about 10 nm to about 100 nm, about 10 nm to about 125 nm, about 10 nm to about 150 nm, about 10 nm to about 200 nm, about 20 nm to about 30 nm, about 20 nm to about 40 nm, about 20 nm to about 50 nm, about 20 nm to about 75 nm, about 20 nm to about 100 nm, about 20 nm to about 125 nm, about 20 nm to about 150 nm, about 20 nm to about 200 nm, about 30 nm to about 40 nm, about 30 nm to about 50 nm, about 30 nm to about 75 nm, about 30 nm to about 100 nm, about 30 nm to about 125 nm, about 30 nm to about 150 nm, about 30 nm to about 200 nm, about 40 nm to about 50 nm, about 40 nm to about 75 nm, about 40 nm to about 100 nm, about 40 nm to about 125 nm, about 40 nm to about 150 nm, about 40 nm to about 200 nm, about 50 nm to about 75 nm, about 50 nm to about 100 nm, about 50 nm to about 125 nm, about 50 nm to about 150 nm, about 50 nm to about 200 nm, about 75 nm to about 100 nm, about 75 nm to about 125 nm, about 75 nm to about 150 nm, about 75 nm to about 200 nm, about 100 nm to about 125 nm, about 100 nm to about 150 nm, about 100 nm to about 200 nm, about 125 nm to about 150 nm, about 125 nm to about 200 nm, or about 150 nm to about 200 nm. In some embodiments, compounds and methods herein are used as imaging agents to detect their fluorophores have an absorption wavelength of about 10 nm, about 20 nm, about 30 nm, about 40 nm, about 50 nm, about 75 nm, about 100 nm, about 125 nm, about 150 nm, or about 200 nm. In some embodiments, compounds and methods herein are used as imaging agents to detect their fluorophores have an absorption wavelength of at least about 10 nm, about 20 nm, about 30 nm, about 40 nm, about 50 nm, about 75 nm, about 100 nm, about 125 nm, or about 150 nm. In some embodiments, compounds and methods herein are used as imaging agents to detect their fluorophores have an absorption wavelength of at most about 20 nm, about 30 nm, about 40 nm, about 50 nm, about 75 nm, about 100 nm, about 125 nm, about 150 nm, or about 200 nm.

In some embodiments, the compounds and methods herein are used as imaging agents to detect their fluorophore emissions. The fluorophores emissions can comprise an ultraviolet emission. The ultraviolet emissions can have a wavelength from 10 nm to 400 nm, and up to 450 nm or 460 nm into the blue light spectrum, including fluorophores with absorption wavelengths in the ranges disclosed herein, including 10-20 nm, 20-30 nm, 30-40 nm, 40-50 nm, 50-60 nm, 60-70 nm, 70-80 nm, 80-90 nm, 90-100 nm, 100-110 nm, 110-120 nm, 120-130 nm, 130-140 nm, 140-150 nm, 150-160 nm, 160-170 nm, 170-180 nm, 180-190 nm, 190-200 nm, 200-210 nm, 210-220 nm, 220-230 nm, 230-240 nm, 240-250 nm, 250-260 nm, 260-270 nm, 270-280 nm, 280-290 nm, 290-300 nm, 300-310 nm, 310-320 nm, 320-330 nm, 330-340 nm, 340-350 nm, 350-360 nm, 360-370 nm, 370-380 nm, 380-390 nm, 390-400 nm, 400-410 nm, 410-420 nm, 420-430 nm, 430-440 nm, 440-450 nm, 450-460 nm, 300-350 nm, 325-375 nm, 350-400 nm, 400-450 nm, a wavelength in the range of 340 nm to 400 nm, 360 to 420 nm, 380 nm to 440 nm, 400 nm to 450 nm, 400 nm to 460 nm or any wavelength within any of these foregoing ranges.

In some embodiments, compounds and methods herein are used as imaging agents to detect their fluorophores have an absorption wavelength of about 200 nm to about 1,000 nm. In some embodiments, compounds and methods herein are used as imaging agents to detect their fluorophores have an absorption wavelength of about 200 nm to about 250 nm, about 200 nm to about 300 nm, about 200 nm to about 350 nm, about 200 nm to about 400 nm, about 200 nm to about 450 nm, about 200 nm to about 500 nm, about 200 nm to about 600 nm, about 200 nm to about 700 nm, about 200 nm to about 800 nm, about 200 nm to about 900 nm, about 200 nm to about 1,000 nm, about 250 nm to about 300 nm, about 250 nm to about 350 nm, about 250 nm to about 400 nm, about 250 nm to about 450 nm, about 250 nm to about 500 nm, about 250 nm to about 600 nm, about 250 nm to about 700 nm, about 250 nm to about 800 nm, about 250 nm to about 900 nm, about 250 nm to about 1,000 nm, about 300 nm to about 350 nm, about 300 nm to about 400 nm, about 300 nm to about 450 nm, about 300 nm to about 500 nm, about 300 nm to about 600 nm, about 300 nm to about 700 nm, about 300 nm to about 800 nm, about 300 nm to about 900 nm, about 300 nm to about 1,000 nm, about 350 nm to about 400 nm, about 350 nm to about 450 nm, about 350 nm to about 500 nm, about 350 nm to about 600 nm, about 350 nm to about 700 nm, about 350 nm to about 800 nm, about 350 nm to about 900 nm, about 350 nm to about 1,000 nm, about 400 nm to about 450 nm, about 400 nm to about 500 nm, about 400 nm to about 600 nm, about 400 nm to about 700 nm, about 400 nm to about 800 nm, about 400 nm to about 900 nm, about 400 nm to about 1,000 nm, about 450 nm to about 500 nm, about 450 nm to about 600 nm, about 450 nm to about 700 nm, about 450 nm to about 800 nm, about 450 nm to about 900 nm, about 450 nm to about 1,000 nm, about 500 nm to about 600 nm, about 500 nm to about 700 nm, about 500 nm to about 800 nm, about 500 nm to about 900 nm, about 500 nm to about 1,000 nm, about 600 nm to about 700 nm, about 600 nm to about 800 nm, about 600 nm to about 900 nm, about 600 nm to about 1,000 nm, about 700 nm to about 800 nm, about 700 nm to about 900 nm, about 700 nm to about 1,000 nm, about 800 nm to about 900 nm, about 800 nm to about 1,000 nm, or about 900 nm to about 1,000 nm. In some embodiments, compounds and methods herein are used as imaging agents to detect their fluorophores have an absorption wavelength of about 200 nm, about 250 nm, about 300 nm, about 350 nm, about 400 nm, about 450 nm, about 500 nm, about 600 nm, about 700 nm, about 800 nm, about 900 nm, or about 1,000 nm. In some embodiments, compounds and methods herein are used as imaging agents to detect their fluorophores have an absorption wavelength of at least about 200 nm, about 250 nm, about 300 nm, about 350 nm, about 400 nm, about 450 nm, about 500 nm, about 600 nm, about 700 nm, about 800 nm, or about 900 nm. In some embodiments, compounds and methods herein are used as imaging agents to detect their fluorophores have an absorption wavelength of at most about 250 nm, about 300 nm, about 350 nm, about 400 nm, about 450 nm, about 500 nm, about 600 nm, about 700 nm, about 800 nm, about 900 nm, or about 1,000 nm.

In some embodiments, compounds and methods herein are used as imaging agents to detect their fluorophores have an absorption wavelength of about 1,000 nm to about 4,000 nm. In some embodiments, compounds and methods herein are used as imaging agents to detect their fluorophores have an absorption wavelength of about 1,000 nm to about 1,250 nm, about 1,000 nm to about 1,500 nm, about 1,000 nm to about 1,750 nm, about 1,000 nm to about 2,000 nm, about 1,000 nm to about 2,250 nm, about 1,000 nm to about 2,500 nm, about 1,000 nm to about 2,750 nm, about 1,000 nm to about 3,000 nm, about 1,000 nm to about 3,250 nm, about 1,000 nm to about 3,500 nm, about 1,000 nm to about 4,000 nm, about 1,250 nm to about 1,500 nm, about 1,250 nm to about 1,750 nm, about 1,250 nm to about 2,000 nm, about 1,250 nm to about 2,250 nm, about 1,250 nm to about 2,500 nm, about 1,250 nm to about 2,750 nm, about 1,250 nm to about 3,000 nm, about 1,250 nm to about 3,250 nm, about 1,250 nm to about 3,500 nm, about 1,250 nm to about 4,000 nm, about 1,500 nm to about 1,750 nm, about 1,500 nm to about 2,000 nm, about 1,500 nm to about 2,250 nm, about 1,500 nm to about 2,500 nm, about 1,500 nm to about 2,750 nm, about 1,500 nm to about 3,000 nm, about 1,500 nm to about 3,250 nm, about 1,500 nm to about 3,500 nm, about 1,500 nm to about 4,000 nm, about 1,750 nm to about 2,000 nm, about 1,750 nm to about 2,250 nm, about 1,750 nm to about 2,500 nm, about 1,750 nm to about 2,750 nm, about 1,750 nm to about 3,000 nm, about 1,750 nm to about 3,250 nm, about 1,750 nm to about 3,500 nm, about 1,750 nm to about 4,000 nm, about 2,000 nm to about 2,250 nm, about 2,000 nm to about 2,500 nm, about 2,000 nm to about 2,750 nm, about 2,000 nm to about 3,000 nm, about 2,000 nm to about 3,250 nm, about 2,000 nm to about 3,500 nm, about 2,000 nm to about 4,000 nm, about 2,250 nm to about 2,500 nm, about 2,250 nm to about 2,750 nm, about 2,250 nm to about 3,000 nm, about 2,250 nm to about 3,250 nm, about 2,250 nm to about 3,500 nm, about 2,250 nm to about 4,000 nm, about 2,500 nm to about 2,750 nm, about 2,500 nm to about 3,000 nm, about 2,500 nm to about 3,250 nm, about 2,500 nm to about 3,500 nm, about 2,500 nm to about 4,000 nm, about 2,750 nm to about 3,000 nm, about 2,750 nm to about 3,250 nm, about 2,750 nm to about 3,500 nm, about 2,750 nm to about 4,000 nm, about 3,000 nm to about 3,250 nm, about 3,000 nm to about 3,500 nm, about 3,000 nm to about 4,000 nm, about 3,250 nm to about 3,500 nm, about 3,250 nm to about 4,000 nm, or about 3,500 nm to about 4,000 nm. In some embodiments, compounds and methods herein are used as imaging agents to detect their fluorophores have an absorption wavelength of about 1,000 nm, about 1,250 nm, about 1,500 nm, about 1,750 nm, about 2,000 nm, about 2,250 nm, about 2,500 nm, about 2,750 nm, about 3,000 nm, about 3,250 nm, about 3,500 nm, or about 4,000 nm. In some embodiments, compounds and methods herein are used as imaging agents to detect their fluorophores have an absorption wavelength of at least about 1,000 nm, about 1,250 nm, about 1,500 nm, about 1,750 nm, about 2,000 nm, about 2,250 nm, about 2,500 nm, about 2,750 nm, about 3,000 nm, about 3,250 nm, or about 3,500 nm. In some embodiments, compounds and methods herein are used as imaging agents to detect their fluorophores have an absorption wavelength of at most about 1,250 nm, about 1,500 nm, about 1,750 nm, about 2,000 nm, about 2,250 nm, about 2,500 nm, about 2,750 nm, about 3,000 nm, about 3,250 nm, about 3,500 nm, or about 4,000 nm. It is understood that the absorption spectra of fluorophores and fluorescent dyes may vary when conjugated to a peptide and one of skill in the art would appreciate that any of the foregoing absorption values of the fluorophore or dye could be +/−10 nm, +/−3 nm, +/−2 nm, or +/−1 nm, or +/−10%, +/−5%, +/−1% in the context of the compounds and methods herein. In some embodiments, depending on the environment that the fluorophore molecule is in (e.g., surgical bed, vascular lesion tissue, solution, and the like), the fluorophore molecule has an optimal excitation spectrum) from 600 nm to 900 nm. Some other exemplary dyes used in the present disclosure can include near-infrared dyes, such as, but not limited to, DyLight-680, DyLight-750, VivoTag-750, DyLight-800, IRDye-800, VivoTag-680, Cy5.5, or an indocyanine green (ICG). In some aspects, near infrared dyes often include cyanine dyes. Additional non-limiting examples of fluorescent dyes for use as a conjugating molecule in the present disclosure can include acradine orange or yellow, Alexa Fluors and any derivative thereof, 7-actinomycin D, 8-anilinonaphthalene-1-sulfonic acid, ATTO dye and any derivative thereof, auramine-rhodamine stain and any derivative thereof, bensantrhone, bimane, 9-10-bis(phenylethynyl)anthracene, 5,12-bis(phenylethynyl)naththacene, bisbenzimide, brainbow, calcein, carbodyfluorescein and any derivative thereof, 1-chloro-9,10-bis(phenylethynyl)anthracene and any derivative thereof, DAPI, DiOC6, DyLight Fluors and any derivative thereof, epicocconone, ethidium bromide, FlAsH-EDT2, Fluo dye and any derivative thereof, FluoProbe and any derivative thereof, Fluorescein and any derivative thereof, Fura and any derivative thereof, GelGreen and any derivative thereof, GelRed and any derivative thereof, fluorescent proteins and any derivative thereof, m isoform proteins and any derivative thereof such as for example mCherry, hetamethine dye and any derivative thereof, hoeschst stain, iminocoumarin, indian yellow, indo-1 and any derivative thereof, laurdan, lucifer yellow and any derivative thereof, luciferin and any derivative thereof, luciferase and any derivative thereof, mercocyanine and any derivative thereof, nile dyes and any derivative thereof, perylene, phloxine, phyco dye and any derivative thereof, propium iodide, pyranine, rhodamine and any derivative thereof, ribogreen, RoGFP, rubrene, stilbene and any derivative thereof, sulforhodamine and any derivative thereof, SYBR and any derivative thereof, synapto-pHluorin, tetraphenyl butadiene, tetrasodium tris, Texas Red, Titan Yellow, TSQ, umbelliferone, violanthrone, yellow fluroescent protein, YOYO-1 and ZW800. Other suitable fluorescent dyes include, but are not limited to, fluorescein and fluorescein dyes (e.g., fluorescein isothiocyanine or FITC, naphthofluorescein, 4′,5′-dichloro-2′,7′-dimethoxyfluorescein, 6-carboxyfluorescein or FAM, etc.), carbocyanine, merocyanine, styryl dyes, oxonol dyes, phycoerythrin, erythrosin, eosin, rhodamine dyes (e.g., carboxytetramethyl-rhodamine or TAMRA, carboxyrhodamine 6G, carboxy-X-rhodamine (ROX), lissamine rhodamine B, rhodamine 6G, rhodamine Green, rhodamine Red, tetramethylrhodamine (TMR), etc.), coumarin and coumarin dyes (e.g., methoxycoumarin, dialkylaminocoumarin, hydroxycoumarin, aminomethylcoumarin (AMCA), etc.), Oregon Green Dyes (e.g., Oregon Green 488, Oregon Green 500, Oregon Green 514., etc.), Texas Red, Texas Red-X, SPECTRUM RED, SPECTRUM GREEN, cyanine dyes (e.g., CY-3, Cy-5, CY-3.5, CY-5.5, etc.), ALEXA FLUOR dyes (e.g., ALEXA FLUOR 350, ALEXA FLUOR 488, ALEXA FLUOR 532, ALEXA FLUOR 546, ALEXA FLUOR 568, ALEXA FLUOR 594, ALEXA FLUOR 633, ALEXA FLUOR 660, ALEXA FLUOR 680, etc.), BODIPY dyes (e.g., BODIPY FL, BODIPY R6G, BODIPY TMR, BODIPY TR, BODIPY 530/550, BODIPY 558/568, BODIPY 564/570, BODIPY 576/589, BODIPY 581/591, BODIPY 630/650, BODIPY 650/665, etc.), IRDyes (e.g., IRD40, IRD 700, IRD 800, etc.), and the like. In some aspects, complexes of the present disclosure comprise other dyes, including but not limited to those provided below in TABLE 1.

Regarding TABLE 1, the peak absorption and emission values for a given fluorophore can vary depending on the environment (e.g. solution, tissue, etc.) that the fluorophore is present in as well as the concentration of fluorophore or fluorophore conjugate utilized.

TABLE 1
Exemplary Fluorescent Reporter Molecules with Peak Absorbance
(Abs.) and Emission (Em.) Wavelengths Specified (nm)

| | Peak | Peak |
|---|---|---|
| Dye | Abs. | Em. |
| Methoxycoumarin | 360 | 410 |
| Fluospheres Blue | 356 | 412 |
| Cascade Blue | 377 | 420 |
| PBFI | 360 | 420 |
| DyeLight 405 | 400 | 420 |
| Cascade Blue | 400 | 420 |
| Alexa Fluor 405 | 401 | 421 |
| Alexa Fluor 405 | 401 | 421 |
| LysoTracker Blue | 373 | 422 |
| LysoSensorBlue | 374 | 424 |
| AMCA | 345 | 425 |
| True Blue | 365 | 425 |
| 7-amino-4-methylcoumarin | 351 | 430 |
| (AMC) | | |
| Phorwite AR | 360 | 430 |
| DyLight 350 | 353 | 432 |
| Uvitex SFC | 365 | 435 |
| 4-methylumbelliferone | 360 | 440 |
| CellTrace Calcein Blue | 373 | 440 |
| Calcofluor White | 350 | 440 |
| Fast Blue | 360 | 440 |
| LysoSensor Yellow/Blue (pH | 329 | 440 |
| 8.0) | | |
| LysoSensor Yellow/Blue (pH | 329 | 440 |
| 8.0) | | |
| LysoSensor Yellow/Blue (pH | 329 | 440 |
| 8.0) | | |
| LysoSensor Yellow/Blue (pH | 329 | 440 |
| 8.0) | | |
| Alexa Fluor 350 | 346 | 442 |
| AMCA-X | 353 | 442 |
| LIVE/DEAD Fixable Blue D | 344 | 442 |
| ead Cell Stain | | |
| Y66H | 360 | 442 |
| ABQ | 344 | 445 |
| BFP | 382 | 448 |
| BFP | 382 | 448 |
| 7-hydroxy-4-methylcoumarin | 360 | 449 |
| SpectrumBlue | 405 | 449 |
| DiFMU (pH 9.0) | 357 | 450 |
| sgBFP (Super Glow BFP) | 387 | 450 |
| SpectrumBlue | 400 | 450 |
| CellTrace Calcein Violet | 401 | 451 |
| DAPI | 345 | 455 |
| NucBlue Fixed Cell Stain | 345 | 455 |
| Pacific Blue | 405 | 455 |
| LysoSensor Green (pH 5) | 442 | 505 |
| SYTO 13 | 487 | 505 |
| SYTO 13 | 487 | 505 |
| SYTO 13 | 487 | 505 |
| SYTO 13 | 487 | 505 |
| DiO (Vybrant DiO) | 489 | 506 |
| HCS LipidTox Green | 498 | 506 |
| LIVE/DEAD Fixable Green | 498 | 506 |
| LIVE/DEAD Fixable Green | 498 | 506 |
| ATTO 465 | 453 | 507 |
| CellLights GFP | 488 | 507 |
| CellEvent Caspase-3/7 Green | 488 | 507 |
| Diversa Green-FP | 484 | 507 |
| GFP (EGFP) | 488 | 507 |
| S65C | 479 | 507 |
| YO-PRO-1 | 491 | |
| GFP | 488 | 507 |
| YO-PRO-1 | 491 | 507 |
| GFP | 488 | 507 |
| YO-PRO-1 | 491 | 507 |
| GFP | 488 | 507 |
| YO-PRO-1 | 491 | 507 |
| Premo FUCCI Cell Cycle | 474 | 507 |
| Sensor (S/G2/M phases) | | 509 |
| sgGFP (Super Glow GFP) | 474 | 509 |
| wtGFP (wild type GFP, non- | 475 | 509 |
| UV excitation) | | |
| YOYO-1 | 491 | 509 |
| YOYO-1 | 491 | 509 |
| YOYO-1 | 491 | 509 |
| YOYO-1 | 491 | 509 |
| YOYO-1 | 491 | 509 |
| HPTS (Solvent Green 7) | 455 | 510 |
| Nitrobenzoxadiazole | 465 | 510 |
| S65L | 484 | 510 |
| LysoTracker Green | 504 | 511 |
| S65T | 488 | 511 |
| LysoTracker Green | 504 | 511 |
| LysoTracker Green | 504 | 511 |
| MitoTracker Green FM | 490 | 512 |
| MitoTracker Green FM | 490 | 512 |
| MitoTracker Green FM | 490 | 512 |
| MitoTracker Green FM | 490 | 512 |
| Fluo Spheres Yellow-Green | 501 | 513 |
| Spectra mgreen | 498 | 522 |
| NucGreen Dead Cell Stain | 504 | 523 |
| Rhodamine Green | 497 | 523 |
| Rhodol Green | 496 | 523 |
| SYTOX Green | 504 | 523 |
| Rhodamine Green | 497 | 523 |
| Rhodamine Green | 497 | 523 |
| Rhodamine Green | 497 | 523 |
| Neurotrace 500/525 Green | 497 | 524 |
| Oregon Green 488 | 498 | 524 |
| SYBR Safe | 507 | 524 |
| NeuroTrace 500/525 Nissl stain | 497 | 524 |
| Oregon Green 488 | 498 | 524 |
| NeuroTrace 500/525 Nissl stain | 497 | 524 |
| Oregon Green 488 | 498 | 524 |
| NeuroTrace 500/525 Nissl stain | 497 | 524 |
| NeuroTrace 500/525 Nissl stain | 497 | 524 |
| Oregon Green 488 | 498 | 524 |
| Dansyl | 335 | 525 |
| Fluoro-Jade B | 480 | 525 |
| Qdot 525 | UV | 525 |
| SYTO 11 | 506 | 525 |
| Qdot 525 | UV | 525 |
| Qdot 525 | UV | 525 |
| Acridine Orange + DNA | 500 | 526 |
| LIVE/DEAD Fixable Green | 498 | 526 |
| Surf Green EX | 469 | 526 |
| Acridine Orange + DNA | 500 | 526 |
| Acridine Orange + DNA | 500 | 526 |
| Acridine Orange + DNA | 500 | 526 |
| Acridine Orange (+DNA) | 500 | 526 |
| ThiolTracker Violet | 405 | 526 |
| ThiolTracker Violet | 405 | 526 |
| ThiolTracker Violet | 405 | 526 |
| ThiolTracker Violet | 405 | 526 |
| Acridine Orange (+DNA) | 500 | 526 |
| ThiolTracker Violet | 405 | 526 |
| SYTO RNASelect | 503 | 527 |
| EYFP | 514 | 527 |
| SYTO RNASelect | 503 | 527 |
| SYTO RNASelect | 503 | 527 |
| Calcein | 494 | 540 |
| Calcium Green-1 | 506 | 540 |
| Catskill Green 540 | 482 | 540 |
| CellTracker Green | 490 | 540 |
| CFDA | 494 | 540 |
| CFP | 434 | 540 |
| Cy2 | 492 | 540 |
| CyQUANT Direct | 500 | 540 |
| (CyQUANT GR) | | |
| DAF-FM | 493 | 540 |
| Emerald Green | 490 | 540 |
| Fluo-3 | 506 | 540 |
| Fluo-4 | 494 | 540 |
| H2DCFDA (H2-DCF, DCFR) | 504 | 540 |
| Alexa Fluor 430 | 434 | 540 |
| Alexa Fluor 430 | 432 | 540 |
| BCECF (pH 5.2) | 499 | 540 |
| Calcein | 494 | 540 |
| CellTracker Green CMFDA | 490 | 540 |
| CFP | 434 | 540 |
| Cy2 | 492 | 540 |
| CyQUANT Direct | 500 | 540 |
| DAF-FM | 493 | 540 |
| Fluo-4 | 494 | 540 |
| Alexa Fluor 430 | 432 | 540 |
| BCECF (pH 5.2) | 499 | 540 |
| Calcein | 494 | 540 |
| CellTracker Green CMFDA | 490 | 540 |
| CFP | 434 | 540 |
| Cy2 | 492 | 540 |
| CyQUANT Direct | 500 | 540 |
| Alexa Fluor 430 | 432 | 540 |
| BCECF (pH 5.2) | 499 | 540 |
| CFP | 434 | 540 |
| Cy2 | 492 | 540 |
| Alexa Fluor 430 | 432 | 540 |
| BCECF (pH 5.2) | 499 | 540 |
| Alexa Fluor 430 | 432 | 540 |
| BCECF (pH 5.2) | 499 | 540 |
| Alexa Fluor 430 | 432 | 540 |
| BCECF (pH 5.2) | 499 | 540 |
| Calcein | 494 | 540 |
| CellTracker Green CMFDA | 490 | 540 |
| Qdot 565 | UV | 565 |
| DiI (CellTracker DiI) | 551 | 568 |
| mOrange | 548 | 568 |
| OFP | 546 | 568 |
| Bodipy TMR | 544 | 569 |
| Cy3 | 552 | 570 |
| PO-PRO-3 | 539 | 570 |
| SYTOX Orange | 567 | 570 |
| CellMask Orange | 556 | 571 |
| Alexa Fluor 546 | 561 | 572 |
| POPO-3 | 532 | 573 |
| TurboRFP | 553 | 574 |
| Calcium Orange | 549 | 575 |
| CellTracker Orange | 547 | 575 |
| LIVE/DEAD Fixable Yellow | 405 | 575 |
| LIVE/DEAD Fixable Yellow | 405 | 575 |
| LIVE/DEAD Fixable Yellow | 405 | 575 |
| LIVE/DEAD Fixable Yellow | 405 | 575 |
| LIVE/DEAD Fixable Yellow | 405 | 575 |
| LIVE/DEAD Fixable Yellow | 405 | 575 |
| DyLight 594 | 562 | 576 |
| MitoTracker Orange | 551 | 576 |
| CMTMRos(MitoTracker | | |
| Orange CM-H2TMRos) | | |
| Phycoerythrin (PE, R- | 567 | 576 |
| phycoerythrin) | | |
| Rhod-2 | 551 | 576 |
| Rhodamine Phalloidin | 557 | 576 |
| X-Rhod-1 | 570 | 576 |
| DsRed-Express | 557 | 579 |
| Rhodamine Red | 560 | 580 |
| TAMRA | 565 | 580 |
| Tetramethylrhodamine (TRI | 555 | 580 |
| TC) | | |
| dTomato | 554 | 581 |
| DsRed2 | 563 | 582 |
| Amplex Ultra Red | 567 | 582 |
| Amplex Red | 571 | 583 |
| Amplex UltraRed | 568 | 583 |
| Amplex Red | 570 | 583 |
| Premo FUCCI Cell Cycle | 555 | 584 |
| Sensor (G1 phase) | | |
| TagRFP | 555 | 584 |
| CellLights RFP | 552 | 585 |
| mTangerine | 568 | 585 |
| CellTracker Red | 585 | 612 |
| LIVE/DEAD Fixable Red | 593 | 613 |
| CellTracker Red CMTPX | 584 | 613 |
| LIVE/DEAD | 595 | 613 |
| Fixable Red Dead Cell stain | | |
| DiA (FAST DiA) | 491 | 613 |
| DiA | 491 | 613 |
| HCS CellMask Red stain | 587 | 614 |
| HCS LipidTox Red | 582 | 615 |
| HCS LipidTOX Red | 582 | 615 |
| mCherry | 587 | 615 |
| Texas Red | 592 | 615 |
| Ethidium Homodimer-1 | 530 | 618 |
| (EthD-1) | | |
| Propidium Iodide (PI) | 530 | 618 |
| Alexa Fluor 594 | 590 | 618 |
| Click-iT Alexa Fluor 594 | 590 | 618 |
| DyLight 594 | 593 | 618 |
| SYPRO Ruby | 450 | 618 |
| SYPRO Ruby | 450 | 618 |
| SYPRO Ruby | 450 | 618 |
| SYPRO Ruby | 450 | 618 |
| SYPRO Ruby | 450 | 618 |
| SYPRO Ruby | 450 | 618 |
| Bodipy TR-X | 588 | 621 |
| CellTrace BODIPY TR methyl | 597 | 625 |
| esther | | |
| mRaspberry | 598 | 625 |
| Qdot 625 | UV | 625 |
| Qdot 625 | UV | 625 |
| FM 1-43 | 510 | 626 |
| FM 1-43 | 510 | 626 |
| FM 1-43 | 510 | 626 |
| FM 1-43 | 510 | 626 |
| FM 1-43 | 510 | 626 |
| FM 1-43 | 510 | 626 |
| FM 1-43 | 510 | 626 |
| FM 1-43 | 510 | 626 |
| YO-PRO-3 | 612 | 628 |
| Alexa Fluor 610 | 610 | 629 |
| Magic Red | 570 | 630 |
| CTC Formazan | 450 | 630 |
| CTC Formazan | 450 | 630 |
| YOYO-3 | 612 | 631 |
| Fura Red (+Ca2+) | 436 | 655 |
| Fura Red (+Ca2+) | 436 | 655 |
| Qdot 655 | UV | 655 |
| Fura Red (+Ca2+) | 436 | 655 |
| Fura Red (+Ca2+) | 436 | 655 |
| Qdot 655 | UV | 655 |
| FxCycle Far Red | 641 | 657 |
| TO-PRO-3 | 642 | 657 |
| DDAO | 648 | 658 |
| DyLight 633 | 638 | 658 |
| SYTOX Red | 640 | 658 |
| ATTO 635 | 635 | 658 |
| APC (Allophycocyanin) | 651 | 660 |
| MitoTracker Deep Red FM | 641 | 661 |
| NucRed Dead 647 | 642 | 661 |
| TOTO-3 | 642 | 661 |
| BODIPY 650/665 | 647 | 665 |
| CellRox Deep Red | 640 | 665 |
| LIVE/DEAD Fixable Far | 650 | 665 |
| Red | | |
| Cy5 | 648 | 666 |
| Lysotracker Deep Red | 647 | 668 |
| Alexa Fluor 647 | 650 | 670 |
| Click-iT Alexa Fluor 647 | 650 | 670 |
| DiD (Vybrant DiD) | 645 | 670 |
| HCS CellMask Deep Red stain | 649 | 670 |
| ATTO 647 | 644 | 670 |
| Fura Red (−Ca2+) | 473 | 670 |
| Fura Red (−Ca2+) | 473 | 670 |
| Fura Red (−Ca2+) | 473 | 670 |
| Fura Red (−Ca2+) | 473 | 670 |
| Fura Red (−Ca2+) | 473 | 670 |
| DyLight 649 | 654 | 673 |
| Carboxynaphthofluorescein | 600 | 674 |
| PerCP | 488 | 675 |
| CellMask Deep Red plasma | 658 | 676 |
| membrane stain | | |
| DRAQ5 | 650 | 680 |
| SYTO 60 | 649 | 681 |
| SYTO 62 | 650 | 681 |
| SYTO 60 | 650 | 681 |
| FluoSpheres dark red | 657 | 683 |
| microspheres | | |
| Pacific Blue | 410 | 455 |
| PO-PRO-1 | 435 | 455 |
| PO-PRO-1 | 435 | 455 |
| POPO-1 | 434 | 456 |
| POPO-1 | 434 | 456 |
| TagBFP | 402 | 457 |
| Marina Blue | 365 | 460 |
| SITS | 365 | 460 |
| Thioflavin TCN | 350 | 460 |
| Monochlorobimane(mBCI) | 380 | 461 |
| Quinine Sulfate | 349 | 461 |
| Acridine | 362 | 462 |
| CellLights CFP | 434 | 477 |
| ECFP | 434 | 477 |
| CFP | 434 | 477 |
| 1,8-ANS | 372 | 480 |
| SYTOX Blue | 444 | 480 |
| SYTOX Blue | 444 | 480 |
| Hoechst 33342 | 347 | 483 |
| NucBlue Live Cell Stain | 347 | 483 |
| Thiolyte | 378 | 483 |
| SYTO 45 | 452 | 484 |
| SYTO 45 | 452 | 484 |
| SYTO 45 | 452 | 484 |
| SYTO 45 | 452 | 484 |
| SYTO 45 | 452 | 484 |
| Hoechst 33258 | 345 | 487 |
| AmCyan | 548 | 489 |
| Auramine 0 | 445 | 500 |
| SYTO 9 | 482 | 500 |
| SYTO 9 | 482 | 500 |
| SYTO 9 | 482 | 500 |
| SYTO 9 | 482 | 500 |
| SYTO 9 | 482 | 500 |
| DiO | 484 | 501 |
| DiO | 484 | 501 |
| DiO | 484 | 501 |
| LysoSensor Green | 448 | 503 |
| LysoSensor Green | 448 | 503 |
| LysoSensor Green | 448 | 503 |
| LysoSensor Green | 448 | 503 |
| LysoSensor Green | 448 | 503 |
| SYTO 13 | 487 | 505 |
| Evans Blue | 460 | 515 |
| Evans Blue | 460 | 515 |
| rsGFP (red shifted GFP, S65 T) | 498 | 516 |
| CellTracker Violet BMQC | 415 | 516 |
| HCS CellMask Green | 493 | 516 |
| CellTracker Violet BMQC | 415 | 516 |
| CellTracker Violet BMQC | 415 | 516 |
| CellTracker Violet BMQC | 415 | 516 |
| CellTracker Violet BMQC | 415 | 516 |
| HCS CellMask Green | 493 | 516 |
| 5-carboxyfluorescein(5- | 492 | 518 |
| FAM) | | |
| ActinGreen (Alexa Fluor 488 | 496 | 518 |
| phalloidin) | | |
| Alexa Fluor 488 | 496 | 518 |
| Click-iT EdU Alexa Fluor | 496 | 518 |
| 488 | | |
| DyLight + CHO 488 | 493 | 518 |
| Fluoro-Emerald | 494 | 518 |
| Aiexa Fluor 488 | 496 | 518 |
| Carboxyfluorescein (5-FAM) | 492 | 518 |
| Aiexa Fluor 488 | 496 | 518 |
| Carboxyfluorescein (5-FAM) | 492 | 518 |
| CellRox Green | 485 | 520 |
| FITC (Fluorescein) | 492 | 520 |
| Fluor-X | 494 | 520 |
| Rhodamine 110 | 496 | 520 |
| SYTO 16 | 490 | 520 |
| FITC | 492 | 520 |
| Rhodamine 110 | 496 | 520 |
| SYTO 16 | 490 | 520 |
| FITC | 492 | 520 |
| Rhodamine 110 | 496 | 520 |
| SYTO 16 | 490 | 520 |
| SYTO 16 | 490 | 520 |
| FITC | 492 | 520 |
| Rhodamine 110 | 496 | 520 |
| SYTO 16 | 490 | 520 |
| SYBR Green I | 497 | 521 |
| SYBR Green I | 497 | 521 |
| SYBR Green I | 497 | 521 |
| SYBR Green I | 497 | 521 |
| SYBR Green I | 497 | 521 |
| Quant-iT PicoGreen | 502 | 522 |
| SYTO RNASelect | 503 | 527 |
| SYTO RNASelect | 503 | 527 |
| Rhodamine 123 | 507 | 529 |
| YFP | 512 | 529 |
| F2N12S | 405 | 530, |
| | | 585 |
| F2N12S | 405 | 530, |
| | | 585 |
| F2N12S | 405 | 530, |
| | | 585 |
| F2N12S | 405 | 530, |
| | | 585 |
| F2N12S | 405 | 530, |
| | | 585 |
| F2N12S | 405 | 530, |
| | | 585 |
| F2N12S | 405 | 530, |
| | | 585 |
| Magnesium Green | 506 | 530 |
| NBD Amine | 450 | 530 |
| TO-PRO-1 | 515 | 530 |
| TOTO-1 | 513 | 531 |
| Oregon Green 514 | 512 | 532 |
| Sodium Green | 506 | 532 |
| Vybrant DyeCycle Green | 505 | 532 |
| pHrodo Green | 509 | 533 |
| NBD-X | 467 | 538 |
| NBD-X | 467 | 538 |
| NBD-X | 467 | 538 |
| NBD-X | 467 | 538 |
| NBD-X | 467 | 538 |
| NBD-X | 467 | 538 |
| NBD-X | 467 | 538 |
| SYBR Gold | 495 | 539 |
| SYBR Gold | 495 | 539 |
| SYBR Gold | 495 | 539 |
| SYBR Gold | 495 | 539 |
| SYBR Gold | 495 | 539 |
| Alexa Fluor 430 | 432 | 540 |
| Auramine | 460 | 540 |
| Aurophosphine | 470 | 540 |
| BCECF | 499 | 540 |
| BODIPY 492/515 | 490 | 540 |
| BODIPY 505/515 | 502 | 540 |
| BODIPY FL | 502 | 540 |
| BTC | 464 | 540 |
| CFP | 434 | 540 |
| Cy2 | 492 | 540 |
| CyQUANT Direct | 500 | 540 |
| DAF-FM | 493 | 540 |
| Fluo-4 | 494 | 540 |
| TET | 520 | 541 |
| TET | 521 | 542 |
| Lucifer Yellow | 423 | 543 |
| Qdot 545 | UV | 543 |
| Lucifer Yellow | 423 | 543 |
| Lucifer Yellow | 423 | 543 |
| Lucifer Yellow | 423 | 543 |
| Lucifer Yellow | 423 | 543 |
| Lucifer Yellow | 423 | 543 |
| Lucifer Yellow | 423 | 543 |
| Lucifer Yellow | 423 | 543 |
| Lucifer yellow | 428 | 544 |
| Lucifer Yellow | 428 | 544 |
| Lucifer yellow | 428 | 544 |
| Eosin | 524 | 545 |
| JOJO-1 | 529 | 545 |
| Qdot 545 | UV | 545 |
| Qdot 545 | UV | 545 |
| Auramine 0 | 460 | 550 |
| Pacific Orange | 440 | 551 |
| Pacific Orange | 440 | 551 |
| Pacific Orange | 440 | 551 |
| Pacific Orange | 440 | 551 |
| Pacific Orange | 440 | 551 |
| Pacific Orange | 440 | 551 |
| mBanana | 540 | 553 |
| ER-Tracker Blue-White DPX | 371 | 554 |
| Alexa Fluor 532 | 532 | 554 |
| FocalCheck Double Orange | 540 | 555 |
| HEX | 533 | 558 |
| Fluospheres Orange | 539 | 560 |
| mHoneydew | 478 | 561 |
| Vybrant DyeCycle Orange | 518 | 562 |
| ActinRed 555 (rhodamin | 540 | 565 |
| pphalloidin) | | |
| Alexa Fluor 555 | 555 | 565 |
| CellRox Orange | 545 | 565 |
| Qdot 565 | UV | 565 |
| Resorufin | 570 | 585 |
| RFP | 552 | 585 |
| Qdot 585 | UV | 585 |
| Qdot 585 | UV | 585 |
| DsRed Monomer | 556 | 586 |
| pHrodo Red | 559 | 586 |
| Carboxy SNARF-1 | 548 | 587 |
| pHrodo Red | 559 | 587 |
| SpectrumOrange | 559 | 588 |
| DsRed2 | 563 | 588 |
| DiA | 456 | 590 |
| DiA | 456 | 590 |
| DiA | 456 | 590 |
| DiA | 456 | 590 |
| DiA | 456 | 590 |
| DiA | 456 | 590 |
| DiA | 456 | 590 |
| DiA | 456 | 590 |
| rhodamine Red-X | 572 | 591 |
| CellTrace calcein red-orange | 575 | 592 |
| LysoTracker Red | 573 | 592 |
| Sulforhodamine 101 | 578 | 593 |
| sulforhodamine 101 | 577 | 593 |
| ROX (6-ROX) | 568 | 595 |
| 2-dodecylresorufin | 582 | 595 |
| Cy3.5 | 579 | 597 |
| Cy 3.5 | 581 | 597 |
| MitoTracker Red CMXRos | 578 | 597 |
| BOBO-3 | 570 | 602 |
| Ethidium Bromide | 521 | 602 |
| X-rhod-1 | 579 | 602 |
| BOBO-1 | 570 | 602 |
| BOBO-1 | 570 | 602 |
| BOBO-1 | 570 | 602 |
| 5-ROX | 577 | 603 |
| Alexa Fluor 568 | 578 | 603 |
| Qdot 605 | UV | 605 |
| Qdot 605 | UV | 605 |
| BOBO-3 | 571 | 606 |
| Calcium Crimson | 589 | 608 |
| Fluospheres Red microspheres | 577 | 608 |
| ReAsH (TC-ReAsH) | 593 | 608 |
| Katushka (Turbo FP635) | 588 | 635 |
| mKate | 588 | 635 |
| SYTO 17 | 620 | 635 |
| Di-8 ANEPPS | 468 | 635 |
| Di-8 ANEPPS | 468 | 635 |
| Di-8-ANEPPS | 465 | 635 |
| Di-8-ANEPPS | 465 | 635 |
| Di-8-ANEPPS | 465 | 635 |
| Di-8-ANEPPS | 465 | 635 |
| Di-8-ANEPPS | 465 | 635 |
| Di-8-ANEPPS | 465 | 635 |
| Di-8-ANEPPS | 465 | 635 |
| Nile Red | 551 | 636 |
| Nile red (triglyceride) | 552 | 636 |
| Nile red (triglyceride) | 552 | 636 |
| Nile red (triglyceride) | 552 | 636 |
| Fura Red (high Ca2+) | 436 | 637 |
| Nile Red phospholipid | 551 | 638 |
| SYTO 17 | 619 | 638 |
| Bodipy 630/650-X | 625 | 641 |
| BODIPY 630/650X | 626 | 641 |
| 7-AAD | 549 | 644 |
| HCS NuclearMask Red | 624 | 644 |
| HCS NuclearMask Red | 622 | 644 |
| SYTO 59 | 621 | 644 |
| SYTO 59 | 622 | 645 |
| Fluospheres Crimson | 620 | 646 |
| microspheres | | |
| FluoSpheres crimson | 621 | 646 |
| microspheres | | |
| SYTOX AADvanced dead | 546 | 647 |
| cell stain | | |
| Alexa Fluor 635 | 634 | 647 |
| HcRed | 594 | 649 |
| mPlum | 590 | 649 |
| SYTO 61 | 619 | 649 |
| Alexa Fluor 633 | 631 | 650 |
| Acridine Orange + RNA | 460 | 650 |
| Acridine Orange + RNA | 460 | 650 |
| Acridine Orange (+RNA) | 460 | 650 |
| Acridine Orange (+RNA) | 460 | 650 |
| HCS LipidTOX Deep Red | 634 | 652 |
| Fura Red (+Ca2+) | 436 | 655 |
| Fura Red (+Ca2+) | 436 | 655 |
| ATTO 655 | 663 | 683 |
| FluoSpheres Dark Red | 656 | 683 |
| fluorescent microspheres | | |
| NucRed Live 647 | 638 | 686 |
| Vybrant DyeCycle Ruby | 638 | 686 |
| HCS NuclearMask Deep Red | 635 | 687 |
| Cy5.5 | 672 | 690 |
| Alexa Fluor 660 | 663 | 691 |
| Alexa Fluor 660 | 663 | 691 |
| Cy5.5 | 678 | 696 |
| DY-675 | 675 | 699 |
| IRDye 700 Phosphoramidite | 691 | 699 |
| ATTO 680 | 680 | 700 |
| Alexa Fluor 680 | 679 | 702 |
| HiLyte Fluor 680 | 688 | 702 |
| Qdot 705 Nanocrystals | 300 | 702 |
| Alexa Fluor 680 | 679 | 704 |
| DyLight 680 | 676 | 705 |
| Qdot 705 | UV | 705 |
| Qdot 705 | UV | 705 |
| Quasa 705 | 688 | 706 |
| IRDye 680 NHS Ester | 683 | 710 |
| RH795 | 530 | 712 |
| RH795 | 530 | 712 |
| RH795 | 530 | 712 |
| RH795 | 530 | 712 |
| RH795 | 530 | 712 |
| Alexa Fluor 700 | 696 | 719 |
| ATTO 700 | 699 | 719 |
| FM 4-64 | 558 | 734 |
| FM 4-64 | 558 | 734 |
| FM 4-64 | 558 | 734 |
| FM 4-64 | 558 | 734 |
| Cy7 | 745 | 766 |
| LIVE/DEAD Fixable near-IR | 750 | 775 |
| CellVue NIR780 | 743 | 776 |
| DyLight 750 | 752 | 778 |
| IRDye 800CW | 774 | 789 |
| XenoLight CF770 | 770 | 797 |
| Qdot 800 | UV | 800 |
| Qdot 800 | UV | 800 |
| Indocyanine Green | 768 | 807 |

In some other aspects, the conjugate compounds used include a chemiluminescent compound, colloidal metal, luminescent compound, phosphorescent compound, enzyme, radioisotope, nanoparticle, or paramagnetic labels.

In certain aspects, complexes used in the present disclosure can be conjugated or associated with a microscopic particle or nanoparticle (also referred to as nanopowder or nanocluster or nanocrystal) as a detectable agent or therapeutic agent. Such complexes employing nanoparticles can be used to to deliver drugs, heat, light or other substances to specific types of tissues and cells (such as cancer cells or vascular lesions) and reduces damage to healthy cells in the body and allows for earlier detection of disease. Exemplary nanoparticle compositions have been used in preclinical nuclear imaging of cardiac and vascular structures, include but are not limited to micelles, liposomes, polymeric particles, dendrimers, lipoprotein particles, gold particles, iron oxide particles, perfluorocarbon emulsions, carbon nanotubes, and upconversion nanophosphors.

In certain aspects, the complexes used in the present disclosure can be conjugated to radioactive isotopes instead of or in addition to other types of detectable agents. Certain isotopes suitable for use in the present compounds can include, but are not limited to, iodine-131, iodine-125, bismuth-212, bismuth-213, lutetium-177, rhenium-186, rhenium-188, yttrium-90, astatine-211, phosphorus-32 and/or samarium-153. In some aspects, the complexes of the present disclosure contain one or more atoms having an atomic mass or mass number different from the atomic mass or mass number usually found in nature, including but not limited to hydrogen, carbon, fluorine, phosphorous, copper, gallium, yttrium, technetium, indium, iodine, rhenium, thallium, bismuth, astatine, samarium, and lutetium (for example, ³H, ³H, ³C, ¹⁴C, ¹⁸F, ³²P, ¹⁵S, ⁶⁴Cu, ⁶⁷Ga, ⁹⁰Y, ^{99m}Tc, ¹¹¹In, ¹²⁵, ¹²³, ¹³¹L, ¹³⁵I, ¹⁸⁶Re, ¹⁸⁷Re, ²⁰¹Tl, ²¹²Bi, ²¹¹At, ¹⁵Sm and/or ¹⁷⁷Lu). In other aspects, the complexes of the present disclosure are labeled with a paramagnetic metal ion that is a good contrast enhancer in Magnetic Resonance Imaging (MRI). Examples of such paramagnetic metal ions include, but are not limited to, gadolinium III (Gd³⁺), chromium 111 (Cr³⁺), dysprosium III (Dy³⁺), iron 111 (Fe³⁺), manganese II (Mn²⁺), and ytterbium III (Yb³⁺). In certain embodiments, the labeling moiety comprises gadolinium III (Gd³⁺).

In some aspects, the complexes used in the present disclosure can be conjugated to biotin. In addition of extension of half-life, biotin can also act as an affinity handle for retrieval of the peptides from tissues or other locations. In one aspect, the complexes are conjugated, e.g., to a biotinidase resistant biotin with a PEG linker (e.g., NHS-dPEG4-Biotinidase resistant biotin). In some aspects, fluorescent biotin complexes that can act both as a detectable label and an affinity handle are used. Non-limiting examples of commercially available fluorescent biotin complexes can include Atto 425-Biotin, Atto 488-Biotin, Atto 520-Biotin, Atto-550 Biotin, Atto 565-Biotin, Atto 590-Biotin, Atto 610-Biotin, Atto 620-Biotin, Atto 655-Biotin, Atto 680-Biotin, Atto 700-Biotin, Atto 725-Biotin, Atto 740-Biotin, fluorescein biotin, biotin-4-fluorescein, biotin-(5-fluorescein) conjugate, and biotin-B-phycoerythrin, alexa fluor 488 biocytin, alexa flour 546, alexa fluor 549, lucifer yellow cadaverine biotin-X, Lucifer yellow biocytin, Oregon green 488 biocytin, biotin-rhodamine, and tetramethylrhodamine biocytin.

For example, human serum albumin (HSA) can be conjugated to the peptide conjugates or peptide-fluorophore complexes the present invention and thereby increase retention within the vasculature and its half-life. Peptides, antibodies, or antibody fragments can be engineered to target specific tissues of interest, for example vascular endothelium or nerves, so that these structures are stably labeled for the duration of a surgical or diagnostic procedure. Complexes can be created that are non-fluorescent until they are activated in the presence of the diseased tissue or other condition to be detected. Examples include peptide moieties that are cleaved by cathepsins or matrix metalloproteinases that can be used to detect vascular lesions including areas of abnormal tissue or inflammation.

In certain embodiments, the peptide and peptide variants can be conjugated to moieties, such as detectable labels (e.g., dyes) that can be detected (e.g., visualized) in a subject. In some embodiments, the peptide and/or peptide variants can be conjugated to detectable labels to enable tracking of the bio-distribution of a conjugated peptide. The detectable labels can include fluorescent dyes. Non-limiting examples of fluorescent dyes that can be used as a conjugating molecule in the present disclosure include rhodamine, rhodol, fluorescein, thiofluorescein, aminofluorescein, carboxyfluorescein, chlorofluorescein, methylfluorescein, sulfofluorescein, aminorhodol, carboxyrhodol, chlororhodol, methylrhodol, sulforhodol; aminorhodamine, carboxyrhodamine, chlororhodamine, methylrhodamine, sulforhodamine, and thiorhodamine, cyanine, indocarbocyanine, oxacarbocyanine, thiacarbocyanine, merocyanine, a cyanine dye (e.g., cyanine 2, cyanine 3, cyanine 3.5, cyanine 5, cyanine 5.5, cyanine 7), oxadiazole derivatives, pyridyloxazole, nitrobenzoxadiazole, benzoxadiazole, pyrene derivatives, cascade blue, oxazine derivatives, Nile red, Nile blue, cresyl violet, oxazine 170, acridine derivatives, proflavin, acridine orange, acridine yellow, arylmethine derivatives, auramine, xanthene dyes, sulfonated xanthenes dyes, Alexa Fluors (e.g., Alexa Fluor 594, Alexa Fluor 633, Alexa Fluor 647, Alexa Fluor 700), crystal violet, malachite green, tetrapyrrole derivatives, porphyrin, phtalocyanine, and bilirubin. Some other example dyes include near-infrared dyes, such as, but not limited to, Cy5.5, an indocyanine green (ICG), DyLight 750 or IRdye 800. In some embodiments, near infrared dyes can include cyanine dyes.

In other embodiments, therapeutic agents and anti-vascular lesion drugs, and agents, include, but are not limited to: radioisotopes, nanoparticle, toxins, enzymes, sensitizing drugs, radiosensitizers, photosensitizers, nucleic acids, including interfering RNAs, antibodies, antibody fragments, anti-angiogenic agents, aptamer, anti-angiogenic agent, anti-metabolite, mitotic inhibitor, growth factor inhibitor, anti-metabolites, mitotic inhibitors, growth factor inhibitors, and their equivalents, as well as photo-ablation.

As used herein, the terms “about” and “approximately,” in reference to a number, is used herein to include numbers that fall within a range of 10%, 5%, or 1% in either direction (greater than or less than) the number unless otherwise stated or otherwise evident from the context (except where such number would exceed 100% of a possible value).

Suitable diagnostic agents can include agents that provide for the detection by fluorescence methods as well as methods other than fluorescence imaging. Other suitable diagnostic agents can include radiolabels (e.g., radio isotopically labeled compounds) such as ¹²⁵I, ¹⁴C, and ³¹P, among others; and magnetic resonance imaging agents.

Suitable targeting agents can include antibodies, polypeptides, polysaccharides, nucleic acids, fatty acids, lipids, glycolipids, sterols, vitamins, cofactors, hormones, neurotransmitters, and metabolites.

In another aspect of the invention, compositions used can include the peptide complexes as provided. In yet another aspect of the invention, compositions used can include peptide complexes as discussed herein. The composition used can include a pharmaceutically acceptable carrier or diluent for delivery of the peptide conjugate. Suitable pharmaceutically acceptable carriers or diluents can include saline or dextrose for injection.

In various aspects, the presently described compounds used can further comprise a detectable label, which can be used for the detection of the peptide-label conjugate and the vascular lesion tissues or cells to which they are bound or accumulated.

In various aspects, compounds used in the present disclosure can have the structure of Formula (I), or a pharmaceutically acceptable salt thereof:

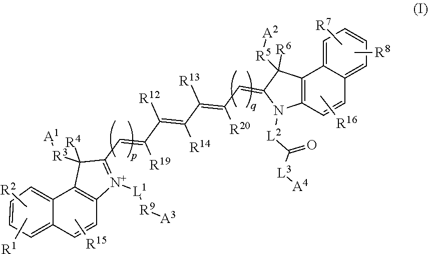

wherein:

R¹, R², R³, R⁴, R⁵, R⁶, R⁷, R⁸, R¹⁵, and R¹⁶ are each independently selected from hydrogen, C₁-C₆ alkyl, C₁-C₆ alkylene-COOH, sulfonate, —COOH, —SO₂—NH₂, C₁-C₆ alkoxy, C₁-C₁₀ alkylene-(C(═O))ₓ—, C₁-C₁₀ alkylene-(C(═O))ₓ—O—, or C₁-C₁₀ alkylene-(C(═O))ₓ—NR¹⁰—.

R⁹ is hydrogen, sulfonate, —COOH, C₁-C₁₀ alkylene-(C(═O))ₓ—, C₁-C₁₀ alkylene-(C(═O))ₓ—O—, or C₁-C₁₀ alkylene-(C(═O))ₓ—NR¹⁰—;

L¹ is C₃-C₆ alkylene;

L² is C₁-C₁₀ alkylene;

L³ is a bond, —O—, —NR¹⁰—, —NR¹⁰—C₁-C₆ alkylene-, —O—NR¹⁰—, —NR¹⁰—C₁-C₆ alkylene-(O—C₁-C₆ alkylene)ₓ-, —NR¹⁰-L⁴-, —NR¹⁰—C₁-C₆ alkylene-NR¹⁰— (C(═O)—C₁-C₆ alkylene-O))ₓ—, or —NR¹⁰—C₁-C₆ alkylene-NR¹⁰—C₁-C₆ alkylene-NR¹⁰—C₁-C₆ alkylene-;

L⁴ is a bond, -heterocyclyl-, or -heterocyclyl-C₁-C₆ alkylene-;

R¹⁰ is hydrogen or C₁-C₆ alkyl;

R¹¹ is hydrogen or C₁-C₆ alkyl;

R¹² and R¹³ are each independently selected from hydrogen, C₁-C₆ alkyl, or R¹² and R¹³ are joined together along with the other atoms to which they are attached to form a 5-membered or 6-membered carbocyclic or heterocyclic ring;

R¹⁴ is hydrogen or C₁-C₆ alkylene, -(L⁵)-aryl, -(L⁵)-aryl-A⁵, -(L⁵)-heteroaryl, -(L⁵)-heteroaryl-A⁵, —NR¹⁷R¹⁸, R¹⁴ and R¹⁹ are joined together along with the other atoms to which they are attached to form a 5-membered or 6-membered carbocyclic or heterocyclic ring, or R⁴ and R²⁰ are joined together along with the other atoms to which they are attached to form a 5-membered or 6-membered carbocyclic or heterocyclic ring;

L⁵ is a bond, C₁-C₁₀ alkylene, —O—, or —NR¹⁰—;

R¹⁷ and R¹⁸ are each independently hydrogen or aryl;

R¹⁹ and R²⁰ are each independently selected from hydrogen, C₁-C₆ alkyl, R¹⁴ and R¹⁹ are joined together along with the other atoms to which they are attached to form a 5-membered or 6-membered carbocyclic or heterocyclic ring, or R¹⁴ and R²⁰ are joined together along with the other atoms to which they are attached to form a 5-membered or 6-membered carbocyclic or heterocyclic ring;

n is 0, 1, 2, or 3;

m is 0, 1, 2, or 3;

p is 0, 1, 2, or 3;

q is 0, 1, 2, or 3;

x is 0 or 1; and

one of A¹, A², A³, A⁴, or A⁵ is a polypeptide having at least 85% sequence identity with MCMPCFTTDHQMARRCDDCCGGRGRGKCYGPQCLCR (SEQ ID NO: 9) or a fragment thereof and the others of A¹, A², A³, A⁴, or A⁵ are each independently absent, hydrogen, —COOH, or sulfonate.

In various aspects, the presently described compounds used can further comprise a detectable label, which can be used for the detection of the peptide-label conjugate and the vascular lesion tissues or cells to which they are bound or accumulated.

In various aspects, compounds used in the present disclosure have the structure of Formula (II), or a pharmaceutically acceptable salt thereof:

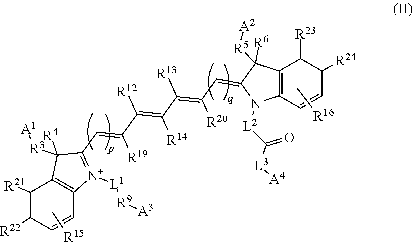

wherein:

R³, R⁴, R⁵, R⁶, R¹⁵, and R¹⁶ are each independently selected from hydrogen, C₁-C₆ alkyl, C₁-C₆ alkylene-COOH, sulfonate, —COOH, —SO₂—NH₂, C₁-C₆ alkoxy, C₁-C₁₀ alkylene-(C(═O))ₓ—, C₁-C₁₀ alkylene-(C(═O))ₓ—O—, or C₁-C₁₀ alkylene-(C(═O))ₓ—NR¹⁰—;

R⁹ is hydrogen, sulfonate, —COOH, C₁-C₁₀ alkylene-(C(═O))ₓ, C₁-C₁₀ alkylene-(C(═O))ₓ—O—, or C₁-C₁₀ alkylene-(C(═O))ₓ—NR¹⁰—;

L¹ is C₃-C₆ alkylene;

L² is C₁-C₁₀ alkylene;

L³ is a bond, —O—, —NR¹⁰—, —NR¹⁰—C₁-C₆ alkylene-, —O—NR¹⁰—, —NR¹⁰—C₁-C₆ alkylene-(O—C₁-C₆ alkylene)ₓ-, —NR¹⁰-L⁴-, —NR¹¹—C₁-C₆ alkylene-NR¹¹—(C(═O)—C₁-C₆ alkylene-O))ₓ—, or —NR¹⁰—C₁-C₆ alkylene-NR¹⁰—C₁-C₆ alkylene-NR¹⁰—C₁-C₆ alkylene-;

L⁴ is a bond, -heterocyclyl-, or -heterocyclyl-C₁-C₆ alkylene-;

R¹⁰ is hydrogen or C₁-C₆ alkyl;

R¹¹ is hydrogen or C₁-C₆ alkyl;

R¹² and R¹³ are each independently selected from hydrogen, C₁-C₆ alkyl, or R¹² and R¹³ are joined together along with the other atoms to which they are attached to form a 5-membered or 6-membered carbocyclic or heterocyclic ring;

R¹⁴ is hydrogen or C₁-C₆ alkylene, -(L⁵)-aryl, -(L⁵)-aryl-A⁵, -(L⁵)-heteroaryl, -(L⁵)-heteroaryl-A⁵, —NR¹⁷R¹⁸, R¹⁴ and R¹⁹ are joined together along with the other atoms to which they are attached to form a 5-membered or 6-membered carbocyclic or heterocyclic ring, or R⁴ and R²⁰ are joined together along with the other atoms to which they are attached to form a 5-membered or 6-membered carbocyclic or heterocyclic ring;

L⁵ is a bond, C₁-C₁₀ alkylene, —O—, or —NR¹⁰—;

R¹⁷ and R¹⁸ are each independently hydrogen or aryl;

R¹⁹ and R²⁰ are each independently selected from hydrogen, C₁-C₆ alkyl, R¹⁴ and R¹⁹ are joined together along with the other atoms to which they are attached to form a 5-membered or 6-membered carbocyclic or heterocyclic ring, or R¹⁴ and R²⁰ are joined together along with the other atoms to which they are attached to form a 5-membered or 6-membered carbocyclic or heterocyclic ring;

R²¹ and R²² are each independently selected from hydrogen, C₁-C₆ alkyl, sulfonate, or

R²¹ and R²² are joined together along with the other atoms to which they are attached to form a 5-membered or 6-membered aryl;

R²³ and R²⁴ are each independently selected from hydrogen, C₁-C₆ alkyl, sulfonate, or

R²³ and R²⁴ are joined together along with the other atoms to which they are attached to form a 5-membered or 6-membered aryl;

n is 0, 1, 2, or 3;

m is 0, 1, 2, or 3;

p is 0, 1, 2, or 3;

q is 0, 1, 2, or 3;

x is 0 or 1; and

one of A¹, A², A³, A⁴, or A⁵ is a polypeptide having at least 85% sequence identity with MCMPCFTTDHQMARRCDDCCGGRGRGKCYGPQCLCR (SEQ ID NO: 9) or a fragment thereof and the others of A¹, A², A³, A⁴, or A⁵ are each independently absent, hydrogen, —COOH, or sulfonate.

In some aspects, the compounds used in the present disclosure have a structure of Formula (III), or a pharmaceutically acceptable salt thereof:

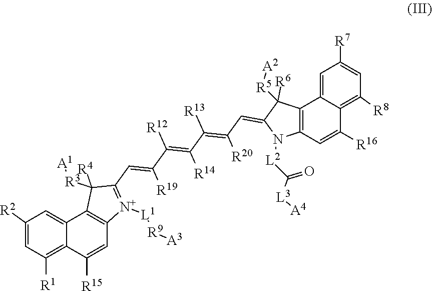

In certain aspects, the present compounds have a structure of Formula (IV), or a pharmaceutically acceptable salt thereof:

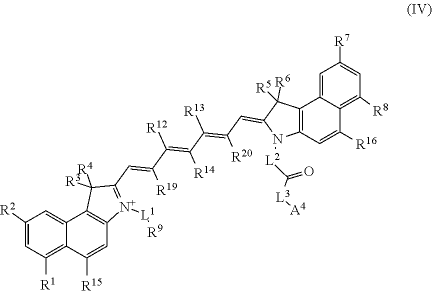

wherein:

R¹, R², R³, R⁴, RV, R⁶, R⁷, R⁸, R¹⁵, and R¹⁶ are each independently selected from hydrogen, C₁-C₆ alkyl, C₁-C₆ alkylene-COOH, sulfonate, C₁-C₆ alkylene-sulfonate, —COOH, —SO₂—NH₂, or C₁-C₆ alkoxy;

R⁹ is hydrogen, sulfonate, amine or —COOH;

L¹ is C₃-C₆ alkylene;

L² is C₁-C₁₀ alkylene;

L³ is a bond, —O—, —NR¹⁰—, —NR¹¹—C₁-C₆ alkylene-, —O—NR¹⁰—, —NR¹¹—C₁-C₆ alkylene-(O—C₁-C₆ alkylene)ₓ-, —NR¹⁰-L⁴-, —NR¹¹—C₁-C₆ alkylene-NR¹¹— (C(═O)—C₁-C₆ alkylene-O))ₓ—, or —NR¹⁰—C₁-C₆ alkylene-NR¹⁰—C₁-C₆ alkylene-NR¹⁰—C₁-C₆ alkylene-;

L⁴ is a bond, -heterocyclyl-, or -heterocyclyl-C₁-C₆ alkylene-;

R¹⁰ is hydrogen or C₁-C₆ alkyl;

R¹¹ is hydrogen or C₁-C₆ alkyl;

R¹² and R¹³ are independently selected from hydrogen, C₁-C₆ alkyl, or R¹² and R¹³ are joined together along with the other atoms to which they are attached to form a 5-membered or 6-membered carbocyclic or heterocyclic ring;

R¹⁴ is hydrogen or C₁-C₆ alkylene, -(L⁵)-aryl, -(L⁵)-aryl-R²¹, -(L⁵)-heteroaryl, -(L⁵)-heteroaryl-R²¹, —NR¹⁷R¹⁸, R¹⁴ and R¹⁹ are joined together along with the other atoms to which they are attached to form a 5-membered or 6-membered carbocyclic or heterocyclic ring, or R¹⁴ and R²⁰ are joined together along with the other atoms to which they are attached to form a 5-membered or 6-membered carbocyclic or heterocyclic ring;

L⁵ is a bond, C₁-C₁₀ alkylene, —O—, —NR¹¹—;

R¹⁷ and R¹⁸ are each independently hydrogen or aryl;

R¹⁹ and R²⁰ are independently selected from hydrogen, C₁-C₆ alkyl, R¹⁴ and R¹⁹ are joined together along with the other atoms to which they are attached to form a 5-membered or 6-membered carbocyclic or heterocyclic ring, or R¹⁴ and R²⁰ are joined together along with the other atoms to which they are attached to form a 5-membered or 6-membered carbocyclic or heterocyclic ring;

R²¹ is hydrogen, sulfonate, or —COOH;

n is 0, 1, 2, or 3;

m is 0, 1, 2, or 3;

p is 0, 1, 2, or 3;

q is 0, 1, 2, or 3; and

A⁴ is a polypeptide having at least 80% sequence identity with MCMPCFTTDHQMARRCDDCCGGRGRGKCYGPQCLCR (SEQ ID NO: 9) or a fragment thereof.

In other aspects, compounds used in the present disclosure have a structure of Formula (V), or a pharmaceutically acceptable salt thereof:

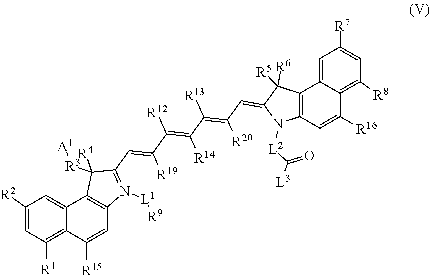

wherein:

R¹, R², R⁴, R⁵, R⁶, R⁷, R⁸, R¹⁵, and R¹⁶ are each independently selected from hydrogen, C₁-C₆ alkyl, C₁-C₆ alkylene-COOH, sulfonate, —COOH, —SO₂—NH₂, or C₁-C₆ alkoxy;

R³ is selected from C₁-C₁₀ alkylene-(C(═O))ₓ—, C₁-C₁₀ alkylene-(C(═O))ₓ—O—, or C₁-C₁₀ alkylene-(C(═O))ₓ—NR¹¹—;

R⁹ is hydrogen, sulfonate, or —COOH, or C₁-C₁₀ alkyl;

L¹ is C₃-C₆ alkylene;

L² is C₁-C₁₀ alkylene;

L³ is hydrogen, sulfonate, —COOH, C₁-C₁₀ alkyl;

L⁴ is a bond, -heterocyclyl-, or -heterocyclyl-C₁-C₆ alkylene-;

R¹⁰ is hydrogen or C₁-C₆ alkyl;

R¹¹ is hydrogen or C₁-C₆ alkyl;

R¹² and R¹³ are independently selected from hydrogen, C₁-C₆ alkyl, or R¹² and R¹³ are joined together along with the other atoms to which they are attached to form a 5-membered or 6-membered carbocyclic or heterocyclic ring;

R¹⁴ is hydrogen or C₁-C₆ alkylene, -(L⁵)-aryl, -(L⁵)-heteroaryl, —NR¹⁷R¹⁸, R¹⁴ and R¹⁹ are joined together along with the other atoms to which they are attached to form a 5-membered or 6-membered carbocyclic or heterocyclic ring, or R¹⁴ and R²⁰ are joined together along with the other atoms to which they are attached to form a 5-membered or 6-membered carbocyclic or heterocyclic ring;

L⁵ is a bond, C₁-C₁₀ alkylene, —O—, —NR¹⁰—;

R¹⁷ and R¹⁸ are each independently hydrogen or aryl;

R¹⁹ and R²⁰ are independently selected from hydrogen, C₁-C₆ alkyl, R¹⁴ and R¹⁹ are joined together along with the other atoms to which they are attached to form a 5-membered or 6-membered carbocyclic or heterocyclic ring, or R¹⁴ and R²⁰ are joined together along with the other atoms to which they are attached to form a 5-membered or 6-membered carbocyclic or heterocyclic ring;

n is 0, 1, 2, or 3;

m is 0, 1, 2, or 3;

p is 0, 1, 2, or 3;

q is 0, 1, 2, or 3;

x is 0 or 1; and

A¹ is a polypeptide having at least 85% sequence identity with MCMPCFTTDHQMARRCDDCCGGRGRGKCYGPQCLCR (SEQ ID NO: 9) or a fragment thereof.

In other aspects, compounds used in the present disclosure have a structure of Formula (VI), or a pharmaceutically acceptable salt thereof:

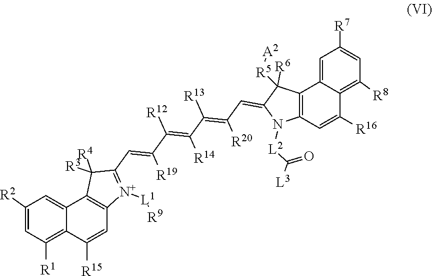

wherein:

R¹, R², R³, R⁴, R⁶, R⁷, R⁸, R⁵, and R¹⁶ are each independently selected from hydrogen, C₁-C₆ alkyl, C₁-C₆ alkylene-COOH, sulfonate, —COOH, —SO₂—NH₂, or C₁-C₆ alkoxy;

R⁵ is selected from C₁-C₁₀ alkylene-(C(═O))ₓ—, C₁-C₁₀ alkylene-(C(═O))ₓ—O—, or C₁-C₁₀ alkylene-(C(═O))ₓ—NR¹⁰—;

R⁹ is hydrogen, sulfonate, or —COO, or C₁-C₁₀ alkyl;

L¹ is C₃-C₆ alkylene;

L² is C₁-C₁₀ alkylene;

L³ is hydrogen, sulfonate, —COOH, or C₁-C₁₀ alkyl;

L⁴ is a bond, -heterocyclyl-, or -heterocyclyl-C₁-C₆ alkylene-;

R¹⁰ is hydrogen or C₁-C₆ alkyl;

R¹¹ is hydrogen or C₁-C₆ alkyl;

R¹² and R¹³ are independently selected from hydrogen, C₁-C₆ alkyl, or R¹² and R¹³ are joined together along with the other atoms to which they are attached to form a 5-membered or 6-membered carbocyclic or heterocyclic ring;

R¹⁴ is hydrogen or C₁-C₆ alkylene, -(L⁵)-aryl, -(L⁵)-heteroaryl, —NR¹⁷R¹⁸, R¹⁴ and R¹⁹ are joined together along with the other atoms to which they are attached to form a 5-membered or 6-membered carbocyclic or heterocyclic ring, or R¹⁴ and R²⁰ are joined together along with the other atoms to which they are attached to form a 5-membered or 6-membered carbocyclic or heterocyclic ring;

L⁵ is a bond, C₁-C₁₀ alkylene, —O—, —NR¹¹—;

R¹⁷ and R¹⁸ are each independently hydrogen or aryl;

R¹⁹ and R²⁰ are independently selected from hydrogen, C₁-C₆ alkyl, R¹⁴ and R¹⁹ are joined together along with the other atoms to which they are attached to form a 5-membered or 6-membered carbocyclic or heterocyclic ring, or R¹⁴ and R²⁰ are joined together along with the other atoms to which they are attached to form a 5-membered or 6-membered carbocyclic or heterocyclic ring;

n is 0, 1, 2, or 3;

m is 0, 1, 2, or 3;

p is 0, 1, 2, or 3;

q is 0, 1, 2, or 3;

x is 0 or 1; and

A² is a polypeptide having at least 85% sequence identity with MCMPCFTTDHQMARRCDDCCGGRGRGKCYGPQCLCR (SEQ ID NO: 9) or a fragment thereof.

In some aspects, compounds used in the present disclosure have a structure of Formula (VII), or a pharmaceutically acceptable salt thereof:

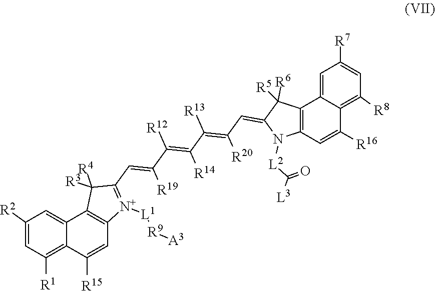

wherein:

R¹, R², R³, R⁴, R⁵, R⁶, R⁷, R⁸, R¹⁵, and R¹⁶ are each independently selected from hydrogen, C₁-C₆ alkyl, C₁-C₆ alkylene-COOH, sulfonate, —COOH, —SO₂—NH₂, or C₁-C₆ alkoxy;

R⁹ is selected from C₁-C₁₀ alkylene-(C(═O))ₓ—, C₁-C₁₀ alkylene-(C(═O))ₓ—O—, or C₁-C₁₀ alkylene-(C(═O))ₓ—NR¹¹—;

L¹ is C₃-C₆ alkylene;

L² is C₁-C₁₀ alkylene;

L³ is hydrogen, sulfonate, —COOH, or C₁-C₁₀ alkyl;

L⁴ is a bond, -heterocyclyl-, or -heterocyclyl-C₁-C₆ alkylene-;

R¹⁰ is hydrogen or C₁-C₆ alkyl;

R¹¹ is hydrogen or C₁-C₆ alkyl;

R¹² and R¹³ are independently selected from hydrogen, C₁-C₆ alkyl, or R¹² and R¹³ are joined together along with the other atoms to which they are attached to form a 5-membered or 6-membered carbocyclic or heterocyclic ring;

R¹⁴ is hydrogen or C₁-C₆ alkylene, -(L⁵)-aryl, -(L⁵)-heteroaryl, —NR¹⁷R¹⁵, R¹⁴ and R¹⁹ are joined together along with the other atoms to which they are attached to form a 5-membered or 6-membered carbocyclic or heterocyclic ring, or R¹⁴ and R²⁰ are joined together along with the other atoms to which they are attached to form a 5-membered or 6-membered carbocyclic or heterocyclic ring;

R¹⁷ and R¹⁸ are each independently hydrogen or aryl;

R¹⁹ and R²⁰ are independently selected from hydrogen, C₁-C₆ alkyl, R¹⁴ and R¹⁹ are joined together along with the other atoms to which they are attached to form a 5-membered or 6-membered carbocyclic or heterocyclic ring, or R¹⁴ and R²⁰ are joined together along with the other atoms to which they are attached to form a 5-membered or 6-membered carbocyclic or heterocyclic ring;

n is 0, 1, 2, or 3;

m is 0, 1, 2, or 3;

p is 0, 1, 2, or 3;

q is 0, 1, 2, or 3;

x is 0 or 1;

L⁵ is a bond, C₁-C₁₀ alkylene, —O—, —NR¹⁰—;

A³ is a polypeptide having at least 85% sequence identity with MCMPCFTTDHQMARRCDDCCGGRGRGKCYGPQCLCR (SEQ ID NO: 9) or a fragment thereof.

In additional aspects, compounds used in the present disclosure have a structure Formula (VIII), or a pharmaceutically acceptable salt thereof:

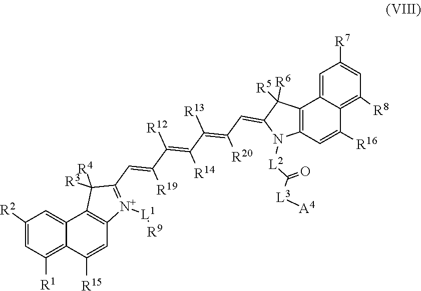

wherein:

R¹, R², R³, R⁴, R⁵, R⁶, R⁷, R⁸, R¹⁵, and R¹⁶ are each independently selected from hydrogen, C₁-C₆ alkyl, C₁-C₆ alkylene-COOH, sulfonate, —COOH, —SO₂—NH₂, or C₁-C₆ alkoxy;

R⁹ is hydrogen, sulfonate, or —COOH;

L¹ is C₃-C₆ alkylene;

L² is C₁-C₁₀ alkylene;

L³ is a bond, —O—, —NR¹⁰—, —NR¹¹—C₁-C₆ alkylene-, —O—NR¹⁰—, —NR¹¹—C₁-C₆ alkylene-(O—C₁-C₆ alkylene)-, —NR¹⁰-L⁴-, —NR¹⁰—C₁-C₆ alkylene-NR¹¹—(C(═O)—C₁-C₆ alkylene-O))ₓ—, or —NR¹⁰—C₁-C₆ alkylene-NR¹⁰—C₁-C₆ alkylene-NR¹⁰—C₁-C₆ alkylene-;

L⁴ is a bond, -heterocyclyl-, or -heterocyclyl-C₁-C₆ alkylene-;

R¹⁰ is hydrogen or C₁-C₆ alkyl;

R¹¹ is hydrogen or C₁-C₆ alkyl;

R¹² and R¹³ are independently selected from hydrogen, C₁-C₆ alkyl, or R¹² and R¹³ are joined together along with the other atoms to which they are attached to form a 5-membered or 6-membered carbocyclic or heterocyclic ring;

R¹⁴ is -(L)-aryl-A⁵, or -(L⁵)-heteroaryl-A⁵;

L⁵ is a bond, C₁-C₁₀ alkylene, —O—, —NR¹⁰—;

R¹⁷ and R¹⁸ are each independently hydrogen or aryl;

R¹⁹ and R²⁰ are independently selected from hydrogen, C₁-C₆ alkyl, R¹⁴ and R¹⁹ are joined together along with the other atoms to which they are attached to form a 5-membered or 6-membered carbocyclic or heterocyclic ring, or R¹⁴ and R²⁰ are joined together along with the other atoms to which they are attached to form a 5-membered or 6-membered carbocyclic or heterocyclic ring;

n is 0, 1, 2, or 3;

m is 0, 1, 2, or 3;

p is 0, 1, 2, or 3;

q is 0, 1, 2, or 3;

x is 0 or 1;

A⁴ is hydrogen, —COOH, or sulfonate; and

A⁵ is a polypeptide having at least 85% sequence identity with MCMPCFTTDHQMARRCDDCCGGRGRGKCYGPQCLCR (SEQ ID NO: 9) or a fragment thereof.

In certain aspects, A¹, A², and A³ are absent. In some aspects, A⁵ is hydrogen. In certain aspects, R³, R⁴, R⁵, and R⁶ are each independently C₁-C₆ alkyl. In some aspects, R³, R⁴, R⁵, and R⁶ are each independently methyl. In certain aspects, R¹, R², R⁷, R⁸, R¹³, and R¹⁶ are each independently selected from hydrogen or sulfonate. In further aspects, R¹, R², R⁷, R⁸, R¹⁵, and R¹⁶ are each independently hydrogen. In some aspects, R¹², R³, R¹⁴, R¹⁹, R²⁰ are each independently hydrogen.

In certain aspects, R¹² and R¹³ join together along with the atoms to which they are attached to form a six-membered carbocyclic ring. In other aspects, R¹² and R¹³ join together along with the atoms to which they are attached to form a five-membered carbocyclic ring. In certain aspects, R¹⁴ and R¹⁹ join together along with the atoms to which they are attached to form a six-membered carbocyclic ring. In some aspects, R¹⁴ and R²⁰ join together along with the atoms to which they are attached to form a six-membered carbocyclic ring. In certain aspects, L¹ is C₃-C₆ alkylene. In other aspects, L¹ is C₃-C₈ alkylene. In still other aspects, L¹ is propylene. In still other aspects, L¹ is butylene. In other aspects, L¹ is pentylene. In some aspects, L² is C₃-C₆ alkylene. In other aspects, L² is propylene. In still other aspects, L² is butylene. In other aspects, L² is pentylene. In some aspects, R⁹ is sulfonate. In other aspects, R⁹ is hydrogen. In some aspects, R¹⁴ is hydrogen. In other aspects, R¹⁴ is -(L⁵)-aryl. In still other aspects, R¹⁴ is -(L⁵)-aryl-A⁵.

In some aspects, R¹ is hydrogen. In certain aspects, R² is hydrogen. In some aspects, R³ is methyl. In certain aspects, R⁴ is methyl. In some aspects, R⁵ is methyl. In certain aspects R⁶ is methyl. In some aspects, R⁷ is hydrogen. In certain aspects, R¹¹ is hydrogen. In some aspects, R¹² is hydrogen. In certain aspects, R¹³ is hydrogen. In some aspects, R¹⁴ is hydrogen. In certain aspects, R¹⁹ is hydrogen. In some aspects, R²⁰ is hydrogen. In certain aspects, R¹⁰ is hydrogen. In some aspects, R¹¹ is hydrogen.

In some aspects, R¹⁷ and R¹⁸ are independently phenyl. In some aspects, L¹ is butylene. In some aspects, L² is pentylene. In some aspects, L³ is selected from a bond, —O—, —NR¹¹—, —NR¹¹—C₁-C₆ alkylene-, —O—NR¹⁰—, or —NR¹⁰-L⁴-. In further aspects, L³ is a bond.

In some aspects, L⁴ is -heterocyclyl- or -heterocyclyl-C₁-C₆ alkylene-. In further aspects, L⁴ is -piperizinyl-(C₁-C₆ alkylene)-. In still further aspects, L⁴ is

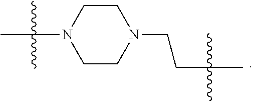 [SMILES: *CCN1CCN(*)CC1]

In some aspects, p is 1. In certain aspects, q is 1.

In some aspects, the compound used has the structure of any one of Formulas (IX), (X), (XI), (XII), (XIII), (XIV), (XV), or (XVI):

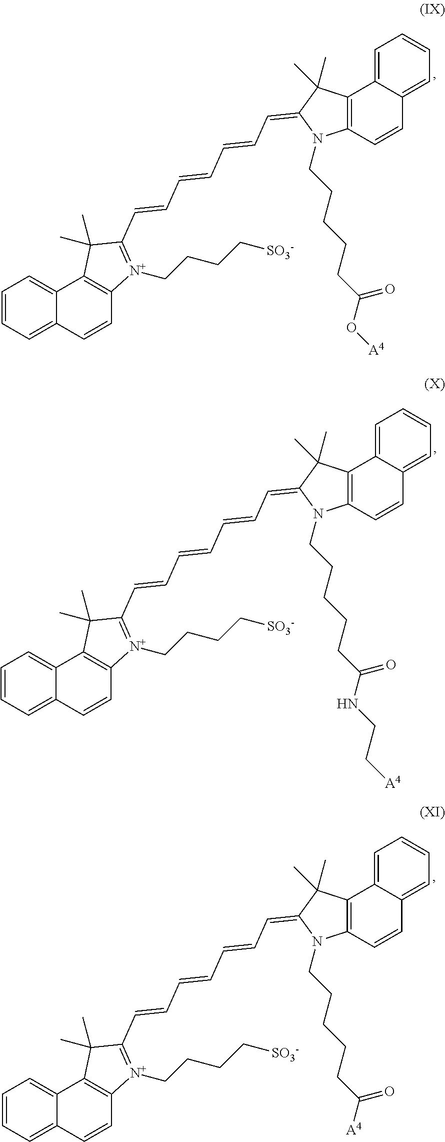

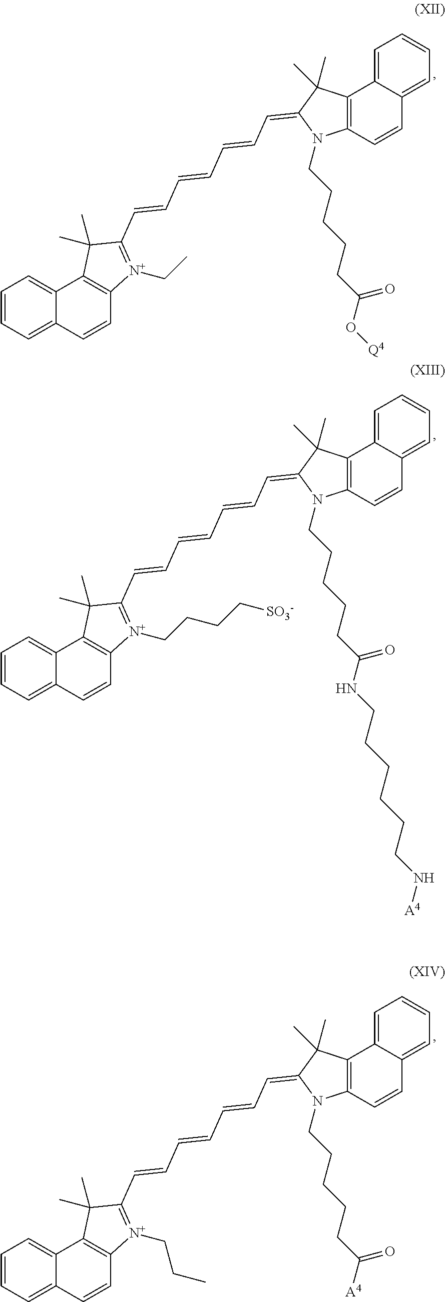

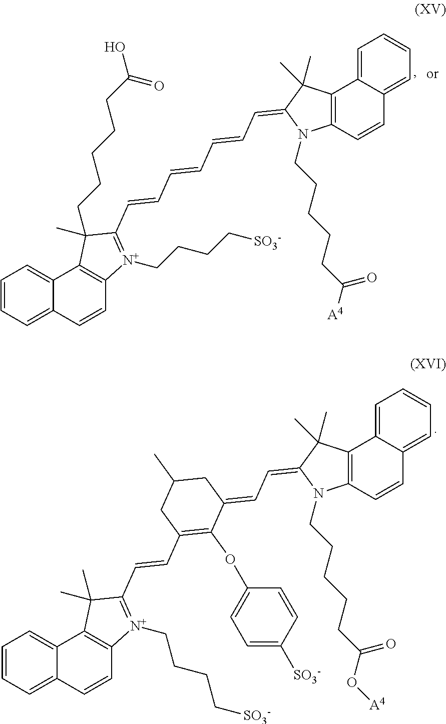

In some aspects, the compound has the structures of any one of Formulas (IX), (X), (XI), (XII), (XIII), (XIV), (XV), or (XVI), wherein A⁴ is a polypeptide.

In some aspects, one of A¹, A², A³, A⁴, or A⁵ is a polypeptide having at least 87% sequence identity with MCMPCFTTDHQMARRCDDCCGGRGRGKCYGPQCLCR (SEQ ID NO: 9) or a fragment thereof. In further aspects, one of A¹, A², A³, A⁴, or A⁵ is a polypeptide having at least 90% sequence identity with MCMPCFTTDHQMARRCDDCCGGRGRGKCYGPQCLCR (SEQ ID NO: 9) or a fragment thereof. In still further aspects, one of A¹, A², A³, A⁴, or A⁵ is a polypeptide having at least 92% sequence identity with MCMPCFTTDHQMARRCDDCCGGRGRGKCYGPQCLCR (SEQ ID NO: 9) or a fragment thereof. In still further aspects, one of A¹, A², A³, A⁴, or A⁵ is a polypeptide having at least 95% sequence identity with MCMPCFTTDHQMARRCDDCCGGRGRGKCYGPQCLCR (SEQ ID NO: 9) or a fragment thereof. In still further aspects, one of A¹, A², A³, A⁴, or A⁵ is a polypeptide having at least 97% sequence identity with MCMPCFTTDHQMARRCDDCCGGRGRGKCYGPQCLCR (SEQ ID NO: 9) or a fragment thereof. In still further aspects, one of A¹, A², A³, A⁴, or A⁵ is a polypeptide having 100% sequence identity with MCMPCFTTDHQMARRCDDCCGGRGRGKCYGPQCLCR (SEQ ID NO: 9) or a fragment thereof. In still further aspects, one of A¹, A², A³, A⁴, or A⁵ is a polypeptide having the sequence MCMPCFTTDHQMARRCDDCCGGRGRGKCYGPQCLCR (SEQ ID NO: 9) or a fragment thereof.

In some aspects, the fragment of A¹, A², A³, A⁴, or A⁵ has a length of at least 25 amino acid residues. In further aspects, the fragment of A¹, A², A³, A⁴, or A⁵ has a length of at least 27 amino acid residues. In still further aspects, the fragment of A¹, A², A³, A⁴, or A⁵ has a length of at least 29 amino acid residues. In still further aspects, the fragment of A¹, A², A³, A⁴, or A⁵ has a length of at least 31 amino acid residues. In still further aspects, the fragment of A¹, A², A³, A⁴, or A⁵ has a length of at least 33 amino acid residues.

In some aspects, one of A¹, A², A³, A⁴, or A⁵ is a polypeptide having at least 85% sequence identity with MCMPCFTTDHQMARRCDDCCGGRGRGKCYGPQCLCR (SEQ ID NO: 9) or a fragment thereof having the vascular lesion cell binding affinity of native chlorotoxin. In certain aspects, one of A¹, A², A³, A⁴, or A⁵ is a polypeptide having at least 85% sequence identity with MCMPCFTTDHQMARRCDDCCGGRGRGKCYGPQCLCR (SEQ ID NO: 9) or a fragment thereof having about the same the vascular lesion cell binding affinity of native chlorotoxin. In some aspects, one of A¹, A², A³, A⁴, or A⁵ is a polypeptide having at least 85% sequence identity with MCMPCFTTDHQMARRCDDCCGGRGRGKCYGPQCLCR (SEQ ID NO: 9) or a fragment thereof having the vascular lesion cell binding affinity of native chlorotoxin wherein one of A¹, A², A³, A⁴, or A⁵ has a sequence selected from SEQ ID NO: 1-SEQ ID NO: 485.

In some aspect, the polypeptide contains no lysine residues. In some aspects, the polypeptide used comprises at least one lysine amino acid residue. In certain aspects, the polypeptide comprises a single lysine amino acid residue. In some aspects, the polypeptide comprises one, two, or three lysine amino acid residues. In some aspects, the polypeptide comprises a lysine residue at the position corresponding to K-27 of native chlorotoxin. In some aspects, the polypeptide comprises a lysine residue at the position corresponding to K-23 of native chlorotoxin. In some aspects, the polypeptide comprises a lysine residue at the position corresponding to K-15 of native chlorotoxin.

In some aspects, one or more of the amino acids of the polypeptide used is substituted with a non-naturally occurring amino acid residue. In further aspects the non-naturally occurring amino acid residue is a citrulline amino acid residue. In still further aspects, L³ is attached to A⁴ at a citrulline amino acid residue of the polypeptide.

In some aspects, L³ is attached to A⁴ at a lysine amino acid residue of the polypeptide. In certain aspects, L³ is attached to A⁴ at the N-terminus of the polypeptide. In some aspects, L³ is attached to A⁴ at the C-terminus of the polypeptide. In some aspects, the R³ is attached to A¹ at a lysine amino acid residue of the peptide, a citrulline amino acid residue of the polypeptide, the N-terminus of the polypeptide, or the C-terminus of the polypeptide. In some aspects, the R⁵ is attached to A² at a lysine amino acid residue of the polypeptide, a citrulline amino acid residue of the polypeptide, the N-terminus of the polypeptide, or the C-terminus of the polypeptide. In some aspects, the R⁹ is attached to A³ at a lysine amino acid residue of the polypeptide, a citrulline amino acid residue of the polypeptide, the N-terminus of the polypeptide, or the C-terminus of the polypeptide. In some aspects, the aryl is attached to A⁵ at a lysine amino acid residue of the polypeptide, a citrulline amino acid residue of the polypeptide, the N-terminus of the polypeptide, or the C-terminus of the polypeptide.

In some aspects, the compound used has the structure of any one of compounds 1 to 60 as found in TABLE 2, in which A is a peptide portion and can comprise any of the peptides described herein, such as any one of SEQ ID NO: 1-SEQ ID NO: 485. In other aspects, the compound used has the structure of any one of compounds 1 to 60 as found in TABLE 2, in which A is a peptide fragment and can comprise a fragment of any of the peptides described herein, such as any one of SEQ ID NO: 1-SEQ ID NO: 485. In some embodiments, the fragment of the polypeptide has a length of at least 25 residues.

In some aspects, the compound used is conjugated to polyethylene glycol (PEG), hydroxyethyl starch, polyvinyl alcohol, a water soluble polymer, a zwitterionic water soluble polymer, a water soluble poly(amino acid), an albumin derivative, or a fatty acid.

In some aspects, the polypeptide used has an isoelectric point of from 5.5 to 9.5. In some aspects, the polypeptide has an isoelectric point of from 7.5 to 9.0. In some aspects, the polypeptide has an isoelectric point of from 8.0 to 9.0. In some aspects, the polypeptide has an isoelectric point of from 8.5 to 9.0. In some aspects, the polypeptide is basic and has an isoelectric point of greater than 7.5. In some aspects, the polypeptide has an isoelectric point of about 6.0, about 6.1, about 6.2, about 6.3, about 6.4, about 6.5, about 6.6, about 6.7, about 6.8, about 6.9, about 7.0, about 7.1, about 7.2, about 7.3, about 7.4, about 7.5, about 7.6, about 7.7, about 7.8, about 7.9, about 8.0, about 8.1, about 8.2, about 8.3, about 8.4, about 8.5, about 8.6, about 8.7, about 8.8, about 8.9, or about 9.0. In other aspects, the polypeptide comprises an isoelectric point of at least 5.5, at least 6.0, at least 6.5, at least 7.0, at least 7.5, at least 8.0, at least 8.5, at least 9.0, or at least 9.5.

In some aspects, the polypeptide used comprises at least eight cysteine amino acid residues. In some aspects, the polypeptide comprises eight cysteine amino acid residues. In some aspects, the polypeptide comprises four disulfide bonds. In some aspects, the polypeptide comprises from six to seven cysteine amino acid residues. In some aspects, the polypeptide comprises three disulfide bonds. In some aspects, the polypeptide comprises at least 1 disulfide bond, at least 2 disulfide bonds, at least 3 disulfide bonds, at least 4 disulfide bonds, at least 5 disulfide bonds, or at least 6 disulfide bonds. In some aspects, the spacing between the cysteine amino acid residues in the polypeptide is about the same as in native chlorotoxin. In some aspects, the distribution of charge on the surface of the polypeptide is about the same as in native chlorotoxin.

In some aspects, the N-terminus of the polypeptide is blocked by acetylation or cyclization.

In some aspects, one or more of the methionine amino acid residues used is replaced with an amino acid residue selected from isoleucine, threonine, valine, leucine, serine, glycine, alanine, or a combination thereof. In other aspects, one, two, or three methionine residues of the polypeptide are replaced with other amino acids.

In some aspects, each amino acid of the polypeptide is independently selected as an L- or D-enantiomer.

In some aspects, an imaging agent is conjugated to a SEQ ID NO: 9 peptide at K27, or any one of SEQ ID NO: 1-SEQ ID NO: 481 peptide, SEQ ID NO: 482-SEQ ID NO: 485 peptide, or a fragment of the foregoing. The compounds, whether peptides or peptide-complexes can act as targeting moieties for one or more of a cavernoma (a.k.a., cavernous angiomas, cavernous hemangiomas, or cerebral cavernous malformation (CCM)), an arteriovenous malformation (a.k.a., arteriovenous angiomas, arteriovenous hemangiomas, or cerebral arteriovenous malformation (CAM)), an aneurysm (e.g., including abdominal aortic, thoracic aortic, and cerebral aneurysm), venous malformation, lymphatic malformation, capillary telangiectasia, mixed vascular malformation, or a spinal dural arteriovenous fistula.

In some aspects, the compound used is capable of passing across the blood brain barrier. In some aspects, the compound used further comprises a therapeutic agent. In some aspects, the polypeptide is conjugated to the therapeutic agent. In some aspects, the compound used further comprises a therapeutic agent attached to A. In further aspects, the therapeutic agent is a cytotoxic agent. In still other aspects, the therapeutic agent comprises a comprises a radioisotope, nanoparticle, toxin, enzyme, sensitizing drug, radiosensitizer, photosensitizer, nucleic acid, interfering RNA, antibody, antibody fragment, aptamer, anti-angiogenic agent, anti-metabolite, mitotic inhibitor, growth factor inhibitor, or a combination thereof.

In some aspects, the compound of the composition used is any suitable compound described herein. In other aspects, the compound of the composition further comprises an agent. In some aspects, the compound comprises a detectable agent. In one embodiment, the polypeptide is conjugated to an agent. In another embodiment, the polypeptide is conjugated to a detectable agent. In some embodiments, a detectable agent is a detectable label. In some embodiments, a detectable agent comprises a dye, a fluorophore, a fluorescent biotin compound, a luminescent compound, a chemiluminescent compound, a radioisotope, nanoparticle, a paramagnetic metal ion, or a combination thereof. In some embodiments, the polypeptide comprises a single lysine residue and the agent is conjugated to the polypeptide at the single lysine residue. In some embodiments, the polypeptide comprises no lysine residues and the agent is conjugated to the polypeptide at the N-terminus of the polypeptide.

Certain exemplary compounds falling within the scope of these genuses are provided below in TABLE 2 and further described herein, including both the peptide portion (indicated by A) and the detectable label portion.

TABLE 2
Compounds According to the Present Disclosure

| No. | Structure |
|---|---|
| 1 | 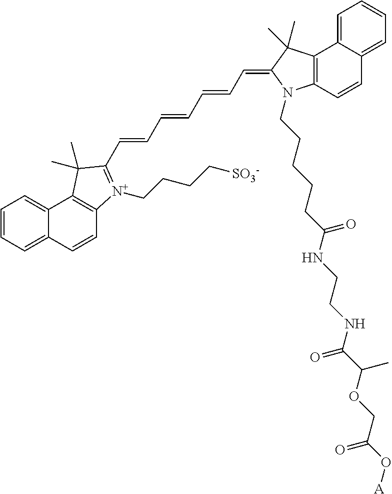 |
| 2 | 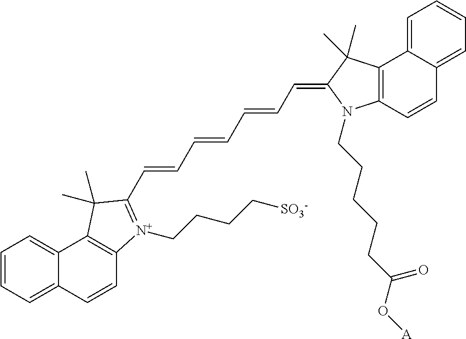 |
| 3 | 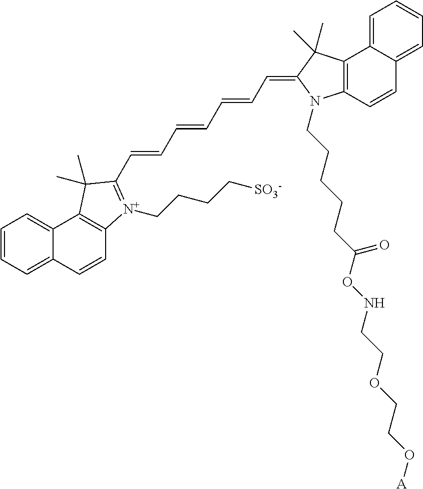 |
| 4 | 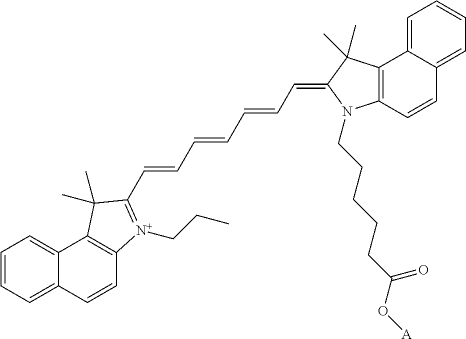 |
| 5 | 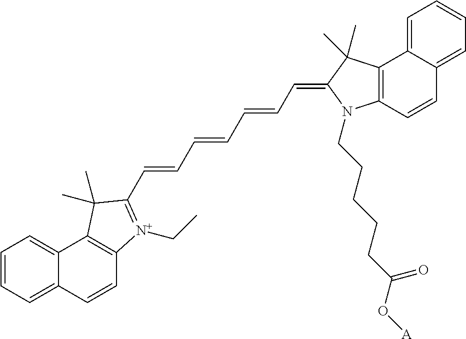 |
| 6 | 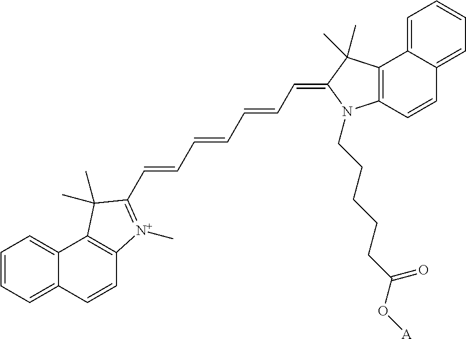 |
| 7 | 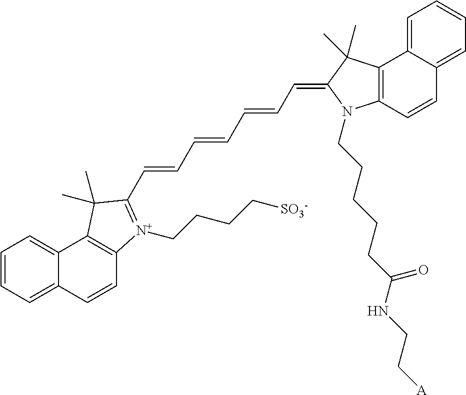 |
| 8 | 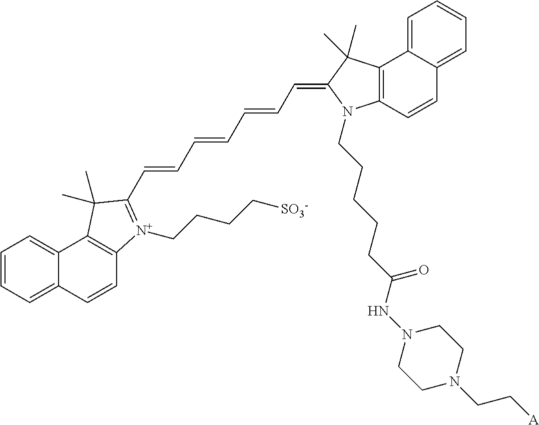 |
| 9 | 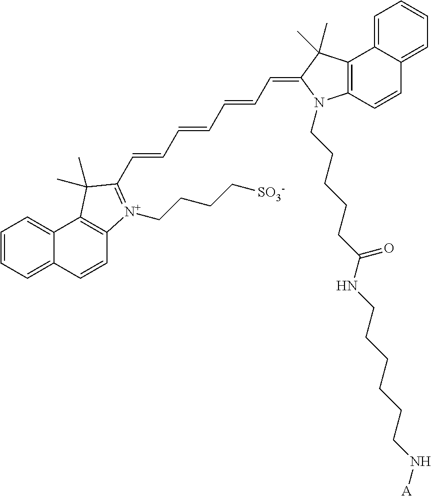 |
| 10 | 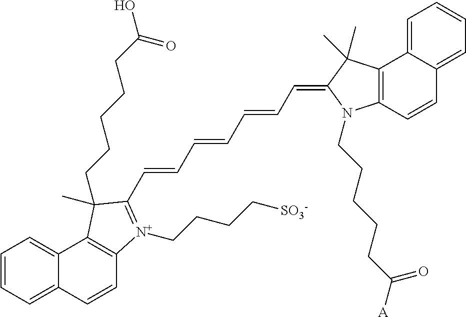 |
| 11 | 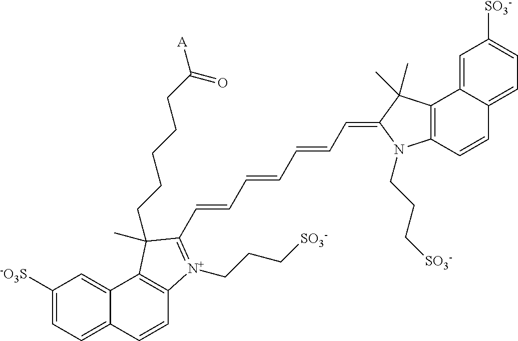 |
| 12 | 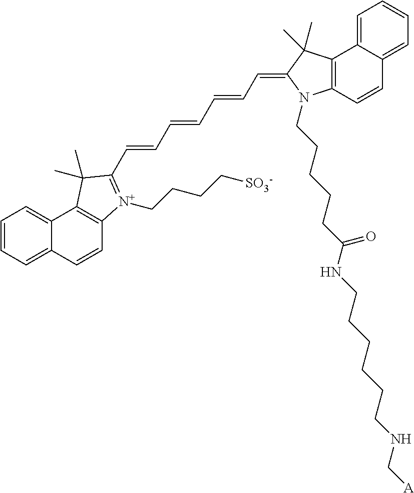 |
| 13 | 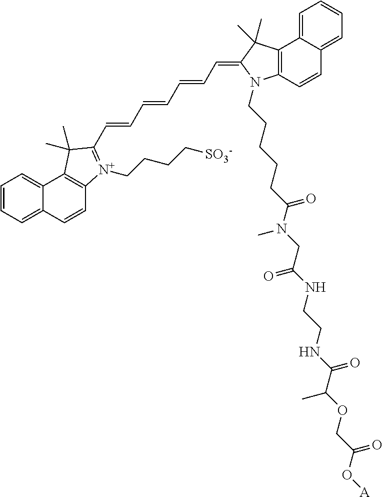 |
| 14 | 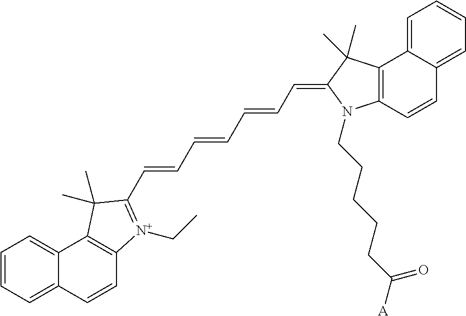 |
| 15 | 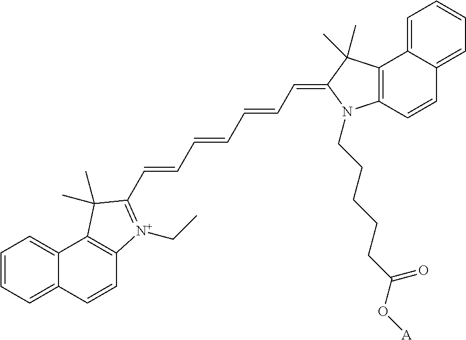 |
| 16 | 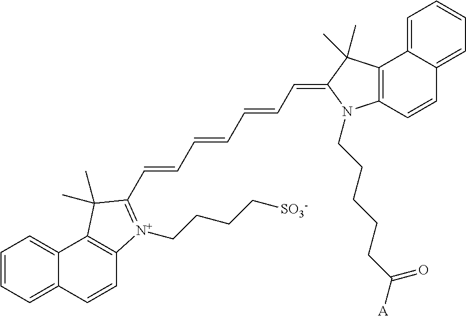 |
| 17 | 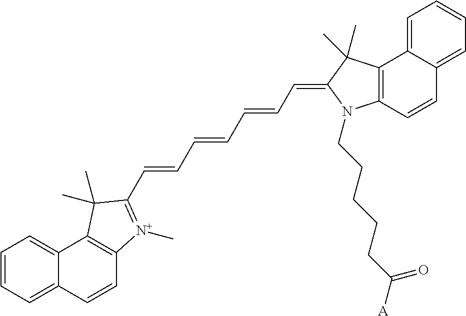 |
| 18 | 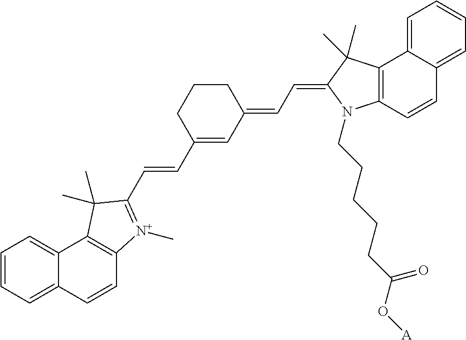 |
| 19 | 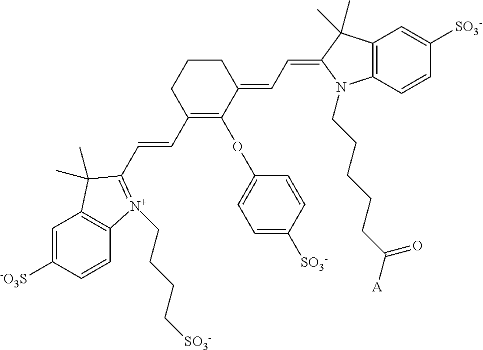 |
| 20 | 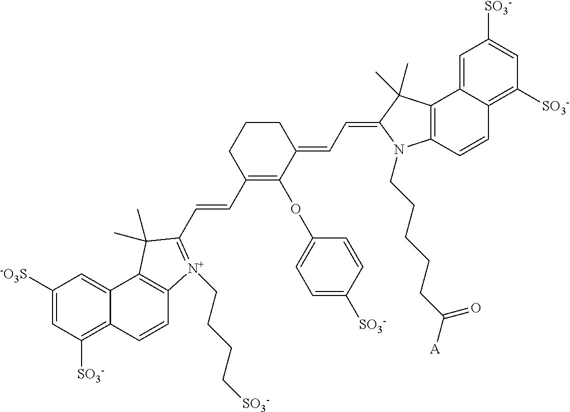 |
| 21 | 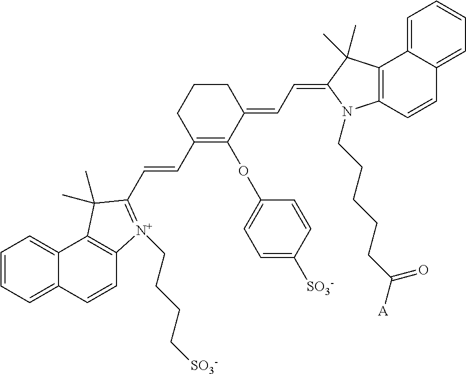 |
| 22 | 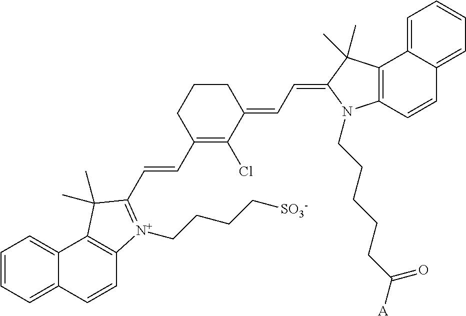 |
| 23 | 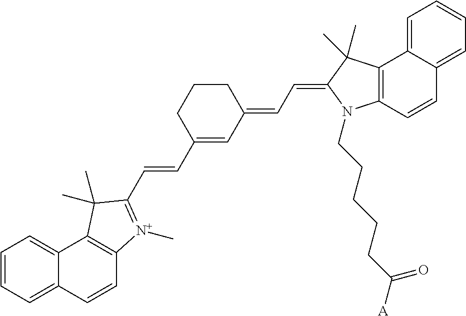 |
| 24 | 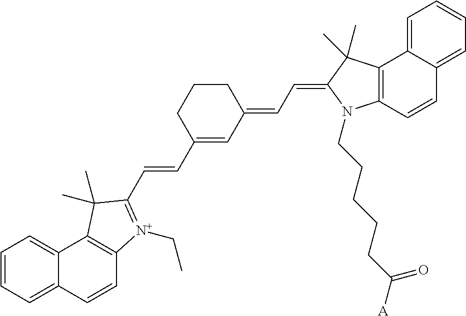 |
| 25 | 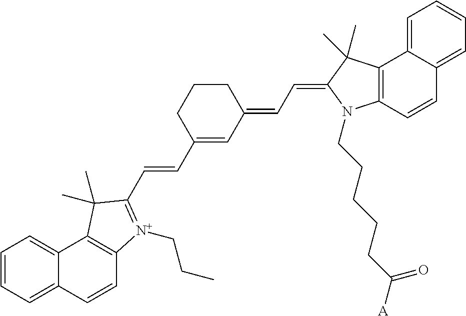 |
| 26 | 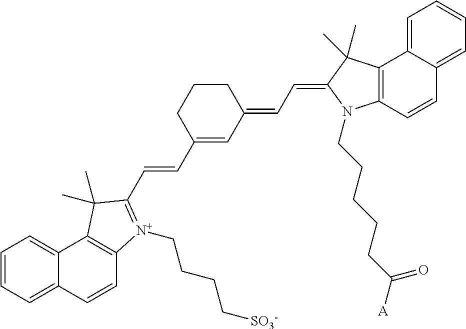 |
| 27 | 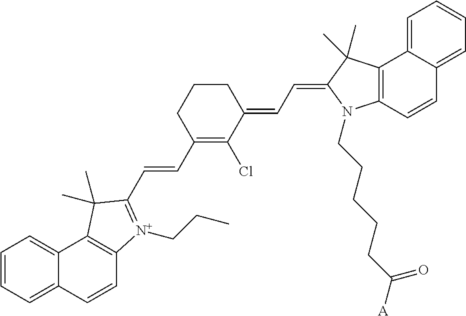 |
| 28 | 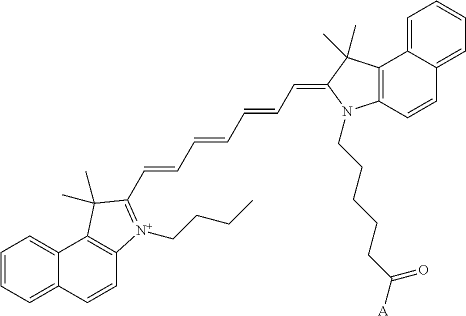 |
| 29 | 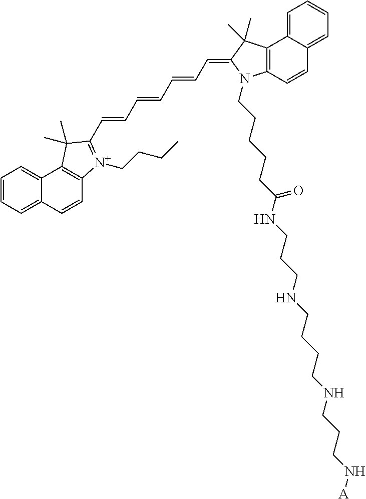 |
| 30 | 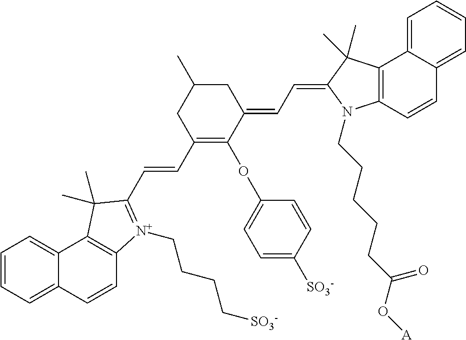 |
| 31 | 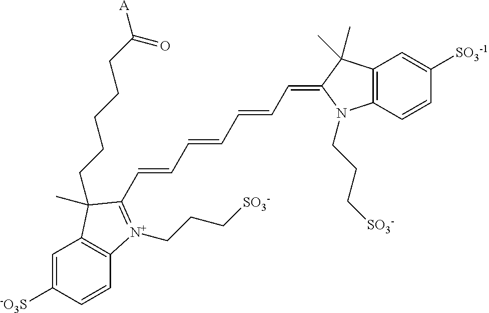 |
| 32 | 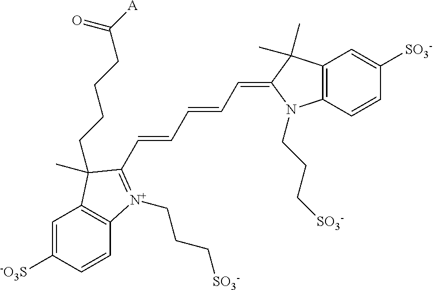 |
| 33 | 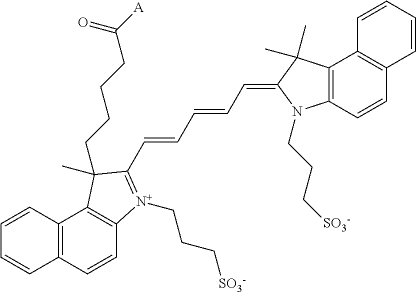 |
| 34 | 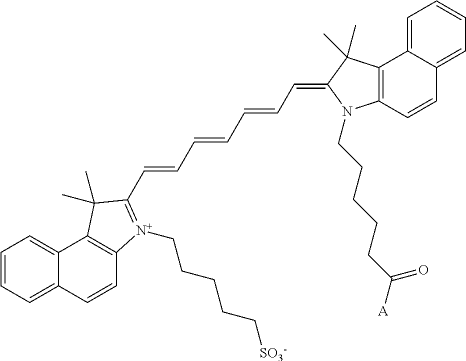 |
| 35 | 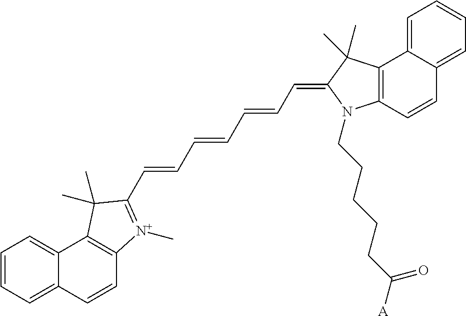 |
| 36 | 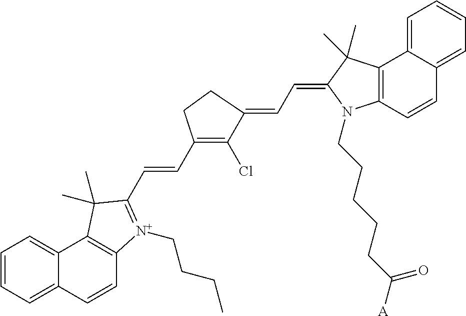 |
| 37 | 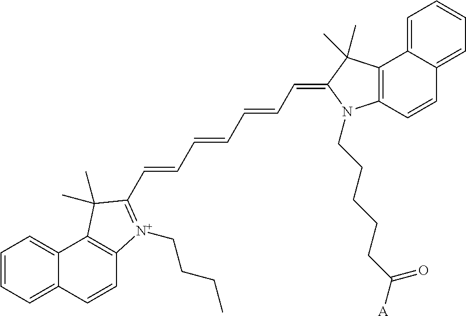 |
| 38 | 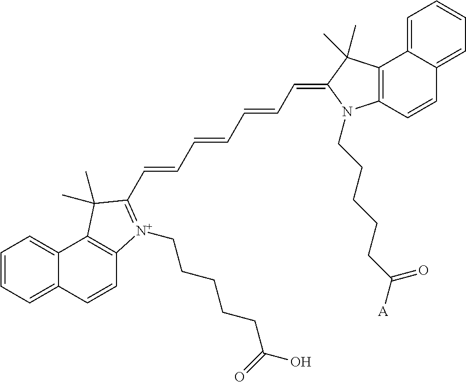 |
| 39 | 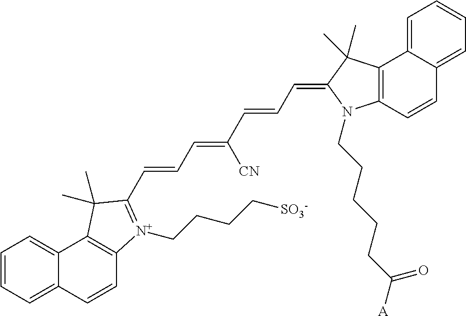 |
| 40 | 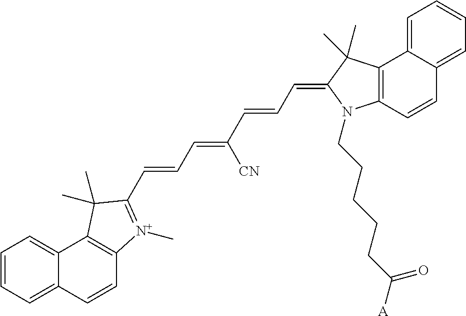 |
| 41 | 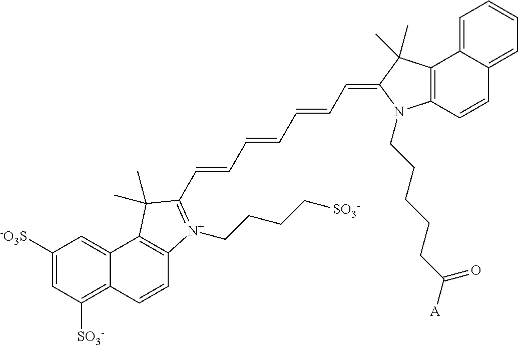 |
| 42 | 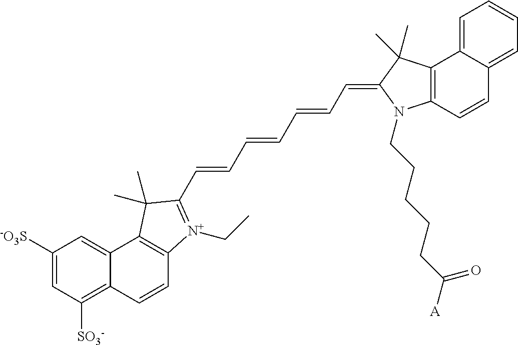 |
| 43 | 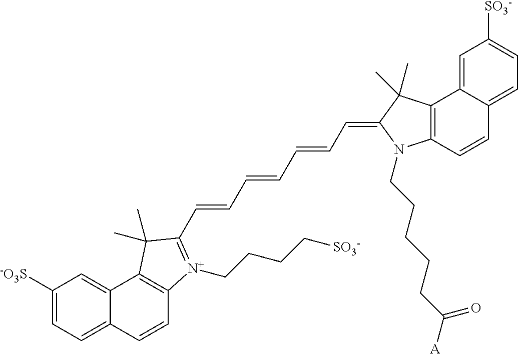 |
| 44 | 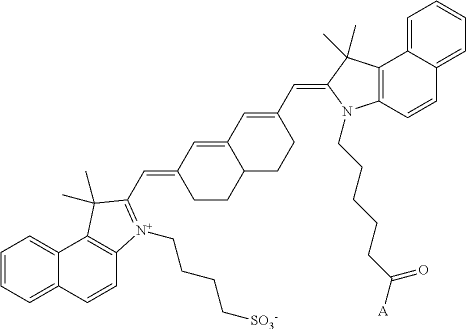 |
| 45 | 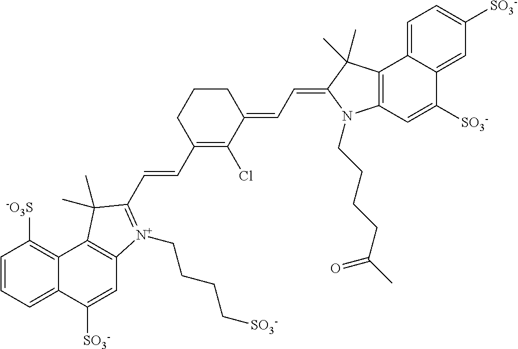 |
| 46 | 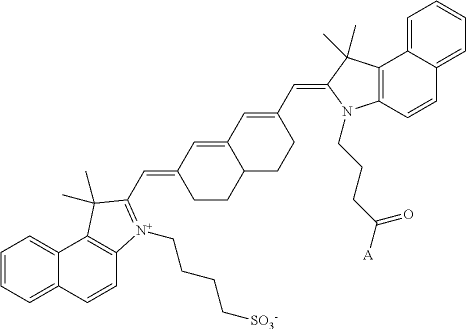 |
| 47 | 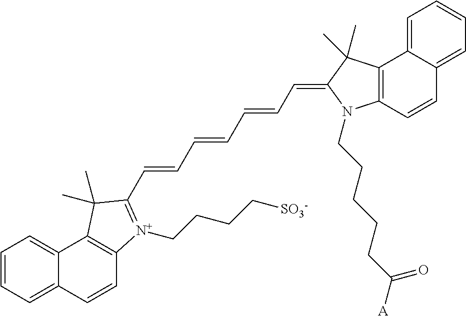 |
| 48 | 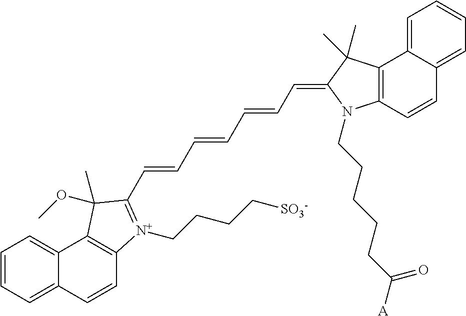 |
| 49 | 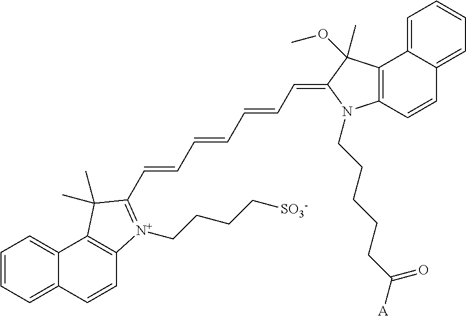 |
| 50 | 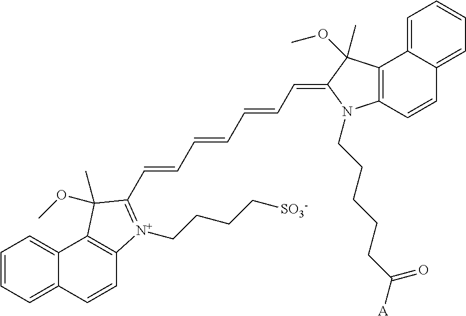 |
| 51 | 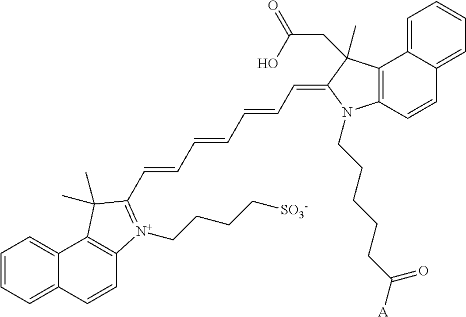 |
| 52 | 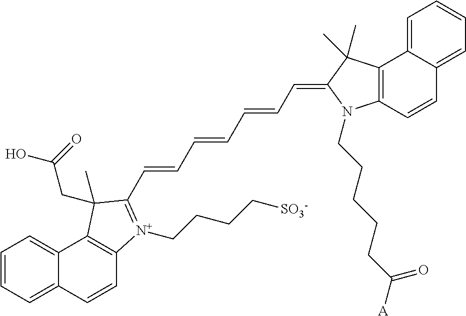 |
| 53 | 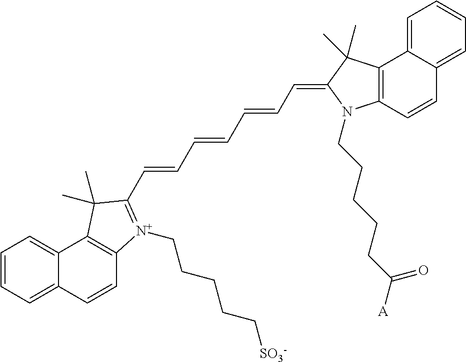 |
| 54 | 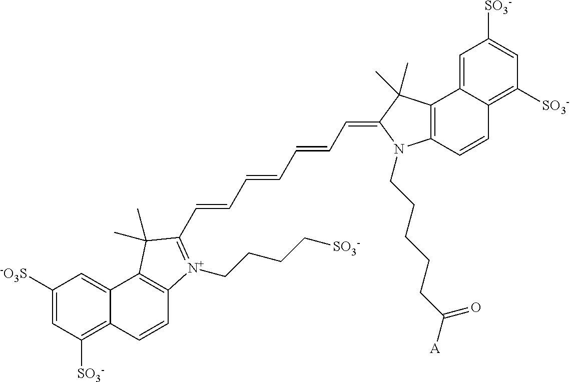 |
| 55 | 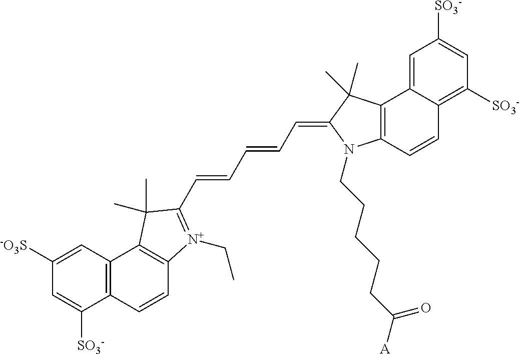 |
| 56 | 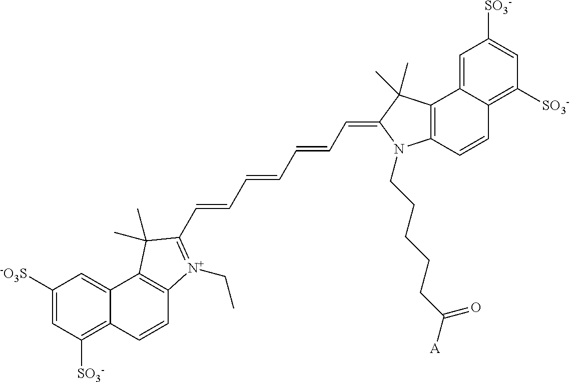 |
| 57 | 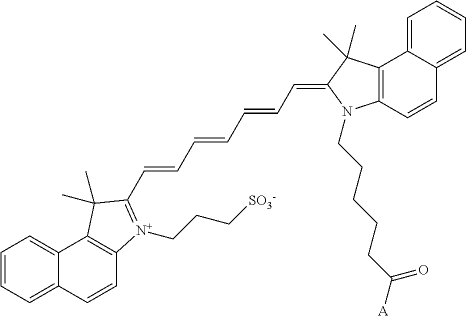 |
| 58 | 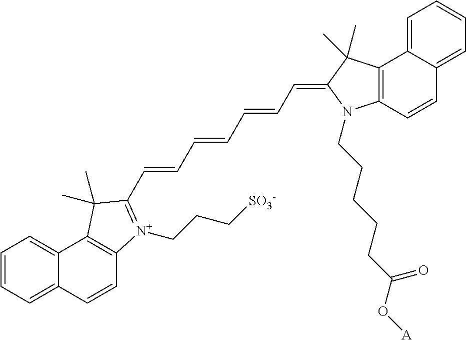 |
| 59 | 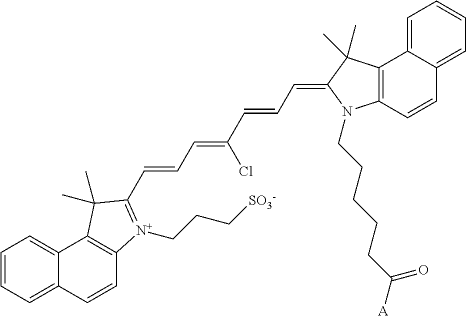 |
| 60 | 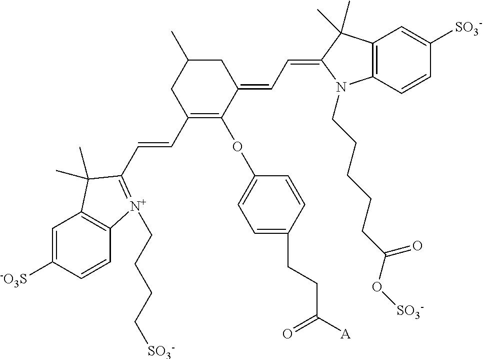 |

The peptide portion “A” in compounds 1-60 can comprise any of the peptides described herein, such as any one of SEQ ID NO: 1-SEQ ID NO: 485. In some embodiments, the peptide portion A is SEQ ID NO: 5 attached at K-27 to any one of compounds 1-60. In some embodiments, the peptide portion A is SEQ ID NO: 6 attached at K-27 to any one of compounds 1-60. In some embodiments, the peptide portion A is SEQ ID NO: 8 attached at K-27 to ay one of compounds 1-60. In some embodiments, the peptide portion A is SEQ ID NO: 9 attached at K-27 to any one of compounds 1-60. In some embodiments, the peptide portion A is SEQ ID NO: 11 attached at K-23 to any one of compounds 1-60. In some embodiments, the peptide portion A is SEQ ID NO: 12 attached at K-23 to any one of compounds 1-60. In some embodiments, the peptide portion A is SEQ ID NO: 13 attached at K-15 to any one of compounds 1-60. In some embodiments, the peptide portion A is SEQ ID NO: 16 attached at K-15 to any one of compounds 1-60. In some embodiments, the peptide portion A is SEQ ID NO: 20 attached at K-23 to any one of compounds 1-60. In some embodiments, the peptide portion A is SEQ ID NO: 21 attached at K-23 to any one of compounds 1-60. In some embodiments, the peptide portion A is SEQ ID NO. 22 attached at K-15 to any one of compounds 1-60. In some embodiments, the peptide portion A is SEQ ID NO: 25 attached at K-15 to any one of compounds 1-60.

TABLE 3 below sets forth certain polypeptide sequences for use with the present disclosure. Citrulline is designated as “Cit” in the sequences.

TABLE 3
Compounds of the Exemplary Peptide Sequence Suitable for Use in
the Compounds of the Present Disclosure. Cit = Citrulline.

| SEQ ID NO | Polypeptide Sequence |
|---|---|
| 1 | MCMPCFTTDHQMARKCDDCCGGKGRGKCYGPQCLCR |
| 2 | MCMPCFTTDHQMARACDDCCGGKGRGKCYGPQCLCR |
| 3 | MCMPCFTTDHQMARRCDDCCGGKGRGKCYGPQCLCR |
| 4 | MCMPCFTTDHQMARKCDDCCGGAGRGKCYGPQCLCR |
| 5 | MCMPCFTTDHQMARACDDCCGGAGRGKCYGPQCLCR |
| 6 | MCMPCFTTDHQMARRCDDCCGGAGRGKCYGPQCLCR |
| 7 | MCMPCFTTDHQMARKCDDCCGGRGRGKCYGPQCLCR |
| 8 | MCMPCFTTDHQMARACDDCCGGRGRGKCYGPQCLCR |
| 9 | MCMPCFTTDHQMARRCDDCCGGRGRGKCYGPQCLCR |
| 10 | MCMPCFTTDHQMARKCDDCCGGKGRGACYGPQCLCR |
| 11 | MCMPCFTTDHQMARACDDCCGGKGRGACYGPQCLCR |
| 12 | MCMPCFTTDHQMARRCDDCCGGKGRGACYGPQCLCR |
| 13 | MCMPCFTTDHQMARKCDDCCGGAGRGACYGPQCLCR |
| 14 | MCMPCFTTDHQMARACDDCCGGAGRGACYGPQCLCR |
| 15 | MCMPCFTTDHQMARRCDDCCGGAGRGACYGPQCLCR |
| 16 | MCMPCFTTDHQMARKCDDCCGGRGRGACYGPQCLCR |
| 17 | MCMPCFTTDHQMARACDDCCGGRGRGACYGPQCLCR |
| 18 | MCMPCFTTDHQMARRCDDCCGGRGRGACYGPQCLCR |
| 19 | MCMPCFTTDHQMARKCDDCCGGKGRGRCYGPQCLCR |
| 20 | MCMPCFTTDHQMARACDDCCGGKGRGRCYGPQCLCR |
| 21 | MCMPCFTTDHQMARRCDDCCGGKGRGRCYGPQCLCR |
| 22 | MCMPCFTTDHQMARKCDDCCGGAGRGRCYGPQCLCR |
| 23 | MCMPCFTTDHQMARACDDCCGGAGRGRCYGPQCLCR |
| 24 | MCMPCFTTDHQMARRCDDCCGGAGRGRCYGPQCLCR |
| 25 | MCMPCFTTDHQMARKCDDCCGGRGRGRCYGPQCLCR |
| 26 | MCMPCFTTDHQMARACDDCCGGRGRGRCYGPQCLCR |
| 27 | MCMPCFTTDHQMARRCDDCCGGRGRGRCYGPQCLCR |
| 28 | MCMPCFTTDHQMARRCDDCCGGRGRGRCYGPQCLCR |
| 29 | KCMPCFTTDHQMARRCDDCCGGRGRGRCYGPQCLCR |
| 30 | ACAPCFTTDHQAARRCDDCCGGRGRGRCYGPQCLCR |
| 31 | KCAPCFTTDHQAARRCDDCCGGRGRGRCYGPQCLCR |
| 32 | MCMPCFTTDHQMAR(Cit)CDDCCGG(Cit)GRGKCYGPQCLCR |
| 33 | MCMPCFTTDHQMAR(Cit)CDDCCGG(Cit)GRG(Cit)CYGPQCLCR |
| 34 | KCMPCFTTDHQMAR(Cit)CDDCCGG(Cit)GRG(Cit)CYGPQCLCR |
| 35 | ACAPCFTTDHQAAR(Cit)CDDCCGG(Cit)GRGKCYGPQCLCR |
| 36 | ACAPCFTTDHQAAR(Cit)CDDCCGG(Cit)GRG(Cit)CYGPQCLCR |
| 37 | KCAPCFTTDHQAAR(Cit)CDDCCGG(Cit)GRG(Cit)CYGPQCLCR |
| 38 | MCMPCFTTDHQMARKCDDCCGGKGRGKCYGPQCLCRGAGAAGG |
| 39 | MCMPCFTTDHQMARACDDCCGGKGRGKCYGPQCLCRGAGAAGG |
| 40 | MCMPCFTTDHQMARRCDDCCGGKGRGKCYGPQCLCRGAGAAGG |
| 41 | MCMPCFTTDHQMARKCDDCCGGAGRGKCYGPQCLCRGAGAAGG |
| 42 | MCMPCFTTDHQMARACDDCCGGAGRGKCYGPQCLCRGAGAAGG |
| 43 | MCMPCFTTDHQMARRCDDCCGGAGRGKCYGPQCLCRGAGAAGG |
| 44 | MCMPCFTTDHQMARKCDDCCGGRGRGKCYGPQCLCRGAGAAGG |
| 45 | MCMPCFTTDHQMARACDDCCGGRGRGKCYGPQCLCRGAGAAGG |
| 46 | MCMPCFTTDHQMARRCDDCCGGRGRGKCYGPQCLCRGAGAAGG |
| 47 | MCMPCFTTDHQMARKCDDCCGGKGRGACYGPQCLCRGAGAAGG |
| 48 | MCMPCFTTDHQMARACDDCCGGKGRGACYGPQCLCRGAGAAGG |
| 49 | MCMPCFTTDHQMARRCDDCCGGKGRGACYGPQCLCRGAGAAGG |
| 50 | MCMPCFTTDHQMARKCDDCCGGAGRGACYGPQCLCRGAGAAGG |
| 51 | MCMPCFTTDHQMARACDDCCGGAGRGACYGPQCLCRGAGAAGG |
| 52 | MCMPCFTTDHQMARRCDDCCGGAGRGACYGPQCLCRGAGAAGG |
| 53 | MCMPCFTTDHQMARKCDDCCGGRGRGACYGPQCLCRGAGAAGG |
| 54 | MCMPCFTTDHQMARACDDCCGGRGRGACYGPQCLCRGAGAAGG |
| 55 | MCMPCFTTDHQMARRCDDCCGGRGRGACYGPQCLCRGAGAAGG |
| 56 | MCMPCFTTDHQMARKCDDCCGGKGRGRCYGPQCLCRGAGAAGG |
| 57 | MCMPCFTTDHQMARACDDCCGGKGRGRCYGPQCLCRGAGAAGG |
| 58 | MCMPCFTTDHQMARRCDDCCGGKGRGRCYGPQCLCRGAGAAGG |
| 59 | MCMPCFTTDHQMARKCDDCCGGAGRGRCYGPQCLCRGAGAAGG |
| 60 | MCMPCFTTDHQMARACDDCCGGAGRGRCYGPQCLCRGAGAAGG |
| 61 | MCMPCFTTDHQMARRCDDCCGGAGRGRCYGPQCLCRGAGAAGG |
| 62 | MCMPCFTTDHQMARKCDDCCGGRGRGRCYGPQCLCRGAGAAGG |
| 63 | MCMPCFTTDHQMARACDDCCGGRGRGRCYGPQCLCRGAGAAGG |
| 64 | MCMPCFTTDHQMARRCDDCCGGRGRGRCYGPQCLCRGAGAAGG |
| 65 | MCMPCFTTDHQMARRCDDCCGGRGRGRCYGPQCLCRGAGAAGG |
| 66 | KCMPCFTTDHQMARRCDDCCGGRGRGRCYGPQCLCRGAGAAGG |
| 67 | ACAPCFTTDHQAARRCDDCCGGRGRGRCYGPQCLCRGAGAAGG |
| 68 | KCAPCFTTDHQAARRCDDCCGGRGRGRCYGPQCLCRGAGAAGG |
| 69 | MCMPCFTTDHQMAR(Cit)CDDCCGG(Cit)GRGKCYGPQCLCRGAGAAGG |
| 70 | MCMPCFTTDHQMAR(Cit)CDDCCGG(Cit)GRG(Cit)CYGPQCLCRGAGAAGG |
| 71 | KCMPCFTTDHQMAR(Cit)CDDCCGG(Cit)GRG(Cit)CYGPQCLCRGAGAAGG |
| 72 | ACAPCFTTDHQAAR(Cit)CDDCCGG(Cit)GRGKCYGPQCLCRGAGAAGG |
| 73 | ACAPCFTTDHQAAR(Cit)CDDCCGG(Cit)GRG(Cit)CYGPQCLCRGAGAAGG |
| 74 | KCAPCFTTDHQAAR(Cit)CDDCCGG(Cit)GRG(Cit)CYGPQCLCRGAGAAGG |
| 75 | MCMPCFTTDHQMVRKCDDCCGGKGRGKCYGPQCLCR |
| 76 | MCMPCFTTDHQMVRVCDDCCGGKGRGKCYGPQCLCR |
| 77 | MCMPCFTTDHQMVRRCDDCCGGKGRGKCYGPQCLCR |
| 78 | MCMPCFTTDHQMVRKCDDCCGGVGRGKCYGPQCLCR |
| 79 | MCMPCFTTDHQMVRVCDDCCGGVGRGKCYGPQCLCR |
| 80 | MCMPCFTTDHQMVRRCDDCCGGVGRGKCYGPQCLCR |
| 81 | MCMPCFTTDHQMVRKCDDCCGGRGRGKCYGPQCLCR |
| 82 | MCMPCFTTDHQMVRVCDDCCGGRGRGKCYGPQCLCR |
| 83 | MCMPCFTTDHQMVRRCDDCCGGRGRGKCYGPQCLCR |
| 84 | MCMPCFTTDHQMVRKCDDCCGGKGRGVCYGPQCLCR |
| 85 | MCMPCFTTDHQMVRVCDDCCGGKGRGVCYGPQCLCR |
| 86 | MCMPCFTTDHQMVRRCDDCCGGKGRGVCYGPQCLCR |
| 87 | MCMPCFTTDHQMVRKCDDCCGGVGRGVCYGPQCLCR |
| 88 | MCMPCFTTDHQMVRVCDDCCGGVGRGVCYGPQCLCR |
| 89 | MCMPCFTTDHQMVRRCDDCCGGVGRGVCYGPQCLCR |
| 90 | MCMPCFTTDHQMVRKCDDCCGGRGRGVCYGPQCLCR |
| 91 | MCMPCFTTDHQMVRVCDDCCGGRGRGVCYGPQCLCR |
| 92 | MCMPCFTTDHQMVRRCDDCCGGRGRGVCYGPQCLCR |
| 93 | MCMPCFTTDHQMVRKCDDCCGGKGRGRCYGPQCLCR |
| 94 | MCMPCFTTDHQMVRVCDDCCGGKGRGRCYGPQCLCR |
| 95 | MCMPCFTTDHQMVRRCDDCCGGKGRGRCYGPQCLCR |
| 96 | MCMPCFTTDHQMVRKCDDCCGGVGRGRCYGPQCLCR |
| 97 | MCMPCFTTDHQMVRVCDDCCGGVGRGRCYGPQCLCR |
| 98 | MCMPCFTTDHQMVRRCDDCCGGVGRGRCYGPQCLCR |
| 99 | MCMPCFTTDHQMVRKCDDCCGGRGRGRCYGPQCLCR |
| 100 | MCMPCFTTDHQMVRVCDDCCGGRGRGRCYGPQCLCR |
| 101 | MCMPCFTTDHQMVRRCDDCCGGRGRGRCYGPQCLCR |
| 102 | MCMPCFTTDHQMVRRCDDCCGGRGRGRCYGPQCLCR |
| 103 | KCMPCFTTDHQMVRRCDDCCGGRGRGRCYGPQCLCR |
| 104 | VCVPCFTTDHQVVRRCDDCCGGRGRGRCYGPQCLCR |
| 105 | KCVPCFTTDHQVVRRCDDCCGGRGRGRCYGPQCLCR |
| 106 | MCMPCFTTDHQMVR(Cit)CDDCCGG(Cit)GRGKCYGPQCLCR |
| 107 | MCMPCFTTDHQMVR(Cit)CDDCCGG(Cit)GRG(Cit)CYGPQCLCR |
| 108 | KCMPCFTTDHQMVR(Cit)CDDCCGG(Cit)GRG(Cit)CYGPQCLCR |
| 109 | VCVPCFTTDHQVVR(Cit)CDDCCGG(Cit)GRGKCYGPQCLCR |
| 110 | VCVPCFTTDHQVVR(Cit)CDDCCGG(Cit)GRG(Cit)CYGPQCLCR |
| ill | KCVPCFTTDHQVVR(Cit)CDDCCGG(Cit)GRG(Cit)CYGPQCLCR |
| 112 | MCMPCFTTDHQMVRKCDDCCGGKGRGKCYGPQCLCRGAGAAGG |
| 113 | MCMPCFTTDHQMVRVCDDCCGGKGRGKCYGPQCLCRGAGAAGG |
| 114 | MCMPCFTTDHQMVRRCDDCCGGKGRGKCYGPQCLCRGAGAAGG |
| 115 | MCMPCFTTDHQMVRKCDDCCGGVGRGKCYGPQCLCRGAGAAGG |
| 116 | MCMPCFTTDHQMVRVCDDCCGGVGRGKCYGPQCLCRGAGAAGG |
| 117 | MCMPCFTTDHQMVRRCDDCCGGVGRGKCYGPQCLCRGAGAAGG |
| 118 | MCMPCFTTDHQMVRKCDDCCGGRGRGKCYGPQCLCRGAGAAGG |
| 119 | MCMPCFTTDHQMVRVCDDCCGGRGRGKCYGPQCLCRGAGAAGG |
| 120 | MCMPCFTTDHQMVRRCDDCCGGRGRGKCYGPQCLCRGAGAAGG |
| 121 | MCMPCFTTDHQMVRKCDDCCGGKGRGVCYGPQCLCRGAGAAGG |
| 122 | MCMPCFTTDHQMVRVCDDCCGGKGRGVCYGPQCLCRGAGAAGG |
| 123 | MCMPCFTTDHQMVRRCDDCCGGKGRGVCYGPQCLCRGAGAAGG |
| 124 | MCMPCFTTDHQMVRKCDDCCGGVGRGVCYGPQCLCRGAGAAGG |
| 125 | MCMPCFTTDHQMVRVCDDCCGGVGRGVCYGPQCLCRGAGAAGG |
| 126 | MCMPCFTTDHQMVRRCDDCCGGVGRGVCYGPQCLCRGAGAAGG |
| 127 | MCMPCFTTDHQMVRKCDDCCGGRGRGVCYGPQCLCRGAGAAGG |
| 128 | MCMPCFTTDHQMVRVCDDCCGGRGRGVCYGPQCLCRGAGAAGG |
| 129 | MCMPCFTTDHQMVRRCDDCCGGRGRGVCYGPQCLCRGAGAAGG |
| 130 | MCMPCFTTDHQMVRKCDDCCGGKGRGRCYGPQCLCRGAGAAGG |
| 131 | MCMPCFTTDHQMVRVCDDCCGGKGRGRCYGPQCLCRGAGAAGG |
| 132 | MCMPCFTTDHQMVRRCDDCCGGKGRGRCYGPQCLCRGAGAAGG |
| 133 | MCMPCFTTDHQMVRKCDDCCGGVGRGRCYGPQCLCRGAGAAGG |
| 134 | MCMPCFTTDHQMVRVCDDCCGGVGRGRCYGPQCLCRGAGAAGG |
| 135 | MCMPCFTTDHQMVRRCDDCCGGVGRGRCYGPQCLCRGAGAAGG |
| 136 | MCMPCFTTDHQMVRKCDDCCGGRGRGRCYGPQCLCRGAGAAGG |
| 137 | MCMPCFTTDHQMVRVCDDCCGGRGRGRCYGPQCLCRGAGAAGG |
| 138 | MCMPCFTTDHQMVRRCDDCCGGRGRGRCYGPQCLCRGAGAAGG |
| 139 | MCMPCFTTDHQMVRRCDDCCGGRGRGRCYGPQCLCRGAGAAGG |
| 140 | KCMPCFTTDHQMVRRCDDCCGGRGRGRCYGPQCLCRGAGAAGG |
| 141 | VCVPCFTTDHQVVRRCDDCCGGRGRGRCYGPQCLCRGAGAAGG |
| 142 | KCVPCFTTDHQVVRRCDDCCGGRGRGRCYGPQCLCRGAGAAGG |
| 143 | MCMPCFTTDHQMVR(Cit)CDDCCGG(Cit)GRGKCYGPQCLCRGAGAAGG |
| 144 | MCMPCFTTDHQMVR(Cit)CDDCCGG(Cit)GRG(Cit)CYGPQCLCRGAGAAGG |
| 145 | KCMPCFTTDHQMVR(Cit)CDDCCGG(Cit)GRG(Cit)CYGPQCLCRGAGAAGG |
| 146 | VCVPCFTTDHQVVR(Cit)CDDCCGG(Cit)GRGKCYGPQCLCRGAGAAGG |
| 147 | VCVPCFTTDHQVVR(Cit)CDDCCGG(Cit)GRG(Cit)CYGPQCLCRGAGAAGG |
| 148 | KCVPCFTTDHQVVR(Cit)CDDCCGG(Cit)GRG(Cit)CYGPQCLCRGAGAAGG |
| 149 | MCMPCFTTDHQMLRKCDDCCGGKGRGKCYGPQCLCR |
| 150 | MCMPCFTTDHQMLRLCDDCCGGKGRGKCYGPQCLCR |
| 151 | MCMPCFTTDHQMLRRCDDCCGGKGRGKCYGPQCLCR |
| 152 | MCMPCFTTDHQMLRKCDDCCGGLGRGKCYGPQCLCR |
| 153 | MCMPCFTTDHQMLRLCDDCCGGLGRGKCYGPQCLCR |
| 154 | MCMPCFTTDHQMLRRCDDCCGGLGRGKCYGPQCLCR |
| 155 | MCMPCFTTDHQMLRKCDDCCGGRGRGKCYGPQCLCR |
| 156 | MCMPCFTTDHQMLRLCDDCCGGRGRGKCYGPQCLCR |
| 157 | MCMPCFTTDHQMLRRCDDCCGGRGRGKCYGPQCLCR |
| 158 | MCMPCFTTDHQMLRKCDDCCGGKGRGLCYGPQCLCR |
| 159 | MCMPCFTTDHQMLRLCDDCCGGKGRGLCYGPQCLCR |
| 160 | MCMPCFTTDHQMLRRCDDCCGGKGRGLCYGPQCLCR |
| 161 | MCMPCFTTDHQMLRKCDDCCGGLGRGLCYGPQCLCR |
| 162 | MCMPCFTTDHQMLRLCDDCCGGLGRGLCYGPQCLCR |
| 163 | MCMPCFTTDHQMLRRCDDCCGGLGRGLCYGPQCLCR |
| 164 | MCMPCFTTDHQMLRKCDDCCGGRGRGLCYGPQCLCR |
| 165 | MCMPCFTTDHQMLRLCDDCCGGRGRGLCYGPQCLCR |
| 166 | MCMPCFTTDHQMLRRCDDCCGGRGRGLCYGPQCLCR |
| 167 | MCMPCFTTDHQMLRKCDDCCGGKGRGRCYGPQCLCR |
| 168 | MCMPCFTTDHQMLRLCDDCCGGKGRGRCYGPQCLCR |
| 169 | MCMPCFTTDHQMLRRCDDCCGGKGRGRCYGPQCLCR |
| 170 | MCMPCFTTDHQMLRKCDDCCGGLGRGRCYGPQCLCR |
| 171 | MCMPCFTTDHQMLRLCDDCCGGLGRGRCYGPQCLCR |
| 172 | MCMPCFTTDHQMLRRCDDCCGGLGRGRCYGPQCLCR |
| 173 | MCMPCFTTDHQMLRKCDDCCGGRGRGRCYGPQCLCR |
| 174 | MCMPCFTTDHQMLRLCDDCCGGRGRGRCYGPQCLCR |
| 175 | MCMPCFTTDHQMLRRCDDCCGGRGRGRCYGPQCLCR |
| 176 | MCMPCFTTDHQMLRRCDDCCGGRGRGRCYGPQCLCR |
| 177 | KCMPCFTTDHQMLRRCDDCCGGRGRGRCYGPQCLCR |
| 178 | LCLPCFTTDHQLLRRCDDCCGGRGRGRCYGPQCLCR |
| 179 | KCLPCFTTDHQLLRRCDDCCGGRGRGRCYGPQCLCR |
| 180 | MCMPCFTTDHQMLR(Cit)CDDCCGG(Cit)GRGKCYGPQCLCR |
| 181 | MCMPCFTTDHQMLR(Cit)CDDCCGG(Cit)GRG(Cit)CYGPQCLCR |
| 182 | KCMPCFTTDHQMLR(Cit)CDDCCGG(Cit)GRG(Cit)CYGPQCLCR |
| 183 | LCLPCFTTDHQLLR(Cit)CDDCCGG(Cit)GRGKCYGPQCLCR |
| 184 | LCLPCFTTDHQLLR(Cit)CDDCCGG(Cit)GRG(Cit)CYGPQCLCR |
| 185 | KCLPCFTTDHQLLR(Cit)CDDCCGG(Cit)GRG(Cit)CYGPQCLCR |
| 186 | MCMPCFTTDHQMLRKCDDCCGGKGRGKCYGPQCLCRGAGAAGG |
| 187 | MCMPCFTTDHQMLRLCDDCCGGKGRGKCYGPQCLCRGAGAAGG |
| 188 | MCMPCFTTDHQMLRRCDDCCGGKGRGKCYGPQCLCRGAGAAGG |
| 189 | MCMPCFTTDHQMLRKCDDCCGGLGRGKCYGPQCLCRGAGAAGG |
| 190 | MCMPCFTTDHQMLRLCDDCCGGLGRGKCYGPQCLCRGAGAAGG |
| 191 | MCMPCFTTDHQMLRRCDDCCGGLGRGKCYGPQCLCRGAGAAGG |
| 192 | MCMPCFTTDHQMLRKCDDCCGGRGRGKCYGPQCLCRGAGAAGG |
| 193 | MCMPCFTTDHQMLRLCDDCCGGRGRGKCYGPQCLCRGAGAAGG |
| 194 | MCMPCFTTDHQMLRRCDDCCGGRGRGKCYGPQCLCRGAGAAGG |
| 195 | MCMPCFTTDHQMLRKCDDCCGGKGRGLCYGPQCLCRGAGAAGG |
| 196 | MCMPCFTTDHQMLRLCDDCCGGKGRGLCYGPQCLCRGAGAAGG |
| 197 | MCMPCFTTDHQMLRRCDDCCGGKGRGLCYGPQCLCRGAGAAGG |
| 198 | MCMPCFTTDHQMLRKCDDCCGGLGRGLCYGPQCLCRGAGAAGG |
| 199 | MCMPCFTTDHQMLRLCDDCCGGLGRGLCYGPQCLCRGAGAAGG |
| 200 | MCMPCFTTDHQMLRRCDDCCGGLGRGLCYGPQCLCRGAGAAGG |
| 201 | MCMPCFTTDHQMLRKCDDCCGGRGRGLCYGPQCLCRGAGAAGG |
| 202 | MCMPCFTTDHQMLRLCDDCCGGRGRGLCYGPQCLCRGAGAAGG |
| 203 | MCMPCFTTDHQMLRRCDDCCGGRGRGLCYGPQCLCRGAGAAGG |
| 204 | MCMPCFTTDHQMLRKCDDCCGGKGRGRCYGPQCLCRGAGAAGG |
| 205 | MCMPCFTTDHQMLRLCDDCCGGKGRGRCYGPQCLCRGAGAAGG |
| 206 | MCMPCFTTDHQMLRRCDDCCGGKGRGRCYGPQCLCRGAGAAGG |
| 207 | MCMPCFTTDHQMLRKCDDCCGGLGRGRCYGPQCLCRGAGAAGG |
| 208 | MCMPCFTTDHQMLRLCDDCCGGLGRGRCYGPQCLCRGAGAAGG |
| 209 | MCMPCFTTDHQMLRRCDDCCGGLGRGRCYGPQCLCRGAGAAGG |
| 210 | MCMPCFTTDHQMLRKCDDCCGGRGRGRCYGPQCLCRGAGAAGG |
| 211 | MCMPCFTTDHQMLRLCDDCCGGRGRGRCYGPQCLCRGAGAAGG |
| 212 | MCMPCFTTDHQMLRRCDDCCGGRGRGRCYGPQCLCRGAGAAGG |
| 213 | MCMPCFTTDHQMLRRCDDCCGGRGRGRCYGPQCLCRGAGAAGG |
| 214 | KCMPCFTTDHQMLRRCDDCCGGRGRGRCYGPQCLCRGAGAAGG |
| 215 | LCLPCFTTDHQLLRRCDDCCGGRGRGRCYGPQCLCRGAGAAGG |
| 216 | KCLPCFTTDHQLLRRCDDCCGGRGRGRCYGPQCLCRGAGAAGG |
| 217 | MCMPCFTTDHQMLR(Cit)CDDCCGG(Cit)GRGKCYGPQCLCRGAGAAGG |
| 218 | MCMPCFTTDHQMLR(Cit)CDDCCGG(Cit)GRG(Cit)CYGPQCLCRGAGAAGG |
| 219 | KCMPCFTTDHQMLR(Cit)CDDCCGG(Cit)GRG(Cit)CYGPQCLCRGAGAAGG |
| 220 | LCLPCFTTDHQLLR(Cit)CDDCCGG(Cit)GRGKCYGPQCLCRGAGAAGG |
| 221 | LCLPCFTTDHQLLR(Cit)CDDCCGG(Cit)GRG(Cit)CYGPQCLCRGAGAAGG |
| 222 | KCLPCFTTDHQLLR(Cit)CDDCCGG(Cit)GRG(Cit)CYGPQCLCRGAGAAGG |
| 223 | GCGPCFTTDHQGARKCDDCCGGKGRGKCYGPQCLCR |
| 224 | GCGPCFTTDHQGARACDDCCGGKGRGKCYGPQCLCR |
| 225 | GCGPCFTTDHQGARRCDDCCGGKGRGKCYGPQCLCR |
| 226 | GCGPCFTTDHQGARKCDDCCGGAGRGKCYGPQCLCR |
| 227 | GCGPCFTTDHQGARACDDCCGGAGRGKCYGPQCLCR |
| 228 | GCGPCFTTDHQGARRCDDCCGGAGRGKCYGPQCLCR |
| 229 | GCGPCFTTDHQGARKCDDCCGGRGRGKCYGPQCLCR |
| 230 | GCGPCFTTDHQGARACDDCCGGRGRGKCYGPQCLCR |
| 231 | GCGPCFTTDHQGARRCDDCCGGRGRGKCYGPQCLCR |
| 232 | GCGPCFTTDHQGARKCDDCCGGKGRGACYGPQCLCR |
| 233 | GCGPCFTTDHQGARACDDCCGGKGRGACYGPQCLCR |
| 234 | GCGPCFTTDHQGARRCDDCCGGKGRGACYGPQCLCR |
| 235 | GCGPCFTTDHQGARKCDDCCGGAGRGACYGPQCLCR |
| 236 | GCGPCFTTDHQGARACDDCCGGAGRGACYGPQCLCR |
| 237 | GCGPCFTTDHQGARRCDDCCGGAGRGACYGPQCLCR |
| 238 | GCGPCFTTDHQGARKCDDCCGGRGRGACYGPQCLCR |
| 239 | GCGPCFTTDHQGARACDDCCGGRGRGACYGPQCLCR |
| 240 | GCGPCFTTDHQGARRCDDCCGGRGRGACYGPQCLCR |
| 241 | GCGPCFTTDHQGARKCDDCCGGKGRGRCYGPQCLCR |
| 242 | GCGPCFTTDHQGARACDDCCGGKGRGRCYGPQCLCR |
| 243 | GCGPCFTTDHQGARRCDDCCGGKGRGRCYGPQCLCR |
| 244 | GCGPCFTTDHQGARKCDDCCGGAGRGRCYGPQCLCR |
| 245 | GCGPCFTTDHQGARACDDCCGGAGRGRCYGPQCLCR |
| 246 | GCGPCFTTDHQGARRCDDCCGGAGRGRCYGPQCLCR |
| 247 | GCGPCFTTDHQGARKCDDCCGGRGRGRCYGPQCLCR |
| 248 | GCGPCFTTDHQGARACDDCCGGRGRGRCYGPQCLCR |
| 249 | GCGPCFTTDHQGARRCDDCCGGRGRGRCYGPQCLCR |
| 250 | GCGPCFTTDHQGARRCDDCCGGRGRGRCYGPQCLCR |
| 251 | KCGPCFTTDHQGARRCDDCCGGRGRGRCYGPQCLCR |
| 252 | ACAPCFTTDHQAARRCDDCCGGRGRGRCYGPQCLCR |
| 253 | KCAPCFTTDHQAARRCDDCCGGRGRGRCYGPQCLCR |
| 254 | GCGPCFTTDHQGAR(Cit)CDDCCGG(Cit)GRGKCYGPQCLCR |
| 255 | GCGPCFTTDHQGAR(Cit)CDDCCGG(Cit)GRG(Cit)CYGPQCLCR |
| 256 | KCGPCFTTDHQGAR(Cit)CDDCCGG(Cit)GRG(Cit)CYGPQCLCR |
| 257 | ACAPCFTTDHQAAR(Cit)CDDCCGG(Cit)GRGKCYGPQCLCR |
| 258 | ACAPCFTTDHQAAR(Cit)CDDCCGG(Cit)GRG(Cit)CYGPQCLCR |
| 259 | KCAPCFTTDHQAAR(Cit)CDDCCGG(Cit)GRG(Cit)CYGPQCLCR |
| 260 | ACAPCFTTDHQAARKCDDCCGGKGRGKCYGPQCLCR |
| 261 | ACAPCFTTDHQAARACDDCCGGKGRGKCYGPQCLCR |
| 262 | ACAPCFTTDHQAARRCDDCCGGKGRGKCYGPQCLCR |
| 263 | ACAPCFTTDHQAARKCDDCCGGAGRGKCYGPQCLCR |
| 264 | ACAPCFTTDHQAARACDDCCGGAGRGKCYGPQCLCR |
| 265 | ACAPCFTTDHQAARRCDDCCGGAGRGKCYGPQCLCR |
| 266 | ACAPCFTTDHQAARKCDDCCGGRGRGKCYGPQCLCR |
| 267 | ACAPCFTTDHQAARACDDCCGGRGRGKCYGPQCLCR |
| 268 | ACAPCFTTDHQAARRCDDCCGGRGRGKCYGPQCLCR |
| 269 | ACAPCFTTDHQAARKCDDCCGGKGRGACYGPQCLCR |
| 270 | ACAPCFTTDHQAARACDDCCGGKGRGACYGPQCLCR |
| 271 | ACAPCFTTDHQAARRCDDCCGGKGRGACYGPQCLCR |
| 272 | ACAPCFTTDHQAARKCDDCCGGAGRGACYGPQCLCR |
| 273 | ACAPCFTTDHQAARACDDCCGGAGRGACYGPQCLCR |
| 274 | ACAPCFTTDHQAARRCDDCCGGAGRGACYGPQCLCR |
| 275 | ACAPCFTTDHQAARKCDDCCGGRGRGACYGPQCLCR |
| 276 | ACAPCFTTDHQAARACDDCCGGRGRGACYGPQCLCR |
| 277 | ACAPCFTTDHQAARRCDDCCGGRGRGACYGPQCLCR |
| 278 | ACAPCFTTDHQAARKCDDCCGGKGRGRCYGPQCLCR |
| 279 | ACAPCFTTDHQAARACDDCCGGKGRGRCYGPQCLCR |
| 280 | ACAPCFTTDHQAARRCDDCCGGKGRGRCYGPQCLCR |
| 281 | ACAPCFTTDHQAARKCDDCCGGAGRGRCYGPQCLCR |
| 282 | ACAPCFTTDHQAARACDDCCGGAGRGRCYGPQCLCR |
| 283 | ACAPCFTTDHQAARRCDDCCGGAGRGRCYGPQCLCR |
| 284 | ACAPCFTTDHQAARKCDDCCGGRGRGRCYGPQCLCR |
| 285 | ACAPCFTTDHQAARACDDCCGGRGRGRCYGPQCLCR |
| 286 | ACAPCFTTDHQAARRCDDCCGGRGRGRCYGPQCLCR |
| 287 | ACAPCFTTDHQAARRCDDCCGGRGRGRCYGPQCLCR |
| 288 | KCAPCFTTDHQAARRCDDCCGGRGRGRCYGPQCLCR |
| 289 | ACAPCFTTDHQAARRCDDCCGGRGRGRCYGPQCLCR |
| 290 | KCAPCFTTDHQAARRCDDCCGGRGRGRCYGPQCLCR |
| 291 | ACAPCFTTDHQAAR(Cit)CDDCCGG(Cit)GRGKCYGPQCLCR |
| 292 | ACAPCFTTDHQAAR(Cit)CDDCCGG(Cit)GRG(Cit)CYGPQCLCR |
| 293 | KCAPCFTTDHQAAR(Cit)CDDCCGG(Cit)GRG(Cit)CYGPQCLCR |
| 294 | ACAPCFTTDHQAAR(Cit)CDDCCGG(Cit)GRGKCYGPQCLCR |
| 295 | ACAPCFTTDHQAAR(Cit)CDDCCGG(Cit)GRG(Cit)CYGPQCLCR |
| 296 | KCAPCFTTDHQAAR(Cit)CDDCCGG(Cit)GRG(Cit)CYGPQCLCR |
| 297 | ICIPCFTTDHQIARKCDDCCGGKGRGKCYGPQCLCR |
| 298 | ICIPCFTTDHQIARACDDCCGGKGRGKCYGPQCLCR |
| 299 | ICIPCFTTDHQIARRCDDCCGGKGRGKCYGPQCLCR |
| 300 | ICIPCFTTDHQIARKCDDCCGGAGRGKCYGPQCLCR |
| 301 | ICIPCFTTDHQIARACDDCCGGAGRGKCYGPQCLCR |
| 302 | ICIPCFTTDHQIARRCDDCCGGAGRGKCYGPQCLCR |
| 303 | ICIPCFTTDHQIARKCDDCCGGRGRGKCYGPQCLCR |
| 304 | ICIPCFTTDHQIARACDDCCGGRGRGKCYGPQCLCR |
| 305 | ICIPCFTTDHQIARRCDDCCGGRGRGKCYGPQCLCR |
| 306 | ICIPCFTTDHQIARKCDDCCGGKGRGACYGPQCLCR |
| 307 | ICIPCFTTDHQIARACDDCCGGKGRGACYGPQCLCR |
| 308 | ICIPCFTTDHQIARRCDDCCGGKGRGACYGPQCLCR |
| 309 | ICIPCFTTDHQIARKCDDCCGGAGRGACYGPQCLCR |
| 310 | ICIPCFTTDHQIARACDDCCGGAGRGACYGPQCLCR |
| 311 | ICIPCFTTDHQIARRCDDCCGGAGRGACYGPQCLCR |
| 312 | ICIPCFTTDHQIARKCDDCCGGRGRGACYGPQCLCR |
| 313 | ICIPCFTTDHQIARACDDCCGGRGRGACYGPQCLCR |
| 314 | ICIPCFTTDHQIARRCDDCCGGRGRGACYGPQCLCR |
| 315 | ICIPCFTTDHQIARKCDDCCGGKGRGRCYGPQCLCR |
| 316 | ICIPCFTTDHQIARACDDCCGGKGRGRCYGPQCLCR |
| 317 | ICIPCFTTDHQIARRCDDCCGGKGRGRCYGPQCLCR |
| 318 | ICIPCFTTDHQIARKCDDCCGGAGRGRCYGPQCLCR |
| 319 | ICIPCFTTDHQIARACDDCCGGAGRGRCYGPQCLCR |
| 320 | ICIPCFTTDHQIARRCDDCCGGAGRGRCYGPQCLCR |
| 321 | ICIPCFTTDHQIARKCDDCCGGRGRGRCYGPQCLCR |
| 322 | ICIPCFTTDHQIARACDDCCGGRGRGRCYGPQCLCR |
| 323 | ICIPCFTTDHQIARRCDDCCGGRGRGRCYGPQCLCR |
| 324 | ICIPCFTTDHQIARRCDDCCGGRGRGRCYGPQCLCR |
| 325 | KCIPCFTTDHQIARRCDDCCGGRGRGRCYGPQCLCR |
| 326 | ACAPCFTTDHQAARRCDDCCGGRGRGRCYGPQCLCR |
| 327 | KCAPCFTTDHQAARRCDDCCGGRGRGRCYGPQCLCR |
| 328 | ICIPCFTTDHQIAR(Cit)CDDCCGG(Cit)GRGKCYGPQCLCR |
| 329 | ICIPCFTTDHQIAR(Cit)CDDCCGG(Cit)GRG(Cit)CYGPQCLCR |
| 330 | KCIPCFTTDHQIAR(Cit)CDDCCGG(Cit)GRG(Cit)CYGPQCLCR |
| 331 | ACAPCFTTDHQAAR(Cit)CDDCCGG(Cit)GRGKCYGPQCLCR |
| 332 | ACAPCFTTDHQAAR(Cit)CDDCCGG(Cit)GRG(Cit)CYGPQCLCR |
| 333 | KCAPCFTTDHQAAR(Cit)CDDCCGG(Cit)GRG(Cit)CYGPQCLCR |
| 334 | TCTPCFTTDHQTARKCDDCCGGKGRGKCYGPQCLCR |
| 335 | TCTPCFTTDHQTARACDDCCGGKGRGKCYGPQCLCR |
| 336 | TCTPCFTTDHQTARRCDDCCGGKGRGKCYGPQCLCR |
| 337 | TCTPCFTTDHQTARKCDDCCGGAGRGKCYGPQCLCR |
| 338 | TCTPCFTTDHQTARACDDCCGGAGRGKCYGPQCLCR |
| 339 | TCTPCFTTDHQTARRCDDCCGGAGRGKCYGPQCLCR |
| 340 | TCTPCFTTDHQTARKCDDCCGGRGRGKCYGPQCLCR |
| 341 | TCTPCFTTDHQTARACDDCCGGRGRGKCYGPQCLCR |
| 342 | TCTPCFTTDHQTARRCDDCCGGRGRGKCYGPQCLCR |
| 343 | TCTPCFTTDHQTARKCDDCCGGKGRGACYGPQCLCR |
| 344 | TCTPCFTTDHQTARACDDCCGGKGRGACYGPQCLCR |
| 345 | TCTPCFTTDHQTARRCDDCCGGKGRGACYGPQCLCR |
| 346 | TCTPCFTTDHQTARKCDDCCGGAGRGACYGPQCLCR |
| 347 | TCTPCFTTDHQTARACDDCCGGAGRGACYGPQCLCR |
| 348 | TCTPCFTTDHQTARRCDDCCGGAGRGACYGPQCLCR |
| 349 | TCTPCFTTDHQTARKCDDCCGGRGRGACYGPQCLCR |
| 350 | TCTPCFTTDHQTARACDDCCGGRGRGACYGPQCLCR |
| 351 | TCTPCFTTDHQTARRCDDCCGGRGRGACYGPQCLCR |
| 352 | TCTPCFTTDHQTARKCDDCCGGKGRGRCYGPQCLCR |
| 353 | TCTPCFTTDHQTARACDDCCGGKGRGRCYGPQCLCR |
| 354 | TCTPCFTTDHQTARRCDDCCGGKGRGRCYGPQCLCR |
| 355 | TCTPCFTTDHQTARKCDDCCGGAGRGRCYGPQCLCR |
| 356 | TCTPCFTTDHQTARACDDCCGGAGRGRCYGPQCLCR |
| 357 | TCTPCFTTDHQTARRCDDCCGGAGRGRCYGPQCLCR |
| 358 | TCTPCFTTDHQTARKCDDCCGGRGRGRCYGPQCLCR |
| 359 | TCTPCFTTDHQTARACDDCCGGRGRGRCYGPQCLCR |
| 360 | TCTPCFTTDHQTARRCDDCCGGRGRGRCYGPQCLCR |
| 361 | TCTPCFTTDHQTARRCDDCCGGRGRGRCYGPQCLCR |
| 362 | KCTPCFTTDHQTARRCDDCCGGRGRGRCYGPQCLCR |
| 363 | ACAPCFTTDHQAARRCDDCCGGRGRGRCYGPQCLCR |
| 364 | KCAPCFTTDHQAARRCDDCCGGRGRGRCYGPQCLCR |
| 365 | TCTPCFTTDHQTAR(Cit)CDDCCGG(Cit)GRGKCYGPQCLCR |
| 366 | TCTPCFTTDHQTAR(Cit)CDDCCGG(Cit)GRG(Cit)CYGPQCLCR |
| 367 | KCTPCFTTDHQTAR(Cit)CDDCCGG(Cit)GRG(Cit)CYGPQCLCR |
| 368 | ACAPCFTTDHQAAR(Cit)CDDCCGG(Cit)GRGKCYGPQCLCR |
| 369 | ACAPCFTTDHQAAR(Cit)CDDCCGG(Cit)GRG(Cit)CYGPQCLCR |
| 370 | KCAPCFTTDHQAAR(Cit)CDDCCGG(Cit)GRG(Cit)CYGPQCLCR |
| 371 | VCVPCFTTDHQVARKCDDCCGGKGRGKCYGPQCLCR |
| 372 | VCVPCFTTDHQVARACDDCCGGKGRGKCYGPQCLCR |
| 373 | VCVPCFTTDHQVARRCDDCCGGKGRGKCYGPQCLCR |
| 374 | VCVPCFTTDHQVARKCDDCCGGAGRGKCYGPQCLCR |
| 375 | VCVPCFTTDHQVARACDDCCGGAGRGKCYGPQCLCR |
| 376 | VCVPCFTTDHQVARRCDDCCGGAGRGKCYGPQCLCR |
| 377 | VCVPCFTTDHQVARKCDDCCGGRGRGKCYGPQCLCR |
| 378 | VCVPCFTTDHQVARACDDCCGGRGRGKCYGPQCLCR |
| 379 | VCVPCFTTDHQVARRCDDCCGGRGRGKCYGPQCLCR |
| 380 | VCVPCFTTDHQVARKCDDCCGGKGRGACYGPQCLCR |
| 381 | VCVPCFTTDHQVARACDDCCGGKGRGACYGPQCLCR |
| 382 | VCVPCFTTDHQVARRCDDCCGGKGRGACYGPQCLCR |
| 383 | VCVPCFTTDHQVARKCDDCCGGAGRGACYGPQCLCR |
| 384 | VCVPCFTTDHQVARACDDCCGGAGRGACYGPQCLCR |
| 385 | VCVPCFTTDHQVARRCDDCCGGAGRGACYGPQCLCR |
| 386 | VCVPCFTTDHQVARKCDDCCGGRGRGACYGPQCLCR |
| 387 | VCVPCFTTDHQVARACDDCCGGRGRGACYGPQCLCR |
| 388 | VCVPCFTTDHQVARRCDDCCGGRGRGACYGPQCLCR |
| 389 | VCVPCFTTDHQVARKCDDCCGGKGRGRCYGPQCLCR |
| 390 | VCVPCFTTDHQVARACDDCCGGKGRGRCYGPQCLCR |
| 391 | VCVPCFTTDHQVARRCDDCCGGKGRGRCYGPQCLCR |
| 392 | VCVPCFTTDHQVARKCDDCCGGAGRGRCYGPQCLCR |
| 393 | VCVPCFTTDHQVARACDDCCGGAGRGRCYGPQCLCR |
| 394 | VCVPCFTTDHQVARRCDDCCGGAGRGRCYGPQCLCR |
| 395 | VCVPCFTTDHQVARKCDDCCGGRGRGRCYGPQCLCR |
| 396 | VCVPCFTTDHQVARACDDCCGGRGRGRCYGPQCLCR |
| 397 | VCVPCFTTDHQVARRCDDCCGGRGRGRCYGPQCLCR |
| 398 | VCVPCFTTDHQVARRCDDCCGGRGRGRCYGPQCLCR |
| 399 | KCVPCFTTDHQVARRCDDCCGGRGRGRCYGPQCLCR |
| 400 | ACAPCFTTDHQAARRCDDCCGGRGRGRCYGPQCLCR |
| 401 | KCAPCFTTDHQAARRCDDCCGGRGRGRCYGPQCLCR |
| 402 | VCVPCFTTDHQVAR(Cit)CDDCCGG(Cit)GRGKCYGPQCLCR |
| 403 | VCVPCFTTDHQVAR(Cit)CDDCCGG(Cit)GRG(Cit)CYGPQCLCR |
| 404 | KCVPCFTTDHQVAR(Cit)CDDCCGG(Cit)GRG(Cit)CYGPQCLCR |
| 405 | ACAPCFTTDHQAAR(Cit)CDDCCGG(Cit)GRGKCYGPQCLCR |
| 406 | ACAPCFTTDHQAAR(Cit)CDDCCGG(Cit)GRG(Cit)CYGPQCLCR |
| 407 | KCAPCFTTDHQAAR(Cit)CDDCCGG(Cit)GRG(Cit)CYGPQCLCR |
| 408 | LCLPCFTTDHQLARKCDDCCGGKGRGKCYGPQCLCR |
| 409 | LCLPCFTTDHQLARACDDCCGGKGRGKCYGPQCLCR |
| 410 | LCLPCFTTDHQLARRCDDCCGGKGRGKCYGPQCLCR |
| 411 | LCLPCFTTDHQLARKCDDCCGGAGRGKCYGPQCLCR |
| 412 | LCLPCFTTDHQLARACDDCCGGAGRGKCYGPQCLCR |
| 413 | LCLPCFTTDHQLARRCDDCCGGAGRGKCYGPQCLCR |
| 414 | LCLPCFTTDHQLARKCDDCCGGRGRGKCYGPQCLCR |
| 415 | LCLPCFTTDHQLARACDDCCGGRGRGKCYGPQCLCR |
| 416 | LCLPCFTTDHQLARRCDDCCGGRGRGKCYGPQCLCR |
| 417 | LCLPCFTTDHQLARKCDDCCGGKGRGACYGPQCLCR |
| 418 | LCLPCFTTDHQLARACDDCCGGKGRGACYGPQCLCR |
| 419 | LCLPCFTTDHQLARRCDDCCGGKGRGACYGPQCLCR |
| 420 | LCLPCFTTDHQLARKCDDCCGGAGRGACYGPQCLCR |
| 421 | LCLPCFTTDHQLARACDDCCGGAGRGACYGPQCLCR |
| 422 | LCLPCFTTDHQLARRCDDCCGGAGRGACYGPQCLCR |
| 423 | LCLPCFTTDHQLARKCDDCCGGRGRGACYGPQCLCR |
| 424 | LCLPCFTTDHQLARACDDCCGGRGRGACYGPQCLCR |
| 425 | LCLPCFTTDHQLARRCDDCCGGRGRGACYGPQCLCR |
| 426 | LCLPCFTTDHQLARKCDDCCGGKGRGRCYGPQCLCR |
| 427 | LCLPCFTTDHQLARACDDCCGGKGRGRCYGPQCLCR |
| 428 | LCLPCFTTDHQLARRCDDCCGGKGRGRCYGPQCLCR |
| 429 | LCLPCFTTDHQLARKCDDCCGGAGRGRCYGPQCLCR |
| 430 | LCLPCFTTDHQLARACDDCCGGAGRGRCYGPQCLCR |
| 431 | LCLPCFTTDHQLARRCDDCCGGAGRGRCYGPQCLCR |
| 432 | LCLPCFTTDHQLARKCDDCCGGRGRGRCYGPQCLCR |
| 433 | LCLPCFTTDHQLARACDDCCGGRGRGRCYGPQCLCR |
| 434 | LCLPCFTTDHQLARRCDDCCGGRGRGRCYGPQCLCR |
| 435 | LCLPCFTTDHQLARRCDDCCGGRGRGRCYGPQCLCR |
| 436 | KCLPCFTTDHQLARRCDDCCGGRGRGRCYGPQCLCR |
| 437 | ACAPCFTTDHQAARRCDDCCGGRGRGRCYGPQCLCR |
| 438 | KCAPCFTTDHQAARRCDDCCGGRGRGRCYGPQCLCR |
| 439 | LCLPCFTTDHQLAR(Cit)CDDCCGG(Cit)GRGKCYGPQCLCR |
| 440 | LCLPCFTTDHQLAR(Cit)CDDCCGG(Cit)GRG(Cit)CYGPQCLCR |
| 441 | KCLPCFTTDHQLAR(Cit)CDDCCGG(Cit)GRG(Cit)CYGPQCLCR |
| 442 | ACAPCFTTDHQAAR(Cit)CDDCCGG(Cit)GRGKCYGPQCLCR |
| 443 | ACAPCFTTDHQAAR(Cit)CDDCCGG(Cit)GRG(Cit)CYGPQCLCR |
| 444 | KCAPCFTTDHQAAR(Cit)CDDCCGG(Cit)GRG(Cit)CYGPQCLCR |
| 445 | SCSPCFTTDHQSARKCDDCCGGKGRGKCYGPQCLCR |
| 446 | SCSPCFTTDHQSARACDDCCGGKGRGKCYGPQCLCR |
| 447 | SCSPCFTTDHQSARRCDDCCGGKGRGKCYGPQCLCR |
| 448 | SCSPCFTTDHQSARKCDDCCGGAGRGKCYGPQCLCR |
| 449 | SCSPCFTTDHQSARACDDCCGGAGRGKCYGPQCLCR |
| 450 | SCSPCFTTDHQSARRCDDCCGGAGRGKCYGPQCLCR |
| 451 | SCSPCFTTDHQSARKCDDCCGGRGRGKCYGPQCLCR |
| 452 | SCSPCFTTDHQSARACDDCCGGRGRGKCYGPQCLCR |
| 453 | SCSPCFTTDHQSARRCDDCCGGRGRGKCYGPQCLCR |
| 454 | SCSPCFTTDHQSARKCDDCCGGKGRGACYGPQCLCR |
| 455 | SCSPCFTTDHQSARACDDCCGGKGRGACYGPQCLCR |
| 456 | SCSPCFTTDHQSARRCDDCCGGKGRGACYGPQCLCR |
| 457 | SCSPCFTTDHQSARKCDDCCGGAGRGACYGPQCLCR |
| 458 | SCSPCFTTDHQSARACDDCCGGAGRGACYGPQCLCR |
| 459 | SCSPCFTTDHQSARRCDDCCGGAGRGACYGPQCLCR |
| 460 | SCSPCFTTDHQSARKCDDCCGGRGRGACYGPQCLCR |
| 461 | SCSPCFTTDHQSARACDDCCGGRGRGACYGPQCLCR |
| 462 | SCSPCFTTDHQSARRCDDCCGGRGRGACYGPQCLCR |
| 463 | SCSPCFTTDHQSARKCDDCCGGKGRGRCYGPQCLCR |
| 464 | SCSPCFTTDHQSARACDDCCGGKGRGRCYGPQCLCR |
| 465 | SCSPCFTTDHQSARRCDDCCGGKGRGRCYGPQCLCR |
| 466 | SCSPCFTTDHQSARKCDDCCGGAGRGRCYGPQCLCR |
| 467 | SCSPCFTTDHQSARACDDCCGGAGRGRCYGPQCLCR |
| 468 | SCSPCFTTDHQSARRCDDCCGGAGRGRCYGPQCLCR |
| 469 | SCSPCFTTDHQSARKCDDCCGGRGRGRCYGPQCLCR |
| 470 | SCSPCFTTDHQSARACDDCCGGRGRGRCYGPQCLCR |
| 471 | SCSPCFTTDHQSARRCDDCCGGRGRGRCYGPQCLCR |
| 472 | SCSPCFTTDHQSARRCDDCCGGRGRGRCYGPQCLCR |
| 473 | KCSPCFTTDHQSARRCDDCCGGRGRGRCYGPQCLCR |
| 474 | ACAPCFTTDHQAARRCDDCCGGRGRGRCYGPQCLCR |
| 475 | KCAPCFTTDHQAARRCDDCCGGRGRGRCYGPQCLCR |
| 476 | SCSPCFTTDHQSAR(Cit)CDDCCGG(Cit)GRGKCYGPQCLCR |
| 477 | SCSPCFTTDHQSAR(Cit)CDDCCGG(Cit)GRG(Cit)CYGPQCLCR |
| 478 | KCSPCFTTDHQSAR(Cit)CDDCCGG(Cit)GRG(Cit)CYGPQCLCR |
| 479 | ACAPCFTTDHQAAR(Cit)CDDCCGG(Cit)GRGKCYGPQCLCR |
| 480 | ACAPCFTTDHQAAR(Cit)CDDCCGG(Cit)GRG(Cit)CYGPQCLCR |
| 481 | KCAPCFTTDHQAAR(Cit)CDDCCGG(Cit)GRG(Cit)CYGPQCLCR |

Peptide complexes used in this disclosure can comprise a peptide and a labeling agent or detectable label. In an embodiment, peptide is a variant comprising at least 60%, 65%, 70%, 75%, 80%, 83%, 85%, 86%, 89%, 90%, 92%, 93%, 94%, 95%, 96%, 97%, 98%, or 99% identical to the sequence of the native peptide of peptide or a fragment thereof.

In another embodiment, the compound comprises a polypeptide having at least at least 60%, 65%, 70%, 75%, 80%, 83%, 85%, 86%, 89%, 90%, 92%, 93%, 94%, 95%, 96%, 97%, 98%, or 99% sequence identity with any one of SEQ ID NO: 1-SEQ ID NO: 481, or any fragment thereof.

In another embodiment, the present disclosure provides a peptide having the following amino acid sequence: MCMPCFTTDHQMARKCDDCCGGKGRGKCYGPQCLCR (SEQ ID NO: 1) or a fragment thereof. In a further embodiment, the present disclosure provides peptide variants comprising at least 60%, 65%, 70%, 75%, 80%, 83%, 85%, 86%, 89%, 90%, 92%, 93%, 94%, 95%, 96%, 97%, 98%, or 99% identical to the following amino acid sequence: MCMPCFTTDHQMARKCDDCCGGKGRGKCYGPQCLCR (SEQ ID NO: 1) or a fragment thereof.

In another embodiment, the present disclosure provides a peptide having the following amino acid sequence: MCMPCFTTDHQMARRCDDCCGGRGRGKCYGPQCLCR (SEQ ID NO: 9) or a fragment thereof. In a further embodiment, the present disclosure provides peptide variants comprising at least 60%, 65%, 70%, 75%, 80%, 83%, 85%, 86%, 89%, 90%, 92%, 93%, 94%, 95%, 96%, 97%, 98%, or 99% identical to the following amino acid sequence: MCMPCFTTDHQMARRCDDCCGGRGRGKCYGPQCLCR (SEQ ID NO: 9) or a fragment thereof.

In a further embodiment, the present disclosure provides peptide variants comprising at least 80%, identical to the following amino acid sequence: MCMPCFTTDHQMARRCDDCCGGRGRGKCYGPQCLCR (SEQ ID NO: 9) or a fragment thereof.

In a further embodiment, the present disclosure provides peptide variants comprising at least 83% identical to the following amino acid sequence: MCMPCFTTDHQMARRCDDCCGGRGRGKCYGPQCLCR (SEQ ID NO: 9) or a fragment thereof.

In a still further embodiment, the present disclosure provides peptide variants comprising at least 86% identical to the following amino acid sequence: MCMPCFTTDHQMARRCDDCCGGRGRGKCYGPQCLCR (SEQ ID NO: 9) or a fragment thereof.

In another embodiment, the present disclosure provides peptide variants comprising at least 88% identical to the following amino acid sequence: MCMPCFTTDHQMARRCDDCCGGRGRGKCYGPQCLCR (SEQ ID NO: 9) or a fragment thereof.

In a further embodiment, the present disclosure provides peptide variants comprising at least 90% identical to the following amino acid sequence: MCMPCFTTDHQMARRCDDCCGGRGRGKCYGPQCLCR (SEQ ID NO: 9) or a fragment thereof.

In a still further embodiment, the present disclosure provides peptide variants comprising at least 91% identical to the following amino acid sequence: MCMPCFTTDHQMARRCDDCCGGRGRGKCYGPQCLCR (SEQ ID NO: 9) or a fragment thereof.

In a still further embodiment, the present disclosure provides peptide variants comprising at least 94% identical to the following amino acid sequence: MCMPCFTTDHQMARRCDDCCGGRGRGKCYGPQCLCR (SEQ ID NO: 9) or a fragment thereof.

In yet another embodiment, the present disclosure provides peptide variants comprising at least 97% identical to the following amino acid sequence: MCMPCFTTDHQMARRCDDCCGGRGRGKCYGPQCLCR (SEQ ID NO: 9) or a fragment thereof.

In another embodiment, the present disclosure provides a peptide having the following amino acid sequence: MCMPCFTTDHQMARXCDDCCGGXGRGXCYGPQCLCR (SEQ ID NO: 482) or a fragment thereof, wherein each X can each independently be any amino acid. In another embodiment, the present disclosure provides a peptide having the following amino acid sequence: MCMPCFTTDHQMARXCDDCCGGXGRGXCYGPQCLCR (SEQ ID NO: 483) or a fragment thereof, wherein X is selected from K, A and R.

In another embodiment, the chlorotoxin is a peptide or variant thereof having the following amino acid sequence: MCMPCFTTDHQMARXCDDCCGGXGRGXCYGPQCLCR (SEQ ID NO: 484) or a fragment thereof, wherein each X can independently be R or A.

In another embodiment, the chlorotoxin is a peptide or variant thereof having the following amino acid sequence: MCMPCFTTDHQMARXCDDCCGGXGRGKCYGPQCLCR (SEQ ID NO: 485) or a fragment thereof, wherein each X can independently be R or A.

In still other instances, the variant nucleic acid molecules of a peptide of any one of SEQ ID NO: 1-SEQ ID NO: 485 can be identified by either a determination of the sequence identity of the encoded peptide amino acid sequence with the amino acid sequence of any one of SEQ ID NO: 1-SEQ ID NO: 481, or by a nucleic acid hybridization assay. Such peptide variants can include nucleic acid molecules (1) that remain hybridized with a nucleic acid molecule having the nucleotide sequence of any one of SEQ ID NO: 1-SEQ ID NO: 481 (or its complement) under stringent washing conditions, in which the wash stringency is equivalent to 0.5×-2×SSC with 0.1% SDS at 55-65° C., and (2) that encode a peptide having at least 70%, at least 80%, at least 90%, at least 95% or greater than 95% sequence identity to the amino acid sequence of any one of SEQ ID NO: 1-SEQ ID NO: 481. Alternatively, peptide variants of any one of SEQ ID NO: 1-SEQ ID NO: 481 can be characterized as nucleic acid molecules (1) that remain hybridized with a nucleic acid molecule having the nucleotide sequence of any one of SEQ ID NO: 1-SEQ ID NO: 481 (or its complement) under highly stringent washing conditions, in which the wash stringency is equivalent to 0.1×-0.2×SSC with 0.1% SDS at 50-65° C., and (2) that encode a peptide having at least 70%, at least 80%, at least 90%, at least 95% or greater than 95% sequence identity to the amino acid sequence of any one of SEQ ID NO: 1-SEQ ID NO: 481.

The term “engineered,” when applied to a polynucleotide, denotes that the polynucleotide has been removed from its natural genetic milieu and is thus free of other extraneous or unwanted coding sequences, and is in a form suitable for use within genetically engineered protein production systems. Such engineered molecules are those that are separated from their natural environment and include cDNA and genomic clones (i.e., a prokaryotic or eukaryotic cell with a vector containing a fragment of DNA from a different organism). Engineered DNA molecules of the present invention are free of other genes with which they are ordinarily associated but may include naturally occurring or non-naturally occurring 5′ and 3′ untranslated regions such as enhancers, promoters and terminators.

An “engineered” polypeptide or protein is a polypeptide or protein that is found in a condition other than its native environment, such as apart from blood and animal tissue. In a preferred form, the engineered polypeptide is substantially free of other polypeptides, particularly other polypeptides of animal origin. It is preferred to provide the polypeptides in a highly purified form, e.g., greater than 95% pure, more preferably greater than 98% pure or greater than 99% pure. When used in this context, the term “engineered” does not exclude the presence of the same polypeptide in alternative physical forms, such as dimers, heterodimers and multimers, or alternatively glycosylated, carboxylated, modified, or derivatized forms.

An “engineered” peptide or protein is a polypeptide that is distinct from a naturally occurring polypeptide structure, sequence, or composition. Engineered peptides include non-naturally occurring, artificial, isolated, synthetic, designed, modified, or recombinantly expressed peptides. Provided herein are engineered TfR-binding peptides, variants, or fragments thereof. These engineered TfR-binding peptides can be further linked to an active agent or a detectable agent. The active agent can be a half-life extending moiety.

Polypeptides of the disclosure include polypeptides that have been modified in any way, for example, to: (1) reduce susceptibility to proteolysis, (2) reduce susceptibility to oxidation, (3) alter binding affinity for forming protein complexes, (4) alter binding affinities, and (5) confer or modify other physicochemical or functional properties. For example, single or multiple amino acid substitutions (e.g., conservative amino acid substitutions) are made in the naturally occurring sequence (e.g., in the portion of the polypeptide outside the domain(s) forming intermolecular contacts). A “conservative amino acid substitution” can refer to the substitution in a polypeptide of an amino acid with a functionally similar amino acid. The following six groups each contain amino acids that can be conservative substitutions for one another: i) Alanine (A), Serine (S), and Threonine (T); ii) Aspartic acid (D) and Glutamic acid (E); iii) Asparagine (N) and Glutamine (Q); iv) Arginine (R) and Lysine (K); v) Isoleucine (I), Leucine (L), Methionine (M), and Valine (V); vi) Phenylalanine (F), Tyrosine (Y), and Tryptophan (W).

The terms “polypeptide fragment” and “truncated polypeptide” as used herein can refer to a polypeptide that has an amino-terminal and/or carboxy-terminal deletion as compared to a corresponding full-length peptide or protein. In various embodiments, fragments are at least 5, at least 10, at least 25, at least 50, at least 100, at least 150, at least 200, at least 250, at least 300, at least 350, at least 400, at least 450, at least 500, at least 600, at least 700, at least 800, at least 900 or at least 1000 amino acids in length. In various embodiments, fragments can also be, e.g., at most 1000, at most 900, at most 800, at most 700, at most 600, at most 500, at most 450, at most 400, at most 350, at most 300, at most 250, at most 200, at most 150, at most 100, at most 50, at most 25, at most 10, or at most 5 amino acids in length. A fragment can further comprise, at either or both of its ends, one or more additional amino acids, for example, a sequence of amino acids from a different naturally-occurring protein (e.g., an Fc or leucine zipper domain) or an artificial amino acid sequence (e.g., an artificial linker sequence).

Percent sequence identity is determined by conventional methods. See, for example, Altschul et al., Bull. Math. Bio. 48:603 (1986), and Henikoff and Henikoff, Proc. Natl. Acad.

Sci. USA 89:10915 (1992). Briefly, two amino acid sequences are aligned to optimize the alignment scores using a gap opening penalty of 10, a gap extension penalty of 1, and the “BLOSUM62” scoring matrix of Henikoff and Henikoff (Id). The sequence identity is then calculated as: ([Total number of identical matches]/[length of the longer sequence plus the number of gaps introduced into the longer sequence in order to align the two sequences])(100).

Additionally, there are many established algorithms available to align two amino acid sequences. For example, the “FASTA” similarity search algorithm of Pearson and Lipman is a suitable protein alignment method for examining the level of sequence identity or homology shared by an amino acid sequence of a peptide disclosed herein and the amino acid sequence of a peptide variant. The FASTA algorithm is described by Pearson and Lipman, Proc. Nat'l Acad. Sci. USA 85:2444 (1988), and by Pearson, Meth. Enzymol. 183:63 (1990). Briefly, FASTA first characterizes sequence similarity by identifying regions shared by the query sequence (e.g., SEQ ID NO: 9) and a test sequence that has either the highest density of identities (if the ktup variable is 1) or pairs of identities (if ktup=2), without considering conservative amino acid substitutions, insertions, or deletions. The ten regions with the highest density of identities are then rescored by comparing the similarity of all paired amino acids using an amino acid substitution matrix, and the ends of the regions are “trimmed” to include only those residues that contribute to the highest score. If there are several regions with scores greater than the “cutoff” value (calculated by a predetermined formula based upon the length of the sequence and the ktup value), then the trimmed initial regions are examined to determine whether the regions can be joined to form an approximate alignment with gaps. Finally, the highest scoring regions of the two amino acid sequences are aligned using a modification of the Needleman-Wunsch-Sellers algorithm (Needleman and Wunsch, J. Mol. Biol. 48:444 (1970); Sellers, Siam J. Appl. Math. 26:787 (1974)), which allows for amino acid insertions and deletions. Illustrative parameters for FASTA analysis are: ktup=1, gap opening penalty=10, gap extension penalty=1, and substitution matrix=BLOSUM62. These parameters can be introduced into a FASTA program by modifying the scoring matrix file (“SMATRIX”), as explained in Appendix 2 of Pearson, Meth. Enzymol. 183:63 (1990).

FASTA can also be used to determine the sequence identity of nucleic acid molecules using a ratio as disclosed above. For nucleotide sequence comparisons, the ktup value can range between one to six, preferably from three to six, most preferably three, with other parameters set as described above.

Some examples of common amino acids that are a “conservative amino acid substitution” are illustrated by a substitution among amino acids within each of the following groups: (1) glycine, alanine, valine, leucine, and isoleucine, (2) phenylalanine, tyrosine, and tryptophan, (3) serine and threonine, (4) aspartate and glutamate, (5) glutamine and asparagine, and (6) lysine, arginine and histidine. The BLOSUM62 table is an amino acid substitution matrix derived from about 2,000 local multiple alignments of protein sequence segments, representing highly conserved regions of more than 500 groups of related proteins (Henikoffand Henikoff, Proc. Nat'I Acad. Sci. USA 89:10915 (1992)). Accordingly, the BLOSUM62 substitution frequencies can be used to define conservative amino acid substitutions that may be introduced into the amino acid sequences of the present invention.

Although it is possible to design amino acid substitutions based solely upon chemical properties (as discussed above), the language “conservative amino acid substitution” preferably refers to a substitution represented by a BLOSUM62 value of greater than −1. For example, an amino acid substitution is conservative if the substitution is characterized by a BLOSUM62 value of 0, 1, 2, or 3. According to this system, preferred conservative amino acid substitutions are characterized by a BLOSUM62 value of at least 1 (e.g., 1, 2 or 3), while more preferred conservative amino acid substitutions are characterized by a BLOSUM62 value of at least 2 (e.g., 2 or 3).

Determination of amino acid residues that are within regions or domains that are critical to maintaining structural integrity can be determined. Within these regions one can determine specific residues that can be more or less tolerant of change and maintain the overall tertiary structure of the molecule. Methods for analyzing sequence structure include, but are not limited to, alignment of multiple sequences with high amino acid or nucleotide identity and computer analysis using available software (e.g., the Insight II® viewer and homology modeling tools; MSI, San Diego, Calif.), secondary structure propensities, binary patterns, complementary packing and buried polar interactions (Barton, G. J., Current Opin. Struct. Biol. 5:372-6 (1995) and Cordes, M. H. et al., Current Opin. Struct. Biol. 6:3-10 (1996)). In general, when designing modifications to molecules or identifying specific fragments determination of structure can typically be accompanied by evaluating activity of modified molecules.

In another embodiment, the peptide is Compound 76, which is a chlorotoxin variant comprising the sequence of MCMPCFTTDHQMARRCDDCCGGRGRGKCYGPQCLCR (SEQ ID NO: 9), wherein the lysine residue is conjugated to a cyanine fluorescent label. The structure of Compound 76 is shown below:

The structure of tozuleristide is shown below:

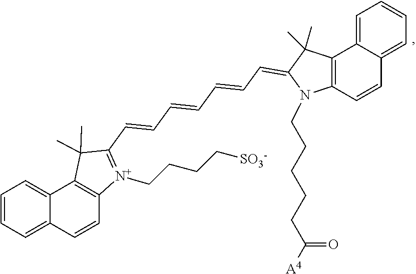

wherein A⁴ is a peptide of

(SEQ ID NO: 9)
MCMPCFTTDHQMARRCDDCCGGRGRGKCYGPQCLCR.

The peptide can be further cross-linked by our disulfide bonds formed among the cysteine residues present in the sequence.

TABLE 4
Exemplary Compounds According to the Present Disclosure
A = MCMPCFTTDHQMARRCDDCCGGRGRGKCYGPQCLCR (SEQ ID NO: 9)

| No. | Structure |
|---|---|
| 61 | 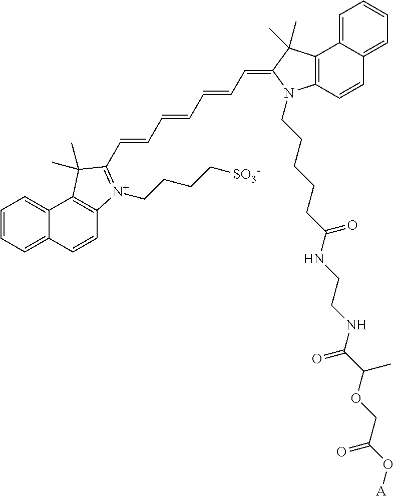 |
| 62 | 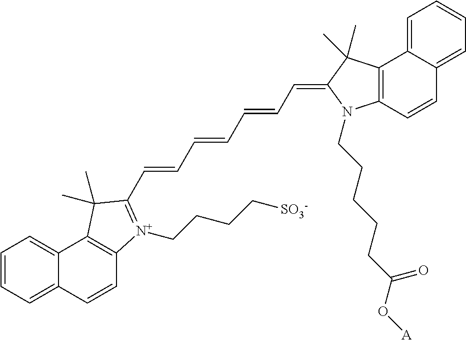 |
| 63 | 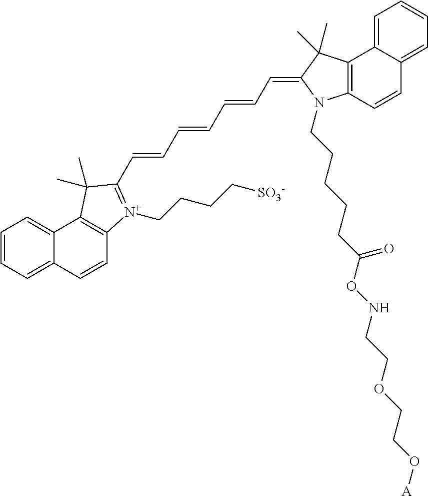 |
| 64 | 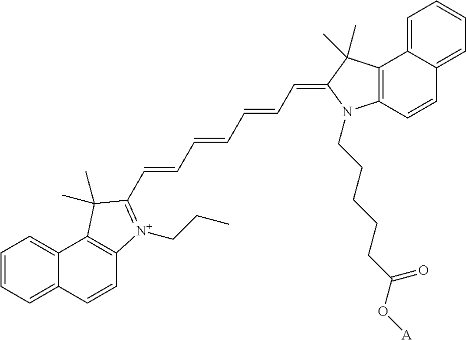 |
| 65 | 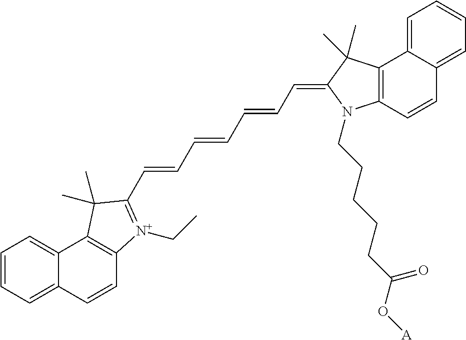 |
| 66 | 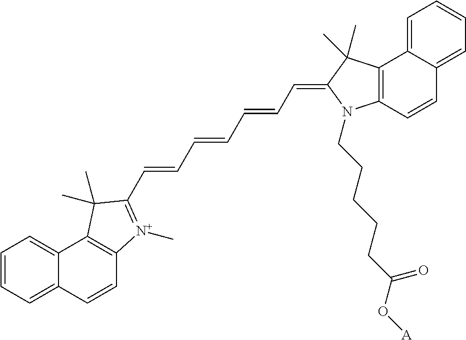 |
| 67 | 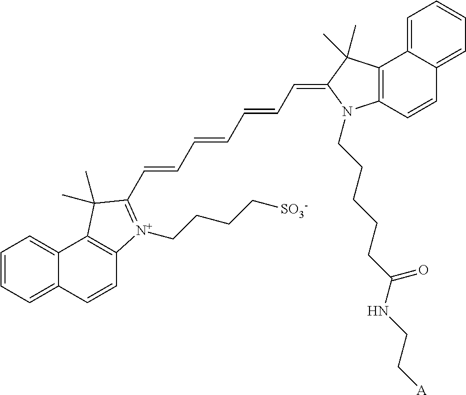 |
| 68 | 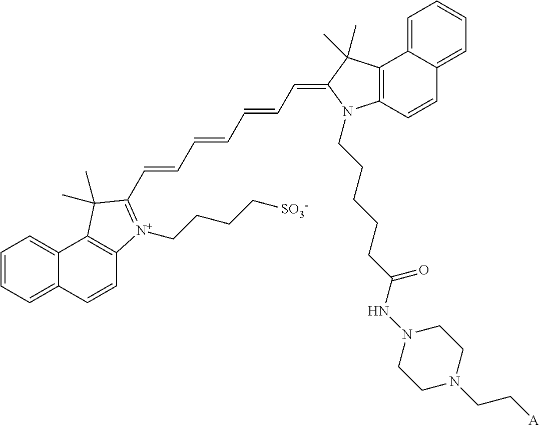 |
| 69 | 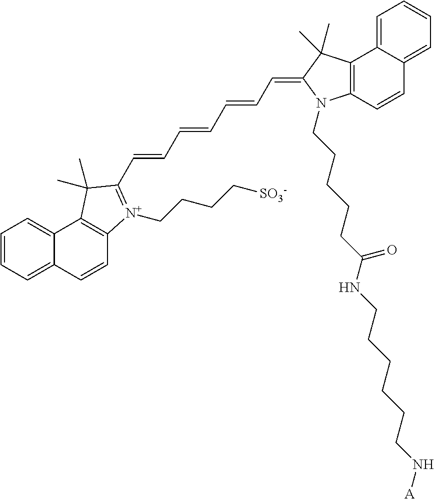 |
| 70 | 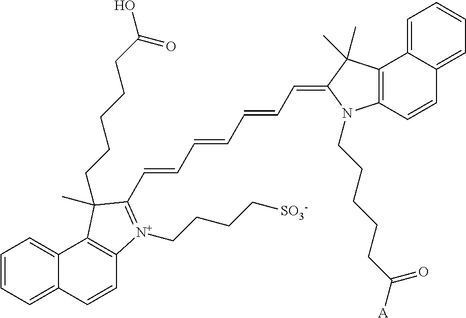 |
| 71 | 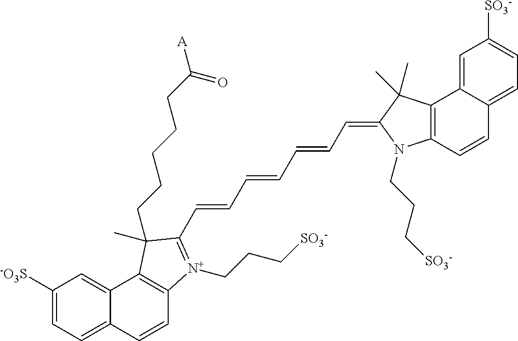 |
| 72 | 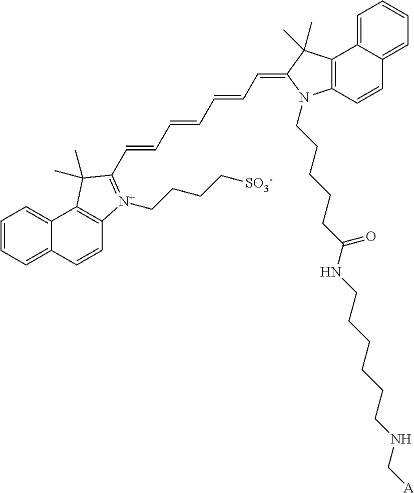 |
| 73 | 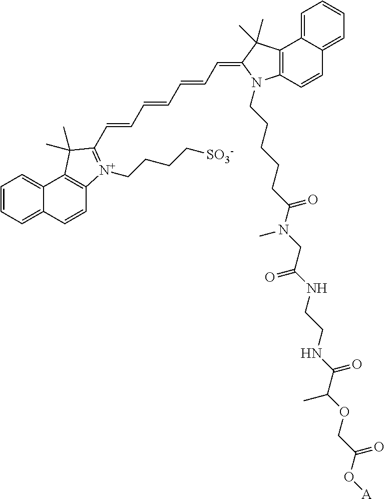 |
| 74 | 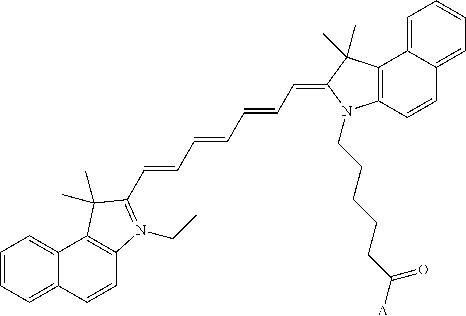 |
| 75 | 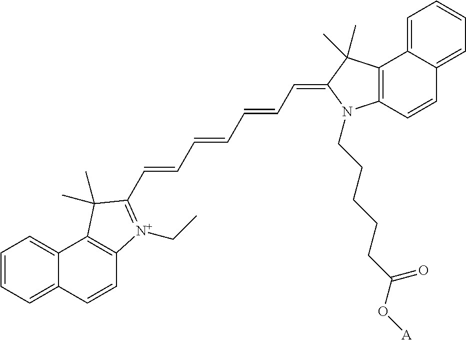 |
| 76 | 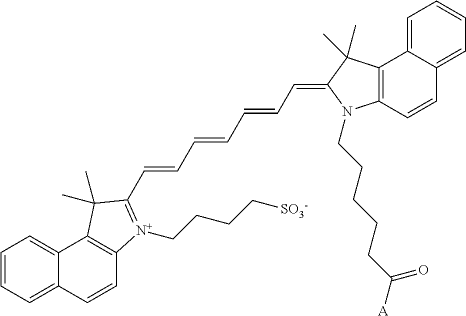 |
| 77 | 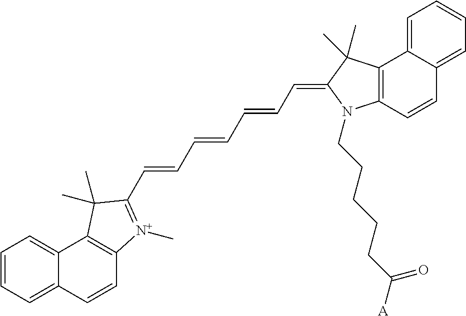 |
| 78 | 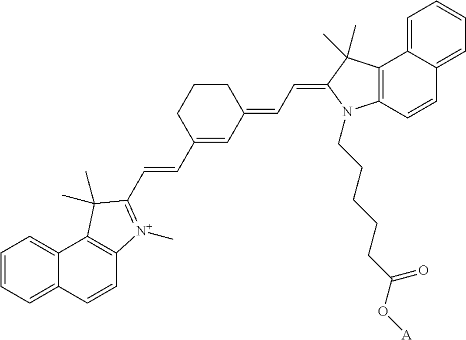 |
| 79 | 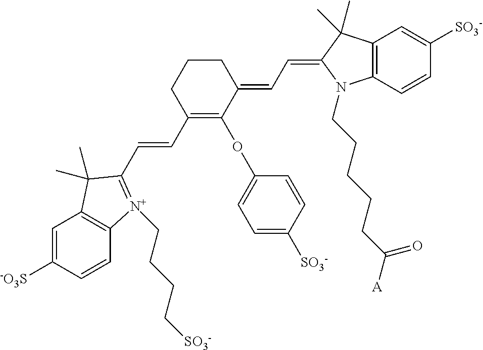 |
| 80 | 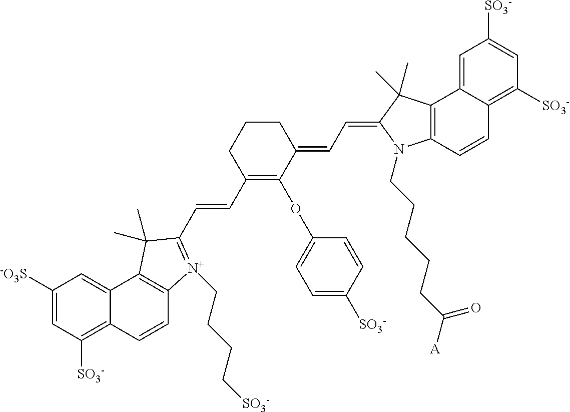 |
| 81 | 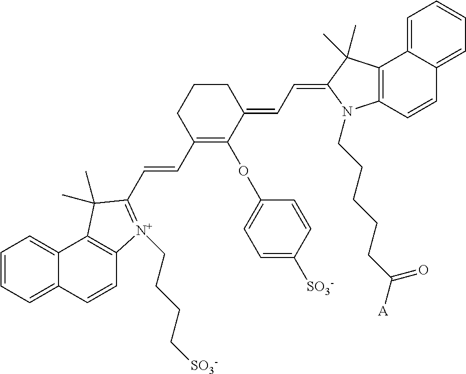 |
| 82 | 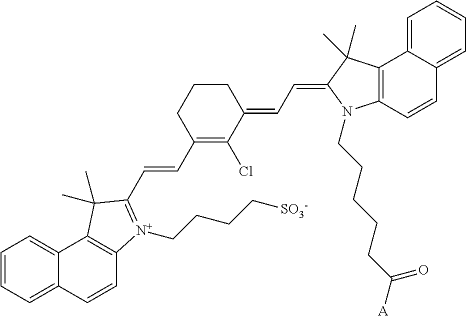 |
| 83 | 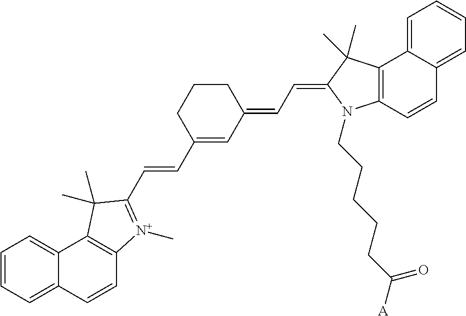 |
| 84 | 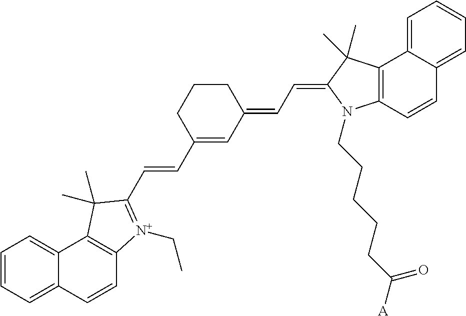 |
| 85 | 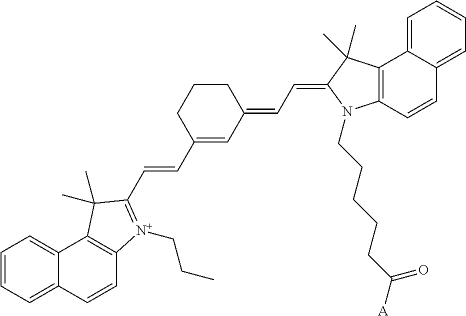 |
| 86 | 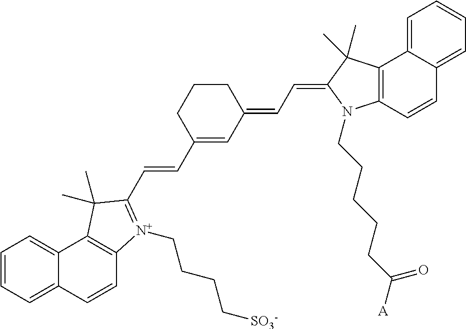 |
| 87 | 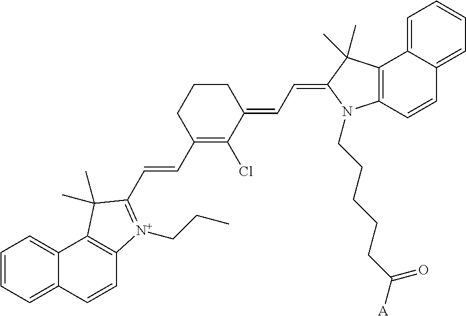 |
| 88 | 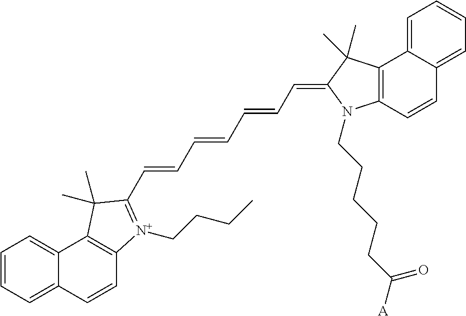 |
| 89 | 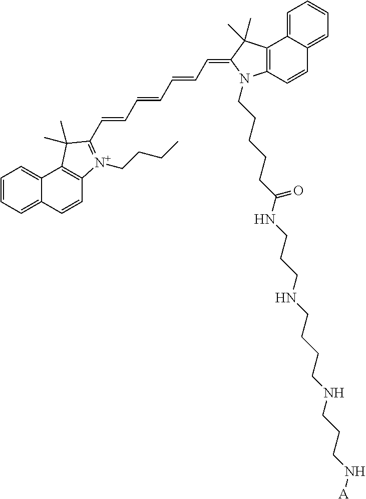 |
| 90 | 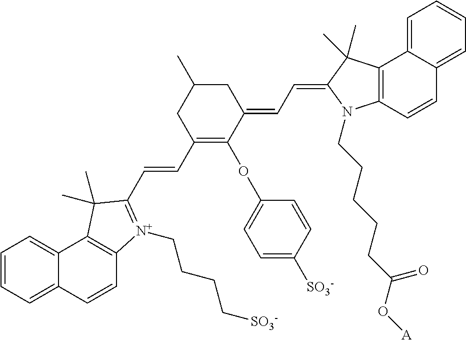 |
| 91 | 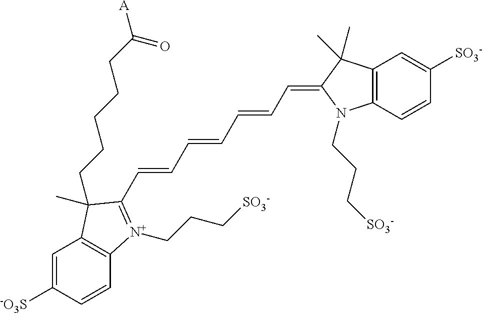 |
| 92 | 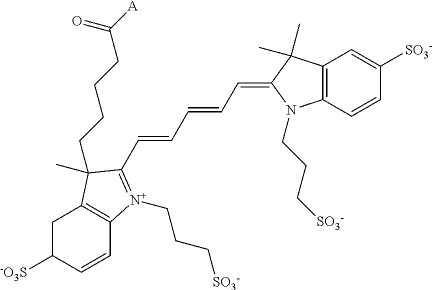 |
| 93 | 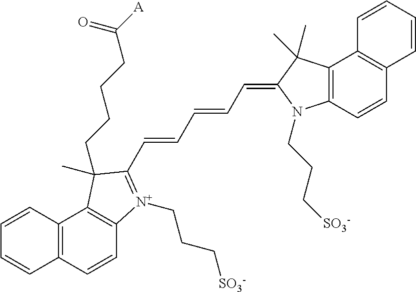 |
| 94 | 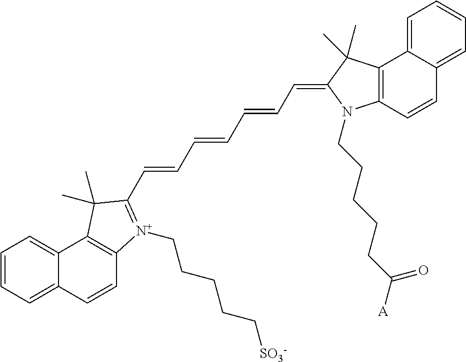 |
| 95 | 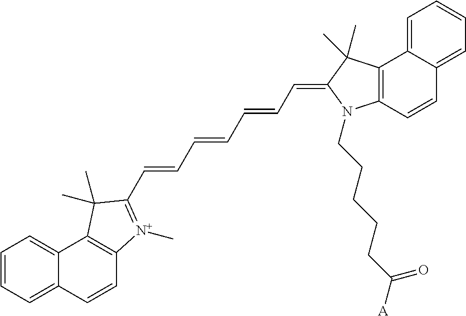 |
| 96 | 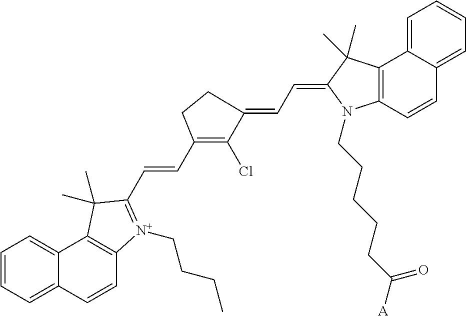 |
| 97 | 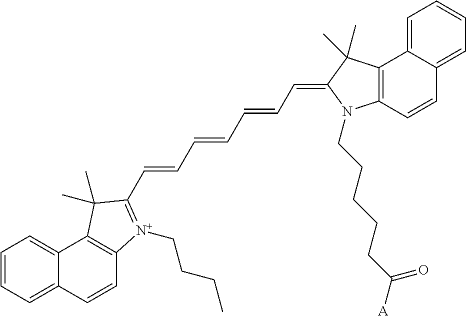 |
| 98 | 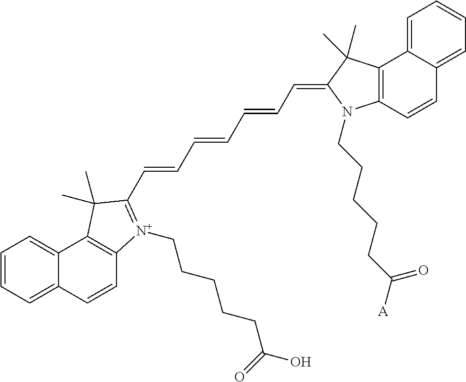 |
| 99 | 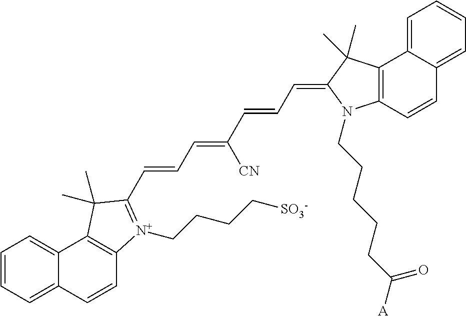 |
| 100 | 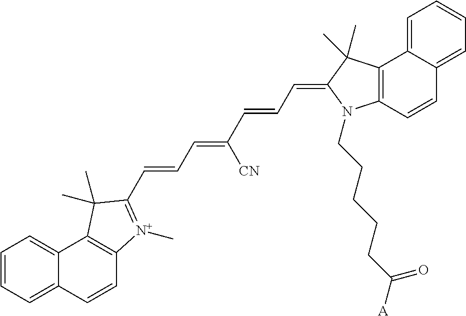 |
| 101 | 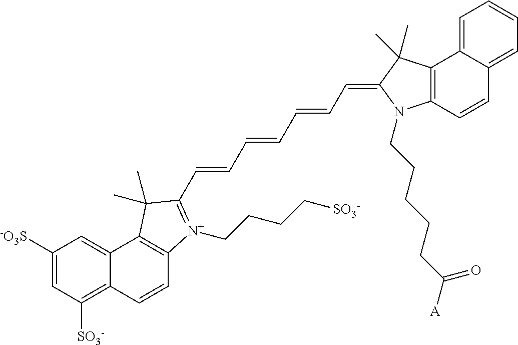 |
| 102 | 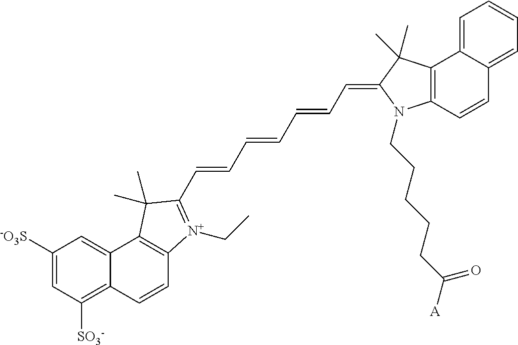 |
| 103 | 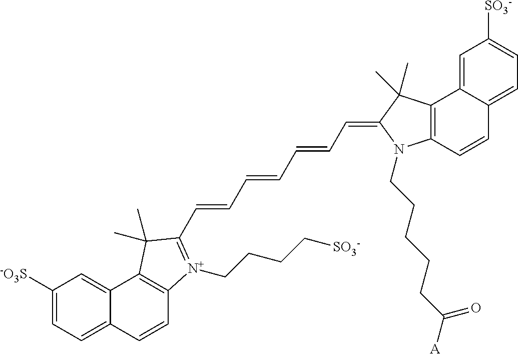 |
| 104 | 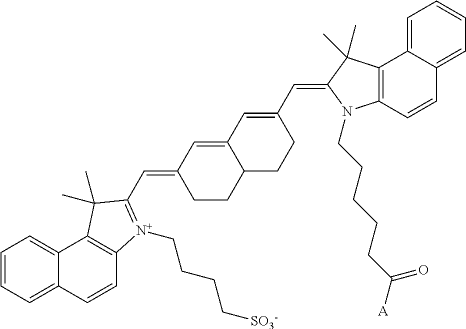 |
| 105 | 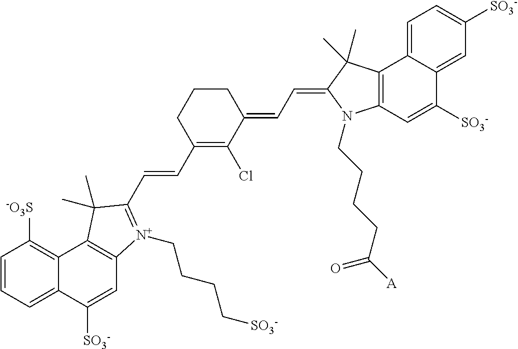 |
| 106 | 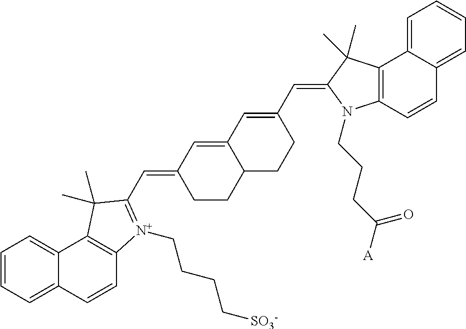 |
| 107 | 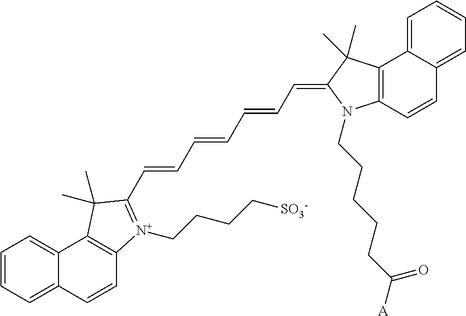 |
| 108 | 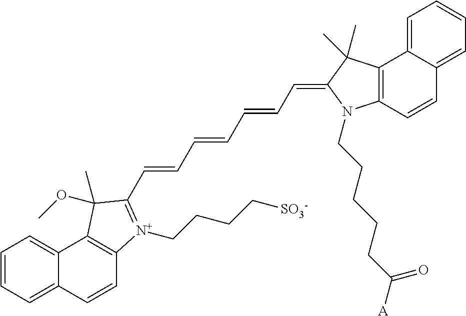 |
| 109 | 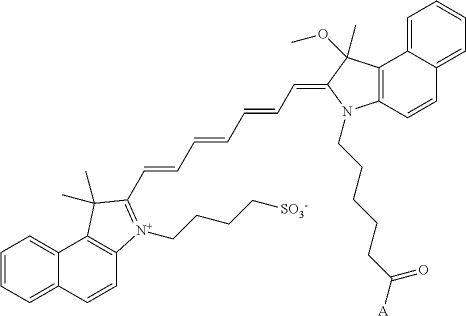 |
| 110 | 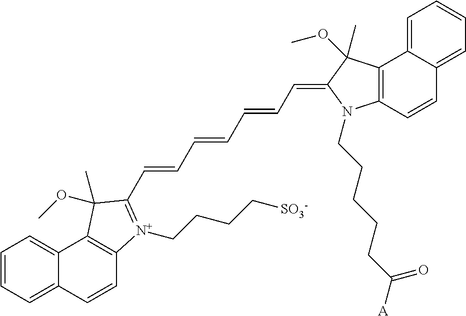 |
| 111 | 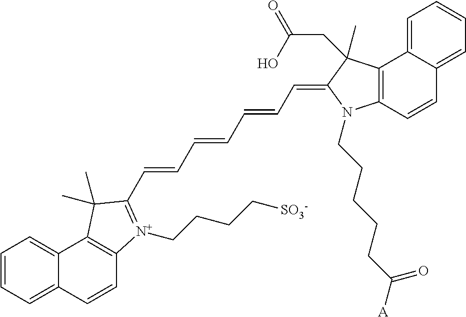 |
| 112 | 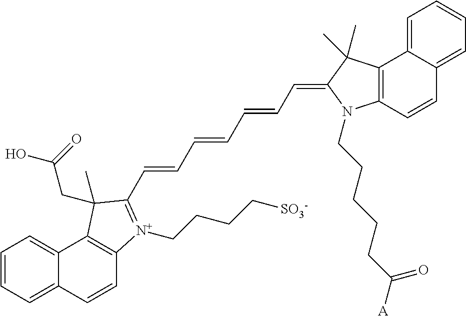 |
| 113 | 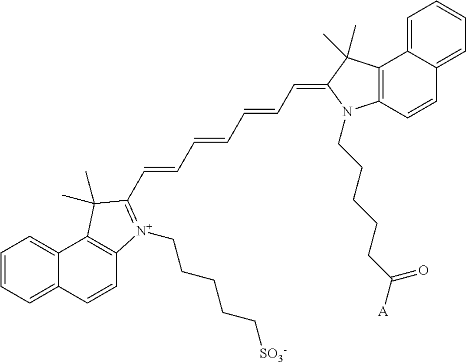 |
| 114 | 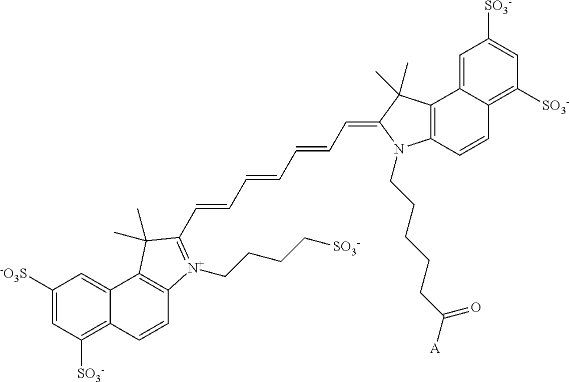 |
| 115 | 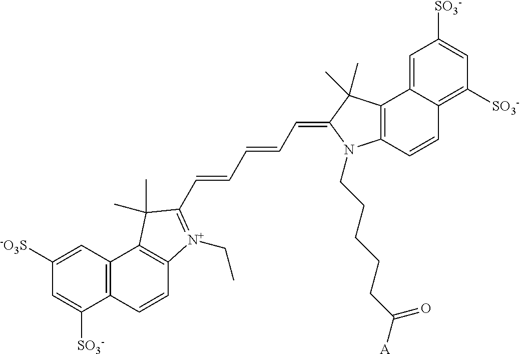 |
| 116 | 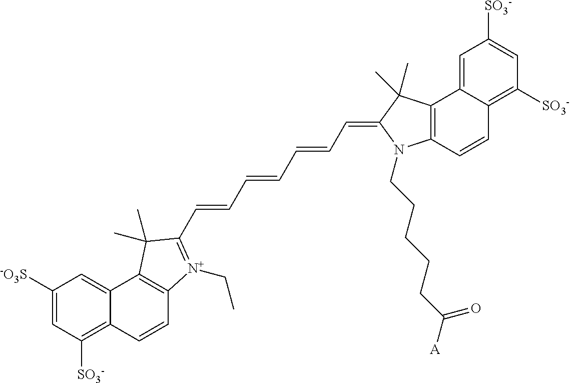 |
| 117 | 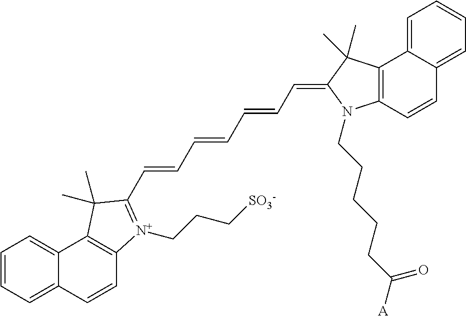 |
| 118 | 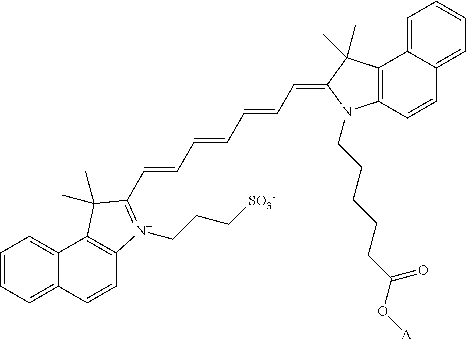 |
| 119 | 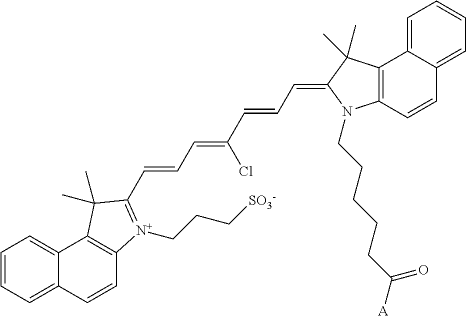 |
| 120 | 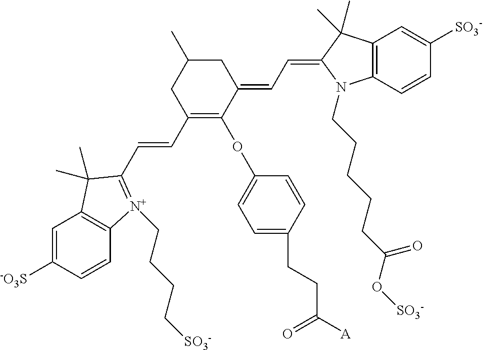 |

In some aspects, the peptide is a variant of the native peptide of chlorotoxin but retains all eight cysteine residues of the native peptide, enabling cross-linking by up to four disulfide bonds. Conservation of cysteine residues helps to preserve the secondary structure and other features of the native chlorotoxin peptide because of the disulfide bonds that form between the cysteine residues. In some aspects, the chlorotoxin peptide variant retains all eight cysteine residues of the native peptide and has at least 40%, 45%, 50%, 55%, 60%, 65%, 70%, 75%, 80%, 83%, 85%, 86%, 89%, 90%, 92%, 93%, 94%, 95%, 96%, 97%, 98%, or 99% sequence identity with the native chlorotoxin peptide.

In some aspects, the chlorotoxin peptide variant has eight cysteine residues positioned so that the distances between pairs of cysteines is the same as the distances between pairs of cysteines found in the native peptide, and the chlorotoxin peptide variant has at least 40%, 45%, 50%, 55%, 60%, 65%, 70%, 75%, 80%, 83%, 85%, 86%, 89%, 90%, 92%, 93%, 94%, 95%, 96%, 97%, 98%, or 99% sequence identity with the native chlorotoxin peptide.

In some aspects, the chlorotoxin peptide variant has eight cysteine residues positioned so that the distances between pairs of cysteines is functionally equivalent or functionally similar to the distances between pairs of cysteines found in the native peptide, and the chlorotoxin peptide variant has at least 40%, 45%, 50%, 55%, 60%, 65%, 70%, 75%, 80%, 83%, 85%, 86%, 89%, 90%, 92%, 93%, 94%, 95%, 96%, 97%, 98%, or 99% sequence identity with the native chlorotoxin peptide.

In some aspects, the chlorotoxin peptide variant has eight cysteine residues positioned so that the distances between pairs of cysteines allows for secondary structure and isoelectric point of the native chlorotoxin peptide to be preserved, and the chlorotoxin peptide variant has at least 40%, 45%, 50%, 55%, 60%, 65%, 70%, 75%, 80%, 83%, 85%, 86%, 89%, 90%, 92%, 93%, 94%, 95%, 96%, 97%, 98%, or 99% sequence identity with the native chlorotoxin peptide.

In some aspects, the chlorotoxin peptide variant has eight cysteine residues positioned so that the distances between pairs of cysteines is sufficient to allow disulfide bonds to form, and the chlorotoxin peptide variant has at least 40%, 45%, 50%, 55%, 60%, 65%, 70%, 75%, 80%, 83%, 85%, 86%, 89%, 90%, 92%, 93%, 94%, 95%, 96%, 97%, 98%, or 99% sequence identity with the native chlorotoxin peptide.

In some aspects, one or more methionines of the chlorotoxin peptide variant are replaced with other amino acids. In some aspects, one or more methionines of the chlorotoxin peptide variant are replaced with other amino acids selected from glycine, alanine, isoleucine, threonine, valine, leucine, serine or a combination thereof.

In some embodiments, the chlorotoxin can be a chlorotoxin variant. Peptides are further described in PCT Patent Application Publication Numbers WO2006115633 and WO2011142858, which are incorporated in their entirety herein by reference.

In one embodiment, the peptide can have the following formula: H-Met-Cys-Met-Pro-Cys-Phe-Thr-Thr-Asp-His-Gln-Met-Ala-Arg-X₁-Cys-Asp-Asp-Cys-Cys-Gly-Gly-X2-Gly-Arg-Gly-X₃-Cys-Tyr-Gly-Pro-Gln-Cys-Leu-Cys-Arg-OH (SEQ ID NO: 482) acetate salt (disulfide bonds, air oxidized), wherein X₁, X₂, and X₃ can each independently be any amino acid.

In one embodiment, the peptide can have the following formula: H-Met-Cys-Met-Pro-Cys-Phe-Thr-Thr-Asp-His-Gln-Met-Ala-Arg-X₁-Cys-Asp-Asp-Cys-Cys-Gly-Gly-X₂-Gly-Arg-Gly-X₃-Cys-Tyr-Gly-Pro-Gln-Cys-Leu-Cys-Arg-OH (SEQ ID NO: 483) acetate salt (disulfide bonds, air oxidized), wherein X₁, X₂, and X₃ can each independently be Arg, Ala, or Lys.

In another embodiment, the all peptide can have the following formula: H-Met-Cys-Met-Pro-Cys-Phe-Thr-Thr-Asp-His-Gln-Met-Ala-Arg-X₁-Cys-Asp-Asp-Cys-Cys-Gly-Gly-X₂-Gly-Arg-Gly-X₃-Cys-Tyr-Gly-Pro-Gln-Cys-Leu-Cys-Arg-OH (SEQ ID NO: 484) acetate salt (disulfide bonds, air oxidized), wherein X₁, X₂, and X₃ can each independently be Arg or Ala.

In another embodiment, the all peptide can have the following formula: H-Met-Cys-Met-Pro-Cys-Phe-Thr-Thr-Asp-His-Gln-Met-Ala-Arg-X₁-Cys-Asp-Asp-Cys-Cys-Gly-Gly-X₂-Gly-Arg-Gly-Lys-Cys-Tyr-Gly-Pro-Gln-Cys-Leu-Cys-Arg-OH (SEQ ID NO: 485) acetate salt (disulfide bonds, air oxidized), wherein X₁ and X₂ can each independently be Arg or Ala.

In another embodiment, the peptide can have the following formula: H-Met-Cys-Met-Pro-Cys-Phe-Thr-Thr-Asp-His-Gln-Met-Ala-Arg-Arg-Cys-Asp-Asp-Cys-Cys-Gly-Gly-Arg-Gly-Arg-Gly-Lys-Cys-Tyr-Gly-Pro-Gln-Cys-Leu-Cys-Arg-OH (SEQ ID NO: 9) acetate salt (disulfide bonds, air oxidized).

In another embodiment, the peptide can have the following formula: H-Met-Cys-Met-Pro-Cys-Phe-Thr-Thr-Asp-His-Gln-Met-Ala-Arg-Arg-Cys-Asp-Asp-Cys-Cys-Gly-Gly-Ala-Gly-Arg-Gly-Lys-Cys-Tyr-Gly-Pro-Gln-Cys-Leu-Cys-Arg-OH (SEQ ID NO: 6) acetate salt (disulfide bonds, air oxidized).

In another embodiment, the peptide can have the following formula: H-Met-Cys-Met-Pro-Cys-Phe-Thr-Thr-Asp-His-Gln-Met-Ala-Arg-Ala-Cys-Asp-Asp-Cys-Cys-Gly-Gly-Arg-Gly-Arg-Gly-Lys-Cys-Tyr-Gly-Pro-Gln-Cys-Leu-Cys-Arg-OH (SEQ ID NO: 8) acetate salt (disulfide bonds, air oxidized).

In another embodiment, the peptide can have the following formula: H-Met-Cys-Met-Pro-Cys-Phe-Thr-Thr-Asp-His-Gln-Met-Ala-Arg-Ala-Cys-Asp-Asp-Cys-Cys-Gly-Gly-Ala-Gly-Arg-Gly-Lys-Cys-Tyr-Gly-Pro-Gln-Cys-Leu-Cys-Arg-OH (SEQ ID NO: 5) acetate salt (disulfide bonds, air oxidized).

### Linkers

In some aspects, the peptides of the present disclosure are directly conjugated to a detectable label, such as a dye, fluorescent moiety or the like such that no additional amino acids, carbohydrates, nucleic acids, polymers, organic chains, or the like are added to the peptide or peptide variant and/or the dye, fluorescent moiety or the like to comprise the peptide complexes described herein. In some other aspects, a linker is used to conjugate the peptide or peptide variant is not directly conjugated to a dye, fluorescent moiety or the like such that additional amino acids, carbohydrates, nucleic acids or the like are added to the peptide or peptide variant and/or the dye, fluorescent moiety or the like to comprise the peptide complexes described herein. A “linker” as used herein refers to at least one compound comprising two functional groups that are capable of reacting specifically with other moieties to form covalent or non-covalent linkages. Such moieties can include, but are not limited to, the side groups on naturally occurring amino acids or non-natural amino acids or peptides which contain such natural or non-natural amino acids. By way of example, a linker has a functional group reactive with a group on a first peptide, and another functional group which is reactive with a group on a second peptide, whereby forming a conjugate that includes the first peptide, the linker and the second peptide. Many procedures and linker molecules for attachment of various compounds to peptides are known. See, e.g., European Patent Application No. 188,256; U.S. Pat. Nos. 4,671,958, 4,659,839, 4,414,148, 4,699,784; 4,680,338; and 4,569,789 which are incorporated by reference herein in their entirety. Linker moieties can include cleavable (e.g., pH sensitive or enzyme-labile linkers) or stable linkers.

The term “linkage,” as used herein refers to a bond or a chemical moiety formed from a chemical reaction between the functional group of a linker and another molecule. Such bonds can include, but are not limited to, covalent linkages and non-covalent bonds, while such chemical moieties include, but are not limited to, esters, carbonates, imines phosphate esters, hydrazones, acetals, orthoesters, peptide linkages, and oligonucleotide linkages. Hydrolytically stable linkages means that the linkages are substantially stable in water and do not react with water at neutral pH values, including but not limited to, under physiological conditions for an extended period of time, perhaps even indefinitely. Hydrolytically unstable or degradable linkages mean that the linkages are degradable in water or in aqueous solutions, including for example, blood. Enzymatically unstable or degradable linkages mean that the linkage is often degraded by one or more enzymes. By way of example, PEG and related polymers include degradable linkages in the polymer backbone or in the linker group between the polymer backbone and one or more of the terminal functional groups of the polymer molecule. Such degradable linkages can include, but are not limited to, ester linkages formed by the reaction of PEG carboxylic acids or activated PEG carboxylic acids with alcohol groups on a biologically active agent, wherein such ester groups generally hydrolyze under physiological conditions to release the biologically active agent. Other hydrolytically degradable linkages can include but are not limited to carbonate linkages; imine linkages resulted from reaction of an amine and an aldehyde; phosphate ester linkages formed by reacting an alcohol with a phosphate group; hydrazone linkages which are reaction product of a hydrazide and an aldehyde; acetal linkages that are the reaction product of an aldehyde and an alcohol; orthoester linkages that are the reaction product of a formate and an alcohol; peptide linkages formed by an amine group, including but not limited to, at an end of a polymer such as PEG, and a carboxyl group of a peptide; and oligonucleotide linkages formed by a phosphoramidite group, including but not limited to, at the end of a polymer, and a 5′ hydroxyl group of an oligonucleotide.

The complexes for use in the method described herein can be conjugated by using any art-recognized method forming a complex including covalent, ionic, or hydrogen bonding of the ligand to the imaging agent, either directly or indirectly via a linking group such as a linker. The conjugate can typically be formed by covalent bonding of the ligand to the imaging agent through the formation of amide, ester or imino bonds between acid, aldehyde, hydroxy, amino, or hydrazo groups on the respective components of the complex or, for example, by the formation of disulfide bonds.

In addition, structural modifications of a linker portion of the complexes are contemplated herein. For example, a number of amino acid substitutions are often made to the linker portion of the conjugate, including but not limited to naturally occurring amino acids, as well as those available from conventional synthetic methods. In one aspect, beta, gamma, and longer chain amino acids are used in place of one or more alpha amino acids. In another aspect, the stereochemistry of the chiral centers found in such molecules is selected to form various mixture of optical purity of the entire molecule, or only of a subset of the chiral centers present. In another aspect, the length of the peptide chain included in the linker is shortened or lengthened, either by changing the number of amino acids included therein, or by including more or fewer beta, gamma, or longer chain amino acids. In another aspect, the selection of amino acid side chains in the peptide portion is made to increase or decrease the relative hydrophilicity of the linker portion specifically or of the overall molecule generally.

Similarly, the length and shape of other chemical fragments of the linkers described herein can often be modified. In some aspects, the linker includes an alkylene chain. The alkylene chain can often vary in length, or can include branched groups, or can include a cyclic portion, which can be in line or spiro relative to the allylene chain. In another aspect, where the linker includes a beta thiol releasable fragment, it is appreciated that other intervening groups connecting the thiol end to the hydroxy or carbonate end are used in place of the ethylene bridge, such as but not limited to optionally substituted benzyl groups, where the hydroxy end is connected at the benzyl carbon and the thiol end is connected through the ortho or para phenyl position, and vice versa.

Direct attachment can be achieved by covalent attachment of a peptide to another molecule. For example, the peptide is attached to a terminus of the amino acid sequence of a larger polypeptide or peptide molecule, or could be attached to a side chain, such as the side chain of a lysine, serine, threonine, cysteine, tyrosine, aspartic acid, a non-natural amino acid residue, or glutamic acid residue. The attachment can be via an amide bond, an ester bond, an ether bond, a carbamate bond, a carbon-nitrogen bond, a triazole, a macrocycle, an oxime bond, a hydrazone bond, a carbon-carbon single double or triple bond, a disulfide bond, or a thioether bond. In some embodiments, similar regions of the disclosed peptide(s) itself (such as a terminus of the amino acid sequence, an amino acid side chain, such as the side chain of a lysine, serine, threonine, cysteine, tyrosine, aspartic acid, a non-natural amino acid residue, or glutamic acid residue, via an amide bond, an ester bond, an ether bond, a carbamate bond, a carbon-nitrogen bond, a triazole, a macrocycle, an oxime bond, a hydrazone bond, a carbon-carbon single double or triple bond, a disulfide bond, or a thioether bond, or linker as described herein) may be used to link other molecules.

Attachment via a linker can involve incorporation of a linker moiety between the other molecule and the peptide. The peptide and the other molecule can both be covalently attached to the linker. The linker can be cleavable, stable, self-immolating, hydrophilic, or hydrophobic. The linker can have at least two functional groups, one bonded to the other molecule, one bonded to the peptide, and a linking portion between the two functional groups. The use of a cleavable linker can permit release of the conjugated moiety (e.g., a detectable agent or a therapeutic agent) from the peptide, e.g., after targeting to a tissue of interest. The cleavable linker can comprise a cleavage site for matrix metalloproteinases, thrombin, cathepsins, or beta-glucuronidase. In other aspects, the linker can be a hydrolytically labile linker. A hydrolytically labile linker, (amongst other cleavable linkers described herein) can be advantageous in terms of releasing a fluorophore molecule or other detectable or therapeutic agents from the peptide. For example, an agent (e.g., a detectable agent or a therapeutic agent) in a conjugate form with the peptide may not be active, but upon release from the conjugate after targeting to the cartilage, the agent can be active. In some cases, the linker can be enzyme cleavable, e.g., a valine-citrulline linker. Alternatively or in combination, the linker can be cleavable by other mechanisms, such as via pH, reduction, or hydrolysis. Other cleavable linkers can include an ester bond using standard 1-ethyl-3-(3-dimethylaminopropyl)carbodiimide (EDC)-, dicylcohexylcarbodiimide (DCC)-, thionyl chloride-, or phosphorous chloride-based bioconjugation chemistries. These linkers can be cleaved by esterases, MMP, cathepsin B, a protease, or thrombin. In still other aspects, the peptide can be linked to the detectable agent via a stable linker.

Non-limiting examples of the functional groups for attachment can include functional groups capable of forming, for example, an amide bond, an ester bond, an ether bond, a carbonate bond, a carbamate bond, or a thioether bond. Non-limiting examples of functional groups capable of forming such bonds can include amino groups; carboxyl groups; hydroxyl groups; aldehyde groups; azide groups; alkyne and alkene groups; ketones; hydrazides; acid halides such as acid fluorides, chlorides, bromides, and iodides; acid anhydrides, including symmetrical, mixed, and cyclic anhydrides; carbonates; carbonyl functionalities bonded to leaving groups such as cyano, succinimidyl, and N-hydroxysuccinimidyl; hydroxyl groups; sulfhydryl groups; and molecules possessing, for example, alkyl, alkenyl, alkynyl, allylic, or benzylic leaving groups, such as halides, mesylates, tosylates, triflates, epoxides, phosphate esters, sulfate esters, and besylates.

Non-limiting examples of the linking portion can include alkylene, alkenylene, alkynylene, polyether, such as polyethylene glycol (PEG), hydroxy carboxylic acids, polyester, polyamide, polyamino acids, polypeptides, cleavable peptides, valine-citrulline, aminobenzylcarbamates, D-amino acids, and polyamine, any of which being unsubstituted or substituted with any number of substituents, such as halogens, hydroxyl groups, sulfhydryl groups, amino groups, nitro groups, nitroso groups, cyano groups, azido groups, sulfoxide groups, sulfone groups, sulfonamide groups, carboxyl groups, carboxaldehyde groups, imine groups, alkyl groups, halo-alkyl groups, alkenyl groups, halo-alkenyl groups, alkynyl groups, halo-alkynyl groups, alkoxy groups, aryl groups, aryloxy groups, aralkyl groups, arylalkoxy groups, heterocyclyl groups, acyl groups, acyloxy groups, carbamate groups, amide groups, urethane groups, epoxides, and ester groups.

Non-limiting examples of linkers can include:

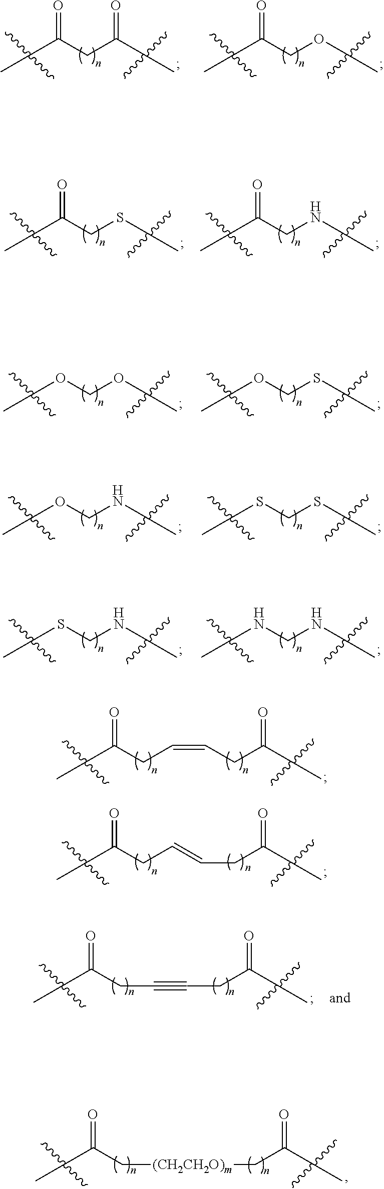

wherein each n is independently 0 to about 1,000; 1 to about 1,000; 0 to about 500; 1 to about 500; 0 to about 250; 1 to about 250; 0 to about 200; 1 to about 200; 0 to about 150; 1 to about 150; 0 to about 100; 1 to about 100; 0 to about 50; 1 to about 50; 0 to about 40; 1 to about 40; 0 to about 30; 1 to about 30; 0 to about 25; 1 to about 25; 0 to about 20; 1 to about 20; 0 to about 15; 1 to about 15; 0 to about 10; 1 to about 10; 0 to about 5; or 1 to about 5. In some embodiments, each n is independently 0, about 1, about 2, about 3, about 4, about 5, about 6, about 7, about 8, about 9, about 10, about 11, about 12, about 13, about 14, about 15, about 16, about 17, about 18, about 19, about 20, about 21, about 22, about 23, about 24, about 25, about 26, about 27, about 28, about 29, about 30, about 31, about 32, about 33, about 34, about 35, about 36, about 37, about 38, about 39, about 40, about 41, about 42, about 43, about 44, about 45, about 46, about 47, about 48, about 49, or about 50. In some embodiments, m is 1 to about 1,000; 1 to about 500; 1 to about 250; 1 to about 200; 1 to about 150; 1 to about 100; 1 to about 50; 1 to about 40; 1 to about 30; 1 to about 25; 1 to about 20; 1 to about 15; 1 to about 10; or 1 to about 5. In some embodiments, m is 0, about 1, about 2, about 3, about 4, about 5, about 6, about 7, about 8, about 9, about 10, about 11, about 12, about 13, about 14, about 15, about 16, about 17, about 18, about 19, about 20, about 21, about 22, about 23, about 24, about 25, about 26, about 27, about 28, about 29, about 30, about 31, about 32, about 33, about 34, about 35, about 36, about 37, about 38, about 39, about 40, about 41, about 42, about 43, about 44, about 45, about 46, about 47, about 48, about 49, or about 50.

### Formulations of Peptide Complexes

In various aspects, the present disclosure provides compositions comprising the above-described compounds and a pharmaceutically acceptable carrier. In some aspects, the composition is formulated for parenteral administration. In further aspects, the composition is formulated for intravenous administration, intramuscular administration, subcutaneous administration, intravascular lesion administration, or a combination thereof.

Certain methods described herein comprise administering to the subject an intravenous pharmaceutical composition comprising a peptide conjugate, for example, as described herein. Intravenous pharmaceutical compositions of peptide complexes can include any formulation suitable for administration to a subject via any intravenous method, including a bolus, a slow-bolus, an infusion which occurs over time, or any other intravenous method known in the art, as discussed further herein. “Product” or “dosage form” as used herein refers to any solid, semi-solid, lyophilized, aqueous, liquid or frozen formulation or preparation used for administration. Upon administration, the rate of release of an active moiety from a product can often be greatly influenced by the excipients and/or product characteristics which make up the product itself. For example, an enteric coat on a tablet is designed to separate that tablet's contents from the stomach contents to prevent, for example, degradation of the stomach which often induces gastrointestinal discomfort or injury. According to the currently accepted conventional understanding, systemic exposure of the active moiety can be relatively insensitive to the small formulation changes.

As used herein “pharmaceutically acceptable” or “pharmacologically acceptable” includes molecular entities and compositions that do not produce an adverse, allergic or other untoward reaction when administered to a subject, as appropriate. “Pharmaceutically acceptable carrier” includes any and all solvents, dispersion media, coatings, antibacterial and antifungal agents, isotonic and absorption delaying agents and the like. The use of such media and agents for pharmaceutical active substances is well known in the art. Except insofar as any conventional media or agent is incompatible with the active ingredient, its use in the therapeutic compositions is contemplated. Supplementary active ingredients can often also be incorporated into the compositions.

In various aspects, the present compositions comprise a concentration of the compound as an active pharmaceutical ingredient having a concentration from 0.1 mg/mL to 100 mg/mL. In some aspects, the concentration of the compound is from 0.1 mg/mL to 5 mg/mL, from 0.1 mg/mL to 10 mg/mL, from 0.1 mg/mL to 15 mg/mL, from 0.1 mg/mL to 20 mg/mL, from 0.1 mg/mL to 30 mg/mL, from 0.1 mg/mL to 40 mg/mL, from 0.1 mg/mL to 50 mg/mL, from 0.1 mg/mL to 60 mg/mL, from 0.1 mg/mL to 70 mg/mL, from 0.1 mg/mL to 80 mg/mL, or from 0.1 mg/mL to 90 mg/mL. In further aspects, the concentration of the compound is from 1 mg/mL to 20 mg/mL. In still other aspects, the concentration of the compound is from 4 mg/mL to 10 mg/mL. In additional aspects, the concentration of the compound is from 5 mg/mL to 8 mg/mL. In yet further aspects, the concentration of the compound is from 5 mg/mL to 6 mg/mL. In other aspects, the concentration of the compound is from 15 mg/mL to 35 mg/mL. In still other aspects, the concentration of the compound is from 15 mg/mL to 25 mg/mL. In yet other aspects, the concentration of the compound is from 15 mg/mL to 50 mg/mL, from 15 mg/mL to 60 mg/mL, 15 mg/mL to 70 mg/mL, 15 mg/mL to 80 mg/mL, or 15 mg/mL to 90 mg/mL.

In some embodiments, the pharmaceutically acceptable carrier has a pH of about 6.0, about 6.1, about 6.2, about 6.3, about 6.4, about 6.5, about 6.6, about 6.7, about 6.8, about 6.9, about 7.0, about 7.1, about 7.2, about 7.3, about 7.4, about 7.5, about 7.6, about 7.7, about 7.8, about 7.9, or about 8.0. In still other embodiments, the pharmaceutically acceptable carrier has a pH within a range from about 6.0 to about 7.5. In other embodiments, the pharmaceutically acceptable carrier has a pH within a range from about 5.0 to about 9.0.

In some embodiments, the composition has a pH of about 6.0, about 6.1, about 6.2, about 6.3, about 6.4, about 6.5, about 6.6, about 6.7, about 6.8, about 6.9, about 7.0, about 7.1, about 7.2, about 7.3, about 7.4, about 7.5, about 7.6, about 7.7, about 7.8, about 7.9, or about 8.0. In still other embodiments, the composition has a pH within a range from about 6.0 to about 7.5. In other embodiments, the composition has a pH within a range from about 5.0 to about 9.0.

In some aspects, a pharmaceutically acceptable carrier comprises tris, D-mannitol, L-histidine, L-methionine, polysorbate 20, or a combination thereof. For example, in some aspects, a pharmaceutically acceptable carrier comprises tris and D-mannitol. In some aspects, a pharmaceutically acceptable carrier comprises L-histidine and D-mannitol. In some aspects, the pharmaceutically acceptable carrier comprises L-histidine and D-mannitol with polysorbate 20. In some aspects, the pharmaceutically acceptable carrier comprises L-histidine, D-mannitol, and L-methionine.

In some aspects, the pharmaceutically acceptable carrier comprises L-histidine, D-mannitol, polysorbate 20, and a pH of about 6.8. In some aspects, the pharmaceutically acceptable carrier comprises L-histidine, D-mannitol, polysorbate 20, and a pH within a range of about 6 to about 7.5. In some aspects, the pharmaceutically acceptable carrier comprises L-histidine, D-mannitol, polysorbate 20, and a pH within a range of about 5 to about 9. In some aspects, the pharmaceutically acceptable carrier comprises L-histidine, D-mannitol, and a pH of about 6.8. In some aspects, the pharmaceutically acceptable carrier comprises L-histidine, D-mannitol, and a pH within a range of about 6 to about 7.5. In some aspects, the pharmaceutically acceptable carrier comprises L-histidine, D-mannitol, and a pH within a range of about 5 to about 9. In some aspects, the pharmaceutically acceptable carrier comprises L-histidine, D-mannitol, polysorbate 20, trehalose, and a pH of about 6.8. In some aspects, the pharmaceutically acceptable carrier comprises L-histidine, D-mannitol, polysorbate 20, trehalose, and a pH within a range of about 6 to about 7.5. In some aspects, the pharmaceutically acceptable carrier comprises L-histidine, D-mannitol, polysorbate 20, trehalose, and a pH within a range of about 5 to about 9.

A pharmaceutical composition comprising a peptide conjugate can be formulated according to known methods to prepare pharmaceutically useful compositions, for example, as found in “Excipient Selection in Parenteral Formulation Development” Pramanick et. al., Pharma Times, Vol. 45., No. 3, March 2013, incorporated in its entirety herein by reference. In some aspects, the peptide conjugate is combined with a pharmaceutically acceptable carrier. A composition is said to be a pharmaceutically acceptable carrier if its administration is tolerated by a recipient patient. Sterile phosphate-buffered saline is one example of a pharmaceutically acceptable carrier. Other suitable carriers are well-known to those in the art. See, for example, Gennaro (ed.), Remington's Pharmaceutical Sciences, 19th Edition (Mack Publishing Company 1995).

Formulations for administration of peptide complexes are typically provided but are not limited to as liquid, solid or semi-solid products or dosage forms, exemplified by tablets, capsules, pellets, a powder or a lyophilized product. In some aspects, the peptide conjugate is formulated to comprise no additional materials except for a pharmaceutical carrier. In some other aspects, the peptide conjugate is formulated such that it comprises a core “matrix material” which encapsulates, binds to or accumulates in, coats or is adjacent to the peptide conjugate. In some other aspects, the peptide conjugate and matrix material further comprises a protective coating. Various formulations are well-known to those in the art. See, for example, Gennaro (ed.), Remington's Pharmaceutical Sciences, 19th Edition (Mack Publishing Company 1995).

Suitable excipients for use with peptide complexes are often included in formulations for intravenous use, for example, an injection. Injections are sterile, pyrogen-free solutions or dispersions (emulsions or suspensions) of one or more active ingredients in a suitable vehicle or carrier. Injections that are dispersions should remain sufficiently stable so that, after shaking, a homogeneous dose can be withdrawn. More specifically, formulations which can include peptide complexes and one or more but not limited to suitable excipients, exemplified by matrix materials, binders, lubricants, glidants or disintegrants which aid in modulating the PK profile of administered peptide complexes are preferred. In some aspects, compositions comprise peptide complexes in combination with one or more suitable excipients and one or more specific product characteristics (such as dissolution or water content) which result in improved pharmacokinetic profiles of peptide complexes in vivo. Thus, the in vivo performance of peptide complexes dosage forms/products included herein can be based upon the composition of the excipients added during manufacturing and/or the final product characteristics generated through specific processing parameters and methods. Other excipients are well-known to those in the art. See, for example, Gennaro (ed.), Remington's Pharmaceutical Sciences, 19th Edition (Mack Publishing Company 1995).

Suitable carriers for intravenous administration can include, for example, but are not limited to, physiological saline or phosphate buffered saline (PBS), Tris, and solutions containing solubilizing agents, such as glucose, polyethylene glycol, polypropylene glycol, additional agents such as histidine, dextrose, mannitol and mixtures thereof. In some aspects, carriers for intravenous administration include a mixture of histidine and dextrose, Tris and dextrose or Tris and mannitol. Other carriers are well-known to those in the art. See, for example, Gennaro (ed.), Remington's Pharmaceutical Sciences, 19th Edition (Mack Publishing Company 1995).

The formulation can often include an aqueous vehicle. Aqueous vehicles include, by way of example and without limitation, sodium chloride solution, Ringers solution, isotonic dextrose solution, sterile water solution, dextrose and lactated Ringers solution. Nonaqueous vehicles can include, by way of example and without limitation, fixed oils of vegetable origin, cottonseed oil, corn oil, sesame oil and peanut oil, benzyl benzoate, castor oil, N,N-dimethylacetamide, ethanol, dehydrated ethanol, glycerin, glycerol, N-methyl-2-pyrrolidone, polyethylene glycol and any derivative thereof, propylene glycol, safflower oil and soybean oil. Other vehicles are well-known to those in the art. See, for example, Gennaro (ed.), Remington's Pharmaceutical Sciences, 19th Edition (Mack Publishing Company 1995).

In some aspects, the composition the pharmaceutically acceptable carrier comprises an osmolyte. In some aspects, the osmolyte comprises a sugar, a sugar alcohol, or a combination thereof.

In certain aspects, the composition comprises a sugar alcohol. In certain aspects, the composition comprises a sugar alcohol selected from sorbitol, inositol, mannitol, xylitol, glycerol, or a combination thereof. In further aspects, the sugar alcohol comprises mannitol.

In certain aspects, the composition comprises from about 2% to about 20% (wt/vol %) sugar alcohol. In some aspects, the composition comprises from about 2% to about 10% (wt/vol %) sugar alcohol. In some aspects, the composition comprises from about 3% to about 10% (wt/vol %) sugar alcohol. In further aspects, the composition comprises about 5% (wt/vol %) sugar alcohol. In certain aspects, the composition comprises from about 2% to about 20% (wt/vol %) mannitol. In some aspects, the composition comprises from about 2% to about 10% (wt/vol %) mannitol. In further aspects, the composition comprises about 5% (wt/vol %) mannitol.

In other aspects, the composition comprises a sugar. In certain aspects, the sugar is selected from trehalose, lactose, sucrose, glucose, galactose, maltose, mannose, fructose, dextrose, or a combination thereof. In additional aspects, the sugar is selected from trehalose, sucrose, or a combination thereof. In some aspects, the composition comprises from about 1% to about 40% (wt/vol %) of sugar. In other aspects, the composition comprises from about 1% to about 20% (wt/vol %) of sugar. In additional aspects, the composition comprises about 2% (wt/vol %) of sugar. In some aspects, the composition comprises from about 1% to about 40% (wt/vol %) of trehalose, sucrose, or a combination of trehalose and sucrose. In other aspects, the composition comprises from about 1% to about 20% (wt/vol %) of trehalose, sucrose, or a combination of trehalose and sucrose. In additional aspects, the composition comprises about 2% (wt/vol %) of trehalose, sucrose, or a combination of trehalose and sucrose.

In certain aspects, the composition further comprises an osmolyte selected from glycine, carnitine, ethanolamine, their phosphates, mono sugars, or a combination thereof.

In some aspects, the present compositions are isotonic. In other aspects, the compositions are about isotonic.

In certain aspects, the ionic strength of the composition is less than or equal to 60 mM. In certain aspects, the composition comprises an ionic strength less than or equal to 50 mM. In certain aspects, the ionic strength of the composition is less than or equal to 40 mM. In certain aspects, the ionic strength of the composition is less than or equal to 30 mM. In certain aspects, the ionic strength of the composition is less than or equal to 20 mM. In other aspects, the ionic strength of the composition is less than or equal to 10 mM.

Antimicrobial agents in bacteriostatic or fungistatic concentrations can typically be added to preparations packaged in multiple dose containers which can include, by way of example and without limitation, phenols or cresols, mercurials, benzyl alcohol, chlorobutanol, methyl and propyl p-hydroxybenzoic acid esters, thimerosal, benzalkonium chloride and benzethonium chloride. Other antimicrobial agents are well-known to those in the art. See, for example, Gennaro (ed.), Remington's Pharmaceutical Sciences, 19th Edition (Mack Publishing Company 1995).

Buffers can include, by way of example and without limitation, acetate, ammonium sulfate, ammonium hydroxide, arginine, aspartic acid, benzene sulfonic acid, benzoate sodium, benzoate acid, carbonate, sodium carbonate, carbon dioxide, citrate, diethanolamine, glucono delta lactone, glycine, glycine HCl, histidine, histidine HCl, hydrochloric acid, hydrobromic acid, lysine maleic acid, meglumine, methanesulfonic acid, monoethanolamine, phosphate, sodium phosphate, citrate, succinate sodium, sulfuric acid, tartarate sodium, trmethamine, sodium citrate, hydroxide, sodium hydroxide, Tris base, Tris base-65, Tris acetate, Tris HCl, and Tris HCl-65.

In various aspects, the pharmaceutically acceptable carrier comprises a buffer. In some aspects, the buffer is selected from tris, HEPES, histidine, ethylene diamine, or a combination thereof. In other aspects, the buffer is selected from tris, histidine, or a combination thereof. In further aspects, the buffer comprises histidine, which is optionally L-histidine. In another aspect, the composition comprises a buffer comprising histidine, tris, HEPES, ethylene diamine, or a combination thereof. In additional aspects, the composition comprises at least 100 mM histidine. In further aspects, the composition comprises at least or equal to 50 mM histidine. In some aspects, the composition comprises at least or equal to 20 mM histidine. In additional aspects, the composition comprises 10 to 100 mM histidine. In other aspects, the composition comprises 10 to 20 mM histidine. In other aspects, the composition comprises 0 to 50 mM hisitidine. In further aspects, the composition comprises at least 100 mM tris. In some aspects, the composition comprises at least or equal to 50 mM tris. In additional aspects, the composition comprises at least or equal to 20 mM tris. In other aspects, the composition comprises 10 to 20 mM tris. In other aspects, the composition comprises 0 to 20 mM tris. In some aspects, the composition comprises from about 0 mM to about 50 mM histidine, from about 0 mM to about 20 mM tris, about 20 mM methionine, from about 3% to about 10% (wt/vol %) sugar alcohol, and a pH within a range from about 6 to about 7.5.

Antioxidants can include, by way of example and without limitation, sodium bisulfate, acetone sodium bisulfate, argon, ascorbyl palmitate, ascorbate sodium, ascorbate acid, butylated hydroxy anisole, butylated hydroxy toluene, cysteine, cystenate HCl, dithionite sodium, gentistic acid, gentistic acid ethanoloamine, glutamate monosodium, glutathione, formaldehyde solfoxylate sodium, metabisulfite potassium, metabisulfite sodium, methionine, monothioglycerol, nitrogen, propyl gallate, sulfite sodium, tocopherol alpha, alpha tocopherol hydrogen succinate, and thioglycolyate sodium.

In some aspects, the compositions comprise an antioxidant, a free radical scavenger, a quencher, an antioxidant synergist, or a combination thereof.

In some aspects, the antioxidant is selected from methionine, butylated hydroxytoluene, butylated hydroxyanisole, propyl gallate, or a combination thereof. In other aspects, the antioxidant comprises methionine. In further aspects, the antioxidant is L-methionine. In certain aspects, the compositions comprise at least or equal to 20 mM methionine. In other aspects, the compositions comprise at least or equal to 5 mM methionine. In still other aspects, the compositions comprise at least or equal to 10 mM methionine. In further aspects, the compositions comprise at least or equal to 50 mM methionine. In other aspects, the compositions comprise 10 to 20 mM methionine. In other aspects, the compositions comprise 0 to 50 mM methionine.

Suspending, emulsifying and/or dispersing agents can include, by way of example and without limitation, sodium carboxymethylcelluose, hydroxypropyl methylcellulose, Polysorbate 80 (TWEEN® 80), and polyvinylpyrrolidone.

In various aspects, the compositions comprise a surfactant. In certain aspects, the surfactant is selected from polysorbate 20, polysorbate 80, a pluronic, polyoxyethylene sorbitan mono-oleate, polyethylene mono-laureate, N-actylglucoside, or a combination thereof. In certain aspects, the surfactant is polysorbate 20. In further aspects, the compositions comprise from 0.0001% to 0.1% (wt/vol %) polysorbate 20. In additional aspects, the compositions comprise cyclodextrin. In further aspects, the cyclodextrin comprises (2-hydroxypropyl)-β-cyclodextrin.

A sequestering or chelating agent of metal ions can include, by way of example and without limitation, calcium disodium EDTA, disodium EDTA, sodium EDTA, calcium versetaminde sodium, calteridol, and DPTA. In some aspects, the present compositions comprise a metal chelator. In certain aspects, the metal chelator is selected from EDTA, deferoxamine mesylate, EGTA, fumaric acid, and malic acid, salts thereof, or combinations thereof. In further aspects, the metal chelator comprises EDTA or salts thereof. In certain aspects, the compositions have an EDTA concentration of about 0.1 mg/ml to about 1.0 mg/ml.

Other isotonic agents, buffers, antioxidants, anesthetics, suspending and dispersing agents, emulsifying agents and chelating agents are well-known to those in the art. See, for example, Gennaro (ed.), Remington's Pharmaceutical Sciences, 19th Edition (Mack Publishing Company 1995).

Pharmaceutical carriers can also include, by way of example and without limitation, ethyl alcohol, polyethylene glycol and propylene glycol for water miscible vehicles and sodium hydroxide, hydrochloric acid, citric acid or lactic acid. Other pharmaceutical carriers are well-known to those in the art. See, for example, Gennaro (ed.), Remington's Pharmaceutical Sciences, 19th Edition (Mack Publishing Company 1995).

The peptide complexes described herein can often be formulated using a variety of parameters, including by way of example and without limitation, pH, molarity, % weight/volume, % volume/volume, and the like. Other factors can be considered in the formulation of, stability of, storage of, shipping of peptide complexes can include by way of example and without limitation, the gas environment, container material, container color, cap material, cap color, presence of additional aspects, such as antioxidants, stabilizers, photoprotective compounds, protectants, sugars, ion chelators, ion donors, or the like. Any factor which serves as any one of the above factors known to one of ordinary skill in the art can often be used with the peptide complexes described herein but not limited as such.

The preparation of pharmaceutical or pharmacological compositions are known to those of skill in the art in light of the present disclosure. General techniques for formulation and administration can be found in “Remington: The Science and Practice of Pharmacy, Twentieth Edition,” Lippincott Williams & Wilkins, Philadelphia, Pa. Tablets, capsules, pills, powders, granules, dragees, gels, slurries, ointments, solutions suppositories, injections, inhalants, and aerosols are examples of such formulations.

The peptide complexes can often be stored at various temperatures, including by way of example and without limitation, freezing, for example at about −20° C., about −70° C., about −80° C., about −100° C., about −120° C., about −150° C., about −200° C. or more than about −200° C., cold storage, for example at about 10° C., about 5° C., about 4° C., about 2° C., about 0° C., about −2° C. or more than about −5° C., or any other suitable temperature such that the composition remains stable.

In some aspects, compositions comprising the compounds described herein are stored as lyophilized solids. In some aspects, the present disclosure provides methods for producing the lyophilized composition, the method comprising providing the composition, and lyophilizing the composition, thereby producing the lyophilized composition.

Using lyophilization, it can be possible to store the compounds in a manner that maintains physiological or otherwise optimal pH, isotonicity and stability. Such materials can include pH buffers, preservatives, tonicity adjusting agents, anti-oxidants, other polymers (e.g., viscosity adjusting agents or extenders) and excipients to stabilize the labile protein against the stresses of drying and storage of the dried product. Specific illustrative examples of such additives can include phosphate, citrate, or borate buffers; thimerosal; sorbic acid; methyl or propyl paraben, and chlorobutanol preservatives; sodium chloride: polyvinyl alcohol, polyvinyl pyrrolidone; mannitol, dextrose, dextran, lactose, sucrose, ethylene diamine tetra-acetic acid, and the like. Suitable formulations, known in the art, can be found in Remington's Pharmaceutical Sciences (latest edition), Mack Publishing Company, Easton, Pa.; Arakawa et al. (1990), supra; Carpenter et al. (1991), supra; and Pikal (1990), supra.

In certain aspects, the pharmaceutically acceptable carrier comprises a reconstitution stabilizer. In other aspects, the reconstitution stabilizer comprises a water-soluble polymer. In additional aspects, the water-soluble polymer is selected from a polaxamer, a polyol, a polyethylene glycol, a polyvinylalcohol, a hydroxyethyl starch, dextran, polyvinylpyrrolidene poly(acrylic acid), or a combination thereof.

The term “reconstitution stabilizer” means any excipient which is capable of preventing aggregation of a reconstituted protein in an aqueous medium. Excipients possessing the necessary characteristics for the present invention are well-known in the art and generally function by the mechanisms of charge replusion, steric hindrance, hydrophobic binding or specific high-affinity binding to the dried protein. Exemplary excipients include various osmolytes, various salts, water soluble synthetic and natural polymers, surfactants, sulfated polysaccharides, carrier proteins, buffers and the like (Manning et al. (1989), Pharmaceutical Research, 6:903-918; and Paborji, et al. (1994), Pharmaceutical Research, 11:764-771).

The present compounds and an effective amount of the reconstitution stabilizer can be admixed under conditions effective to reduce aggregation of present compounds upon reconstitution with the reconstitution medium (e.g., a solvent and optionally other components such as antibacterials). The reconstitution stabilizer can be admixed with the compounds at a suitable time before, during or after reconstitution. In one aspect, the reconstitution stabilizer will be pre-dissolved in the reconstitution medium. The compound can be reconstituted at a temperature which is above the freezing point of the reconstitution medium, but which will not degrade the compound and which will not be deleterious to the reconstitution stabilizer. In one aspect, the temperature will be between about 2° C. to 50° C. The time taken to mix the reconstitution stabilizer and the dried compound should be for a sufficient period to prepare a suitable admixture. In one aspect, the mixing will be for between about 1 to 30 minutes. Generally, the reconstituted formulation can be used soon after reconstitution.

In certain aspects, the present compositions are reconstituted from a lyophilized form. In other aspects, the present disclosure provides methods for producing the reconstituted composition, the method comprising providing a lyophilized composition; and reconstituting the composition with a solution to produce a reconstituted composition. In various aspects, the reconstituting solution comprises water. In some aspects, the reconstituting solution is selected from sterile water, physiological saline solution, glucose solution or other aqueous solvents (e.g., alcohols such as ethyl, n-propyl or isopropyl, butyl alcohol), or a combination thereof, which are capable of dissolving the dried composition and compatible with the selected administration route and which does not negatively interfere with the compound and the reconstitution stabilizers employed.

### Dosages and Methods of Administration of Peptide Complexes

The product or dosage form characteristics which can result from processing methods and/or parameters for generating formulations such as powders, lyophilized compositions, and the like, and can include, but are not limited to, density, water content, friability, disintegration, dissolution profile(s), shape, size, weight, uniformity and composition of the particles. These product characteristics can often be modulated in a number of ways and affect the final in vitro and/or in vivo performance of the formulations. Product or dosage form characteristics can often be a consequence of excipient selection, excipient composition, manufacturing methods applied, or a combination of any of these. The combination of excipients as well as product characteristics (including processing methods or processing parameters) of the final dosage form can ultimately determine the pharmacokinetic profile of the active ingredient in vivo. The administered peptide conjugate formulations described herein can often be processed or manufactured under specific conditions such as, for example, mixing methods (including sieve size, rpm, and milling), drying time, conditions, environmental parameters (e.g., temperature, humidity and combinations thereof) which themselves can modulate the pharmacokinetic profile of compositions in vivo (i.e., increase the average Cₘₐₓ or AUC). In order to quantitatively compare one formulation to another, one can measure several of these product or dosage form characteristics. This can also necessary when attempting to duplicate multiple batches.

Dissolution and drug release from formulations can depend on many factors including the solubility and concentration of the active ingredient, the nature and composition of the excipients, content uniformity, water content, product shape and size, porosity, disintegration time, and other factors. The release of a drug or active ingredient from a final dosage form in vitro is typically characterized by its dissolution profile under standardized conditions (using United States Pharmacopeia (USP) or similar accepted methods for reference) and at the appropriate pH, often a neutral pH. The dissolution profile shows the amount of drug released over time into the test media under specified conditions. Standard conditions make use of buffers at an appropriate pH in order to best mimic the pH of a subject's blood.

Typically, a therapeutically effective dosage can be formulated to contain a dose of at least about 0.1 mg up to about 100 mg or more, such as more than 100 mg of peptide conjugate. In some aspects, the effective dosage is formulated to contain a dose of at least about 0.01 mg, about 0.02 mg, about 0.03 mg, about 0.05 mg, about 0.07 mg, about 0.1 mg, about 0.2 mg, about 0.3 mg, about 0.35 mg, about 0.375 mg, about 0.4 mg, about 0.5 mg, about 0.6 mg, about 0.7 mg, about 0.75 mg, about 0.8 mg, about 0.9 mg, about 1 mg, about 1.3 mg, about 1.4 mg, about 1.5 mg, about 1.8 mg, about 1.9 mg, about 2 mg, about 2.4 mg, about 3 mg about 5 mg, about 6 mg, about 7 mg, about 8 mg, about 9 mg about 10 mg about 11 mg about 12 mg, about 13 mg, about 14 mg, about 15 mg, about 16 mg about 17 mg, about 18 mg, about 19 mg, about 20, about 21 mg, about 22 mg, about 23 mg, about 24 mg, about 25 mg, about 26 mg, about 27 mg, about 28 mg, about 29 mg, about 30 mg, about 31 mg, about 32 mg, about 33 mg, about 34 mg, about 35 mg, about 40 mg, about 45 mg, about 50 mg, about 55 mg, about 60 mg, about 70 mg, about 80 mg, about 90 mg, about 100 mg, about 150 mg or about 200 mg or more of peptide conjugate. In an exemplary aspect, the dose is 0.03 mg for a mouse, 1 mg for a dog, 0.3 mg for a rat, 0.6 mg for a monkey, and 6 mg or 12 mg for a human via intravenous administration.

In some exemplary aspects, a therapeutically effective dosage is formulated to contain a dose of 1 mg to 200 mg or more for a human. In other aspects, the effective dosage is formulated to contain a dose of 1 mg to 5 mg, of 1 mg to 10 mg, of 1 mg to 20 mg, of 1 mg to 30 mg, of 1 mg to 40 mg, of 1 mg to 50 mg, of 1 mg to 60 mg, of 1 mg to 70 mg, of 1 mg to 80 mg, of 1 mg to 90 mg, of 1 mg to 100 mg, of 1 mg to 120 mg, of 1 mg to 140 mg, of 1 mg to 160 mg, of 1 mg to 180 mg, 3 mg to 5 mg, of 3 mg to 10 mg, of 3 mg to 20 mg, of 3 mg to 30 mg, of 3 mg to 40 mg, of 3 mg to 50 mg, of 3 mg to 60 mg, of 3 mg to 70 mg, of 3 mg to 80 mg, of 3 mg to 90 mg, of 3 mg to 100 mg, of 3 mg to 120 mg, of 3 mg to 140 mg, of 3 mg to 160 mg, of 3 mg to 180 mg, of 3 mg to 200 mg, of 10 mg to 20 mg, of 10 mg to 30 mg, of 10 mg to 40 mg, of 10 mg to 50 mg, of 10 mg to 60 mg, of 10 mg to 70 mg, of 10 mg to 80 mg, of 10 mg to 90 mg, of 10 mg to 100 mg, of 10 mg to 120 mg, of 10 mg to 140 mg, of 10 mg to 160 mg, of 10 mg, to 180 mg, of 10 mg to 200 mg, of 20 mg to 50 mg, of 20 mg to 75 mg, of 20 mg to 100 mg, of 20 mg to 120 mg, of 20 mg, to 140 mg, of 20 mg to 160 mg, of 20 mg to 180 mg, of 20 mg to 200 mg, of 30 mg to 50 mg, of 30 mg to 75 mg, of 30 mg to 100 mg, of 30 mg to 120 mg, of 30 mg to 140 mg, of 30 mg to 160 mg, of 30 mg to 180 mg, of 30 mg to 200 mg, of 50 mg to 60 mg, of 50 mg to 75 mg, of 50 mg to 100 mg, of 50 mg to 120 mg, of 50 mg to 140 mg, of 50 mg to 160 mg, of 50 mg to 180 mg, of 50 mg to 200 mg, of 75 mg to 80 mg, of 75 mg to 90 mg, of 75 mg to 100 mg, of 75 mg to 120 mg, of 75 mg to 140 mg, of 75 mg to 160 mg, of 75 mg to 180 mg, of 75 mg to 200 mg, of 100 mg to 120 mg, of 100 mg to 140 mg, of 100 mg to 160 mg, of 100 mg to 180 mg, of 100 mg to 200 mg, of 120 mg to 140 mg, of 120 mg to 160 mg, of 120 mg to 180 mg, of 120 mg to 200 mg, of 140 mg to 160 mg, of 140 mg to 180 mg, of 140 mg to 200 mg, of 160 mg to 180 mg, of 160 mg to 200 mg, or of 180 mg to 200 mg.

The amount of peptide conjugate administered to a subject can often be the total about amount listed herein. In some aspects, the amount of peptide conjugate administered to a subject is often the about per milligram, gram or kilogram of subject weight for each amount listed herein. In other aspects, the amount of peptide conjugate administered to a subject is often the about per milliliter or liter of fluid volume for each amount listed herein. In yet other aspects, the amount of peptide conjugate administered to a subject is often the about per square millimeter, square centimeter or square meter of subject surface body area or subject body area for each amount listed herein.

As used herein a “dosage regimen” refers to the protocol used to administer an intravenous pharmaceutical formulation comprising peptide conjugate to a subject. In some aspects, the dosage regimen comprises a dose amount and dosing interval. In some aspects, the dosage regimen further comprises a dosing duration. As used herein “dosing duration” refers to the period of time over which a dose is administered. Furthermore, the dosage regimen comprises a method of administration. In some aspects, a method of administration comprises a bolus, a slow bolus, or an infusion.

As used herein, a “bolus” may refer to an intravenous injection administered over a short period of time. In one aspect, a bolus is manually administered over a short period of time. In other aspects, a bolus is administered via a pump or other automated mechanism over a short period of time. In some aspects, a bolus is administered over a period of time less than or equal to 5 seconds, less than or equal to 10 seconds, less than or equal to 15 seconds, less than or equal to 20 seconds, less than or equal to 25 seconds, less than or equal to 30 seconds, less than or equal to 35 seconds, less than or equal to 40 seconds, less than or equal to 45 seconds, less than or equal to 50 seconds, less than or equal to 55 seconds, less than or equal to 60 seconds, less than or equal to 65 seconds, less than or equal to 70 seconds, less than or equal to 75 seconds, less than or equal to 80 seconds, less than or equal to 85 seconds, less than or equal to 90 seconds, less than or equal to 95 seconds, less than or equal 100 seconds, less than or equal to 105 seconds, less than or equal to 110 seconds, less than or equal to 115 seconds, or less than or equal to 120 seconds.

As used herein, a “slow bolus” may refer to an intravenous injection administered over longer period of time than a bolus, but a shorter period of time than an infusion. In one aspect, a slow bolus is manually administered over a longer period of time than a bolus, but a shorter period of time than an infusion. In other aspects, a slow bolus is administered via a pump or other automated mechanism over a longer period of time than a bolus, but a shorter period of time than an infusion. In one aspect, a slow bolus is administered over a period of time within a range from about 2 minutes to about 5 minutes. In other aspects, a slow bolus is administered over a period of time within a range from about 2 minutes to about 4.9 minutes, about 2 minutes to about 4.8 minutes, about 2 minutes to about 4.8 minutes, about 2 minutes to about 4.7 minutes, about 2 minutes to about 4.6 minutes, about 2 minutes to about 4.5 minutes, about 2 minutes to about 4.4 minutes, about 2 minutes to about 4.3 minutes, about 2 minutes to about 4.4 minutes, about 2 minutes to about 4.3 minutes, about 2 minutes to about 4.2 minutes, about 2 minutes to about 4.1 minutes, about 2 minutes to about 4 minutes, about 2 minutes to about 3.9 minutes, about 2 minutes to about 3.8 minutes, about 2 minutes to about 3.7 minutes, about 2 minutes to about 3.6 minutes, about 2 minutes to about 3.5 minutes, about 2 minutes to about 3.4 minutes, about 2 minutes to about 3.3 minutes, about 2 minutes to about 3.2 minutes, about 2 minutes to about 3.1 minutes, about 2 minutes to about 3 minutes, about 2 minutes to about 2.9 minutes, about 2 minutes to about 2.8 minutes, about 2 minutes to about 2.7 minutes, about 2 minutes to about 2.6 minutes, about 2 minutes to about 2.5 minutes, about 2 minutes to about 2.4 minutes, about 2 minutes to about 2.3 minutes, about 2 minutes to about 2.2 minutes, or about 2 minutes to about 2.1 minutes. In other aspects, a slow bolus is administered over a period of time within the range of about 2.5 minutes to about 3 minutes, about 2.5 minutes to about 3.5 minutes, about 2.5 minutes to about 4 minutes, about 2.5 minutes to about 4.5 minutes, about 2.5 minutes to about 5 minutes, about 3 minutes to about 3.5 minutes, about 3 minutes to about 4 minutes, about 3 minutes to about 4.5 minutes, about 3 minutes about 5 minutes, about 3.5 minutes to about 4 minutes, about 3.5 minutes to about 4.5 minutes, about 3.5 minutes to about 5 minutes, about 4 minutes to about 4.5 minutes, about 4 minutes about 5 minutes, or about 4.5 minutes to about 5 minutes.

As used herein, an “infusion” may refer to an intravenous injection administered over longer period of time than a bolus or a slow bolus. In one aspect, an infusion is administered via a pump or other automated mechanism over longer period of time than a bolus or a slow bolus. In other aspects, an infusion is manually administered over longer period of time than a bolus or a slow bolus. In other aspects, the infusion is administered over a period of time that is greater than or equal to 5 minutes, greater than or equal to 5.5 minutes, greater than or equal to 6 minutes, greater than or equal to 6.5 minutes, greater than or equal to 7 minutes, greater than or equal to 7.5 minutes, greater than or equal to 8 minutes, greater than or equal to 8.5 minutes, greater than or equal to 9 minutes, greater than or equal to 9.5 minutes, greater than or equal to 10 minutes, greater than or equal to 10.5 minutes, greater than or equal to 11 minutes, greater than or equal to 11.5 minutes, greater than or equal to 12 minutes, greater than or equal to 12.5 minutes, greater than or equal to 13 minutes, greater than or equal to 13.5 minutes, greater than or equal to 14 minutes, greater than or equal to 14.5 minutes, greater than or equal to 15 minutes, greater than or equal to 15.5 minutes greater than or equal to 16 minutes, greater than or equal to 16.5 minutes, greater than or equal to 17 minutes, greater than or equal to 17.5 minutes, greater than or equal to 18 minutes, greater than or equal to 18.5 minutes, greater than or equal to 19 minutes, greater than or equal to 19.5 minutes, greater than or equal to 20 minutes, greater than or equal to 30 minutes, greater than or equal to 45 minutes, greater than or equal to 60 minutes, greater than or equal to 75 minutes, greater than or equal to 90 minutes, greater than or equal to 105 minutes, greater than or equal to 120 minutes, greater than or equal to 150 minutes, greater than or equal to 180 minutes, greater than or equal to 210 minutes, greater than or equal to 240 minutes, greater than or equal to 270 minutes, greater than or equal to 300 minutes. In still other aspects, the infusion is administered over a period of time that is within a range of about 5 minutes to about 20 minutes, about 5 minutes to about 19 minutes, about 5 minutes to about 18 minutes, about 5 minutes to about 17 minutes, about 5 minutes to about 16 minutes, about 5 minutes to about 15 minutes, about 5 minutes to about 14 minutes, about 5 minutes to about 13 minutes, about 5 minutes to about 12 minutes, about 5 minutes to about 10 minutes, about 5 minutes to about 9 minutes, about 5 minutes to about 8 minutes, about 5 minutes to about 7 minutes, or about 5 minutes to about 6 minutes. In yet still further aspects, the infusion is administered over a period of time that is within the range of about 5 minutes to about 10 minutes, about 5 minutes to about 15 minutes, about 5 minutes to about 20 minutes, about 5 minutes to about 25 minutes, about 5 minutes to about 30 minutes, about 5 minutes to about 45 minutes, about 5 minutes to about 60 minutes, about 5 minutes to about 90 minutes, about 5 minutes to about 120 minutes, about 5 minutes to about 150 minutes, about 5 minutes to about 180 minutes, about 5 minutes to about 210 minutes, about 240 minutes to about 270 minutes, about 5 minutes to about 300 minutes, about 30 minutes to about 75 minutes, about 30 minutes to about 90 minutes, about 30 minutes to about 120 minutes, about 30 minutes to about 150 minutes, about 30 minutes to about 180 minutes, about 30 minutes to about 210 minutes, about 30 minutes to about 240 minutes, about 30 minutes to about 270 minutes, about 30 minutes to about 300 minutes, about 60 minutes to about 90 minutes, about 60 minutes to about 120 minutes, about 60 minutes to about 150 minutes, about 60 minutes to about 180 minutes, about 60 minutes to about 210 minutes, about 60 minutes to about 240 minutes, about 60 minutes to about 270 minutes, about 60 minutes to about 300 minutes, about 90 minutes to about 120 minutes, about 90 minutes to about 180 minutes, about 90 minutes to about 240 minutes, about 60 minutes to about 300 minutes, about 120 minutes to about 180 minutes, about 120 minutes to about 240 minutes, about 120 minutes to about 300 minutes, about 180 minutes to about 240 minutes, about 180 minutes to about 300 minutes, or about 240 minutes to about 300 minutes.

In some aspects, the dose of peptide conjugate is administered to a subject using either a fixed or a scaling dosing scheme. For example, a fixed dosing scheme can include administration of a bolus, a slow bolus or an infusion of peptide conjugate to a subject via an intravenous administration route wherein the fixed dose is, for example and without limitation, 0.1 mg to 100 mg and does not account or adjust for a subject's age, weight, height, body mass index, metabolism, or the like, or 1 mg to 30 mg and does not account or adjust for a subject's age, weight, height, body mass index, metabolism, or the like. For example, a scaling dosing scheme can include administration of a bolus, a slow bolus or an infusion of peptide conjugate to a subject via an intravenous administration route wherein the scaled dose is, for example and without limitation, 0.1 mg to 100 mg and accounts or adjusts for a subject's age, weight, height, body mass index, metabolism, or the like, or 1 mg to 30 mg and accounts or adjusts for a subject's age, weight, height, body mass index, metabolism, or the like. In some aspects, the fixed dose and/or the scaled dose are determined for one subject based upon the dose administered to a different subject wherein the subjects are or are not the same species, for example a mouse and a human, a rat and a human, a dog and a human, a monkey and a human, or a non-human primate and a human. Often in a fixed dose, the same dose or about the same dose can be administered to all subjects, for example a mouse and a human, a rat and a human, a dog and a human, a monkey and a human, or a non-human primate and a human. In some aspects, the scaled dose to be administered to a subject is determined from the dose administered to a different subject wherein the subjects are or are not the same species, for example a mouse and a human, a rat and a human, a dog and a human, a monkey and a human, or a non-human primate and a human. The scaled dose can therefore be increased from the dose administered to the mouse, rat, dog, monkey, or non-human primate to the dose administered to the human based upon the difference between the mouse, rat, dog, monkey, or non-human primate and the human, such as subject age, weight, height, body surface area, metabolism, size, physiological influences on pharmacokinetics, or the like. In one aspect, the dose is scaled from a rat to a human.

In some aspects, the compounds and compositions described herein, are used for detecting the presence or absence of the compound in a tissue or cell, wherein the presence of the compound in the tissue or cell indicates the presence of a vascular lesion. In some embodiments, the compound binds to or accumulates in a site expressed by the vascular lesion. In some aspects, the detecting of the vascular lesion is performed using fluorescence imaging. In some aspects, the vascular lesion is associated with one or more of a cavernoma (a.k.a., cavernous angiomas, cavernous hemangiomas, or cerebral cavernous malformation (CCM)), an arteriovenous malformation (a.k.a., arteriovenous angiomas, arteriovenous hemangiomas, or cerebral arteriovenous malformation (CAM)), an aneurysm (e.g., including abdominal aortic, thoracic aortic, and cerebral aneurysm), or a spinal dural arteriovenous fistula.

In further aspects, the compounds and compositions described herein, are used for detecting the presence or absence of the compound in a tissue or cell, wherein the presence of the compound in the tissue or cell indicates the presence of a vascular lesion, and wherein the detecting allows for surgically removing the vascular lesion from the human subject. In some aspects, the compound is administered at a dosage sufficient to treat vascular lesion in the human subject. In some aspects, the compound binds to or accumulates in a site expressed by a vascular lesion. In some aspects, the vascular lesion being treated comprises one or more of one or more of a cavernoma (a.k.a., cavernous angiomas, cavernous hemangiomas, or cerebral cavernous malformation (CCM)), an arteriovenous malformation (a.k.a., arteriovenous angiomas, arteriovenous hemangiomas, or cerebral arteriovenous malformation (CAM)), an aneurysm (e.g., including abdominal aortic, thoracic aortic, and cerebral aneurysm), or a spinal dural arteriovenous fistula. Furthermore, the compounds and compositions described herein can be administered to a subject before surgery and/or during surgery, in which the excised tissue from the subject is contacted with compositions of the peptide complexes. In some aspects, the compositions of the peptide complexes are administered during surgery. In certain aspects, compositions of peptide complexes are intravenously administered to a subject about 0.25 hours, about 0.5 hours, about 0.75 hours, about 1 hour, about 1.5 hours, about 2 hours, about 2.5 hours, about 3 hours, about 3.5 hours, about 4 hours, about 4.5 hours, about 5 hours, about 5.5 hours, about 6 hours, about 6.5 hours, about 7 hours, about 7.5 hours, about 8 hours, about 8.5 hours, about 9 hours, about 9.5 hours, about 10 hours, about 10.5 hours, about 11 hours, about 11.5 hours, about 12 hours, about 13 hours, about 14 hours, about 15 hours, about 16 hours, about 17 hours, about 18 hours, about 19 hours, about 20 hours, about 21 hours, about 22 hours, about 23 hours, about 24 hours, about 36 hours, about 48 hours, about 60 hours, or about 72 hours prior to performing surgery on a human subject. In some aspects, compositions of peptide complexes are intravenously administered to a subject between 0 and 1 hours, between 1 and 2 hours, between 2 and 3 hours, between 3 and 4 hours, between 4 and 5 hours, between 5 and 6 hours, between 6 and 9 hours, between 9 and 12 hours, between 12 and 24 hours, between 24 and 36 hours, between 36 and 48 hours or between 48 and 72 hours (inclusive) before surgery.

Tissue or fluid samples, such as blood, normal tissue, and vascular lesion tissue, can often be isolated from a subject prior to administration of a peptide conjugate, sometimes as a baseline reference. Samples can also be isolated from a subject after administration of the compounds of the present disclosure, often less than about 1 minute after, less than about 2 minutes after, less than about 3 minutes after, less than about 4 minutes after, less than about 5 minutes after, less than about 6 minutes after, less than about 7 minutes after, less than about 8 minutes after, less than about 9 minutes after, less than about 10 minutes after, less than about 11 minutes after, less than about 12 minutes after, less than about 13 minutes after, less than about 14 minutes after, less than about 15 minutes after, less than about 20 minutes after, less than about 30 minutes after, less than about 40 minutes after, less than about 50 minutes after, less than about 60 minutes after, less than about 1 hour after, less than about 2 hours after, less than about 3 hours after, less than about 4 hours after, less than about 5 hours after, less than about 6 hours after, less than about 12 hours after, less than about 18 hours after, less than about 24 hours after, less than about 36 hours after, less than about 48 hours after, less than about 72 hours after, less than about 96 hours after, less than about 5 days after, less than about 7 days after, less than about 10 days after, less than about 14 days after, less than about 21 days after, less than about 4 weeks after, less than about 6 weeks after, less than about 8 weeks after, less than about 12 weeks after, less than about 16 weeks after, less than about 20 weeks after or more than 20 weeks after.

### Imaging and Surgical Methods

The present invention can provide methods for detection, intraoperative imaging, and resection of some types of vascular lesion with a peptide conjugate. The peptide can be a targeting agent that directs the conjugate to the type of vascular lesion tissue or cell. In one embodiment, the peptide of the invention includes one or more labeling agents. In a further embodiment, the labeling agent comprises a fluorescent moiety (e.g., ultraviolet, red or near infrared emitting fluorescent moieties) covalently coupled to the peptide. In another embodiment, the labeling agent comprises a radionuclide. Imaging methods for detection of vascular lesion foci disclosed herein can be applicable to dog and other animal models of vascular lesions as well as to veterinary practice, in addition to human applications.

As used herein, the term “red or near infrared emitting fluorescent moiety” refers to a fluorescent moiety having a fluorescence emission maximum greater than about 600 nm.

In certain embodiments of the peptide conjugate, the fluorescent moieties are derived from fluorescent compounds characterized by emission wavelength maxima greater than about 600 nm to avoid autofluorescence, emission that travels through millimeters to one centimeter of tissue/blood/fluids, emission that is not absorbed by hemoglobin, other blood components, or proteins in human or animal tissue. In some aspects, the emission wavelength maximum is greater than 600 nm, greater than 650 nm, greater than 700 nm, greater than 750 nm, greater than 800 nm, greater than 850 nm, greater than 900 nm, or greater than 950 nm.

The fluorescent moiety can be covalently coupled to the peptide to allow for the visualization of the conjugate by fluorescence imaging. The fluorescent moiety can be derived from a fluorescent compound. Suitable fluorescent compounds can be those that can be covalently coupled to a peptide without substantially adversely affecting the targeting and binding function of the peptide conjugate. Similarly, suitable fluorescent compounds can retain their fluorescent properties after conjugation to the peptide.

The peptide complexes described herein can be used for detection and treatment of certain types of vascular lesions, for example imaging, resection of, diagnosis of and treatment of certain types of vascular lesions. In some aspects, vascular lesions amenable to detection with a peptide conjugate of the present disclosure are one or more of a cavernoma (also referred to as cavernous angiomas, cavernous hemangiomas, or cerebral cavernous malformation (CCM)), an arteriovenous malformation (also referred to as arteriovenous angiomas, arteriovenous hemangiomas, or cerebral arteriovenous malformation (CAM)), an aneurysm (e.g., including abdominal aortic, thoracic aortic, and cerebral aneurysm), or a spinal dural arteriovenous fistula.

Intraoperative resection of vascular lesion types can vary depending on the type of vascular lesion. Intraoperative visualization of vascular lesions in real-time can enable more complete resection while sparing surrounding normal tissue. Improvement in intraoperative vascular lesion visualization can be of benefit for any resectable vascular lesion, as it can enable surgeons to better determine the extent of involvement of nearby tissues such as vasculature, nerve tissue, lymph nodes, and organ tissue. Surgeons who specialize in human brain vascular lesion surgery have indicated that the surgical approach seeks to minimize the excision, and the compounds and methods herein can be used to more accurately visualize the extent of the lesion, enabling the surgeon to fully excise the lesion while minimizing damage to the surrounding normal brain and normal vasculature. This reduces post-operative neurologic symptoms and decreases the likelihood that the lesion will re-grow, hemorrhage, and require additional surgery. Moreover, the compounds and methods herein can be used by surgeons to improve accuracy and precision of operations in sensitive tissues (e.g., brain and organs) by reducing margins in surgery and thus preserving normal tissue. For example, the compounds and methods herein can be used to take margins below 0.2-1 cm margins on all sides of the lesion. It is difficult for surgeons to obtain narrow margins using only white light and preoperative imaging information. In vascular lesion surgeries, failure to obtain clean margins can lead to second surgeries.

The peptide complexes described herein can be used for detection and imaging of vascular lesions in organs or anatomical locations, organs and organ substructures, including the brain and other organs and organ structures such as brain, heart, lung, kidney, liver, CNS (e.g., spine) or pancreas or in the extremities (e.g., legs, neck, and arms). The vascular lesions, can be detected by the peptide complexes described herein. In some aspects, vascular lesion detection includes imaging, resection, diagnostics, and treatment.

In certain aspects, the present compounds are capable of passing across the blood brain barrier. Passing across the blood brain barrier is advantageous when detecting or treating a vascular lesions cell in the brain. For example, the brain is a common location for a cavernoma (e.g., cerebral cavernous malformation (CCM)), an arteriovenous malformation (e.g., a cerebral arteriovenous malformation (CAM)), an aneurysm (e.g., a cerebral aneurysm).

In certain other aspects, the peptide conjugate can be used alone or in combination with other detection agents, to detect, image, visualize, or analyze the vascular lesion in advance of, during, or following treatments, which can include surgery and surgical resection, chemotherapy, phototherapy, heat therapy, and radiation therapy depending on the detectable or therapeutic moiety used. In addition, the peptide conjugate can be used alone or with other detection agents for follow-up monitoring post treatment as well as for general monitoring for full-body screening.

The compounds and methods of the present disclosure can be used alone or in combination with a companion diagnostic, therapeutic or imaging agent (whether such diagnostic, therapeutic or imaging agent is a fluorophore alone, or conjugated, fused, linked, or otherwise attached to a chemical agent or other moiety, small molecule, therapeutic, drug, protein, peptide, antibody protein or fragment of the foregoing, and in any combination of the foregoing; or used as a separate companion diagnostic, therapeutic or imaging agent in conjunction with the fluorophore or other detectable moiety is alone, conjugated, fused, linked, or otherwise attached to a chemical agent or other moiety, small molecule, therapeutic, drug, peptide, antibody protein or fragment of the foregoing, and in any combination of the foregoing). Such companion diagnostics can utilize agents including chemical agents, radiolabel agents, radiosensitizing agents, fluorophores, imaging agents, diagnostic agents, protein, peptide, or small molecule such agent intended for or having diagnostic or imaging effect. Agents used for companion diagnostic agents and companion imaging agents, and therapeutic agents, can include the diagnostic, therapeutic and imaging agents described herein or other known agents. Diagnostic tests can be used to enhance the use of therapeutic products, such as those disclosed herein or other known agents. The development of therapeutic products with a corresponding diagnostic test, such as a test that uses diagnostic imaging (whether in vivo, ex vivo or in vitro) can aid in diagnosis, treatment, identify patient populations for treatment, and enhance therapeutic effect of the corresponding therapy. The compounds and methods of the present disclosure can also be used to detect therapeutic products, such as those disclosed herein or other known agents, to aid in the application of a therapy and to measure it to assess the agent's safety and physiologic effect, e.g. to measure bioavailability, uptake, distribution and clearance, metabolism, pharmacokinetics, localization, blood concentration, tissue concentration, ratio, measurement of concentrations in blood and/or tissues, assessing therapeutic window, extending visibility window, range and optimization, and the like of the therapeutic agent. Such The compounds and methods can be employed in the context of therapeutic, imaging and diagnostic applications of such agents. Tests also aid therapeutic product development to obtain the data FDA uses to make regulatory determinations. For example, such a test can identify appropriate subpopulations for treatment or identify populations who should not receive a particular treatment because of an increased risk of a serious side effect, making it possible to individualize, or personalize, medical therapy by identifying patients who are most likely to respond, or who are at varying degrees of risk for a particular side effect. Thus, the present disclosure, in some embodiments, includes the joint development of therapeutic products and diagnostic devices, including the compounds and methods herein (used to detect the therapeutic and/or imaging agents themselves, or used to detect the companion diagnostic or imaging agent, whether such diagnostic or imaging agent is linked to the therapeutic and/or imaging agents or used as a separate companion diagnostic or imaging agent linked to the peptide for use in conjunction with the therapeutic and/or imaging agents) that are used in conjunction with safe and effective use of the therapeutic and/or imaging agents as therapeutic or imaging products. Non-limiting examples of companion devices include a surgical instrument, such as an operating microscope, confocal microscope, fluorescence scope, exoscope, endoscope, or a surgical robot and devices used in biological diagnosis or imaging or that incorporate radiology, including the imaging technologies of X-ray radiography, magnetic resonance imaging (MRI), medical ultrasonography or ultrasound, endoscopy, elastography, tactile imaging, thermography, medical photography and nuclear medicine functional imaging techniques as positron emission tomography (PET) and single-photon emission computed tomography (SPECT). Companion diagnostics and devices can comprise tests that are conducted ex vivo, including detection of signal from tissues or cells that are removed following administration of the companion diagnostic to the subject, or application of the companion diagnostic or companion imaging agent directly to tissues or cells following their removal from the subject and then detecting signal.

In some embodiments, various fluorescence imaging systems can be used to image excised specimens ex vivo or can be used to image specimens in vivo, and to perform intraoperative imaging. Any system capable of scanning for fluorescence in the infrared and near infrared range can be used, such as the SIRIS or Spectrum instruments or other imaging microscopes. Other systems including devices that interface with, integrate with, or add on to surgical microscopes and other instruments are used in conjunction with, for example, a surgical microscope, including neurosurgical microscopes. Such add-ons can be used in conjunction with an existing surgical microscope, confocal microscope, fluorescence scope, exoscope, endoscope, or surgical robot. In some embodiments, the microscope is stereoscopic. Such exemplary microscope, exoscope, endoscope can include one or more of the following: KINEVO system (e.g., KINEVO 900), QEVO system, CONVIVO system, OMPI PENTERO system (e.g., PENTERO 900, PENTERO 800), INFRARED 800 system, FLOW 800 system, YELLOW 560 system, BLUE 400 system, OMPI LUMERIA systems OMPI Vario system (e.g., OMPI Vario and OMPI VARIO 700), OMPI Pico system, OPMI Sensera, OPMI Movena, OPMI 1 FC, EXTARO 300, TREMON 3DHD system, CIRRUS system (e.g., CIRRUS 6000 and CIRRUS HD-OCT), CLARUS system (e.g., CLARUS 500 and CLARUS 700), PRIMUS 200, PLEX Elite 9000, AngioPlex, VISUCAM 524, VISUSCOUT 100, ARTEVO 800, (and any other surgical microscope, confocal microscope, fluorescence scope, exoscope, endoscope, ophthalmoscope, retinal camera system, optical coherence tomography (OCT) system, and surgical robot systems from Carl Zeiss A/G,); PROVido system, ARvido system, GLOW 800 system, Leica M530 system (e.g., Leica M530 OHX, Leica M530 OH6), Leica M720 system (e.g., Leica M720 OHX5), Leica M525 System (e.g., Leica M525 F50, Leica M525 F40, Leica M525 F20, Leica M525 OH4), Leica M844 system, Leica HD C100 system, Leica FL system (e.g., Leica FL560, Leica FL400, Leica FL800), Leica DI C500, Leica ULT500, Leica Rotatable Beam Splitter, Leica M651 MSD, LIGHTENING, Leica TCS and SP8 systems (e.g., Leica TCS SP8, SP8 FALCON, SP8 DIVE, Leica TCS SP8 STED, Leica TCS SP8 DLS, Leica TCS SP8 X, Leica TCS SP8 CARS, Leica TCS SPE), Leica HyD, Leica HCS A, Leica DCM8, Leica EnFocus, Leica Proveo 8, Leica Envisu C2300, Leica PROvido, and any other surgical microscope, confocal microscope, fluorescence scope, exoscope, endoscope, ophthalmoscope, retinal camera system, OCT system, and surgical robot systems from Leica Microsystems or Leica Biosystems; Haag-Streit 5-1000 system, Haag-Streit 3-1000 system, Haag-Streit HI-R NEO 900, Haag-Streit Allegra 900, Haag-Streit Allegra 90, Haag-Streit EIBOS 2, and any other surgical microscope, confocal microscope, fluorescence scope, exoscope, endoscope, and, surgical robot systems from Haag-Strait; Intuitive Surgical da Vinci surgical robot systems, and any other surgical microscope, confocal microscope, fluorescence scope, exoscope, endoscope, ophthalmoscope, retinal camera system, OCT system, and surgical robot systems from Intuitive Surgical; Heidelberg Engineering Spectralis OCT system, and any other surgical microscope, confocal microscope, fluorescence scope, exoscope, endoscope, ophthalmoscope, retinal camera system, OCT system, and surgical robot systems from Heidelberg Engineering; Topcon 3D OCT 2000, DRI OCT Triton, TRC system (e.g., TRC 50DX, TRC-NW8, TRC-NW8F, TRC-NW8F Plus, TRC-NW400), IMAGEnet Stingray system (e.g., Stingray, Stingray Pike, Stingray Nikon), IMAGEnet Pike system (e.g., Pike, Pike Nikon), and any other surgical microscope, confocal microscope, fluorescence scope, exoscope, endoscope, ophthalmoscope, retinal camera system, OCT system, and surgical robot systems from Topcon; Canon CX-1, CR-2 AF, CR-2 PLUS AF, and any other surgical microscope, confocal microscope, fluorescence scope, exoscope, endoscope, ophthalmoscope, retinal camera system, OCT system, and surgical robot systems from Canon; Welch Allyn 3.5 V system (e.g., 3.5V, 3.5V Autostep), CenterVue DRS, Insight, PanOptic, RetinaVue system (e.g., RetinaVue 100, RetinaVue 700), Elite, Binocular Indirect, PocketScope, Prestige coaxial-plus, and any other surgical microscope, confocal microscope, fluorescence scope, exoscope, endoscope, ophthalmoscope, retinal camera system, OCT system, and surgical robot systems from Welch Allyn; Metronic INVOS system, and any other surgical microscope, confocal microscope, fluorescence scope, exoscope, endoscope, ophthalmoscope, retinal camera system, OCT system, and surgical robot systems from Medtronic; Karl Storz ENDOCAMELEON, IMAGE1 system (e.g., IMAGE1 S, IMAGE1 S 3D, with or without the OPAL1 NIR imaging module), SILVER SCOPE series instrument (e.g., gastroscope, duodenoscope, colonoscope) and any other surgical microscope, confocal microscope, fluorescence scope, exoscope, endoscope, ophthalmoscope, retinal camera system, OCT system, and surgical robot systems from Karl Storz. Moreover, in some embodiments, the imaging, diagnostic, detecting and therapeutic methods herein are performed using the systems described herein alongside, in addition to, combined with, attached to, or integrated into such an existing surgical microscope, confocal microscope, fluorescence scope, exoscope, endoscope, surgical robot, microscope, exoscope, or endoscope as described above.

Any additional surgical microscope, confocal microscope, fluorescence scope, exoscope, endoscope, or surgical robot systems can be used. The surgical microscope, confocal microscope, fluorescence scope, exoscope, endoscope, or surgical robot systems can be provided by, for example, Carl Zeiss A/G, Leica Microsystems, Leica Biosystems, Haag-Streit (5-1000 or 3-1000 systems), or Intuitive Surgical (e.g.: da Vinci surgical robot system), or any other manufacturer of such systems.

### Methods of Treatment

The present disclosure can provide methods for treating some types of vascular lesion by administering a peptide or peptide conjugate described herein. In one embodiment, the method includes administering an effective amount of a peptide or peptide conjugate of the invention to a subject in need thereof. Subjects can include, but are not limited to humans, non-human primates, monkeys, cows, dogs, cats, rabbits, pigs, sheep, horses, guinea pigs, rats, and mice. The methods of treatment of the invention can be applicable to human and animal subjects in need of such treatment.

The term “effective amount,” as used herein, refers to a sufficient amount of an agent or a compound being administered which will relieve to some extent one or more of the symptoms of vascular lesion. The result can be reduction and/or alleviation of the signs, symptoms, or causes of vascular lesion, the ablation, shrinkage, minimization, reduction, inhibition or killing of vascular lesion cells and tissues, or any other desired alteration of a biological system. Compositions containing such agents or compounds can be administered for prophylactic, enhancing, and/or therapeutic treatments. An appropriate “effective” amount in any individual case may be determined using techniques, such as a dose escalation study.

The peptide complexes described herein can be used for treatment of vascular lesions. In some aspects, certain vascular lesions amenable to treatment with a peptide conjugate of the present disclosure include, but are not limited to one or more of a cavernoma (also referred to as cavernous angiomas, cavernous hemangiomas, or cerebral cavernous malformation (CCM)), an arteriovenous malformation (also referred to as arteriovenous angiomas, arteriovenous hemangiomas, or cerebral arteriovenous malformation (CAM)), an aneurysm (e.g., including abdominal aortic, thoracic aortic, and cerebral aneurysm), or a spinal dural arteriovenous fistula.

In certain aspects, the peptide conjugate is administered to an individual having or suspected of having a vascular lesion, such that the conjugate binds specifically to or accumulates in the vascular lesion. Such methods can be useful in reducing the likelihood that the individual will develop a vascular lesion, that one or more vascular lesions in the individual will hemorrhage, or increase in size, and/or that the vascular lesions will progress by some other measure.

The peptide complexes described herein can be used for treatment of vascular lesions in organs and organ substructures, including the brain and other organs and organ structures such as brain, heart, lung, kidney, liver, CNS (e.g., spine) or pancreas or in the extremities (e.g., legs, neck, and arms). In certain aspects, the present compounds are capable of passing across the blood brain barrier. Passing across the blood brain barrier is advantageous when treating a vascular lesion in the brain. In further aspects, vascular lesions of any grade or stage known to one of skill in the art, including low-grade vascular lesions, can be treated by the peptide or peptide complexes described herein. In some aspects, vascular lesion treatment includes the peptide conjugated to a therapeutic agent.

The peptides disclosed herein can be a targeting agent that directs the conjugate to a type of vascular lesion tissue. In one embodiment, the peptide conjugate of the invention includes one or more therapeutic agents. In a further embodiment, a therapeutic agent is covalently coupled to the peptide. The therapeutic agent can be coupled to the peptide to allow for peptide directed delivery of the therapeutic agent to the vascular lesion. Suitable therapeutic agents can be those that can be covalently coupled to a peptide without substantially adversely affecting the targeting and binding function of the peptide conjugate. Similarly, suitable therapeutic agents can retain their therapeutic properties after conjugation to the peptide.

For use with the peptides and peptide complexes herein include one or more therapeutic agent comprising a radioisotope, nanoparticle, toxin, enzyme, sensitizing drug, radiosensitizer, photosensitizer, nucleic acid, interfering RNA, antibody, antibody fragment, aptamer, anti-angiogenic agent, anti-metabolite, mitotic inhibitor, growth factor inhibitor, or combination thereof.

Treatment of the types of vascular lesion with a peptide conjugate as described herein can be combined with other treatments and therapies. Other treatments and therapies can consist of, but are not limited to, radiation therapy, surgery, or any other treatment part of the standard of care for a vascular lesion patient.

Generally, the dosage of administered peptide complexes can vary depending upon such factors as the patient's age, weight, height, sex, general medical condition and previous medical history. Typically, it can be desirable to provide the recipient with a dosage of peptide conjugated to an anti-vascular lesions agent, or an agent or drug that is effective to achieve the ablation, shrinkage, minimization, reduction, inhibition or killing of vascular lesion cells, tissues, or vascular lesions, or prevention of and ablation, shrinkage, minimization, reduction, inhibition or killing of vascular lesion cells, tissues or vascular lesions associated with disease. In many cases, it is desirable to provide the recipient with a dosage of a peptide conjugate that is in the range of from about 0.1 mg to about 100 mg, although a lower or higher dosage also may be administered as circumstances dictate.

Administration of a peptide conjugate to a subject can be topical, inhalant, intravenous, intraarterial, intraperitoneal, intramuscular, subcutaneous, intrapleural, intrathecal, by perfusion through a regional catheter, or by direct intralesional injection. When administering complexes by injection, the administration may be by continuous infusion or by single or multiple boluses.

Additional routes of administration can include oral, mucosal-membrane, pulmonary, and transcutaneous. Oral delivery can be suitable for polyester microspheres, zein microspheres, proteinoid microspheres, polycyanoacrylate microspheres, and lipid-based systems (see, for example, DiBase and Morrel, “Oral Delivery of Microencapsulated Proteins,” in Protein Delivery: Physical Systems, Sanders and Hendren (eds.), pages 255-288 (Plenum Press 1997)). The feasibility of an intranasal delivery can be exemplified by such a mode of insulin administration (see, for example, Hinchcliffe and Ilium, Adv. Drug Deliv. Rev. 35:199 (1999)). Dry or liquid particles comprising a peptide conjugate can be prepared and inhaled with the aid of dry-powder dispersers, liquid aerosol generators, or nebulizers (e.g., Pettit and Gombotz, TIBTECH 16:343 (1998); Patton et al., Adv. Drug Deliv. Rev. 35:235 (1999)). This approach can be illustrated by the AERX diabetes management system, which is a hand-held electronic inhaler that delivers aerosolized insulin into the lungs. Transdermal delivery using electroporation can provide another means to administer a peptide conjugate.

The present invention is not to be limited in scope by the specific embodiments described herein. Indeed, various modifications of the invention in addition to those described herein will become apparent to those skilled in the art from the foregoing description and accompanying figures. Such modifications are intended to fall within the scope of the appended claims.

The particulars shown herein are by way of example and for purposes of illustrative discussion of the preferred embodiments of the present invention only and are presented in the cause of providing what is believed to be the most useful and readily understood description of the principles and conceptual aspects of various embodiments of the invention. In this regard, no attempt is made to show structural details of the invention in more detail than is necessary for the fundamental understanding of the invention, the description taken with the drawings and/or examples making apparent to those skilled in the art how the several forms of the invention may be embodied in practice.

Where a range of values is provided, it is understood that each intervening value, to the tenth of the unit of the lower limit unless the context clearly dictates otherwise, between the upper and lower limit of that range, and any other stated or intervening value in that stated range, is encompassed within the disclosure provided herein. The upper and lower limits of these smaller ranges may independently be included in the smaller ranges, and are also encompassed within the invention, subject to any specifically excluded limit in the stated range. Where the stated range includes one or both of the limits, ranges excluding either or both of those included limits are also included in the disclosure provided herein.

All features discussed in connection with an aspect or embodiment herein can be readily adapted for use in other aspects and embodiments herein. The use of different terms or reference numerals for similar features in different embodiments does not necessarily imply differences other than those expressly set forth. Accordingly, the present invention is intended to be described solely by reference to the appended claims, and not limited to the embodiments disclosed herein.

### Indications and Methods

The compounds and methods herein can be used to identify presence of health or disease, in diagnosis, imaging, health monitoring and the like in a given sample (e.g., organ, organ substructure, tissue, or sample, whether ex-vivo or in situ). Exemplary organs and organ substructures include the brain and other organs and organ structures such as brain, heart, lung, kidney, liver, CNS (e.g., spine) or pancreas or in the extremities (e.g., legs, neck, and arms).

For example, fluorescence angiography is useful during certain neurosurgical procedures in the brain and spinal cord. Repair of blood vessel defects such as aneurysm, arteriovenous malformation, cavernous malformation, spinal dural arteriovenous fistula, venous malformation, lymphatic malformation, capillary telangiectasia, mixed vascular malformation, and the like, requires imaging of the defect architecture, confirmation that the defect is successfully isolated prior to repair, and confirmation that repaired vessels have restored proper blood flow and patency. Vessel patency is particularly crucial in the CNS to avoid neurologic damage or death that can result from undetected bleeding into these tissues.

Neurosurgical microscopes, neuroendoscopes, endovascular endoscopes, and robotic surgical systems including the compounds and methods described herein may all be used in this setting. Removal of CNS tumors such as pituitary adenoma is another setting in which fluorescence angiography can be applied to improve safety and efficacy of treatment. The visualization of vascular flow to the tumor and verification that the tumor has been removed without residual bleeding are both important uses for this technology.

Fluorescence angiography, cholangiography, lymphography, and the like are useful in support of a variety of surgical interventions. The compounds and methods described herein can be used in various cardiovascular and vascular surgeries, including aneurysm repair, valve replacement, arteriovenous malformation, cavernous malformation, repair or bypass, arterial bypass, and the like for visualization of blood flow and vessel patency. The compounds and methods described herein can be used in plastic surgery, trauma surgery, reconstructive surgery, and the like for vascular mapping and for assessment of tissue perfusion. Tissue perfusion is of particular importance in flap reconstructions and in anastomoses of the gastrointestinal tract, for example following colorectal cancer surgery or esophagectomy, as tissue ischemia following such surgeries can result in loss of tissue and graft failure or leaking anastomosis. Fluorescence lymphography using compounds and methods described herein is useful for demonstrating flow of the lymphatic vessels, for example to support re-routing of lymphatic drainage to treat lymphedema.

The compounds and methods described herein are useful for visualization of organs or organ segments in a variety of surgical procedures. Liver segments can be imaged following intra-arterial dye injection during partial hepatectomy. Perfusion and bile production can be assessed following partial or total liver transplantation. Other hepatobiliary surgeries, including resection of liver vascular lesions, are also supported by angiography or cholangiography. Contrast between the kidney and adrenal gland can be achieved using fluorescent dyes or complexes that are cleared through renal filtration. This procedure can help in differentiating the adrenal gland from the kidney, for example to avoid kidney damage during removal of the adrenal glands. The ureters can also be identified using these methods to avoid damage to them during uro-abdominal surgeries. Abnormally vascularized tissues, such as endometriosis or tumors, can be identified and removed using these methods.

Coupled with a targeting moiety that binds specifically to or accumulates in nerves, fluorescence imaging compounds and methods described herein can be used to visualize nerves during surgery to avoid damage. This is important during surgeries in highly innervated areas, particularly where damage to the nerves can result in significant morbidity. Examples include facial nerves, visceral nerves, and cavernous nerves.

Disruptions of the ophthalmic vasculature occur as a result of diseases such as diabetes, glaucoma, or Susac's syndrome, secondary to trauma, or spontaneously. The compounds and methods described herein are useful in diagnosis and in treatment targeting and/or monitoring of such disruptions. These can include macular edema, macular ischemia, age-related macular degeneration, retinal tear, retinal degeneration, retinal artery occlusion, retinal vein occlusion, and the like.

The compounds and methods described herein are useful for fluorescence imaging and identification of appropriate arteries prior to administration of a drug therapy. Exemplary organs and organ substructures include the brain and other organs and organ structures such as brain, heart, lung, kidney, liver, CNS (e.g., spine) or pancreas or in the extremities (e.g., legs, neck, and arms).

Such systems can be useful for endovascular imaging for diagnosis and treatment monitoring in cardiovascular diseases such as atherosclerosis. Examination of features such as lumen dimensions, plaque burden, remodeling, lipid components, cap thickness, neo-angiogenesis, and inflammation are used to diagnose plaque instability; fluorescence imaging in combination with other technologies can improve these assessments. Following stent placement, fluorescence angiography can be used to detect vessel restenosis.

The compounds and methods described herein are useful in non-invasive diagnosis and monitoring of tissue perfusion, for example in chronic wounds or limb/extremity ischemia.

The compounds and methods described herein are useful in microvasculature imaging. For example, oxyhemoglobin and deoxyhemoglobin have sequential two-color, two-photon absorption properties that can serve as endogenous contrasts in microvasculature imaging. Using a sensitive modulation transfer technique, the compounds and methods described herein can image hemoglobin in red blood cells with micrometer resolution, with or without labeling using a fluorophore or other detectable compound.

The compounds and methods described herein can use multispectral images to identify subcutaneous vasculature, with improved contrast in the near infrared spectrum, including detection and methods involving infrared and near-infrared imaging of superficial blood vessels.

The compounds and methods described herein can be used in angiography and coronary catheterization. For example, a coronary angiogram is a procedure that uses imaging to see the heart's blood vessels. The test is generally done to visualize any restrictions in blood flow going to the heart. Coronary angiograms are part of a general group of procedures known as heart (cardiac) catheterizations. Cardiac catheterization procedures can both diagnose and treat heart and blood vessel conditions. A coronary angiogram, which can help diagnose heart conditions, is the most common type of cardiac catheterization procedure. Similarly, such compounds and methods described herein can be applied to other vasculature including lymph, cerebral vasculature, organ vasculature, arteries, capillaries, veins, and the like.

The compounds and methods described herein can be used in imaging and detecting cancers, e.g., for detecting and imaging angiogenesis, (i.e., the formation of new blood vessels) associated with tumors.

The compounds and methods described herein can be used to diagnose, image, and detect blood vessel derived tumors and aid in their treatment through surgery and improve the health of patients through monitoring. Vascular tumors may be benign or malignant. Benign tumors form recognizable vascular channels filled with blood or lymphatic fluid. Malignant tumors are usually more solid and cellular without well-formed vascular channels. Similarly, such compounds and methods described herein can be applied to other vasculature including lymph, cerebral vasculature, organ vasculature, arteries, capillaries, veins, and the like. Exemplary vessel derived tumors include those of endothelial cells, including hemangiomas, lymphangiomas, angiosarcomas, or cells supporting or surrounding blood vessels including glomus tumors, or hemangiopericytomas.

The compounds and methods described herein can be used to diagnose, image, monitor and determine the outcome of heart surgery, including heart valve surgery, and treatment through surgery and improve the health of patients through monitoring.

In some applications the compounds and methods disclosed herein can be used to diagnose, image, and monitor intrinsic fluorescence or autofluorescence in tissues with or without the administration of a fluorescent dye or other fluorescent agent as a contrast agent or an imaging agent per se. Intrinsic protein fluorescence, predominantly derived from tryptophan (λEX˜280 nm, λEM˜350 nm), as well as other aromatic amino acids tyrosine and phenylalanine, in proteins can be used with the compounds and methods herein, for example in label-free Forster resonance energy transfer (FRET) techniques. For example, in terms of wavelength and intensity, tryptophan fluorescence is strongly influenced by its (or the protein's) local environment, which, in addition to fluorescence quenching, has been applied to study protein conformational changes. Intrinsic FRET utilizes the intrinsic fluorescence of tryptophan in conjunction with target-specific fluorescent probes as FRET donors and acceptors, respectively, for real time detection of native proteins. For example, fluorescence intensity profiles measured along the optical axis of human eye lenses can correlate with age-related nuclear cataract showing increasing concentration of fluorescent post-translational modification (PTM) towards the lens center in accord with the increased optical density in the lens nucleolus. The imaging compounds and methods herein can provide spatiotemporal information of PTMs with little perturbation to the cellular environment. Significant differences between fluorescence lifetimes of “free” Trp derivatives hydroxytryptophan (OH-Trp), N-formylkynurenine (NFK), kynurenine (Kyn), hydroxykynurenine (OH-Kyn) and their residues can be measured and used to image, monitor, and diagnose disease in the eye. In addition, fundus autofluorescence (FAF) is a non-invasive retinal imaging modality used in clinical practice to provide a density map of lipofuscin, the predominant ocular fluorophore, in the retinal pigment epithelium. The imaging compounds and methods herein can be used to evaluate, image, diagnose, and monitor various retinal diseases, including age related macular degeneration, macular dystrophies, retinitis pigmentosa, white dot syndromes, retinal drug toxicities, and various other retinal disorders. Moreover, autofluorescence depends on endogenous fluorophores in the tissue, which undergo a change associated with malignant transformation. This change (malignancy) can be detected as an alteration in the spectral profile and intensity of autofluorescence. Consequently, autofluorescence of tumors can be detected using the compounds and methods described herein, making the compounds and methods herein useful for imaging, diagnosing, and monitoring a variety of cancers. For example, bladder cancer is an exemplary cancer that autofluoresces. Fluorescence excitation wavelengths varying from 220 to 500 nm were used to induce tissue autofluorescence, and emission spectra can be measured in the 280-700 nm range. These spectra are combined to construct 2-dimensional fluorescence excitation-emission matrices (EEMs). Significant changes in fluorescence intensity of EEMs observed between normal and tumor bladder tissues are indicative of disease, the most marked differences being at the excitation wavelengths of 280 and 330 nm. Addition of contrast, fluorescent imaging agents, or target-specific fluorescent agents, can be used to further exemplify the application of the compounds and methods in the detection, imaging, diagnosis, and monitoring of intrinsic tissue autofluorescence and tissue autofluorescence various applications.

TABLE 5 shows information of exemplary embodiments of indications and applicable organ vasculature for use with the compounds and methods herein.

TABLE 5
Use of Compounds and methods in Vascular Intervention

| Indication | Intervention type | Organ System |
|---|---|---|
| Arteriovenous malformation | Neurosurgery | CNS |
| Cavernous malformation | Neurosurgery | CNS |
| Intracranial aneurysm | Neurosurgery | CNS |
| Pituitary adenoma | Neurosurgery | CNS |
| Spinal dural arteriovenous fistula | Surgery | CNS |
| Adrenal surgery | Surgery | Endocrine |
| Thyroid surgery (parathyroid preservation) | Surgery | Endocrine |
| Critical limb ischemia | Diagnostic | Extremities |
| Diabetic macular edema | Diagnostic | Eye |
| Diabetic macular ischemia | Diagnostic | Eye |
| Diabetic retinopathy | Diagnostic | Eye |
| Macular degeneration | Diagnostic | Eye |
| Retinal artery occlusion | Diagnostic | Eye |
| Retinal vein occlusion | Diagnostic | Eye |
| Susac's syndrome | Diagnostic | Eye |
| Glaucoma | Diagnostic | Eye |
| Retinal surgery | Surgery | Eye |
| Kidney transplant | Surgery | Genitourinary |
| Ureter visualization (any uro-abdominal surgery) | Surgery | Genitourinary |
| Kidney stones | Surgery | Genitourinary |
| Colorectal surgery | Surgery | GI |
| Esophageal anastomosis | Surgery | GI |
| Craniomaxillofacial trauma | Surgery | Head and neck |
| Liver cancer | Surgery | Hepatobiliary |
| Partial hepatectomy | Surgery | Hepatobiliary |
| Partial liver transplantation | Surgery | Hepatobiliary |
| Hepatobiliary surgery | Surgery | Hepatobiliary |
| Chronic wounds | Diagnostic | Soft tissue |
| Plastic surgery | Surgery | Soft tissue |
| Reconstructive surgery | Surgery | Soft tissue |
| Lymphedema | Diagnostic | Vasculature |
| Atherosclerosis | Diagnostic | Vasculature |
| Endometriosis | Surgery | Viscera |

Exemplary organs and organ substructures include the brain and other organs and organ structures such as brain, heart, lung, kidney, liver, CNS (e.g., spine) or pancreas or in the extremities (e.g., legs, neck, and arms).

While preferred embodiments of the present invention have been shown and described herein, it will be apparent to those skilled in the art that such embodiments are provided by way of example only. Numerous variations, changes, and substitutions will now occur to those skilled in the art without departing from the invention. It should be understood that various alternatives to the embodiments of the invention described herein may be employed in practicing the invention. It is intended that the following claims define the scope of the invention and that methods and structures within the scope of these claims and their equivalents be covered thereby.

### EXAMPLES

The invention is further illustrated by the following non-limiting examples.

### Example 1

### Use of Compounds and Methods During Aneurysm, Arteriovenous Malformation, or Cavernous Malformation Resection

This example describes use of the compounds and methods disclosed herein for coaxial illumination and visualization of Compound 76 fluorescence during surgical resection of an arteriovenous malformation or cavernous malformation in a pediatric patient. An imaging system was used to image brain tissue to detect abnormal vasculature using fluorescence imaging. Surgery was performed to remove the abnormality from the subject.

A pediatric subject with a history of anosmia was found on MRI to have a 3.5 cm T1-hypointense, T2/FLAIR-hyperintense mass in the right middle frontal gyrus with a central enhancing nodule, initially diagnosed pre-operatively to be a low-grade glioma. The subject did not have any prior history of neurosurgery. The patient received 22 mg (15 mg/m²) of a fluorescent conjugate comprising SEQ ID NO:9 conjugated to an ICG (Compound 76) via IV injection approximately 5-6 hours before surgery and image collection. The imaging system head was attached to the Zeiss Pentero surgical microscope along with two eyepieces prior the start of surgery.

After the lesion was exposed, the imaging system was initialized and used continuously. The imaging system enabled the surgeon to view fluorescence and visible imaging together and simultaneously with the operating microscope. A microsurgical resection was performed through a right frontal craniotomy. The abnormal tissue had a dark blue mulberry appearance that fluoresced avidly with Compound 76. The surrounding tissue showed no fluorescence. The abnormal tissue was completely resected. The patient recovered without deficit. Pathology confirmed the absence of tumor. Pathology data suggested the abnormality was vascular in nature and demonstrated that Compound 76 fluorescence was detected in a cerebral non-tumoral lesion. While ICG alone transiently lights up blood vessels immediately after injection, toluzeristide fluorescence is dependent on pathological tissue binding and uptake and can highlight diseased tissue up to 30 hours after injection.

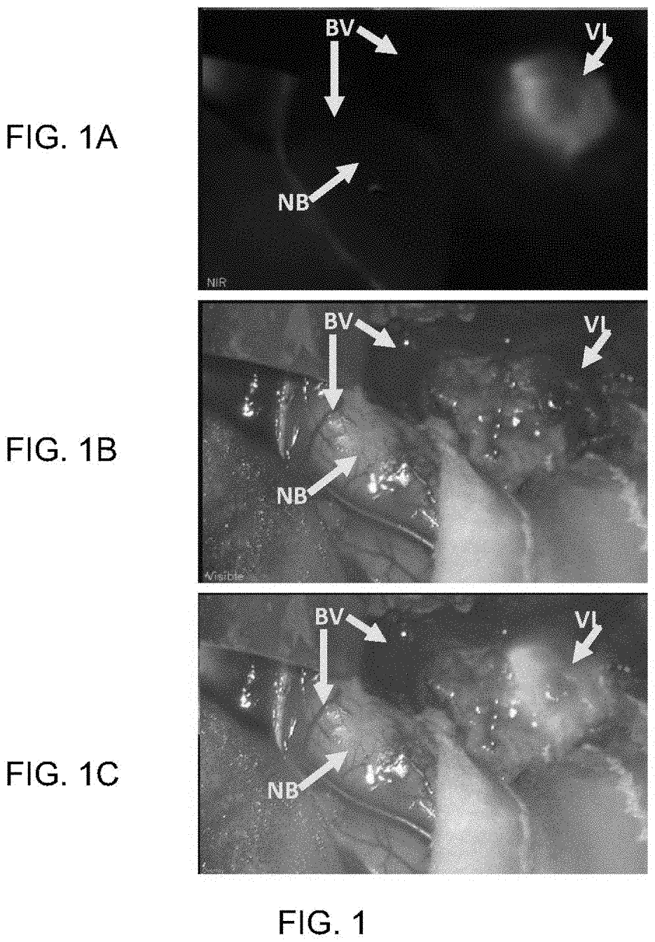

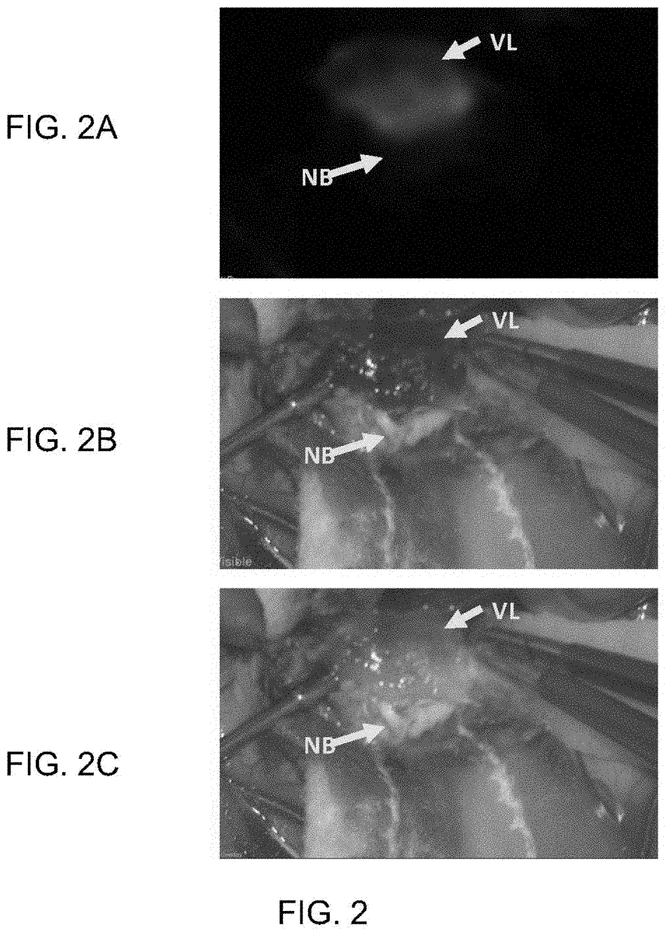

Video was captured for the duration of the resection, and still images were captured of the exposed lesion. Compound 76 fluorescence was observed in situ in the exposed vascular lesion. FIGS. 1 and 2 show images taken of the vascular lesion with the near-infrared (NIR) fluorescence, white light, and composite fluorescence and white light images. The vascular abnormality appeared to the surgeon as a bright blue-green mass (arrows labeled VL) in the NIR fluorescence image and in the overlay image (shown as a bright white mass in grey-scale), while the normal brain tissue (labeled “NB”) and vasculature (labeled “BV”) appeared darker than the vascular lesion in the NIR fluorescence image indicating no discernable background fluorescence in non-lesion or normal brain tissue or in normal vasculature. In the overlay image, the normal brain tissue and normal blood vessels appeared pink or light tan to red, as it does under normal visible light or white light. The surgeon noted that only the abnormal vascular tissue appeared fluorescent.

FIG. 1 and FIG. 2 show representative images of in situ or intra-operative tissue during surgery on a vascular lesion in a patient, wherein 22 mg (15 mg/m²) of Compound 76 was administered to the human subject. FIG. 1A shows a near-infrared (NIR) image of the in situ specimen. Fluorescence signal, corresponding to lighter and brighter areas in the NIR images, is indicative of the presence of Compound 76 in the vascular lesion. Labeled arrows indicate non-fluorescent regions of normal blood vessels (“BV”) and normal brain tissue (“NB”). In contrast, fluorescence signal corresponding to lighter and brighter areas in the NIR image, was indicative of the presence of Compound 76 on the abnormal vascular lesion (“VL”), and not in normal tissue. FIG. 1B shows the white light image corresponding to FIG. 1A that represents what the surgeon would normally see without fluorescence guidance. The arrows mark the same locations as shown in the NIR image in FIG. 1A. The vascular lesion (“VL”) had a similar appearance to the normal blood vessels (“BV”) in this image. FIG. 1C shows the NIR fluorescence and white light composite image of FIG. 1A and FIG. 1B, with arrows marking the same locations as shown in FIG. 1A and FIG. 1B. The fluorescence in the vascular lesion (“VL”) clearly differentiated it from the surrounding normal tissues, including normal blood vessels (“BV”). FIG. 2A shows a near-infrared (NIR) image of the vascular lesion during the surgery. Arrows indicate the vascular lesion (labeled “VL”) and adjacent normal brain (labeled “NB”), which is non-fluorescent. FIG. 2B shows the white light image corresponding to FIG. 2A. While the normal brain has a light tan to pink color (light gray in a gray scale image), it is perfused with normal blood vessels that can be differentiated from the vascular lesion by the absence of fluorescence. FIG. 2C shows the composite white light and NIR image shown in FIG. 2A and FIG. 2B.

The fluorescent tissue samples were demonstrated and confirmed to be non-cancerous and vascular in nature by histopathology. The pathology did not indicate cancer or neoplastic abnormalities but rather confirmed avascular abnormality that did not indicate cancer.

Intraoperative pathology was performed on two specimens. One showed dilated vessels most compatible with vascular malformation, and the other was normal brain parenchyma with no evidence of neoplasm. Post-operative pathology was performed on 19 excised specimens with the numbered annotations for reference are shown below in TABLE 6.

TABLE 6
Pathology Specimens

| Specimen | | |
|---|---|---|
| Number | Description | Observation |
| 1 | Right brain specimen 1 (Excision) | Blood vessels with hyalinization and |
| | | chronic inflammation |
| 2 | Right brain specimen 2 (Excision) | Gray and white matter and blood vessels |
| | | with chronic inflammation, and |
| | | microcalcifications |
| 3 | Right brain specimen 3 (Excision) | Blood vessels, interspersed neuropil, |
| | | chronic inflammation, and calcifications |
| 4 | Right brain specimen 4 (Excision) | Blood vessels with chronic inflammation |
| | | and interspersed gray matter |
| 5 | Right brain specimen 5 (Excision) | Blood vessels with interspersed gray and |
| | | white matter and proteinaceous aggregates |
| 6 | Right brain specimen 6 (Exision) | Minute cluster of blood vessels |
| 7 | Deep lateral equivocal tissue | Minute fragment of white matter |
| | specimen 7 (Excision) | |
| 8 | Posterior equivocal tissue | Gray matter with focus |
| | specimen 8 (Excision) | of blood vessels |
| 9 | Inferior equivocal tissue | Minute fragment of |
| | specimen 9 (Excision) | gray matter |
| 10 | Anterior equivocal tissue | Minute fragment of gray |
| | specimen 10 (Excision) | and white matter |
| 11 | Anterior lateral equivocal tissue | Minute fragments of gray and white matter |
| | specimen 11 (Excision) | with focus of blood vessels |
| 12 | Anterior equivocal tissue | Minute fragment of |
| | specimen 12 (Excision) | gray matter |
| 13 | Posterior equivocal tissue | Minute fragments of gray |
| | specimen 13 (Excision) | and white matter |
| 14 | Deep lateral equivocal tissue | Minute fragment of |
| | specimen 14 (Excision) | white matter |
| 15 | Anterior equivocal tissue | Minute fragments of gray |
| | specimen 15 (Excision) | and white matter |
| 16 | Inferior equivocal tissue | Minute fragments of gray |
| | specimen 16 (Excision) | matter with reactive changes |
| 17 | Anterior lateral equivocal tissue | Small fragments of gray and white matter |
| | specimen 17 (Excision) | |
| 18 | Right deep specimen 18 | Small fragments of gray matter |
| | (Excision) | with reactive surgical changes |
| 19 | Right deep specimen 19 | Small fragment of gray |
| | (Excision) | and white matter |

Specimens with substantial vascular components were considered for examination. In specimen 8 the vessels were not separated by neuropil. In specimens 3, 4, and 5, neuropil intervened between the vessels, indicating an overall diagnosis of vascular malformation.

This case demonstrated that the compounds and methods could be used in an intraoperative setting and enabled the surgeon to visualize and precisely localize fluorescence in non-neoplastic pathologies during surgery and use this information to remove abnormal vascular tissue during resection.

The imaging agent is conjugated to a SEQ ID NO: 9 peptide at K27, or any one of SEQ ID NO: 1-SEQ ID NO: 481 peptide, SEQ ID NO: 482-SEQ ID NO: 485 peptide, or a fragment of the foregoing. The compounds and methods could be used in an intraoperative setting and enabled the surgeon to visualize one or more of a cavernoma (a.k.a., cavernous angiomas, cavernous hemangiomas, or cerebral cavernous malformation (CCM)), an arteriovenous malformation (a.k.a., arteriovenous angiomas, arteriovenous hemangiomas, or cerebral arteriovenous malformation (CAM)), an aneurysm (e.g., including abdominal aortic, thoracic aortic, and cerebral aneurysm), venous malformation, lymphatic malformation, capillary telangiectasia, mixed vascular malformation, or a spinal dural arteriovenous fistula.

### Example 2

### Use of Compounds and Methods for Imaging and Monitoring Occlusion of Veins or Arteries Resulting in Organ Failure or Injury

This example describes use of the compounds and methods herein for imaging and diagnosis of occlusion of arteries or veins or detection of hemorrhage or embolism in a variety of organ systems, including brain, heart, lung, kidney, liver, pancreas, or in the extremities (e.g., legs, neck, and arms). Lack of sufficient blood flow (ischemia) affects tissue and may cause organ damage or organ failure, hemorrhagic stroke, and the like. The compounds and methods disclosed herein are used as imaging agents to image blood flow in a subject. A contrast or imaging agent, including an indocyanine green (ICG) or fluorescein alone or in conjunction with a peptide or active agent, is administered to a subject. The subject is a human or an animal and has a chronic wound or suspected ischemia (e.g. in extremities or limb). Administration is intravenous, subcutaneous, intranasal, oral, intraperitoneal, intramuscular, or intradermal. Upon administration, the agent is targeted to vascular tissues and cells thereof, or is selectively retained within the blood. The agent is then visualized using an imaging system, in conjunction with a surgical microscope, other imaging system, or as an open imaging system. Absence, blockage or hemorrhage of fluorescence signal from the tissue of interest indicates reduced or absent blood flow, and ischemia indicative of organ damage or organ failure, hemorrhagic stroke, and the. Other contrast or imaging agents can be used as described herein.

The imaging agent is conjugated to a SEQ ID NO: 9 peptide at K27, or any one of SEQ ID NO: 1-SEQ ID NO: 481 peptide, SEQ ID NO: 482-SEQ ID NO: 485 peptide, or a fragment of the foregoing.

### Example 3

### Use of Compounds and Methods for Angiography in Repair of CNS Vascular Defects

This example describes using the compounds and methods herein for the imaging, detection, monitoring, diagnosis or treatment of CNS vascular defects (e.g., arteriovenous malformation, cavernous malformation, intracranial aneurysm) in a subject, comprising any contrast or imaging agents including an indocyanine green (ICG) or fluorescein alone or in conjunction with a peptide or active agent. The agent is administered to a subject. The subject is a human or an animal and has a vascular defect. Administration is intravenous, subcutaneous, intranasal, oral, intraperitoneal, intramuscular, or intradermal. Upon administration, the agent is targeted to vascular tissues and cells thereof or is selectively retained within the blood. The compounds are then visualized using an imaging system, such as a neurosurgical operating microscope, a neuroendoscope, a vascular endoscope, or as an open imaging system. The selection of the appropriate imaging system is made by the surgeon and is dependent on the size and location of the vascular defect as well as surgical approach.

The imaging agent is conjugated to a SEQ ID NO: 9 peptide at K27, or any one of SEQ ID NO: 1-SEQ ID NO: 481 peptide, SEQ ID NO: 482-SEQ ID NO: 485 peptide, or a fragment of the foregoing.

### Example 4

### Use of Compounds and Methods for Angiography in Blood or Lymph and Arteriography

This example describes use of the compounds and methods disclosed herein for coaxial illumination and visualization of blood or lymph in a subject. The compounds and methods of the present invention is used to image vascular or lymph vessels to image, monitor, diagnose, or guide treatment of disease. Surgery is performed to remove or bypass occlusions, repair vascular defects, provide for lymphatic drainage into the circulatory system to treat lymphedema, or to remove cancer or other abnormal tissue, such as endometriosis, from the subject. A contrast or imaging agent, including an indocyanine green (ICG) or fluorescein alone or in conjunction with a peptide or active agent, is administered to a subject. The subject is a human or an animal and has an occlusion that necessitates removal or bypass, or a tumor or other abnormal tissue that requires removal. Administration is intravenous, subcutaneous, intranasal, oral, intraperitoneal, intramuscular, intradermal, or by intratumoral injection. Upon administration, the agent is targeted to vascular tissues and cells thereof, lymphatic tissues and cells thereof, tumor or other abnormal vasculature, or is selectively retained within the blood or lymph. The compound is then visualized using an imaging system, such as a neurosurgical operating microscope, a neuroendoscope, a vascular endoscope, an endoscope, thoracoscope, telescope, robotic surgical system, other surgical microscope, or as an open imaging system. The selection of the appropriate imaging system is made by the surgeon and is dependent on the surgical approach.

The imaging agent is conjugated to a SEQ ID NO: 9 peptide at K27, or any one of SEQ ID NO: 1-SEQ ID NO: 481 peptide, SEQ ID NO: 482-SEQ ID NO: 485 peptide, or a fragment of the foregoing.

### Example 5

### Use of Compounds and Methods for Angiography in the Eye

This example describes using the compounds and methods herein for the imaging, detection, monitoring, diagnosis or treatment of disease, injury, or malformation of ocular structures (e.g., diabetic macular edema, diabetic macular ischemia, diabetic retinopathy, macular degeneration, retinal artery occlusion, retinal vein occlusion, Susac's syndrome, glaucoma, retinal detachment) in a subject, comprising any contrast or imaging agents including an indocyanine green (ICG) or fluorescein alone or in conjunction with a peptide or active agent. The agent is administered to a subject. The subject is a human or an animal and has a disease, injury, or malformation of ocular structures. Administration is intravenous, subcutaneous, intranasal, oral, intraperitoneal, intramuscular, intra-ocular, topical, or intradermal. Upon administration, the agent is targeted to vascular tissues and cells thereof or is selectively retained within the blood. The agent is then visualized using an imaging system, such as an ophthalmoscope, retinal or fundus camera system, optical coherence tomography (OCT) system, surgical microscope, or other ophthalmic imaging system. Ophthalmic angiogram of the choroid may similarly utilize the compounds and methods disclosed herein. The imaging system enables the operator to view fluorescence and visible imaging together and simultaneously with the operating microscope or other imaging system. There is no need to reposition the operating microscope or other imaging system to view the fluorescence and visible images thus providing color imaging of the ocular structures together with the fluorescence imaging, which decreases disruption to the surgical or diagnostic workflow. Other contrast or imaging agents can be used as described herein.

The imaging agent is conjugated to a SEQ ID NO: 9 peptide at K27, or any one of SEQ ID NO: 1-SEQ ID NO: 481 peptide, SEQ ID NO: 482-SEQ ID NO: 485 peptide, or a fragment of the foregoing.

### Example 6

### Use of Compounds and Methods for Perfusion Imaging in Surgery

This example describes use of the compounds and methods disclosed herein for coaxial illumination and visualization of tissue perfusion in a subject. The compounds and methods disclosed herein are detected by an imaging system is used to image blood flow in tissues during surgeries requiring adequate perfusion to promote healing of joined tissues (e.g., anastomosis, reconstructive surgery, or plastic surgery). A contrast or imaging agent, including an indocyanine green (ICG) or fluorescein alone or in conjunction with a peptide or active agent, is administered to a subject. The subject is a human or an animal and has a condition such as occlusion, cancer, or trauma. Administration is intravenous, subcutaneous, intranasal, oral, intraperitoneal, intramuscular, or intradermal. Upon administration, the agent is targeted to vascular tissues and cells thereof, or is selectively retained within the blood or lymph. The agent is then visualized using an imaging system, such as a neurosurgical operating microscope, a neuroendoscope, a vascular endoscope, an endoscope, thoracoscope, telescope, robotic surgical system, other surgical microscope, or as an open imaging system. The selection of the appropriate imaging system is made by the surgeon and is dependent on the surgical approach.

The imaging agent is conjugated to a SEQ ID NO: 9 peptide at K27, or any one of SEQ ID NO: 1-SEQ ID NO: 481 peptide, SEQ ID NO: 482-SEQ ID NO: 485 peptide, or a fragment of the foregoing.

### Example 7

### Use of Compounds and Methods for Detection of Plaque Instability and Restenosis in Atherosclerosis

This example describes use of the compounds and methods disclosed herein for coaxial illumination and visualization of atherosclerotic plaques and restenosis in a subject. The compounds and methods disclosed herein are detected by an imaging system is used to image atherosclerotic plaques within blood vessels in order to assess their stability, and to image blood flow through stented blood vessels for diagnosis of restenosis. A contrast or imaging agent, including an indocyanine green (ICG) or fluorescein alone or in conjunction with a peptide or active agent, is administered to a subject. The subject is a human or an animal and has atherosclerosis. Administration is intravenous, subcutaneous, intranasal, oral, intraperitoneal, intramuscular, or intradermal. Upon administration, the agent is targeted to vascular tissues and cells thereof, or is selectively retained within the blood. The agent is then visualized using an imaging system, such as an endovascular endoscope, a vascular endoscope, an endoscope, thoracoscope, telescope, robotic surgical system, other surgical microscope, or as an open imaging system. The selection of the appropriate imaging system is made by the surgeon and is dependent on the surgical or diagnostic approach.

The imaging agent is conjugated to a SEQ ID NO: 9 peptide at K27, or any one of SEQ ID NO: 1-SEQ ID NO: 481 peptide, SEQ ID NO: 482-SEQ ID NO: 485 peptide, or a fragment of the foregoing.

### Example 8

### Use of Compounds and Methods for Imaging Vital Organs or Structures

This example describes use of the compounds and methods disclosed herein for imaging vital organs or structures in a subject during surgery. The compounds and methods disclosed herein are detected by an imaging system is used to image contrast between vital organs or structures (e.g., kidney, ureters, thyroid, liver or liver segments, nerves) and other surrounding tissues. A contrast or imaging agent, including an indocyanine green (ICG), methylene blue, or fluorescein, alone or in conjunction with a peptide or active agent, is administered to a subject. The subject is a human or an animal and has a disease or condition that requires surgical intervention near vital organs or structures. Administration is intravenous, subcutaneous, intranasal, oral, intraperitoneal, intramuscular, intradermal, or by intratumor injection. Upon administration, the agent is targeted to vascular tissues and cells thereof, to vital organ tissues and cells thereof (e.g., nerves), or is selectively retained within the blood. The agent is then visualized using an imaging system, such as a laparoscope, a vascular endoscope, an endoscope, thoracoscope, telescope, robotic surgical system, other surgical microscope, or as an open imaging system. The selection of the appropriate imaging system is made by the surgeon and is dependent on the surgical or diagnostic approach. Contrast results either from differential blood flow to the organ or tissue (e.g., kidney contrasting with adrenal gland, thyroid contrasting with parathyroid, or liver segment following selective injection to an artery supplying the segment), from elimination pathways (e.g. ureters or kidney following administration of a dye or conjugate with renal clearance), or from selective targeting to the organ or structure (e.g., using a peptide that targets proteins found on nerve sheaths). The imaging system enables the surgeon to view fluorescence and visible imaging together and simultaneously with the operating microscope or other imaging system. The contrast enables the surgeon to avoid injury to normal tissues and to selectively remove organs, organ segments, or other tissues as appropriate. Exemplary organs and organ substructures include the brain and other organs and organ structures such as brain, heart, lung, kidney, liver, CNS (e.g., spine) or pancreas or in the extremities (e.g., legs, neck, and arms).

The imaging agent is conjugated to a SEQ ID NO: 9 peptide at K27, or any one of SEQ ID NO: 1-SEQ ID NO: 481 peptide, SEQ ID NO: 482-SEQ ID NO: 485 peptide, or a fragment of the foregoing.

### Example 9

### Use of Compounds and Methods for Diagnosis of Ischemia

This example describes use of the compounds and methods disclosed herein for imaging and diagnosis of tissue ischemia. The compounds and methods disclosed herein are detected by an imaging system is used to image blood flow in a subject. A contrast or imaging agent, including an indocyanine green (ICG) or fluorescein alone or in conjunction with a peptide or active agent, is administered to a subject. The subject is a human or an animal and has a chronic wound or suspected ischemia (e.g. in extremities or limb). Administration is intravenous, subcutaneous, intranasal, oral, intraperitoneal, intramuscular, or intradermal. Upon administration, the agent is targeted to vascular tissues and cells thereof, or is selectively retained within the blood. The agent is then visualized using an imaging system, such as a surgical microscope, other imaging system, or as an open imaging system. Absence of fluorescence signal from the tissue of interest indicates reduced or absent blood flow, and ischemia. Other contrast or imaging agents can be used as described herein.

The imaging agent is conjugated to a SEQ ID NO: 9 peptide at K27, or any one of SEQ ID NO: 1-SEQ ID NO: 481 peptide, SEQ ID NO: 482-SEQ ID NO: 485 peptide, or a fragment of the foregoing.

### Example 10

### Use of Compounds and Methods During Venography

This example describes use of the compounds and methods disclosed herein for imaging of veins and diagnosis of deep vein thrombosis (DVT) or other vein abnormalities. The compounds and methods disclosed herein are detected by an imaging system is used to image blood flow in a subject. A contrast or imaging agent, including an indocyanine green (ICG) or fluorescein alone or in conjunction with a peptide or active agent, is administered to a subject. The subject is a human or an animal and has a chronic wound or suspected ischemia (e.g. in extremities or limb). Administration is intravenous, subcutaneous, intranasal, oral, intraperitoneal, intramuscular, or intradermal. Upon administration, the agent is targeted to vascular tissues and cells thereof, or is selectively retained within the blood. The agent is then visualized using an imaging system, such as a surgical microscope, other imaging system, or as an open imaging system. Absence, blockage or hemorrhage of fluorescence signal from the tissue of interest indicates reduced or absent blood flow, and DVT or other vein abnormalities. Other contrast or imaging agents can be used as described herein.

The imaging agent is conjugated to a SEQ ID NO: 9 peptide at K27, or any one of SEQ ID NO: 1-SEQ ID NO: 481 peptide, SEQ ID NO: 482-SEQ ID NO: 485 peptide, or a fragment of the foregoing.

### Example 11

### Use of Compounds and Methods to Image and Monitor Cerebral Vascular Flow

This example describes use of the compounds and methods disclosed herein for imaging and diagnosis of vessel narrowing (stenosis), clot formation (thrombosis), blockage (embolism) or blood vessel rupture (hemorrhage) in the brain. Lack of sufficient blood flow (ischemia) affects brain tissue and may cause a stroke. The compounds and methods disclosed herein are detected by an imaging system is used to image blood flow in a subject. A contrast or imaging agent, including an indocyanine green (ICG) or fluorescein alone or in conjunction with a peptide or active agent, is administered to a subject. The subject is a human or an animal and has a chronic wound or suspected ischemia (e.g. in extremities or limb). Administration is intravenous, subcutaneous, intranasal, oral, intraperitoneal, intramuscular, or intradermal. Upon administration, the agent is targeted to vascular tissues and cells thereof, or is selectively retained within the blood. The agent is then visualized using an imaging system, such as a surgical microscope, other imaging system, or as an open imaging system. Absence, blockage or hemorrhage of fluorescence signal from the tissue of interest indicates reduced or absent blood flow, and ischemia. Other contrast or imaging agents can be used as described herein.

The imaging agent is conjugated to a SEQ ID NO: 9 peptide at K27, or any one of SEQ ID NO: 1-SEQ ID NO: 481 peptide, SEQ ID NO: 482-SEQ ID NO: 485 peptide, or a fragment of the foregoing.

### Example 12

### Use of Compounds and Methods to Image and Monitor Vascular Flow to Tumors

This example describes use of the compounds and methods disclosed herein for imaging of tumor vasculature for monitoring, diagnosis and treatment of tumors. The compounds and methods disclosed herein are detected by an imaging system is used to image blood flow in a subject. A contrast or imaging agent, including an indocyanine green (ICG) or fluorescein alone or in conjunction with a peptide or active agent, is administered to a subject. The subject is a human or an animal and has a chronic wound or suspected ischemia (e.g. in extremities or limb). Administration is intravenous, subcutaneous, intranasal, oral, intraperitoneal, intramuscular, or intradermal. Upon administration, the agent is targeted to vascular tissues and cells thereof, or is selectively retained within the blood. The agent is then visualized using an imaging system, such as a surgical microscope, other imaging system, or as an open imaging system. Presence of enhanced and abnormal fluorescence signal from the tissue of interest indicates angiogenesis, or stimulation of blood vessel growth indicative of tumors. Other contrast or imaging agents can be used as described herein.

The imaging agent is conjugated to a SEQ ID NO: 9 peptide at K27, or any one of SEQ ID NO: 1-SEQ ID NO: 481 peptide, SEQ ID NO: 482-SEQ ID NO: 485 peptide, or a fragment of the foregoing.

### Example 13

### Use of Compounds and Methods for Coronary Angiography, Angiogram and Cardiac Catheterization

During coronary angiography, a contrast or imaging dye is injected into a subject artery through a catheter or other. Using the system and methods herein blood flow is monitored through the subject's heart. This test is also known as a cardiac angiogram, catheter arteriography, or cardiac catheterization. This example describes use of the compounds and methods disclosed herein for imaging of heart vasculature. The compounds and methods disclosed herein are detected by an imaging system is used to image blood flow in a subject. A contrast or imaging agent, including an indocyanine green (ICG) or fluorescein alone or in conjunction with a peptide or active agent, is administered to a subject. The subject is a human or an animal and has known or suspected coronary artery disease. Administration is intravenous, subcutaneous, intranasal, oral, intraperitoneal, intramuscular, or intradermal. Upon administration, the agent is targeted to vascular tissues and cells thereof, or is selectively retained within the blood. The agent is then visualized using an imaging system, such as a surgical microscope, other imaging system, or as an open imaging system. Absence, blockage or hemorrhage of fluorescence signal from the tissue of interest indicates reduced or absent blood flow, and ischemia. Catheterization, angioplasty, plaque ablation, stent insertion or replacement, or other treatment can accompany the imaging. Other contrast or imaging agents can be used as described herein.

Similarly, during a coronary angiogram, a contrast or imaging dye is injected into the blood vessels of the heart. Using the system and methods herein blood flow is monitored through the subject's heart. The system is used to take a series of images (angiograms), to visualize the cardiovasculature and blood vessels feeding blood to the heart. Concurrent with imaging and monitoring of vessels using this method, clogged heart arteries can be opened (angioplasty) during the coronary angiogram. Coronary computed tomography angiography (CCTA) can also similarly employed.

The imaging agent is conjugated to a SEQ ID NO: 9 peptide at K27, or any one of SEQ ID NO: 1-SEQ ID NO: 481 peptide, SEQ ID NO: 482-SEQ ID NO: 485 peptide, or a fragment of the foregoing.

### Example 14

### Use of Compounds and Methods for Imaging and Monitoring Stroke, Coronary Artery Disease or Congestive Heart Failure

This example describes use of the compounds and methods disclosed herein for imaging and diagnosis of stroke, coronary artery disease or congestive heart failure, or in cardiography. Lack of sufficient blood flow (ischemia) affects brain tissue and may cause a stroke. The imaging system of the present invention is used to image blood flow in a subject. A contrast or imaging agent, including an indocyanine green (ICG) or fluorescein alone or in conjunction with a peptide or active agent, is administered to a subject. The subject is a human or an animal and has a chronic wound or suspected ischemia (e.g. in extremities or limb). Administration is intravenous, subcutaneous, intranasal, oral, intraperitoneal, intramuscular, or intradermal. Upon administration, the agent is targeted to vascular tissues and cells thereof, or is selectively retained within the blood. The agent is then visualized using an imaging system, such as a surgical microscope, other imaging system, or as an open imaging system. Absence, blockage or hemorrhage of fluorescence signal from the tissue of interest indicates reduced or absent blood flow, and ischemia indicative of stroke, coronary artery disease or congestive heart failure. Other contrast or imaging agents can be used as described herein.

The imaging agent is conjugated to a SEQ ID NO: 9 peptide at K27, or any one of SEQ ID NO: 1-SEQ ID NO: 481 peptide, SEQ ID NO: 482-SEQ ID NO: 485 peptide, or a fragment of the foregoing.

### Example 15

### Use of Compounds and Methods During Aneurysm, Arteriovenous Malformation, or Cavernous Malformation Resection

This example describes use of the compounds and methods disclosed herein for coaxial illumination and visualization of Compound 76 fluorescence during surgical resection of a cavernous malformation in a pediatric patient. An imaging system was used to image brain tissue to detect abnormal vasculature using fluorescence imaging. Surgery was performed to remove the abnormality from the subject.

A pediatric subject with anxiety and depression presented to the emergency room. She lost consciousness and awoke with a headache. On exam she was lethargic with a flat affect without focal neurological deficit. Medications included fluoxetine and hydroxyzine. MRI showed a 3 cm mass in the left cerebellar hemisphere with blooming artifact and minimal enhancement. The clinical impression was a cerebral cavernous malformation, likely incidental. The decision was made to have it removed.

The patient received 15 mg/m2 of a fluorescent conjugate comprising SEQ ID NO:9 conjugated to an ICG (Compound 76) via IV injection on the morning of surgery and image collection. The imaging system head was attached to the Zeiss Pentero surgical microscope along with two eyepieces prior the start of surgery.

After the lesion was exposed, the imaging system was initialized and used continuously. The imaging system enabled the surgeon to view fluorescence and visible imaging together and simultaneously with the operating microscope. A microsurgical resection was performed through a left retroaricular craniotomy with frameless stereotactic navigation and intraoperative ultrasonography. A round mulberry appearing vascular lesion that contained areas of thrombosed vessels and firm calcifications was encountered. The lesion fluoresced avidly with Compound 76. The surrounding cerebellum was discolored yellow, presumably from prior hemorrhage, and did not fluoresce. The lesion was removed. The patient awakened with mild left upper extremity dysmetria that had resolved at the time of discharge on postoperative day 5. Postoperative MRI confirmed resection of the mass. Pathology showed a cavernous malformation with abundant abnormal vessels, scattered inflammation and some thrombosed vessels. This case further demonstrated that the compounds and methods could be used in an intraoperative setting and enabled the surgeon to visualize and precisely localize fluorescence in non-neoplastic pathologies during surgery and use this information to remove abnormal vascular tissue during resection.

The imaging agent is conjugated to a SEQ ID NO: 9 peptide at K27, or any one of SEQ ID NO: 1-SEQ ID NO: 481 peptide, SEQ ID NO: 482-SEQ ID NO: 485 peptide, or a fragment of the foregoing.

While preferred embodiments of the present invention have been shown and described herein, it will be apparent to those skilled in the art that such embodiments are provided by way of example only. Numerous variations, changes, and substitutions will now occur to those skilled in the art without departing from the invention. It should be understood that various alternatives to the embodiments of the invention described herein may be employed in practicing the invention. It is intended that the following claims define the scope of the invention and that methods and structures within the scope of these claims and their equivalents be covered thereby.

1-130. (canceled)

131. A method of treating a subject with a vascular lesion, the method comprising, administering to the subject a polypeptide comprising at least 3 disulfide bonds and:
a sequence having at least 90% sequence identity to SEQ ID NO: 9, or
a sequence having at least 90% sequence identity to a fragment of SEQ ID NO: 9 comprising at least 20 amino acid residues,
thereby treating the vascular lesion in the subject.

132. The method of claim 131, wherein the polypeptide comprises a sequence of any one of SEQ ID NO: 482-SEQ ID NO: 485 or a fragment of any one of SEQ ID NO: 482-SEQ ID NO: 485 comprising at least 20 amino acid residues.

133. The method of claim 131, wherein the polypeptide comprises a sequence of SEQ ID NO: 9.

134. The method of claim 131, wherein the vascular lesion comprises a cavernoma, an arteriovenous malformation, a venous malformation, a lymphatic malformation, a capillary telangiectasia, a mixed vascular malformation, an aneurysm, a spinal dural arteriovenous fistula, or combinations thereof.

135. The method of claim 134, wherein the cavernoma comprises a cavernous angioma, a cavernous hemangioma, or a cerebral cavernous malformation.

136. The method of claim 134, wherein the arteriovenous malformation comprises an arteriovenous angioma, an arteriovenous hemangioma, or a cerebral arteriovenous malformation.

137. The method of claim 134, wherein the aneurysm comprises an abdominal aortic aneurysm, a thoracic aortic aneurysm, or a cerebral aneurysm.

138. The method of claim 131, wherein the method further comprises detecting the vascular lesion.

139. The method of claim 138, wherein the detecting is performed using fluorescence imaging.

140. The method of claim 131, wherein the polypeptide is conjugated to a detectable agent.

141. The method of claim 140, wherein the detectable agent is conjugated to the polypeptide at K-27 of SEQ ID NO: 9.

142. The method of claim 140, wherein the detectable agent comprises a dye, a fluorophore, a fluorescent biotin compound, a luminescent compound, a chemiluminescent compound, a radioisotope, a radionuclide, nanoparticle, a paramagnetic metal ion, or combinations thereof.

143. The method of claim 142, wherein the dye comprises a DyLight-680, a DyLight-750, a VivoTag-750, a DyLight-800, an IRDye-800, a VivoTag-680, a Cy5.5, an indocyanine green (ICG), a near infrared dye, an acradine orange, an acradine yellow, a 7-actinomycin D, a 8-anilinonaphthalene-1-sulfonic acid, an ATTO dye, an ATTO dye derivative, an auramine-rhodamine stain, an auramine-rhodamine stain derivative, a bensantrhone, a bimane, a 9-10-bis(phenylethynyl)anthracene, a 5,12-bis(phenylethynyl)naththacene, a bisbenzimide, a brainbow, a calcein, a carbodyfluorescein, a carbodyfluorescein derivative, a 1-chloro-9,10-bis(phenylethynyl)anthracene, a 1-chloro-9,10-bis(phenylethynyl)anthracene derivative, a DAPI, a DiOC6, a DyLight Fluor, a DyLight Fluor derivative, a epicocconone, an ethidium bromide, a FIAsH-EDT2, a Fluo dye, a Fluo dye derivative, a FluoProbe, a FluoProbe derivative, a Fluorescein, a Fluorescein derivative, a Fura, a Fura derivative, a GelGreen, a GelGreen derivative, a GelRed, a GelRed derivative, a fluorescent protein, a fluorescent protein derivative, an m isoform protein, an m isoform protein derivative, a hetamethine dye, a hetamethine dye derivative, a hoeschst stain, an iminocoumarin, an indian yellow, an indo-1, an indo-1 derivative, a laurdan, a lucifer yellow, a lucifer yellow derivative, a luciferin, a luciferin derivative, a luciferase, a luciferase derivative, a mercocyanine, a mercocyanine derivative, a methylene blue, a methylene blue derivative thereof, a nile dye, a nile dye derivative, an OS680, an OS750, a perylene, a phloxine, a phyco dye, a phyco dye derivative, a propium iodide, a pyranine, a rhodamine, a rhodamine derivative, a ribogreen, a RoGFP, a rubrene, a stilbene, a stilbene derivative, a sulforhodamine, a sulforhodamine derivative, a SYBR, a SYBR derivative, a synapto-pHluorin, a tetraphenyl butadiene, a tetrasodium tris, a Titan Yellow, a topotecan, a TSQ, an umbelliferone, a violanthrone, a yellow fluorescent protein, a YOYO-1, a ZW800, a carbocyanine, a merocyanine, a styryl dye, an oxonol dye, a phycoerythrin, an erythrosin, an eosin, a coumarin, an Oregon Green Dye, a Texas Red, a Texas Red-X, a SPECTRUM RED, a SPECTRUM GREEN, a cyanine dye, an ALEXA FLUOR dye, an ALEXA FLUOR dye derivative, a BODIPY dye, an IRDyes, or combinations thereof.

144. The method of claim 140, wherein the detectable agent comprises an indocyanine green.

145. The method of claim 131, wherein the polypeptide comprises an isoelectric point of at least 7.5.

146. The method of claim 131, wherein the polypeptide binds to or accumulates in a vascular lesion tissue or a vascular lesion cell.

147. The method of claim 146, further comprising surgically removing the vascular lesion tissue or the vascular lesion cell from the subject.

148. The method of claim 131, wherein the polypeptide is intravenously administered to the subject between 1 hour and 72 hours, inclusive, prior surgically removing the vascular lesion tissue or the vascular lesion cell from the subject.

149. The method of claim 131, wherein the treating comprises reducing a symptom of the vascular lesion in the subject.

150. The method of claim 131, wherein the polypeptide is conjugated to a therapeutic agent.

151. The method of claim 150, wherein the therapeutic agent comprises a radioisotope, a nanoparticle, a toxin, an enzyme, a sensitizing drug, a radiosensitizer, a photosensitizer, a nucleic acid, an interfering RNA, an antibody, an antibody fragment, an aptamer, an anti-angiogenic agent, an anti-metabolite, a mitotic inhibitor, a growth factor inhibitor, or combinations thereof.

152. A method of imaging an organ, organ substructure, or body region of a subject, the method comprising:
administering to the subject a compound comprising a polypeptide conjugated to a detectable agent, wherein the polypeptide comprises:
a) at least 3 disulfide bonds,
b) a length of no less than 20 amino acid residues,
c) an isoelectric point of no less than 7.5, or
d) combinations thereof, and
imaging an organ or organ substructure comprising a vascular lesion.

153. A method of determining the effect of treating a subject, the method comprising:
treating the subject by administering to the subject a compound comprising a polypeptide conjugated to a detectable agent, wherein the polypeptide comprises:
a) at least 3 disulfide bonds,
b) a length of no less than 20 amino acid residues,
c) an isoelectric point of no less than 7.5, or
d) combinations thereof, and
determining the treatment is efficacious when a signal from the polypeptide is lower compared to a baseline measurement.
